SEQUENCE LISTING (216 sequences)

SEQ ID NO: 1 - protein, 134 aa, Artificial Sequence
  Synthetic polypeptide
MNFGFSLIFLVLVLKGVQCEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSA

SEQ ID NO: 2 - protein, 115 aa, Artificial Sequence
  Synthetic polypeptide
EVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSA

SEQ ID NO: 3 - protein, 5 aa, Artificial Sequence
  Synthetic polypeptide
RYAMS

SEQ ID NO: 4 - protein, 16 aa, Artificial Sequence
  Synthetic polypeptide
SITFGGDPYYPDGLKG

SEQ ID NO: 5 - protein, 7 aa, Artificial Sequence
  Synthetic polypeptide
HESWFAY

SEQ ID NO: 6 - protein, 30 aa, Artificial Sequence
  Synthetic polypeptide
EVKLVESGGGLVKPGGSLKLSCEASGFTFS

SEQ ID NO: 7 - protein, 14 aa, Artificial Sequence
  Synthetic polypeptide
WVRHTPEKRLAWVA

SEQ ID NO: 8 - protein, 32 aa, Artificial Sequence
  Synthetic polypeptide
RFTISRDNTRNILYLQMNSLRSEDTAMYYCVR

SEQ ID NO: 9 - protein, 11 aa, Artificial Sequence
  Synthetic polypeptide
WGQGTLVTVSA

SEQ ID NO: 10 - protein, 131 aa, Artificial Sequence
  Synthetic polypeptide
MKLPVRLLVLMFWIPASSSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIK

SEQ ID NO: 11 - protein, 112 aa, Artificial Sequence
  Synthetic polypeptide
DVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIK

SEQ ID NO: 12 - protein, 16 aa, Artificial Sequence
  Synthetic polypeptide
RSSQTIVHTDGNIYLE

SEQ ID NO: 13 - protein, 7 aa, Artificial Sequence
  Synthetic polypeptide
KISNRFS

SEQ ID NO: 14 - protein, 9 aa, Artificial Sequence
  Synthetic polypeptide
FQASHVPYT

SEQ ID NO: 15 - protein, 23 aa, Artificial Sequence
  Synthetic polypeptide
DVLMTQTPLSLPVSLGEQVSISC

SEQ ID NO: 16 - protein, 15 aa, Artificial Sequence
  Synthetic polypeptide
WYLQNPGQSPRLLIY

SEQ ID NO: 17 - protein, 32 aa, Artificial Sequence
  Synthetic polypeptide
GVPDRFSGSGSGTDFTLKISRVEAEDLGIYYC

SEQ ID NO: 18 - protein, 10 aa, Artificial Sequence
  Synthetic polypeptide
FGGGTKLEIK

SEQ ID NO: 19 - DNA, 402 bp, Artificial Sequence
  Synthetic polynucleotide
atgaacttcgggttcagcttgattttccttgtccttgttttaaaaggtgtccagtgtgaagtgaagctggtggagtctgggggaggcttagtgaagcctggggggtccctgaaactctcctgtgaggcctctgggttcactttcagtcgctatgccatgtcttgggttcgccatactccagagaagagactggcctgggtcgcatccataacttttggtggtgacccgtattatcccgacggtttgaagggccgattcaccatctccagagataataccaggaacatcctgtacctgcaaatgaacagtctgaggtctgaagacacggccatgtattattgtgtacgacacgagtcatggtttgcttactggggccaagggactctggtcactgtctctgca

SEQ ID NO: 20 - DNA, 345 bp, Artificial Sequence
  Synthetic polynucleotide
gaagtgaagctggtggagtctgggggaggcttagtgaagcctggggggtccctgaaactctcctgtgaggcctctgggttcactttcagtcgctatgccatgtcttgggttcgccatactccagagaagagactggcctgggtcgcatccataacttttggtggtgacccgtattatcccgacggtttgaagggccgattcaccatctccagagataataccaggaacatcctgtacctgcaaatgaacagtctgaggtctgaagacacggccatgtattattgtgtacgacacgagtcatggtttgcttactggggccaagggactctggtcactgtctctgca

SEQ ID NO: 21 - DNA, 15 bp, Artificial Sequence
  Synthetic oligonucleotide
cgctatgccatgtct

SEQ ID NO: 22 - DNA, 48 bp, Artificial Sequence
  Synthetic oligonucleotide
tccataacttttggtggtgacccgtattatcccgacggtttgaagggc

SEQ ID NO: 23 - DNA, 21 bp, Artificial Sequence
  Synthetic oligonucleotide
cacgagtcatggtttgcttac

SEQ ID NO: 24 - DNA, 90 bp, Artificial Sequence
  Synthetic oligonucleotide
gaagtgaagctggtggagtctgggggaggcttagtgaagcctggggggtccctgaaactctcctgtgaggcctctgggttcactttcagt

SEQ ID NO: 25 - DNA, 42 bp, Artificial Sequence
  Synthetic oligonucleotide
tgggttcgccatactccagagaagagactggcctgggtcgca

SEQ ID NO: 26 - DNA, 96 bp, Artificial Sequence
  Synthetic oligonucleotide
cgattcaccatctccagagataataccaggaacatcctgtacctgcaaatgaacagtctgaggtctgaagacacggccatgtattattgtgtacga

SEQ ID NO: 27 - DNA, 33 bp, Artificial Sequence
  Synthetic oligonucleotide
tggggccaagggactctggtcactgtctctgca

SEQ ID NO: 28 - DNA, 393 bp, Artificial Sequence
  Synthetic polynucleotide
atgaagttgcctgttaggctgttggtgctgatgttctggattcctgcttccagcagtgatgttttgatgacccaaactccactctccctgcctgtcagtcttggagaacaagtctccatctcttgcagatctagtcagaccattgtacatactgatggcaacatctatttagaatggtacctccagaacccaggccagtctcctaggctcctgatctacaaaatttccaaccgattttctggggtccctgacaggttcagtggcagtggatcagggacagatttcacactcaagatcagcagagtggaggctgaggatctgggaatttattactgctttcaagcttcacatgttccgtacacgttcggaggggggaccaagctggaaataaaa

SEQ ID NO: 29 - DNA, 336 bp, Artificial Sequence
  Synthetic polynucleotide
gatgttttgatgacccaaactccactctccctgcctgtcagtcttggagaacaagtctccatctcttgcagatctagtcagaccattgtacatactgatggcaacatctatttagaatggtacctccagaacccaggccagtctcctaggctcctgatctacaaaatttccaaccgattttctggggtccctgacaggttcagtggcagtggatcagggacagatttcacactcaagatcagcagagtggaggctgaggatctgggaatttattactgctttcaagcttcacatgttccgtacacgttcggaggggggaccaagctggaaataaaa

SEQ ID NO: 30 - DNA, 48 bp, Artificial Sequence
  Synthetic oligonucleotide
agatctagtcagaccattgtacatactgatggcaacatctatttagaa

SEQ ID NO: 31 - DNA, 21 bp, Artificial Sequence
  Synthetic oligonucleotide
aaaatttccaaccgattttct

SEQ ID NO: 32 - DNA, 27 bp, Artificial Sequence
  Synthetic oligonucleotide
tttcaagcttcacatgttccgtacacg

SEQ ID NO: 33 - DNA, 69 bp, Artificial Sequence
  Synthetic oligonucleotide
gatgttttgatgacccaaactccactctccctgcctgtcagtcttggagaacaagtctccatctcttgc

SEQ ID NO: 34 - DNA, 45 bp, Artificial Sequence
  Synthetic oligonucleotide
tggtacctccagaacccaggccagtctcctaggctcctgatctac

SEQ ID NO: 35 - DNA, 96 bp, Artificial Sequence
  Synthetic oligonucleotide
ggggtccctgacaggttcagtggcagtggatcagggacagatttcacactcaagatcagcagagtggaggctgaggatctgggaatttattactgc

SEQ ID NO: 36 - DNA, 30 bp, Artificial Sequence
  Synthetic oligonucleotide
ttcggaggggggaccaagctggaaataaaa

SEQ ID NO: 37 - protein, 134 aa, Artificial Sequence
  Synthetic polypeptide
MNFGFSLIFLVLVLKGVQCEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSA

SEQ ID NO: 38 - protein, 115 aa, Artificial Sequence
  Synthetic polypeptide
EVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSA

SEQ ID NO: 39 - protein, 5 aa, Artificial Sequence
  Synthetic peptide
RYAMS

SEQ ID NO: 40 - protein, 16 aa, Artificial Sequence
  Synthetic polypeptide
SITFGGSAYYLDSVKG

SEQ ID NO: 41 - protein, 7 aa, Artificial Sequence
  Synthetic polypeptide
HQPWFDY

SEQ ID NO: 42 - protein, 30 aa, Artificial Sequence
  Synthetic polypeptide
EVKLVESGGGLVKPGGSLKVSCTASGFTFS

SEQ ID NO: 43 - protein, 14 aa, Artificial Sequence
  Synthetic polypeptide
WVRQTPERRLEWVA

SEQ ID NO: 44 - protein, 32 aa, Artificial Sequence
  Synthetic polypeptide
RFTISRDNAQNILYLQMNSLVSEDTAIYNCAR

SEQ ID NO: 45 - protein, 11 aa, Artificial Sequence
  Synthetic polypeptide
WGQGTLVTVSA

SEQ ID NO: 46 - protein, 131 aa, Artificial Sequence
  Synthetic peptide
  SITE (1)..(131): /note=“Variant residues given in the sequence have no preference with respect to those in the annotations for variant positions”
  VARIANT (49)..(49): /replace=“Val”
MNLPIRLLVLMFWIPASSSDVLLTQTPLSLPVSLGDQASISCRSSQNIIHSDGDTYLDWFLQKPGQSPNLLIYKVSNRFSGVPDRFSGSGSGTDFTLKISRVEADDLGVYYCFQASHVPYTFGGGTKLEVK

SEQ ID NO: 47 - protein, 112 aa, Artificial Sequence
  Synthetic polypeptide
  SITE (1)..(112): /note=“Variant residues given in the sequence have no preference with respect to those in the annotations for variant positions”
  VARIANT (30)..(30): /replace=“Val”
DVLLTQTPLSLPVSLGDQASISCRSSQNIIHSDGDTYLDWFLQKPGQSPNLLIYKVSNRFSGVPDRFSGSGSGTDFTLKISRVEADDLGVYYCFQASHVPYTFGGGTKLEVK

SEQ ID NO: 48 - protein, 16 aa, Artificial Sequence
  Synthetic polypeptide
  SITE (1)..(16): /note=“Variant residues given in the sequence have no preference with respect to those in the annotations for variant positions”
  VARIANT (7)..(7): /replace=“Val”
RSSQNIIHSDGDTYLD

SEQ ID NO: 49 - protein, 7 aa, Artificial Sequence
  Synthetic polypeptide
KVSNRFS

SEQ ID NO: 50 - protein, 9 aa, Artificial Sequence
  Synthetic polypeptide
FQASHVPYT

SEQ ID NO: 51 - protein, 23 aa, Artificial Sequence
  Synthetic polypeptide
DVLLTQTPLSLPVSLGDQASISC

SEQ ID NO: 52 - protein, 15 aa, Artificial Sequence
  Synthetic polypeptide
WFLQKPGQSPNLLIY

SEQ ID NO: 53 - protein, 32 aa, Artificial Sequence
  Synthetic polypeptide
GVPDRFSGSGSGTDFTLKISRVEADDLGVYYC

SEQ ID NO: 54 - protein, 10 aa, Artificial Sequence
  Synthetic polypeptide
FGGGTKLEVK

SEQ ID NO: 55 - DNA, 402 bp, Artificial Sequence
  Synthetic polynucleotide
atgaacttcgggttcagcttgattttccttgtccttgttttaaaaggtgtccagtgcgaagtgaagctggtggagtctgggggaggcttagtgaagcctggagggtccctgaaagtctcctgtacagcctctggattcactttcagtcgttatgccatgtcttgggttcgccagactccagagaggaggctggagtgggtcgcatccattacttttggtggtagcgcctactatttagacagtgtgaagggccgattcaccatctccagagataatgcccagaacatcctgtacctgcaaatgaacagtctggtgtctgaggacacggccatttataactgtgcaagacaccagccctggtttgattactggggccaagggactctggtcactgtctctgca

SEQ ID NO: 56 - DNA, 345 bp, Artificial Sequence
  Synthetic polynucleotide
gaagtgaagctggtggagtctgggggaggcttagtgaagcctggagggtccctgaaagtctcctgtacagcctctggattcactttcagtcgttatgccatgtcttgggttcgccagactccagagaggaggctggagtgggtcgcatccattacttttggtggtagcgcctactatttagacagtgtgaagggccgattcaccatctccagagataatgcccagaacatcctgtacctgcaaatgaacagtctggtgtctgaggacacggccatttataactgtgcaagacaccagccctggtttgattactggggccaagggactctggtcactgtctctgca

SEQ ID NO: 57 - DNA, 15 bp, Artificial Sequence
  Synthetic oligonucleotide
cgttatgccatgtct

SEQ ID NO: 58 - DNA, 48 bp, Artificial Sequence
  Synthetic oligonucleotide
tccattacttttggtggtagcgcctactatttagacagtgtgaagggc

SEQ ID NO: 59 - DNA, 21 bp, Artificial Sequence
  Synthetic oligonucleotide
caccagccctggtttgattac

SEQ ID NO: 60 - DNA, 90 bp, Artificial Sequence
  Synthetic oligonucleotide
gaagtgaagctggtggagtctgggggaggcttagtgaagcctggagggtccctgaaagtctcctgtacagcctctggattcactttcagt

SEQ ID NO: 61 - DNA, 42 bp, Artificial Sequence
  Synthetic oligonucleotide
tgggttcgccagactccagagaggaggctggagtgggtcgca

SEQ ID NO: 62 - DNA, 96 bp, Artificial Sequence
  Synthetic oligonucleotide
cgattcaccatctccagagataatgcccagaacatcctgtacctgcaaatgaacagtctggtgtctgaggacacggccatttataactgtgcaaga

SEQ ID NO: 63 - DNA, 33 bp, Artificial Sequence; identical to SEQ ID NO: 27
  Synthetic oligonucleotide

SEQ ID NO: 64 - DNA, 393 bp, Artificial Sequence
  Synthetic polynucleotide
atgaacttgcctattaggctgttggtgctgatgttctggattcctgcttccagcagtgatgtcttgctgacccagactccactctccctgcctgtcagtcttggagatcaggcctccatctcttgcagatctagtcagaacattrtwcatagtgatggagacacctatttagactggttcctgcagaaaccaggccagtctccaaacctcctaatctacaaagtttccaaccgattttctggggtcccagacaggttcagtggcagtggatcagggacagatttcaccctcaagatcagcagagtggaggctgacgatctgggagtttattactgctttcaagcttcacatgttccgtacacgttcggaggggggaccaagctggaagtaaaa

SEQ ID NO: 65 - DNA, 336 bp, Artificial Sequence
  Synthetic polynucleotide
gatgtcttgctgacccagactccactctccctgcctgtcagtcttggagatcaggcctccatctcttgcagatctagtcagaacattrtwcatagtgatggagacacctatttagactggttcctgcagaaaccaggccagtctccaaacctcctaatctacaaagtttccaaccgattttctggggtcccagacaggttcagtggcagtggatcagggacagatttcaccctcaagatcagcagagtggaggctgacgatctgggagtttattactgctttcaagcttcacatgttccgtacacgttcggaggggggaccaagctggaagtaaaa

SEQ ID NO: 66 - DNA, 48 bp, Artificial Sequence
  Synthetic oligonucleotide
agatctagtcagaacattrtwcatagtgatggagacacctatttagac

SEQ ID NO: 67 - DNA, 21 bp, Artificial Sequence
  Synthetic oligonucleotide
aaagtttccaaccgattttct

SEQ ID NO: 68 - DNA, 27 bp, Artificial Sequence
  Synthetic oligonucleotide
tttcaagcttcacatgttccgtacacg

SEQ ID NO: 69 - DNA, 69 bp, Artificial Sequence
  Synthetic oligonucleotide
gatgtcttgctgacccagactccactctccctgcctgtcagtcttggagatcaggcctccatctcttgc

SEQ ID NO: 70 - DNA, 45 bp, Artificial Sequence
  Synthetic oligonucleotide
tggttcctgcagaaaccaggccagtctccaaacctcctaatctac

SEQ ID NO: 71 - DNA, 96 bp, Artificial Sequence
  Synthetic oligonucleotide
ggggtcccagacaggttcagtggcagtggatcagggacagatttcaccctcaagatcagcagagtggaggctgacgatctgggagtttattactgc

SEQ ID NO: 72 - DNA, 30 bp, Artificial Sequence
  Synthetic oligonucleotide
ttcggaggggggaccaagctggaagtaaaa

SEQ ID NO: 73 - protein, 521 aa, Artificial Sequence
  Synthetic polypeptide
MALPVTALLLPLALLLHAARPGSELPTQGTFSNVSTNVSSPEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSAGGGGSGGGGSGGGGSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1563 bp, Artificial Sequence
  Synthetic polynucleotide
atggctcttccggtaaccgctctgctgcttcctttggctctgcttcttcacgcggctaggccagggagcgaattgcctacgcaggggactttttcaaatgtaagcacgaatgtgagctctccggaagtgaaattggtagagtctggtgggggactggttaaacctggaggcagtctgaagctctcctgcgaagccagcggctttacgttcagtcggtacgctatgtcctgggtgcgacacacaccagaaaagcgactcgcttgggtagcgagcataacgtttgggggcgatccttattacccggatggtttgaaaggtcgcttcactatcagtcgagataatacgcgaaacattctttatcttcaaatgaatagcctgcgctctgaagacaccgcgatgtattattgtgttaggcatgagtcttggtttgcatactggggtcagggtactcttgtcactgtatctgccggaggtgggggtagcggcggcggaggtagtgggggaggcggcagcgacgtgttgatgactcaaactccgcttagcttgccagtgtctcttggagaacaggtctccatatcatgtcgatccagtcaaactatcgttcataccgatgggaatatctatctcgaatggtatctgcaaaatccagggcagagtccccggcttctgatatataagattagcaatagatttagtggtgttcccgacaggttttcaggttctgggtccggaacggattttaccctgaagataagtcgagttgaagcggaagacctgggcatatactattgcttccaggcgtcccacgtcccctatacatttgggggtgggactaaactggaaattaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 75 - protein, 21 aa, Artificial Sequence
  Synthetic polypeptide
MALPVTALLLPLALLLHAARP

SEQ ID NO: 76 - DNA, 63 bp, Artificial Sequence
  Synthetic oligonucleotide
atggctcttccggtaaccgctctgctgcttcctttggctctgcttcttcacgcggctaggcca

SEQ ID NO: 77 - protein, 2 aa, Artificial Sequence
  Synthetic polypeptide
GS

SEQ ID NO: 78 - DNA, 6 bp, Artificial Sequence
  Synthetic oligonucleotide
gggagc

SEQ ID NO: 79 - protein, 16 aa, Artificial Sequence
  Synthetic polypeptide
ELPTQGTFSNVSTNVS

SEQ ID NO: 80 - DNA, 48 bp, Artificial Sequence
  Synthetic oligonucleotide
gaattgcctacgcaggggactttttcaaatgtaagcacgaatgtgagc

SEQ ID NO: 81 - protein, 2 aa, Artificial Sequence
  Synthetic polypeptide
SP

SEQ ID NO: 82 - DNA, 6 bp, Artificial Sequence
  Synthetic oligonucleotide
tctccg

SEQ ID NO: 83 - protein, 115 aa, Artificial Sequence; identical to SEQ ID NO: 2
  Synthetic polypeptide

SEQ ID NO: 84 - DNA, 345 bp, Artificial Sequence
  Synthetic polynucleotide
gaagtgaaattggtagagtctggtgggggactggttaaacctggaggcagtctgaagctctcctgcgaagccagcggctttacgttcagtcggtacgctatgtcctgggtgcgacacacaccagaaaagcgactcgcttgggtagcgagcataacgtttgggggcgatccttattacccggatggtttgaaaggtcgcttcactatcagtcgagataatacgcgaaacattctttatcttcaaatgaatagcctgcgctctgaagacaccgcgatgtattattgtgttaggcatgagtcttggtttgcatactggggtcagggtactcttgtcactgtatctgcc

SEQ ID NO: 85 - protein, 15 aa, Artificial Sequence
  Synthetic polypeptide
GGGGSGGGGSGGGGS

SEQ ID NO: 86 - DNA, 45 bp, Artificial Sequence
  Synthetic oligonucleotide
ggaggtgggggtagcggcggcggaggtagtgggggaggcggcagc

SEQ ID NO: 87 - protein, 112 aa, Artificial Sequence; identical to SEQ ID NO: 11
  Synthetic polypeptide

SEQ ID NO: 88 - DNA, 336 bp, Artificial Sequence
  Synthetic polynucleotide
gacgtgttgatgactcaaactccgcttagcttgccagtgtctcttggagaacaggtctccatatcatgtcgatccagtcaaactatcgttcataccgatgggaatatctatctcgaatggtatctgcaaaatccagggcagagtccccggcttctgatatataagattagcaatagatttagtggtgttcccgacaggttttcaggttctgggtccggaacggattttaccctgaagataagtcgagttgaagcggaagacctgggcatatactattgcttccaggcgtcccacgtcccctatacatttgggggtgggactaaactggaaattaaa

SEQ ID NO: 89 - protein, 1 aa, Artificial Sequence
  Synthetic peptide
R

SEQ ID NO: 90 - DNA, 3 bp, Artificial Sequence
  Synthetic oligonucleotide
cgt

SEQ ID NO: 91 - protein, 45 aa, Artificial Sequence
  Synthetic polypeptide
TTTPAPRPPTPAPTIASQPLSLRPEACRPAAGGAVHTRGLDFACD

SEQ ID NO: 92 - DNA, 135 bp, Artificial Sequence
  Synthetic polynucleotide
acgacgacaccagccccaagacccccaactcccgctcctaccatagcttcccaacccctgtcactgaggccagaggcatgcaggcccgctgcgggtggcgcggtacatacgcggggactggactttgcatgtgac

SEQ ID NO: 93 - protein, 37 aa, Artificial Sequence
  Synthetic polypeptide
IYIWAPLAGTCGVLLLSLVITLYCNHRNRRRVCKCPR

SEQ ID NO: 94 - DNA, 111 bp, Artificial Sequence
  Synthetic polynucleotide
atttacatatgggcgccactggcgggaacatgtggagttttgttgcttagcctggtcataacactgtattgcaatcatcgcaacagacgccgcgtctgcaagtgtcccagg

SEQ ID NO: 95 - protein, 2 aa, Artificial Sequence
  Synthetic polypeptide
VD

SEQ ID NO: 96 - DNA, 6 bp, Artificial Sequence
  Synthetic oligonucleotide
gtcgac

SEQ ID NO: 97 - protein, 41 aa, Artificial Sequence
  Synthetic polypeptide
RSKRSRLLHSDYMNMTPRRPGPTRKHYQPYAPPRDFAAYRS

SEQ ID NO: 98 - DNA, 123 bp, Artificial Sequence
  Synthetic polynucleotide
agatctaagagaagtagacttcttcacagtgattatatgaatatgacgcctcgaagacccggcccgacacgcaaacactatcagccgtatgccccccctcgggattttgctgcctaccgcagc

SEQ ID NO: 99 - protein, 112 aa, Artificial Sequence
  Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 100 - DNA, 336 bp, Artificial Sequence
  Synthetic polynucleotide
cgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 101 - protein, 521 aa, Artificial Sequence
  Synthetic polypeptide
  SITE (1)..(521): /note=“Variant residues given in the sequence have no preference with respect to those in the annotations for variant positions”
  VARIANT (201)..(201): /replace=“Val”
MALPVTALLLPLALLLHAARPGSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIIHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 102 - DNA, 1562 bp, Artificial Sequence
  Synthetic polynucleotide
atggctcttccggtaaccgctctgctgcttcctttggctctgcttcttcacgcggctaggccagggagcgaattgcctacgcaggggactttttcaaatgtaagcacgaatgtgagctctccgaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagccggcggtggcggatctggagggggaggcagtggaggaggtggttcagatgttctcctgacgcaaactcctttgtcattgccggtaagtctgggcgaccaagcgagcatttcctgtcgcagcagccagaacatartwcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaacgtacgacgacaccagccccaagacccccaactcccgctcctaccatagcttcccaacccctgtcactgaggccagaggcatgcaggcccgctgcgggtggcgcggtacatacgcggggactggactttgcatgtgacatttacatatgggcgccactggcgggaacatgtggagttttgttgcttagcctggtcataacactgtattgcaatcatcgcaacagacgccgcgtctgcaagtgtcccagggtcgacagatctaagagaagtagacttcttcacagtgattatatgaatatgacgcctcgaagacccggcccgacacgcaaacactatcagccgtatgccccccctcgggattttgctgcctaccgcagccgcgttaagttctctaggtccgctgacgcccctgcctaccagcagggtcaaaaccaactgtacaatgaattgaatcttgggagacgggaggagtatgacgtactcgacaagcggagggggagagatcctgagatgggtggaaagcctcgacgaaaaaacccacaagaagggttgtataatgaactgcagaaagacaagatggctgaggcatatagtgaaatcgggatgaagggagaacgccggcgcgggaaagggcacgatggtctgtatcaagggcttagtacggctacgaaggacacatatgacgctttgcacatgcaggcccttcctcctagg

SEQ ID NO: 103 - protein, 21 aa, Artificial Sequence
  Synthetic polypeptide
MALPVTALLLPLALLLHAARP

SEQ ID NO: 104 - DNA, 63 bp, Artificial Sequence; identical to SEQ ID NO: 76
  Synthetic oligonucleotide

SEQ ID NO: 105 - protein, 2 aa, Artificial Sequence
  Synthetic polypeptide
GS

SEQ ID NO: 106 - DNA, 6 bp, Artificial Sequence
  Synthetic oligonucleotide
gggagc

SEQ ID NO: 107 - protein, 16 aa, Artificial Sequence
  Synthetic polypeptide
ELPTQGTFSNVSTNVS

SEQ ID NO: 108 - DNA, 48 bp, Artificial Sequence; identical to SEQ ID NO: 80
  Synthetic oligonucleotide

SEQ ID NO: 109 - protein, 2 aa, Artificial Sequence
  Synthetic polypeptide
SP

SEQ ID NO: 110 - DNA, 6 bp, Artificial Sequence
  Synthetic oligonucleotide
tctccg

SEQ ID NO: 111 - protein, 115 aa, Artificial Sequence; identical to SEQ ID NO: 38
  Synthetic polypeptide

SEQ ID NO: 112 - DNA, 345 bp, Artificial Sequence
  Synthetic polynucleotide
gaagtcaaattggtggagagtggcggtgggctcgtaaaaccaggaggcagcttgaaagtaagttgcacagcgtctgggttcactttcagccgatacgctatgagttgggtgcggcaaacgcccgaaagacgattggagtgggtagctagtattacttttgggggttccgcgtattacttggatagcgttaaagggagattcaccatatcacgagataatgcccaaaacatactctacctccagatgaactcattggtgtctgaagatactgcaatttacaactgcgccaggcaccagccatggtttgactattggggtcaaggtacgttggtcacagtctcagcc

SEQ ID NO: 113 - protein, 15 aa, Artificial Sequence
  Synthetic polypeptide
GGGGSGGGGSGGGGS

SEQ ID NO: 114 - DNA, 45 bp, Artificial Sequence
  Synthetic oligonucleotide
ggcggtggcggatctggagggggaggcagtggaggaggtggttca

SEQ ID NO: 115 - protein, 112 aa, Artificial Sequence; identical to SEQ ID NO: 47
  Synthetic polypeptide
  SITE (1)..(112): /note=“Variant residues given in the sequence have no preference with respect to those in the annotations for variant positions”
  VARIANT (30)..(30): /replace=“Val”

SEQ ID NO: 116 - DNA, 336 bp, Artificial Sequence
  Synthetic polynucleotide
gatgttctcctgacgcaaactcctttgtcattgccggtaagtctgggcgaccaagcgagcatttcctgtcgcagcagccagaacatartwcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaa

SEQ ID NO: 117 - protein, 1 aa, Artificial Sequence
  Synthetic peptide
R

SEQ ID NO: 118 - DNA, 3 bp, Artificial Sequence
  Synthetic oligonucleotide
cgt

SEQ ID NO: 119 - protein, 45 aa, Artificial Sequence; identical to SEQ ID NO: 91
  Synthetic polypeptide

SEQ ID NO: 120 - DNA, 135 bp, Artificial Sequence; identical to SEQ ID NO: 92
  Synthetic polynucleotide

SEQ ID NO: 121 - protein, 37 aa, Artificial Sequence; identical to SEQ ID NO: 93
  Synthetic polypeptide

SEQ ID NO: 122 - DNA, 111 bp, Artificial Sequence; identical to SEQ ID NO: 94
  Synthetic polynucleotide

SEQ ID NO: 123 - protein, 2 aa, Artificial Sequence
  Synthetic polypeptide
VD

SEQ ID NO: 124 - DNA, 6 bp, Artificial Sequence
  Synthetic oligonucleotide
gtcgac

SEQ ID NO: 125 - protein, 41 aa, Artificial Sequence; identical to SEQ ID NO: 97
  Synthetic polypeptide

SEQ ID NO: 126 - DNA, 123 bp, Artificial Sequence; identical to SEQ ID NO: 98
  Synthetic polynucleotide

SEQ ID NO: 127 - protein, 112 aa, Artificial Sequence; identical to SEQ ID NO: 99
  Synthetic polypeptide

SEQ ID NO: 128 - DNA, 336 bp, Artificial Sequence; identical to SEQ ID NO: 100
  Synthetic polynucleotide

SEQ ID NO: 129 - protein, 630 aa, Artificial Sequence
  Synthetic polypeptide
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 130 - protein, 622 aa, Artificial Sequence
  Synthetic polypeptide
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 131 - protein, 8 aa, Artificial Sequence
  Synthetic polypeptide
PQAPRRPL

SEQ ID NO: 132 - protein, 12 aa, Artificial Sequence
  Synthetic polypeptide
RPLPQVATLIDR

SEQ ID NO: 133 - protein, 14 aa, Artificial Sequence
  Synthetic polypeptide
GHEMSPQVATLIDR

SEQ ID NO: 134 - DNA, 69 bp, Artificial Sequence
  Synthetic oligonucleotide
atgcagatcccccaggccccctggcccgtggtgtgggccgtgctgcagctgggctggaggcccggctgg

SEQ ID NO: 135 - protein, 23 aa, Artificial Sequence
  Synthetic polypeptide
MQIPQAPWPVVWAVLQLGWRPGW

SEQ ID NO: 136 - DNA, 441 bp, Artificial Sequence
  Synthetic polynucleotide
ttcctggacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggactgcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgaggtgcccaccgcccaccccagccccagccccaggcccgccggccagttccagaccctggtg

SEQ ID NO: 137 - protein, 147 aa, Artificial Sequence
  Synthetic polypeptide
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 138 - DNA, 36 bp, Artificial Sequence
  Synthetic oligonucleotide
tgccccagccccctgttccccggccccagcaagccc

SEQ ID NO: 139 - protein, 12 aa, Artificial Sequence
  Synthetic polypeptide
CPSPLFPGPSKP

SEQ ID NO: 140 - DNA, 81 bp, Artificial Sequence
  Synthetic oligonucleotide
ttctgggtgctggtggtggtgggcggcgtgctggcctgctacagcctgctggtgaccgtggccttcatcatcttctgggtg

SEQ ID NO: 141 - protein, 27 aa, Artificial Sequence
  Synthetic polypeptide
FWVLVVVGGVLACYSLLVTVAFIIFWV

SEQ ID NO: 142 - DNA, 72 bp, Artificial Sequence
  Synthetic oligonucleotide
ctgtgcgccaggcccaggaggagccccgcccaggaggacggcaaggtgtacatcaacatgcccggcaggggc

SEQ ID NO: 143 - protein, 24 aa, Artificial Sequence
  Synthetic polypeptide
LCARPRRSPAQEDGKVYINMPGRG

SEQ ID NO: 144 - DNA, 339 bp, Artificial Sequence
  Synthetic polynucleotide
agggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 145 - protein, 113 aa, Artificial Sequence
  Synthetic polypeptide
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 146 - DNA, 1038 bp, Artificial Sequence
  Synthetic polynucleotide
atgcagatcccccaggccccctggcccgtggtgtgggccgtgctgcagctgggctggaggcccggctggttcctggacagccccgacaggccctggaacccccccaccttcagccccgccctgctggtggtgaccgagggcgacaacgccaccttcacctgcagcttcagcaacaccagcgagagcttcgtgctgaactggtacaggatgagccccagcaaccagaccgacaagctggccgccttccccgaggacaggagccagcccggccaggactgcaggttcagggtgacccagctgcccaacggcagggacttccacatgagcgtggtgagggccaggaggaacgacagcggcacctacctgtgcggcgccatcagcctggcccccaaggcccagatcaaggagagcctgagggccgagctgagggtgaccgagaggagggccgaggtgcccaccgcccaccccagccccagccccaggcccgccggccagttccagaccctggtgtgccccagccccctgttccccggccccagcaagcccttctgggtgctggtggtggtgggcggcgtgctggcctgctacagcctgctggtgaccgtggccttcatcatcttctgggtgctgtgcgccaggcccaggaggagccccgcccaggaggacggcaaggtgtacatcaacatgcccggcaggggcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagccccagaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagg

SEQ ID NO: 147 - protein, 346 aa, Artificial Sequence
  Synthetic polypeptide
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 148 - DNA, 63 bp, Artificial Sequence; identical to SEQ ID NO: 76
  Synthetic oligonucleotide

SEQ ID NO: 149 - protein, 21 aa, Artificial Sequence
  Synthetic polypeptide
MALPVTALLLPLALLLHAARP

SEQ ID NO: 150 - DNA, 6 bp, Artificial Sequence
  Synthetic oligonucleotide
gggagc

SEQ ID NO: 151 - protein, 2 aa, Artificial Sequence
  Synthetic polypeptide
GS

SEQ ID NO: 152 - DNA, 48 bp, Artificial Sequence; identical to SEQ ID NO: 80
  Synthetic oligonucleotide

SEQ ID NO: 153 - protein, 16 aa, Artificial Sequence
  Synthetic polypeptide
ELPTQGTFSNVSTNVS

SEQ ID NO: 154 - DNA, 21 bp, Artificial Sequence
  Synthetic oligonucleotide
tctccgggatccccaggatgg

SEQ ID NO: 155 - protein, 7 aa, Artificial Sequence
  Synthetic polypeptide
SPGSPGW

SEQ ID NO: 156 - DNA, 384 bp, Artificial Sequence
  Synthetic polynucleotide
ttcctggacagcccagatcggccctggaatccccctaccttttcccctgccctgctggtggtgacagagggcgacaacgccaccttcacatgcagcttttccaacacctctgagagcttcgtgctgaattggtacagaatgtccccatctaaccagacagataagctggccgcatttccagaggacaggtcccagcctggacaggattgccgcttccgggtgacccagctgcccaatggcagagactttcacatgagcgtggtgagggcccggagaaacgattccggcacatacctgtgcggagccatctctctggccccaaaggcacagatcaaggagagcctgagggcagagctgagggtgaccgagaggagggcagaggtg

SEQ ID NO: 157 - protein, 147 aa, Artificial Sequence; identical to SEQ ID NO: 137
  Synthetic polypeptide

SEQ ID NO: 158 - DNA, 36 bp, Artificial Sequence
  Synthetic oligonucleotide
tgcccttccccactgttcccaggaccttctaagccc

SEQ ID NO: 159 - protein, 147 aa, Artificial Sequence; identical to SEQ ID NO: 137
  Synthetic polypeptide

SEQ ID NO: 160 - DNA, 81 bp, Artificial Sequence
  Synthetic oligonucleotide
ttttgggtgctggtggtggtgggaggcgtgctggcctgctactctctgctggtgacagtggccttcatcatcttttgggtg

SEQ ID NO: 161 - protein, 27 aa, Artificial Sequence
  Synthetic polypeptide
FWVLVVVGGVLACYSLLVTVAFIIFWV

SEQ ID NO: 162 - DNA, 72 bp, Artificial Sequence
  Synthetic oligonucleotide
ctgtgcgcccgccctcggagaagcccagcacaggaggacggcaaggtgtatatcaatatgccaggcagaggc

SEQ ID NO: 163 - protein, 24 aa, Artificial Sequence
  Synthetic polypeptide
LCARPRRSPAQEDGKVYINMPGRG

SEQ ID NO: 164 - DNA, 339 bp, Artificial Sequence
  Synthetic polynucleotide
agggtgaagttttctcggagcgccgatgcaccagcataccagcagggacagaaccagctgtataacgagctgaatctgggcaggcgcgaggagtacgacgtgctggataagcggagaggcagagacccagagatgggaggcaagccacagaggaggaagaaccctcaggagggcctgtacaatgagctgcagaaggacaagatggccgaggcctattccgagatcggcatgaagggagagcggagaaggggcaagggacacgatggcctgtaccagggcctgtctaccgccacaaaggacacctatgatgccctgcacatgcaggccctgccacccagg

SEQ ID NO: 165 - protein, 113 aa, Artificial Sequence; identical to SEQ ID NO: 145
  Synthetic polypeptide

SEQ ID NO: 166 - DNA, 1107 bp, Artificial Sequence
  Synthetic polynucleotide
atggctcttccggtaaccgctctgctgcttcctttggctctgcttcttcacgcggctaggccagggagcgaattgcctacgcaggggactttttcaaatgtaagcacgaatgtgagctctccgggatccccaggatggttcctggacagcccagatcggccctggaatccccctaccttttcccctgccctgctggtggtgacagagggcgacaacgccaccttcacatgcagcttttccaacacctctgagagcttcgtgctgaattggtacagaatgtccccatctaaccagacagataagctggccgcatttccagaggacaggtcccagcctggacaggattgccgcttccgggtgacccagctgcccaatggcagagactttcacatgagcgtggtgagggcccggagaaacgattccggcacatacctgtgcggagccatctctctggccccaaaggcacagatcaaggagagcctgagggcagagctgagggtgaccgagaggagggcagaggtgcctacagcacacccaagcccttccccaagaccagcaggacagttccagaccctggtgtgcccttccccactgttcccaggaccttctaagcccttttgggtgctggtggtggtgggaggcgtgctggcctgctactctctgctggtgacagtggccttcatcatcttttgggtgctgtgcgcccgccctcggagaagcccagcacaggaggacggcaaggtgtatatcaatatgccaggcagaggcagggtgaagttttctcggagcgccgatgcaccagcataccagcagggacagaaccagctgtataacgagctgaatctgggcaggcgcgaggagtacgacgtgctggataagcggagaggcagagacccagagatgggaggcaagccacagaggaggaagaaccctcaggagggcctgtacaatgagctgcagaaggacaagatggccgaggcctattccgagatcggcatgaagggagagcggagaaggggcaagggacacgatggcctgtaccagggcctgtctaccgccacaaaggacacctatgatgccctgcacatgcaggccctgccacccagg

SEQ ID NO: 167 - protein, 369 aa, Artificial Sequence
  Synthetic polypeptide
MALPVTALLLPLALLLHAARPGSELPTQGTFSNVSTNVSSPGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 168 - protein, 521 aa, Artificial Sequence; identical to SEQ ID NO: 101
  Synthetic polypeptide
  SITE (1)..(521): /note=“Variant residues given in the sequence have no preference with respect to those in the annotations for variant positions”
  VARIANT (201)..(201): /replace=“Val”

SEQ ID NO: 169 - skipped

SEQ ID NO: 170 - protein, 21 aa, Artificial Sequence
  Synthetic polypeptide
MALPVTALLLPLALLLHAARP

SEQ ID NO: 171 - DNA, 63 bp, Artificial Sequence; identical to SEQ ID NO: 76
  Synthetic oligonucleotide

SEQ ID NO: 172 - protein, 2 aa, Artificial Sequence
  Synthetic polypeptide
GS

SEQ ID NO: 173 - DNA, 6 bp, Artificial Sequence
  Synthetic oligonucleotide
gggagc

SEQ ID NO: 174 - protein, 16 aa, Artificial Sequence
  Synthetic polypeptide
ELPTQGTFSNVSTNVS

SEQ ID NO: 175 - DNA, 48 bp, Artificial Sequence; identical to SEQ ID NO: 80
  Synthetic oligonucleotide

SEQ ID NO: 176 - protein, 2 aa, Artificial Sequence
  Synthetic peptide
SP

SEQ ID NO: 177 - DNA, 6 bp, Artificial Sequence
  Synthetic oligonucleotide
tctccg

SEQ ID NO: 178 - protein, 115 aa, Artificial Sequence; identical to SEQ ID NO: 38
  Synthetic polypeptide

SEQ ID NO: 179 - DNA, 345 bp, Artificial Sequence; identical to SEQ ID NO: 112
  Synthetic polynucleotide

SEQ ID NO: 180 - protein, 15 aa, Artificial Sequence
  Synthetic polypeptide
GGGGSGGGGSGGGGS

SEQ ID NO: 181 - DNA, 45 bp, Artificial Sequence; identical to SEQ ID NO: 114
  Synthetic oligonucleotide

SEQ ID NO: 182 - protein, 112 aa, Artificial Sequence; identical to SEQ ID NO: 47
  Synthetic polypeptide
  SITE (1)..(112): /note=“Variant residues given in the sequence have no preference with respect to those in the annotations for variant positions”
  VARIANT (30)..(30): /replace=“Val”

SEQ ID NO: 183 - DNA, 336 bp, Artificial Sequence
  Synthetic polynucleotide
gatgttctcctgacgcaaactcctttgtcattgccggtaagtctgggcgaccaagcgagcatttcctgtcgcagcagccagaacatarthcatagtgatggcgacacttacttggattggttcctccagaaaccggggcaatctccgaacctcttgatatataaggtctcaaatcgctttagcggggttcccgaccgcttttcaggttctggctcaggtaccgatttcacgctgaaaatttctagagtagaggctgacgatctgggtgtttactattgttttcaagcttcacacgttccttatacgttcggaggggggactaaactggaggttaaa

SEQ ID NO: 184 - protein, 1 aa, Artificial Sequence
  Synthetic peptide
R

SEQ ID NO: 185 - DNA, 3 bp, Artificial Sequence
  Synthetic oligonucleotide
cgt

SEQ ID NO: 186 - protein, 45 aa, Artificial Sequence; identical to SEQ ID NO: 91
  Synthetic polypeptide

SEQ ID NO: 187 - DNA, 135 bp, Artificial Sequence; identical to SEQ ID NO: 92
  Synthetic polynucleotide

SEQ ID NO: 188 - protein, 37 aa, Artificial Sequence; identical to SEQ ID NO: 93
  Synthetic polypeptide

SEQ ID NO: 189 - DNA, 111 bp, Artificial Sequence; identical to SEQ ID NO: 94
  Synthetic polynucleotide

SEQ ID NO: 190 - protein, 2 aa, Artificial Sequence
  Synthetic polypeptide
VD

SEQ ID NO: 191 - DNA, 6 bp, Artificial Sequence
  Synthetic oligonucleotide
gtcgac

SEQ ID NO: 192 - protein, 41 aa, Artificial Sequence; identical to SEQ ID NO: 97
  Synthetic polypeptide

SEQ ID NO: 193 - DNA, 123 bp, Artificial Sequence; identical to SEQ ID NO: 98
  Synthetic polynucleotide

SEQ ID NO: 194 - protein, 112 aa, Artificial Sequence; identical to SEQ ID NO: 99
  Synthetic polypeptide

SEQ ID NO: 195 - DNA, 336 bp, Artificial Sequence; identical to SEQ ID NO: 100
  Synthetic polynucleotide

SEQ ID NO: 196 - protein, 500 aa, Artificial Sequence
  Synthetic polypeptide
GSELPTQGTFSNVSTNVSSPEVKLVESGGGLVKPGGSLKLSCEASGFTFSRYAMSWVRHTPEKRLAWVASITFGGDPYYPDGLKGRFTISRDNTRNILYLQMNSLRSEDTAMYYCVRHESWFAYWGQGTLVTVSAGGGGSGGGGSGGGGSDVLMTQTPLSLPVSLGEQVSISCRSSQTIVHTDGNIYLEWYLQNPGQSPRLLIYKISNRFSGVPDRFSGSGSGTDFTLKISRVEAEDLGIYYCFQASHVPYTFGGGTKLEIKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 197 - protein, 500 aa, Artificial Sequence
  Synthetic polypeptide
  SITE (1)..(500): /note=“Variant residues given in the sequence have no preference with respect to those in the annotations for variant positions”
  VARIANT (180)..(180): /replace=“Val”
GSELPTQGTFSNVSTNVSSPEVKLVESGGGLVKPGGSLKVSCTASGFTFSRYAMSWVRQTPERRLEWVASITFGGSAYYLDSVKGRFTISRDNAQNILYLQMNSLVSEDTAIYNCARHQPWFDYWGQGTLVTVSAGGGGSGGGGSGGGGSDVLLTQTPLSLPVSLGDQASISCRSSQNIIHSDGDTYLDWFLQKPGQSPNLLIYKVSNRFSGVPDRFSGSGSGTDFTLKISRVEADDLGVYYCFQASHVPYTFGGGTKLEVKRTTTPAPRPPTPAPTIASQPLSLRPEACRPAAGGAVHTRGLDFACDIYIWAPLAGTCGVLLLSLVITLYCNHRNRRRVCKCPRVDRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 198 - protein, 500 aa, Artificial Sequence; identical to SEQ ID NO: 197
  Synthetic polypeptide
  SITE (1)..(500): /note=“Variant residues given in the sequence have no preference with respect to those in the annotations for variant positions”
  VARIANT (180)..(180): /replace=“Val”

SEQ ID NO: 199 - protein, 323 aa, Artificial Sequence
  Synthetic polypeptide
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 200 - protein, 348 aa, Artificial Sequence
  Synthetic polypeptide
GSELPTQGTFSNVSTNVSSPGSPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVCPSPLFPGPSKPFWVLVVVGGVLACYSLLVTVAFIIFWVLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 201 - protein, 10 aa, Artificial Sequence
  Synthetic polypeptide
EQKLISEEDL

SEQ ID NO: 202 - protein, 8 aa, Artificial Sequence
  Synthetic polypeptide
DYKDDDDK

SEQ ID NO: 203 - protein, 6 aa, Artificial Sequence
  Synthetic polypeptide
HHHHHH

SEQ ID NO: 204 - protein, 9 aa, Artificial Sequence
  Synthetic polypeptide
YPYDVPDYA

SEQ ID NO: 205 - protein, 11 aa, Artificial Sequence
  Synthetic polypeptide
YTDIEMNRLGK

SEQ ID NO: 206 - protein, 11 aa, Artificial Sequence
  Synthetic polypeptide
QPELAPEDPED

SEQ ID NO: 207 - protein, 14 aa, Artificial Sequence
  Synthetic polypeptide
GKPIPNPLLGLDST

SEQ ID NO: 208 - protein, 5 aa, Artificial Sequence
  Synthetic polypeptide
RRRRR

SEQ ID NO: 209 - protein, 8 aa, Artificial Sequence
  Synthetic polypeptide
WSHPQFEK

SEQ ID NO: 210 - protein, 15 aa, Artificial Sequence
  Synthetic polypeptide
KETAAAKFERQHMDS

SEQ ID NO: 211 - protein, 22 aa, Artificial Sequence
  Synthetic polypeptide
DYKDHDGDYKDHDIDYKDDDDK

SEQ ID NO: 212 - protein, 19 aa, Artificial Sequence
  Synthetic polypeptide
KDHLIHNVHKEFHAHAHNK

SEQ ID NO: 213 - protein, 38 aa, Artificial Sequence
  Synthetic polypeptide
MDEKTTGWRGGHVVEGLAGELEQLRARLEHHPQGQREP

SEQ ID NO: 214 - protein, 110 aa, Artificial Sequence
  Synthetic polypeptide
  SITE (1)..(10): /note=“This region may encompass 2-10 residues”
  SITE (1)..(110): /note=“This sequence may encompass 2-10 ′((G)xS)′ repeating units where x is an integer between 2 and 10”
  SITE (12)..(21): /note=“This region may encompass 2-10 residues”
  SITE (23)..(32): /note=“This region may encompass 2-10 residues”
  SITE (34)..(43): /note=“This region may encompass 2-10 residues”
  SITE (45)..(54): /note=“This region may encompass 2-10 residues”
  SITE (56)..(65): /note=“This region may encompass 2-10 residues”
  SITE (67)..(76): /note=“This region may encompass 2-10 residues”
  SITE (78)..(87): /note=“This region may encompass 2-10 residues”
  SITE (89)..(98): /note=“This region may encompass 2-10 residues”
  SITE (100)..(109): /note=“This region may encompass 2-10 residues”
GGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGSGGGGGGGGGGS

SEQ ID NO: 215 - protein, 7 aa, Artificial Sequence
  Synthetic polypeptide
GVILTAL

SEQ ID NO: 216 - DNA, 10 bp, Artificial Sequence
  Synthetic oligonucleotide
gccaccatgc

(19) United States
(12) Patent Application Publication
(10) Pub. No.: US 2024/0279353 A1
(43) Pub. Date: Aug. 22, 2024

(54) MESOTHELIN ISOFORM BINDING MOLECULES AND CHIMERIC PD1 RECEPTOR MOLECULES, CELLS CONTAINING THE SAME AND USES THEREOF

(71) Applicant: KIROMIC BIOPHARMA, INC., Houston, TX (US)

(72) Inventors: Lucia PICCOTTI, Houston, TX (US); Leonardo MIRANDOLA, Plano, TX (US); Maurizio CHIRIVA-INTERNATI, Manvel, TX (US); David SPENCER, Houston, TX (US); Xiaohong WANG, Houston, TX (US); Yibin CHEN, Houston, TX (US)

(21) Appl. No.: 18/004,158

(22) PCT Filed: Jul. 2, 2021
(86) PCT No.: PCT/US21/40348

§ 371 Date: Jan. 3, 2023

Related U.S. Application Data
(60) Provisional application No. 63/185,790, filed on May 7, 2021.
(60) Provisional application No. 63/115,465, filed on Nov. 18, 2020.
(60) Provisional application No. 63/048,488, filed on Jul. 6, 2020.

(51) Int. Cl. C07K 16/30; A61K 39/00; A61P 35/00; C07K 14/705; C07K 14/725; C12N 5/0783; C12N 15/86; G01N 33/574

(52) U.S. Cl. CPC C07K 16/30; A61K 39/4611; A61K 39/4613; A61K 39/4631; A61K 39/464468; A61P 35/00; C07K 14/7051; C07K 14/70517; C12N 5/0636; C12N 5/0646; C12N 15/86; G01N 33/57484; A61K 2039/505; A61K 2239/21; C07K 2317/565; C07K 2317/622; C07K 2317/92; C07K 2319/33; C12N 2510/00; C12N 2740/13043

(57) ABSTRACT

The technology relates in part to binding molecules that specifically bind to a polypeptide that is the Isoform 2 of mesothelin, or that specifically bind to an antigenic determinant (epitope) of the isoform 2 of mesothelin, or that specifically bind to polypeptides containing an antigenic determinant (epitope) of the isoform 2 of mesothelin, chimeric PD1 receptors that bind to PD ligands such as PDLs, to polynucleotides including vectors that encode such binding molecules, to ceils presenting such binding molecules and to methods of making such cells, to humanized forms of the binding molecules, and to methods of using such binding molecules, such as for treating cancers (e.g., ovarian cancers and mesotheliomas), including cancers in which the Isoform 2 of mesothelin is specifically expressed and/or upregulated relative to normal tissues.

### RELATED APPLICATIONS

This patent application is a U.S. National Phase Application of PCT International Application No. PCT/US2021/040348, filed on Jul. 2, 2021, designating the United States Patent Office, which is an International Application of and claims the benefit of priority under 35 U.S.C. § 119(e) to U.S. Provisional Patent Application No. 63/048,488, filed on Jul. 6, 2020; U.S. Provisional Patent Application No. 63/115,465, filed on Nov. 18, 2020; and U.S. Provisional Patent Application No. 63/185,790, filed on May 7, 2021. The entire content of each of the foregoing patent applications is incorporated herein by reference for all purposes, including all text, tables and drawings.

### SEQUENCE LISTING

The material in the accompanying Sequence Listing is hereby incorporated by reference into this application. The accompanying Sequence Listing text file, named “060851-502N01US_SL_ST25.TXT” was created on Sep. 7, 2023 and is 139,319 bytes.

### FIELD

The technology relates in part to binding molecules that specifically bind to an isoform of mesothelin, to chimeric PD1 receptor molecules, to cells presenting such molecules and to methods of using such molecules, such as for the detection and/or treatment of cancers.

### BACKGROUND

Cancer treatments have undergone significant developments in recent years. Cancer however remains a difficult disease to treat, worldwide. Traditional cancer therapies, such as clinical operation, chemotherapy, and radiotherapy, may have a curative effect in the short term but often cause side effects, decreasing the quality of life.

Molecules that bind specifically to polypeptides associated with cancers, such as antibodies, have been used successfully for both hematologic malignancies and solid tumors over the last 20 years. These molecules (e.g., monoclonal antibodies) can exhibit antitumor activity by a variety of mechanisms that include direct cell killing, such as through receptor blockade or agonist activity, induction of apoptosis, the delivery of a drug, radiation, or cytotoxic agent; immune-mediated cell killing mechanisms; regulation of T cell function; and specific effects on tumor vasculature and stroma.

Immunotherapies have been developed for treatment of certain cancers. The use of engineered immune cells, such as chimeric antigen receptor- (CAR-) T cells, combine the expression of a tumor-specific binding molecule with the tumor killing activity of the T cells. CAR-T cells can recognize and kill tumor cells that express a surface antigen to which the CAR binds.

### SUMMARY

The specificity and efficacy of treatments using molecules and cells that bind to antigenic determinants, such as monoclonal antibodies and CAR-T cells, depends on the extent to which their cognate antigenic determinant is specific for a cancerous tissue (e.g., a tumor), i.e., the extent to which it is differentially expressed in the cancer tissue over the normal tissue. The advancement of cures for cancer rely on the development of novel, more efficacious, and more specific antibody-mediated approaches and immunotherapeutic approaches, aided by the discovery of novel target polypeptide candidates that display differential expression between healthy and malignant tissues.

Provided in certain aspects is a binding molecule that specifically binds to a polypeptide of SEQ ID NO:129, where the binding molecule includes the three complementarity-determining regions (CDRs) set forth in SEQ ID NO:2 and the three CDRs set forth in SEQ ID NO:11. Also provided herein, in certain aspects, is a binding molecule that specifically binds to a polypeptide epitope that includes SEQ ID NO:131 or SEQ ID NO:132 and contains the CDR3 of SEQ ID NO:2 and the CDR3 of SEQ ID NO:11. In aspects, the binding molecule contains the CDR1 and CDR2 of SEQ ID NO:2 and the CDR1 and CDR2 of SEQ ID NO:11.

In certain aspects, the binding molecules provided herein contain a heavy chain variable domain that is, or is about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, identical to the heavy chain variable domain of SEQ ID NO:2. In aspects, the binding molecule contains the heavy chain variable domain of SEQ ID NO:2.

In certain aspects, the binding molecules provided herein contain a light chain variable domain that is, or is about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, identical to the light chain variable domain of SEQ ID NO: 11. In aspects, the binding molecule contains the light chain variable domain of SEQ ID NO:11.

In certain aspects, the binding molecules provided herein contain the heavy chain variable domain of SEQ ID NO:2 and the light chain variable domain of SEQ ID NO:11. In aspects, the binding molecules provided herein contain a CDR3 of SEQ ID NO:5 and a CDR3 of SEQ ID NO:14. In certain aspects, the binding molecules provided herein contain a CDR1 of SEQ ID NO:3 and a CDR1 of SEQ ID NO:12. In aspects, the binding molecules provided herein contain a CDR2 of SEQ ID NO:4 and a CDR2 of SEQ ID NO:13.

Also provided herein, in certain aspects, is a binding molecule that specifically binds to a polypeptide of SEQ ID NO:129, where the binding molecule includes the three CDRs set forth in SEQ ID NO:38 and the three CDRs set forth in SEQ ID NO:47. Also provided herein, in certain aspects, is a binding molecule that specifically binds to a polypeptide epitope that includes SEQ ID NO:131 or SEQ ID NO:132 and contains the CDR3 of SEQ ID NO:38 and the CDR3 of SEQ ID NO:47. In aspects, the binding molecule contains the CDR1 and CDR2 of SEQ ID NO:38 and the CDR1 and CDR2 of SEQ ID NO:47.

In certain aspects, the binding molecules provided herein contain a heavy chain variable domain that is, or is about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the heavy chain variable domain of SEQ ID NO:38. In aspects, the binding molecule contains the heavy chain variable domain of SEQ ID NO:38.

In certain aspects, the binding molecules provided herein contain a light chain variable domain that is, or is about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more identical to the light chain variable domain of SEQ ID NO:47. In aspects, the binding molecule contains the light chain variable domain of SEQ ID NO:47.

In certain aspects, the binding molecules provided herein contain the heavy chain variable domain of SEQ ID NO:38 and the light chain variable domain of SEQ ID NO:47. In aspects, the binding molecules provided herein contain a CDR3 of SEQ ID NO:41 and a CDR3 of SEQ ID NO:50. In certain aspects, the binding molecules provided herein contain a CDR1 of SEQ ID NO:39 and a CDR1 of SEQ ID NO:48. In aspects, the binding molecules provided herein contain a CDR2 of SEQ ID NO:40 and a CDR2 of SEQ ID NO:49. In certain aspects, in the binding molecules that contain the light chain variable domain of SEQ ID NO:47 and/or SEQ ID NO:48, the X in SEQ ID NO:47 or SEQ ID NO:48 is isoleucine (I).

In certain aspects, any of the binding molecules provided herein can include an antibody, antibody fragment, single-chain antibody, diabody, or BiTe. In aspects, the antibody is selected from among a monoclonal antibody, a polyclonal antibody, a recombinant antibody, an IgE antibody, an IgD antibody, an IgM antibody, an IgG antibody, an antibody containing at least one amino acid substitution, an antibody containing at least one non-naturally occurring amino acid, or any combination of the foregoing. In certain aspects, the antibody is an IgG antibody. In aspects, the binding molecule is an antibody fragment selected from among an scFv, a Fab, a Fab′, a Fv, and a F(ab′)2.

In certain aspects, particular binding molecules provided herein can specifically bind to a polypeptide of SEQ ID NO:129 with a binding affinity of 100 nM or less. In aspects, certain binding molecules provided herein specifically bind to a polypeptide of SEQ ID NO:129 with a binding affinity of 10 nM or less. In certain aspects, particular binding molecules provided herein specifically bind to a polypeptide of SEQ ID NO:129 with a binding affinity of 1 nM or less.

In certain aspects, provided herein are chimeric PD1 (chPD1) receptor molecules (also referred to herein as “chimeric PD1 (chPD1) molecules”) that are binding molecules for PD ligands (e.g., PDL-1, PDL-2). In aspects, the chimeric PD1 receptor molecule is encoded in a construct and in certain aspects, the construct can be transduced into a cell. In certain aspects, the chimeric PD1 receptor molecule is, or contains a sequence that is, or is about, 90%, 91%, 92%, 93%, 94%, 95%, 96%, 97%, 98%, 99%, 99.5%, or more, identical to the polypeptide sequence set forth in SEQ ID NO:147, SEQ ID NO: 167, SEQ ID NO:199 or SEQ ID NO:200. In certain aspects, a chimeric PD1 molecule has or contains the polypeptide sequence set forth in SEQ ID NO:147, SEQ ID NO: 167, SEQ ID NO:199 or SEQ ID NO:200.

In certain aspects, any of the binding molecules provided herein, including the IsoMSLN binding molecules and the chimeric PD1 molecules, can include an antibody, antibody fragment, single-chain antibody, diabody, or BiTe. In aspects, the antibody is selected from among a monoclonal antibody, a polyclonal antibody, a recombinant antibody, an IgE antibody, an IgD antibody, an IgM antibody, an IgG antibody, an antibody containing at least one amino acid substitution, an antibody containing at least one non-naturally occurring amino acid, or any combination of the foregoing. In certain aspects, the antibody is an IgG antibody.

Also provided herein are chimeric antigen receptor (CAR) molecules (referred to interchangeably herein as “CAR binding molecules,” i.e., antigen-binding molecules that are CARs) that include any of the binding molecules provided herein. In certain aspects, the binding molecule is an scFv antibody fragment. In aspects, the binding molecules, including CAR binding molecules, provided herein include a membrane association polypeptide, and, in certain aspects, the membrane association polypeptide is a region of a native transmembrane polypeptide.

In aspects of the CAR molecules and other binding molecules provided herein, the membrane association polypeptide is a stalk region polypeptide. In certain aspects, the stalk region polypeptide is a CD8 stalk region polypeptide containing the sequence set forth in SEQ ID NO:91.

In aspects, the membrane association polypeptide is a transmembrane region polypeptide, and, in certain aspects, the transmembrane region polypeptide is a CD8 transmembrane region polypeptide containing the sequence set forth in SEQ ID NO:93. In certain aspects, the transmembrane region polypeptide is a CD28 transmembrane region polypeptide containing the sequence set forth in SEQ ID NO:140, which optionally is preceded by a truncated CD28 region polypeptide containing the sequence of SEQ ID NO:139. In aspects, the CAR binding molecules and other binding molecules provided herein can include a stalk region polypeptide and a transmembrane region polypeptide.

In any of the CAR binding molecules and other binding molecules provided herein, in certain aspects, the binding molecules include a signal polypeptide. In aspects, the signal polypeptide is a region of a transmembrane polypeptide. In certain aspects, the signal polypeptide is a signal region polypeptide of CD8 containing the sequence set forth in SEQ ID NO:75, or is a signal region polypeptide of PD1 containing the sequence set forth in SEQ ID NO:135.

In any of the CAR binding molecules and other binding molecules provided herein, in certain aspects, the binding molecule includes a tag polypeptide. In aspects, the tag polypeptide is a portion of an extracellular region of a cell membrane associated polypeptide. In certain aspects, the tag polypeptide is a portion of the extracellular region of a CD34 polypeptide. In aspects, the tag polypeptide contains the sequence set forth in SEQ ID NO:79.

Any of the CAR binding molecules and other binding molecules provided herein can include, in certain aspects, one or more stimulatory polypeptides. In certain aspects, the CAR binding molecules and other binding molecules provided herein include a cytoplasmic region or portion thereof of a native stimulatory polypeptide. In aspects, the stimulatory polypeptide is capable of stimulating an immune cell. In certain aspects, the immune cell is selected from among one or more of a T-cell, NK cell, invariant natural killer T cell (iNKT) and mucosal-associated innate T (MAIT) cell. In aspects, the T-cell is selected from among one or more of a gamma.delta (γδ) T-cell, CD4+ T-cell and CD8+ T-cell.

In certain aspects of the CAR binding molecules and other binding molecules provided herein, the stimulatory polypeptide independently is selected from among CD27, CD28, ICOS, 4-1BB, CD40, RANK/TRANCE-R, CD3-zeta (ζ) chain, OX40, a pattern recognition receptor, TRIF, DNAX activating protein (e.g., DAP10), NOD-like receptor and RIG-like helicase. In aspects, the stimulatory polypeptide includes a cytoplasmic region of the CD3-zeta chain. In certain aspects, the stimulatory polypeptide includes a cytoplasmic region of CD28.

In any of the CAR binding molecules and other binding molecules provided herein, the binding molecule can, in certain aspects, include two stimulatory polypeptides, and, in aspects, the binding molecule contains a cytoplasmic region of the CD3-zeta chain and a cytoplasmic region of CD28.

In certain aspects, a binding molecule can include a cytoplasmic region of the CD3-zeta chain and a cytoplasmic region of DAP10. In aspects, the cytoplasmic region of the CD3-zeta chain contains the sequence set forth in SEQ ID NO:99 or the sequence set forth in SEQ ID NO:145. In certain aspects, the cytoplasmic region of CD28 contains the sequence set forth SEQ ID NO:97. In aspects, the cytoplasmic region of DAP10 contains the sequence set forth in SEQ ID NO:143.

Any of the CAR binding molecules and other binding molecules provided herein can include, in certain aspects, a signal polypeptide and a tag polypeptide and a linker between the signal polypeptide and the tag polypeptide. In aspects, the linker between the signal polypeptide and the tag polypeptide is about 1 amino acid to about 10 consecutive amino acids in length. In certain aspects, the linker between the signal polypeptide and the tag polypeptide contains the sequence set forth in SEQ ID NO:77. Any of the CAR binding molecules and other binding molecules provided herein can include, in certain aspects, a linker appended to the C-terminus of a tag polypeptide. In aspects, a C-terminus of a tag polypeptide is attached to a linker containing the sequence of SEQ ID NO:155.

Any of the CAR binding molecules and other binding molecules provided herein can include, in certain aspects, a tag polypeptide and a heavy chain variable (VH) domain polypeptide and a linker between the tag polypeptide and the VH domain polypeptide. In aspects, the linker between the tag polypeptide and the VH domain polypeptide is about 1 amino acid to about 10 consecutive amino acids in length. In certain aspects, the linker between the tag polypeptide and the VH domain polypeptide contains the sequence set forth in SEQ ID NO:81.

Any of the CAR binding molecules and other binding molecules provided herein can include, in certain aspects, a heavy chain variable (VH) domain polypeptide and a light chain variable (VL) domain polypeptide and a linker between the VH domain polypeptide and the VL domain polypeptide. In aspects, the linker between the VH domain polypeptide and the VL domain polypeptide is about 5 to about 25 consecutive amino acids in length. In certain aspects, the linker between the VH domain polypeptide and the VL domain polypeptide contains two more consecutive glycine amino acids, and optionally contains one or more serine amino acids. In aspects, the linker between the VH domain polypeptide and the VL domain polypeptide comprises ((G)ₘS)ₙ, where m is an integer between 2 and 10 and n independently is an integer between 2 and 10 (see, e.g., SEQ ID NO:214). In certain aspects, the linker between the VH domain polypeptide and the VL domain polypeptide contains the sequence set forth in SEQ ID NO:85.

Any of the CAR binding molecules and other binding molecules provided herein can include, in certain aspects, a light chain variable (VL) domain polypeptide and a stalk region polypeptide and a linker between the VL domain polypeptide and the stalk region polypeptide. In certain aspects, the linker between the VL domain polypeptide and the stalk region polypeptide is about 1 amino acid to about 10 consecutive amino acids in length. In aspects, the linker between the VL domain polypeptide and the stalk region polypeptide contains the sequence set forth in SEQ ID NO:89.

Any of the CAR binding molecules and other binding molecules provided herein can include, in certain aspects, a transmembrane region polypeptide and a stimulatory polypeptide and a linker between the transmembrane region polypeptide and the stimulatory polypeptide. In certain aspects, the linker between the transmembrane region polypeptide and the stimulatory polypeptide is about 1 amino acid to about 10 consecutive amino acids in length. In aspects, the linker between the transmembrane region polypeptide and the stimulatory polypeptide contains the sequence set forth in SEQ ID NO:95.

Any of the CAR binding molecules and other binding molecules provided herein can include, in certain aspects, a VH Domain that contains the sequence set forth in SEQ ID NO:83. In certain aspects, the CAR binding molecules provided herein can include a VL Domain that contains the sequence set forth in SEQ ID NO:87. In aspects, the CAR binding molecules provided herein have or contain the sequence set forth in SEQ ID NO:73.

Any of the CAR binding molecules and other binding molecules provided herein can include, in certain aspects, a VH Domain that contains the sequence set forth in SEQ ID NO:111. In certain aspects, the CAR binding molecules provided herein can include a VL Domain that contains the sequence set forth in SEQ ID NO:115. In certain aspects, the X in SEQ ID NO:115 is valine (V). In aspects, the CAR binding molecules provided herein have or contain the sequence set forth in SEQ ID NO:101.

In certain aspects, any of the CAR or other binding molecules provided herein can have a structure depicted by one or more of the following formulae:

Nterm-(VH Domain)-(VL Domain)-(transmembrane region)-(first stimulatory molecule cytoplasmic region)-(second stimulatory molecule cytoplasmic region)-Cterm, Formula A:

wherein “Nterm” is the N-terminus of the binding molecule and “Cterm” is the C-terminus of the binding molecule.

Nterm-(VH Domain)-(VL Domain)-(transmembrane region)-(CD28 cytoplasmic region)-(CD3-zeta cytoplasmic region)-Cterm, Formula B:

wherein “Nterm” is the N-terminus of the binding molecule and “Cterm” is the C-terminus of the binding molecule.

Nterm-(VH Domain)-(VL Domain)-(CD8 transmembrane region)-(CD28 cytoplasmic region)-(CD3-zeta cytoplasmic region)-Cterm, Formula C:

wherein “Nterm” is the N-terminus of the binding molecule and “Cterm” is the C-terminus of the binding molecule.

Nterm-(VH Domain)-(VL Domain)-(CD8 stalk region)-(CD8 transmembrane region)-(CD28 cytoplasmic region)-(CD3-zeta cytoplasmic region)-Cterm, Formula D:

wherein “Nterm” is the N-terminus of the binding molecule and “Cterm” is the C-terminus of the binding molecule.

Nterm-(CD34 tag)-(VH Domain)-(VL Domain)-(CD8 stalk region)-(CD8 transmembrane region)-(CD28 cytoplasmic region)-(CD3-zeta cytoplasmic region)-Cterm, Formula E:

wherein “Nterm” is the N-terminus of the binding molecule and “Cterm” is the C-terminus of the binding molecule.

Nterm-(CD8 signal)-(Linker 1)-(CD34 tag)-(Linker 2)-(VH Domain)-(Linker 3)-(VL Domain)-(Linker 4)-(CD8 stalk region)-(CD8 transmembrane region)-(Linker 5)-(CD28 cytoplasmic region)-(CD3-zeta cytoplasmic region)-Cterm, Formula F:

wherein “Nterm” is the N-terminus of the binding molecule and “Cterm” is the C-terminus of the binding molecule.

In aspects, a binding molecule having a structure of any one of Formula A-F can include one or more of the following polypeptide regions independently chosen from:
a CD8 signal polypeptide of SEQ ID NO:75, SEQ ID NO:103 or SEQ ID NO:170;
a Linker 1 polypeptide of SEQ ID NO:77, SEQ ID NO:105 or SEQ ID NO:172;
a CD34 tag polypeptide of SEQ ID NO:79, SEQ ID NO:107 or SEQ ID NO:174;
a Linker 2 polypeptide of SEQ ID NO:81, SEQ ID NO:109 or SEQ ID NO:176;
a VH Domain polypeptide of SEQ ID NO:83 or SEQ ID NO:111;
a Linker 3 polypeptide of SEQ ID NO:85, SEQ ID NO:113 or SEQ ID NO:180;
a VL Domain polypeptide of SEQ ID NO:87 or SEQ ID NO: 115;
a Linker 4 of SEQ ID NO:89, SEQ ID NO:117 or SEQ ID NO:184;
a CD8 stalk region polypeptide of SEQ ID NO:91, SEQ ID NO:119 or SEQ ID NO:186;
a CD8 transmembrane region polypeptide of SEQ ID NO:93, SEQ ID NO:121 or SEQ ID NO:188;
a Linker 5 polypeptide of SEQ ID NO:95, SEQ ID NO:123 or SEQ ID NO:190;
a CD28 cytoplasmic region polypeptide of SEQ ID NO:97, SEQ ID NO:125 or SEQ ID NO:192;
a CD3-zeta cytoplasmic region polypeptide of SEQ ID NO:99, SEQ ID NO:127, SEQ ID NO:145, SEQ ID NO:165 or SEQ ID NO:194; and
a combination of the foregoing.

In aspects, any of the chimeric PD1 molecules provided herein can have a structure depicted by one of the following formula:

Nterm-(PD1 region(extracellular))-(truncated CD28 region(extracellular))-(CD28 transmembrane region)-(DAP10 region(cytoplasmic))-(CD3-zeta region(cytoplasmic))-Cterm, Formula G:

wherein “Nterm” is the N-terminus of the binding molecule and “Cterm” is the C-terminus of the binding molecule.

Nterm-(PD1 signal)-(PD1 region (extracellular))-(truncated CD28 region (extracellular))-(CD28 transmembrane region)-(DAP10 region (cytoplasmic))-(CD3-zeta region (cytoplasmic))-Cterm, Formula H:

wherein “Nterm” is the N-terminus of the binding molecule and “Cterm” is the C-terminus of the binding molecule.

Nterm-(linker 1)-(CD34 tag)-(linker 2)-(PD1 region (extracellular))-(truncated CD28 region (extracellular))-(CD28 transmembrane region)-(DAP10 region (cytoplasmic))-(CD3-zeta region (cytoplasmic))-Cterm, Formula I:

wherein “Nterm” is the N-terminus of the binding molecule and “Cterm” is the C-terminus of the binding molecule.

Nterm-(CD8 signal)-(linker 1)-(CD34 tag)-(linker 2)-(PD1 region (extracellular))-(truncated CD28 region (extracellular))-(CD28 transmembrane region)-(DAP10 region (cytoplasmic))-(CD3-zeta region (cytoplasmic))-Cterm, Formula J:

wherein “Nterm” is the N-terminus of the binding molecule and “Cterm” is the C-terminus of the binding molecule.

Nterm-(CD34 tag)-(linker)-(PD1 region (extracellular))-(truncated CD28 region (extracellular))-(CD28 transmembrane region)-(DAP10 region (cytoplasmic))-(CD3-zeta region (cytoplasmic))-Cterm, Formula K:

wherein “Nterm” is the N-terminus of the binding molecule and “Cterm” is the C-terminus of the binding molecule.

In aspects, a chimeric PD1 molecule having a structure of any one of Formula G-K can include one or more of the following polypeptide regions independently chosen from:
a PD1 signal polypeptide of SEQ ID NO:135;
a CD8 signal polypeptide of SEQ ID NO:149;
a linker 1 polypeptide of SEQ ID NO:151;
a CD34 tag polypeptide of SEQ ID NO:153;
a linker 2 polypeptide of SEQ ID NO:155;
a PD1 region (extracellular) polypeptide of SEQ ID NO:137 or SEQ ID NO:157;
a truncated CD28 region (extracellular) polypeptide of SEQ ID NO:139 or SEQ ID NO:159;
a CD28 transmembrane region polypeptide of SEQ ID NO:141 or SEQ ID NO:161;
a DAP10 region (cytoplasmic) polypeptide of SEQ ID NO:143 or SEQ ID NO:163;
a CD3-zeta region (cytoplasmic) polypeptide of SEQ ID NO:99, SEQ ID NO:127, SEQ ID NO:145, SEQ ID NO:165 or SEQ ID NO:194; and
a combination of the foregoing.

In certain aspects, any of the binding molecules provided herein, including any of the CAR binding molecules or chimeric PD1 molecules provided herein, can be isolated.

Also provided herein are nucleic acids that include a polynucleotide that encodes any of the binding molecules provided herein. In certain aspects, the nucleic acid is an isolated nucleic acid. Also provided herein are vectors containing any of the polynucleotides provided herein. In certain aspects, provided herein are cells containing any of the polynucleotides provided herein. In aspects, provided herein are cells containing any of the binding molecules, including the CAR binding molecules, provided herein.

In certain aspects, a cell containing a polynucleotide or a binding molecule, including a CAR binding molecule, is an immune cell in a population of cells. In aspects, the immune cell is selected from among one or more of a T-cell, NK cell, invariant natural killer T cell (iNKT) and mucosal-associated innate T (MAIT) cell. In certain aspects, the T-cell is selected from among one or more of a gamma.delta T-cell, CD4+ T-cell and CD8+ T-cell. In aspects, the cell is isolated and/or a population of cells that includes the cell is isolated. In certain aspects, the cell is in vitro or ex vivo. In aspects, the cell is in vivo.

Also provided herein are methods of making an enriched population of immune cells, such as gamma delta T-cells and iNKT cells. In aspects, the enriched population of immune cells can be modified, e.g., by mutations, insertions or deletions in one or more endogenous genes, by adding one or more exogenous genes. In aspects, the one or more exogenous genes express one or more of the binding molecules provided herein.

Any of the cells provided herein can, in certain aspects, include a switch polypeptide and/or a polynucleotide encoding a switch polypeptide. In certain aspects, the switch polypeptide is capable of inducing cell elimination after the cell is contacted with a multimeric agent capable of binding to the switch polypeptide. In aspects, the switch polypeptide contains, and/or is encoded by nucleic acids that encode, (i) a first polypeptide capable of binding to a multimeric agent, and (ii) a second polypeptide capable of facilitating elimination of the cell upon multimeric agent-induced multimerization of the switch polypeptide. In certain aspects, the switch polypeptide contains, contains, and/or is encoded by nucleic acids that encode, a third polypeptide capable of binding to the multimeric agent to which the first polypeptide is capable of binding, or a third polypeptide capable of binding to a multimeric agent different than the multimeric agent to which the first polypeptide is capable of binding. In aspects, the switch polypeptide contains, contains, and/or is encoded by nucleic acids that encode, (a) a first switch polypeptide containing (i) a first polypeptide capable of binding to a multimeric agent, and (ii) a second polypeptide capable of facilitating elimination of the cell upon multimeric agent-induced multimerization of the switch polypeptide; and (b) a second switch polypeptide containing (1) a third polypeptide capable of binding to the multimeric agent to which the first polypeptide is capable of binding, and (2) the second polypeptide capable of facilitating elimination of the cell upon multimeric agent-induced multimerization of the switch polypeptide. In aspects, the polypeptide capable of facilitating cell elimination is a native polypeptide or functional fragment thereof. In certain aspects, the polypeptide capable of facilitating cell elimination is an apoptosis-facilitating polypeptide. In aspects, the apoptosis-facilitating polypeptide is selected from among Fas, Fas-associated death domain-containing protein (FADD), caspase-1, caspase-3, caspase-8 and caspase-9. In aspects, the apoptosis-facilitating polypeptide is a caspase-9 polypeptide, or a functional fragment thereof. In aspects, the apoptosis-facilitating polypeptide is a caspase-9 polypeptide fragment lacking a CARD domain.

In certain aspects, the cells provided herein include a switch polypeptide or nucleic acid encoding a switch polypeptide capable of inducing cell stimulation after the cell is contacted with a multimeric agent capable of binding to the switch polypeptide. In certain aspects, the switch polypeptide contains, and/or nucleic acids that encode the switch polypeptide encode, (i) a first polypeptide capable of binding to a multimeric agent, and (ii) a second polypeptide capable of stimulating the cell upon multimeric agent-induced multimerization of the switch polypeptide. In aspects, the switch polypeptide contains, and/or nucleic acids that encode the switch polypeptide encode, a third polypeptide capable of binding to the multimeric agent or a third polypeptide capable of binding to a multimeric agent different than the multimeric agent to which the first polypeptide binds.

In certain aspects, the cells provided herein contain, and/or contain one or more nucleic acids that encode, (a) a first switch polypeptide comprising (i) a first polypeptide capable of binding to a multimeric agent, and (ii) a second polypeptide capable of stimulating the cell upon multimeric agent-induced multimerization of the switch polypeptide; and (b) a second switch polypeptide containing (1) a third polypeptide capable of binding to the multimeric agent, and (2) the second polypeptide capable of stimulating the cell upon multimeric agent-induced multimerization of the switch polypeptide. In certain aspects, the switch polypeptide capable of inducing cell stimulation contains one or more polypeptides capable of stimulating a cell. In aspects, the switch polypeptide contains (i) multiple copies of one type of stimulatory polypeptide, or (ii) one or more copies of one type of stimulatory polypeptide and one or more copies of another type of stimulatory polypeptide.

In certain aspects of the cells provided herein, the polypeptide capable of simulating a cell upon multimeric agent-induced multimerization of the switch polypeptide is chosen independently from among CD27, CD28, ICOS, 4-1BB, CD40, RANK/TRANCE-R, CD3 zeta chain, OX40, a pattern recognition receptor, TRIF, NOD-like receptor, RIG-like helicase, or a functional fragment of the foregoing. In aspects, the functional fragment is a cytoplasmic region of a native polypeptide. In certain aspects, the pattern recognition receptor is a native MyD88 or a MyD88 fragment lacking a TIR region. In certain aspects, the polypeptide capable of binding to a multimeric agent is selected from among (i) a FKBP polypeptide, (ii) a modified FKBP polypeptide (e.g., FKBP(F36V)), (iii) a FRB polypeptide, (iv) a modified FRB polypeptide, (v) a cyclophilin receptor polypeptide, (vi) a modified cyclophilin receptor polypeptide, (vii) a steroid receptor polypeptide, (viii) a modified steroid receptor polypeptide, (ix) a tetracycline receptor polypeptide, (x) a modified tetracycline receptor polypeptide, and (xi) a polypeptide containing complementarity determining regions (CDRs) of an antibody capable of immunospecifically binding to a multimeric agent. In aspects, the modified FKBP polypeptide includes a F36V amino acid substitution.

In certain aspects, the polypeptide capable of binding to a multimeric agent binds to the multimeric agent with an affinity of 100 nM or less. In aspects, the polypeptide capable of binding to a multimeric agent binds to the multimeric agent with an affinity of 10 nM or less. In aspects, the polypeptide capable of binding to a multimeric agent binds to the multimeric agent with an affinity of 1 nM or less.

In certain aspects of the cells containing a switch polypeptide provided herein, the switch polypeptide includes one or more membrane-association components.

Any of the cells provided herein can, in certain aspects, include a triple switch system, e.g., for regulating the therapy mediated by a CAR that is expressed by the cell. In aspects, the triple switch comprises polypeptides, or polynucleotides encoding polypeptides, that include: (1) a switch comprising an inhibitory polypeptide for reversible inhibition of CAR activity; (2) a switch comprising an activating polypeptide for reversible activation of CAR activity; and (3) a switch comprising a polypeptide that triggers apoptosis of the cell. In aspects, components (1), (2) and (3) of the triple switch are orthogonal, i.e., each component of the triple switch is regulated by a ligand that is not cross-reactive with the other two components of the triple switch.

Also provided herein are compositions that contain any of the binding molecules, including the CAR binding molecules, provided herein, any of the nucleic acids provided herein, or any of the cells provided herein. In aspects, the compositions provided herein include a pharmaceutically acceptable carrier, excipient or diluent.

Certain aspects provided herein include any of the binding molecules, including CAR binding molecules, and cells provided herein, for use as a medicament. Also provided herein are any of the binding molecules, including CAR binding molecules, and cells provided herein, for treatment of a cancer.

Also provided herein are uses of any of the binding molecules, including CAR binding molecules, and cells provided herein, for treatment of a cancer. Also provided herein are uses of any of the binding molecules, including CAR binding molecules, and cells provided herein, in the manufacture of a medicament for treating a cancer. The binding molecules provided herein, e.g., the IsoMSLN binding molecules and the chPD1 receptor molecules, can be used singly or in any combination for treatment of a cancer.

Also provided herein are methods for treating a cancer in a subject that includes administering, to a subject in need thereof, any of the binding molecules, including CAR binding molecules, and cells provided herein, singly or in any combination, in a therapeutically effective amount to treat the cancer.

Also provided herein are agents that reduce the level of a mesothelin isoform-2 polypeptide (IsoMSLN) in cells of a subject, for treatment of a cancer, where the mesothelin isoform-2 polypeptide has or contains the sequence of amino acids set forth in SEQ ID NO:129. Also provided herein are uses of such agents that reduce a level of a mesothelin isoform-2 polypeptide in cells of a subject, for treatment of a cancer, where the mesothelin isoform-2 polypeptide has or contains the sequence of amino acids set forth in SEQ ID NO:129.

Also provided herein are methods for treating a cancer in a subject, which include administering to a subject in need thereof an agent that reduces a level of mesothelin isoform-2 polypeptide in cells of a subject, in an amount effective to reduce the level of the mesothelin isoform-2 polypeptide in the cells, where the mesothelin isoform-2 polypeptide has or contains the sequence of amino acids set forth in SEQ ID NO:129.

In certain aspects, in any of the agents, uses or methods provided herein, the agent is any of the binding molecules, including CAR binding molecules, provided herein, or any of the cells provided herein, or any of the compositions provided herein. In aspects, the agent (i) deletes or disrupts one or more copies of a gene in DNA of the cells that encodes the mesothelin isoform-2 polypeptide, and/or (ii) reduces a level of a RNA transcript of a gene in the cells that encodes the mesothelin isoform-2 polypeptide.

For any of the binding molecules, including CAR binding molecules, cells, compositions, uses or methods provided herein, a cancer to be treated with such binding molecules, including CAR binding molecules, cells, compositions, uses or methods can be selected from among a cancer of the ovary, cervix, lung, abdomen, heart, pancreas and/or stomach. In aspects, the cancer is selected from among mesothelioma, ovarian cancer, cervical squamous cell carcinoma, endocervical adenocarcinoma, lung adenocarcinoma, pancreatic adenocarcinoma and/or stomach adenocarcinoma. In certain aspects, the cancer is epithelial ovarian cancer or malignant pleural mesothelioma. In aspects, the cancer is isoform mesothelin epithelial ovarian cancer or isoform mesothelin malignant pleural mesothelioma. In certain aspects, the agent reduces the level of the mesothelin isoform-2 polypeptide to a greater extent than another mesothelin isoform polypeptide in the cells.

Also provided herein are methods for determining the presence, absence or amount of a mesothelin isoform-2 polypeptide (IsoMSLN) that includes the sequence set forth SEQ ID NO:129, or a polynucleotide encoding the polypeptide. In certain aspects, the methods include contacting a biological sample or biological preparation with (i) a binding molecule that specifically binds to the mesothelin isoform-2 polypeptide, and/or (ii) a polynucleotide complementary to the polynucleotide encoding the mesothelin isoform-2 polypeptide or complement thereof. In aspects, the binding molecule is any of the binding molecules, including CAR binding molecules, provided herein. In certain aspects, the methods include contacting the biological sample or biological preparation with two different binding molecules, where each of the binding molecules specifically binds to the mesothelin isoform-2 polypeptide.

In aspects, the methods include administering a therapy to a subject for treating a cancer. In certain aspects, the therapy includes administering an agent to the subject that (i) specifically binds to the mesothelin isoform-2 polypeptide, (ii) deletes or disrupts one or more copies of a polynucleotide of the cells that encodes the mesothelin isoform-2 polypeptide, and/or (iii) reduces a level of a RNA polynucleotide in the cells that encodes the mesothelin isoform-2 polypeptide. In certain aspects, the agent includes any of the binding molecules, including CAR binding molecules, and cells provided herein. In aspects, the cancer is selected from among a cancer of the ovary, cervix, lung, abdomen, heart, pancreas and/or stomach. In certain aspects, the cancer is selected from among mesothelioma, ovarian cancer, cervical squamous cell carcinoma, endocervical adenocarcinoma, lung adenocarcinoma, pancreatic adenocarcinoma and/or stomach adenocarcinoma.

Certain implementations are described further in the following description, examples and claims, and in the drawings.

### BRIEF DESCRIPTION OF THE DRAWINGS

The drawings illustrate certain implementations of the technology and are not limiting. For clarity and ease of illustration, the drawings are not made to scale, and, in some instances, various aspects may be shown exaggerated or enlarged to facilitate an understanding of particular implementations.

FIG. 1 shows the SpliceDiff™ generated expression profile of the uc002cjw transcript (whose translation product is Iso-MSLN) in Transcripts per Million (TPM) in tumor tissues from The Cancer Genome Atlas (TCGA). Upper Whisker: 138.89; Upper Quartile: 69.93; Median: 44.42; Lower Quartile: 23.68; Lower Whisker: 0.27. CESC: Cervical Squamous Cell Carcinoma and Endocervical Adenocarcinoma; LUAD: Lung Adenocarcinoma; MESO: Mesothelioma; OV: Ovarian Cancer; PAAD: Pancreatic Adenocarcinoma; STAD: Stomach Adenocarcinoma.

FIG. 2 shows the SpliceDiff™ generated expression profile of the uc002cjw transcript in adjacent normal (healthy) tissues from the TCGA. LUAD: Lung Adenocarcinoma; LUSC: Lung Squamous Cell Carcinoma; PAAD: Pancreatic Adenocarcinoma.

FIG. 3 depicts the difference in median TPM of the uc002cjw transcript in ovarian cancer (OV) tissues relative to the highest median TPM measured in adjacent healthy tissue that is adjacent to various cancer tissues.

FIG. 4 shows SpliceDiff™ generated expression profile of the uc002cjw transcript in TPMin the adjacent healthy tissues from the Genotype-Tissue Expression (GTEX) program.

FIG. 5 depicts flow cytometry staining of anti-IsoMSLN-specific antibodies on 293T cells overexpressing mesothelin (MSLN) Isoform 1.

FIG. 6 depicts flow cytometry staining of anti-IsoMSLN-specific antibodies on 293T cells overexpressing mesothelin (MSLN) Isoform 2 (IsoMSLN).

FIG. 7 shows the detection of IsoMSLN on a cell surface by anti-IsoMSLN-specific monoclonal antibodies.

FIG. 8 depicts a plasmid construct expressing the scFv of the anti-IsoMSLN antibody 1B6.

FIG. 9 depicts a plasmid construct expressing the scFv of the anti-IsoMSLN antibody 11C11.

FIG. 10 shows the effects of treating IsoMLSN-eGFP HeLa cells with CAR γδ-T cells that express an anti-IsoMSLN scFv.

FIG. 11 depicts the extent of expansion and enrichment of iNKT cells from peripheral blood.

FIG. 12 depicts the percentage of CD3⁺ iNKT+ cells over 21-day culture (12A) and the expansion-fold of CD3+iNKT+ T cells (12B).

FIG. 13 shows the cytotoxicity of iNKT cells against Daudi cancer cells. (13A) Day 3 imaging of Daudi-eGFP tumor cells cocultured at various E to T ratios of 14-day expanded iNKT cells. (13B) In vitro Daudi-eGFP tumor cell growth kinetics in the presence of iNKT cells.

FIG. 14 depicts viral transduction of iNKT cells with the anti-isomesothelin (IsoMSLN) 1B6 CAR construct pKB113, depicted in FIG. 8 (14A), and characterization of the transduced iNKT cell population (14B).

FIG. 15 depicts in vitro cytotoxicity of anti-Isomesothelin CAR iNK-T cells against Iso-mesothelin-expressing tumor cells. (15A) Day 3 imaging of human mesothelioma cell line (NCI-H226) cocultured with various E:T ratios of iNK T cells with or without anti-Iso MSLN CAR (pKB113) expression (increased fluorescence depicted as brighter and lighter dots in grayscale). (15B) NCI-H226 tumor cell growth kinetics in the presence of CAR.iNKT cells. Data were analyzed using IncuCyte® S3 Live-Cell Analysis System. (15C) Intracellular staining of Granzyme B and % iNKT+ Granzyme B+ cells in the co-culture.

FIG. 16 depicts a schematic representation of the chimeric PD1 (chPD1) construct.

FIG. 17 depicts a plasmid construct expressing chimeric PD1.

FIG. 18 depicts the effects of various costimulatory domains on cytokine secretion mediated by chPD1, and demonstrates that inclusion of different costimulatory domains alters cytokine secretion. After 24 hr of co-culture, cytokine secretion was measured by ELISA. Data are representative of three replicates.

FIG. 19 depicts the effects of various costimulatory domains on T cell differentiation mediated by chPD1, and demonstrates that inclusion of different costimulatory domains alters T cell differentiation. After co-culture with RMA cells, T cell differentiation markers were measured by A) RT-PCR or B) flow cytometry. Data are representative of three replicates.

FIG. 20 depicts the effects of various costimulatory domains on in vivo efficacy against mouse tumors mediated by chPD1, and demonstrates that inclusion of different costimulatory domains alters in vivo efficacy. Tumor bearing mice were treated with T cells 5 and 8 days after tumor cell injection and survival was measured. Data are representative of three replicates. RMA=mouse leukemia cell line, B16=mouse melanoma cell line.

FIG. 21 depicts cytolysis of the murine OC cell line, ID8, by chPD1-transduced T cells in vitro, and demonstrates that ChPD1 T cells lyse and secrete proinflammatory cytokines in response to ID8 cells. (21A) Murine ID8-GFP cells were stained with anti-PDL1 (lighter tall peak to the right) or isotype (darker short peak to the left) antibodies and were analyzed using flow cytometry. (21B) WtPD1 (circles) or chPD1 (squares) T cells were used as effector cells with tumor cell targets at the indicated E:T ratios (1:1, 5:1, 25:1) and cell lysis was measured using an LDH assay. ChPD1 T cells had significantly higher specific lysis at all E:T ratios compared to wtPD1 T cells (* p<0.001). (21C) ID8 cells were cultured with wtPD1-(grey) or chPD1-(black) expressing T cells. After 24 hr, secretion of cytokines was measured in cell-free supernatants by ELISA or LEGENDPlex analysis. chPD1 T cells produced higher levels of proinflammatory cytokines compared to wtPD1 T cells when cultured with tumor cells (*p<0.0001). Data are presented as mean+SD and are representative of at least three experiments.

FIG. 22 depicts the effects of chPD1-transduced T cells in vivo on tumor burden and an increase in survival of ID8-tumor bearing mice, and demonstrates that treatment with chPD1 T cells leads to a reduction in tumor burden and an increase in survival of ID8-tumor bearing mice. ID8-GFP cells (5×10⁶) were injected i.p. into C57BL/6 mice on day 0. Mice were treated i.p. with two doses of PBS (circles), wtPD1 (squares) or chPD1 (triangles) T cells (5×10⁶) after fourteen and seventeen days and tumor burden was measured by detecting (22A) the number of GFP+ cells in the peritoneal wash after 8 weeks of tumor growth and (22B) the number of visible tumors on the peritoneal cavity (n=6 for all groups). (22C) Survival of tumor-bearing mice was also measured (n=8 for all groups). chPD1 T cells significantly reduced tumor burden and increased survival compared to wtPD1 T cells (*-p<0.01). Data are presented as mean+SD and are representative of three independent experiments.

FIG. 23 depicts flow-cytometry to determine the purity and transduction efficiency of gdT cells, 48 hours after exposure to an anti-IsoMSLN CAR retroviral vector, and demonstrates that transduced human gamma delta T cells express anti-IsoMSLN CAR. Human γδ T cells were transduced to express the CAR molecule. Left, purity of γδ T cells and right, cell surface expression of CAR was measured by flow cytometry. Cells were stained with anti-CD34 antibodies (grey peak to the far right) or isotype control (black peak to the far left) and were analyzed using flow cytometry. Non-transduced γδ T cells (peak to the left represented by dashes) were used as a control (non-transduced γδ T cells stained with isotype control antibodies). Data are representative of one experiment.

FIG. 24 depicts the effects of (anti-IsoMSLN) CAR transduced gdT cells on tumor growth, and demonstrets the in vivo efficacy of anti-IsoMSLN CAR gdT cells. The dotted vertical line indicates the day when the gdT cells were administered (+15). Graphs show the average values out of 10 mice (Saline, gdT cells, CAR gdT cells), or 5 mice (tumor-free), +/−95% C.I.

FIG. 25 depicts the in vivo pharmacokinetics of CAR-expressing human gamma delta T cells in the blood, following administration of the cells. NCI-H226 human mesothelioma cells (10⁶ cells) were mixed with 50% Matrigel and injected subcutaneously into 8-week-old female athymic Balb/c Nude mice. 15 days later, the mice were treated intravenously with CAR-expressing human gamma delta T cells (5×10⁶ cells), (n=10). (25A) CAR-expressing human gamma delta T cell numbers and (25B) cell surface expression of CD34 on gamma delta T cells were monitored in the blood by flow cytometry 3, 7, 12, 19, and 45 days after T cell injection. (25C) Upon sacrifice (19 days after T cell injection), the numbers of CAR-expressing human gamma delta T cells in the spleen, lymph nodes, and bone marrow were determined. Data are shown as the average number of gamma delta T cells+standard deviation.

FIG. 26 depicts the persistence of (anti-IsoMSLN) CAR gdT cells in the blood, as measured by tumor re-challenging. In vivo persistency of CAR-expressing human gamma delta T cells was determined as follows: when the tumors became undetectable in the CAR gdT group, half of the mice were sacrificed for histopathological examinations, while half of the mice were observed without further interventions for an additional 26 days, after which 5 naïve mice and 5 CAR gd T cell-treated survivors were re-challenged with the same methods used for the first tumor implantation. Circulating CD34/CAR+gdT cells were detected by flow cytometry (26A), tumor volumes were measured (26B), and mice weight was measured (26C) at the indicated time points. Data are shown as the average+standard deviation.

FIG. 27 depicts the expansion and characterization of gdT cells transduced with the chPD1-DAP10 receptor, and demonstrates that transduced human gamma delta T cells express chPD1 receptor and expand in vitro. (27A) Fold-expansion of non-transduced (squares), or chPD1-expressing γδ T cells (triangles), was measured in vitro. (27B and 27C) Human γδ T cells were transduced to express the chPD1 receptor. (27B) Purity of chPD1 γδ T cells and (27C) cell surface expression of PD1 was measured by flow cytometry. Cells were stained with anti-PD-1 antibodies (black) or isotype control (grey) and were analyzed using flow cytometry. Non-transduced γδ T cells (squares) were used as a control. Data are representative of one experiment.

FIG. 28 depicts the responses of gdT cells transduced with the chPD1-DAP10 receptor against various human tumor cell lines, by measuring expression of PD-L1 on human cancer cell lines and healthy cells. Expression of PD-L1 was determined on human cancer cell lines and healthy cells using anti-PD-L1 (black) or isotype control (grey) antibodies. Cells were analyzed using flow cytometry. SKOV-3 cells were incubated with TNFα (black—anti-PD-L1, grey—isotype control) or without TNFα (triangles—anti-PD-L1, circles—isotype control) for 48 hr before flow cytometry analysis was performed. Data are representative of one experiment.

FIG. 29 demonstrates lysis of PD-L1-positive tumor cells by gdT cells transduced with the chPD1-DAP10 receptor. Human gamma delta chPD1-expressing T cells were found to lyse tumor cells. Non-transduced (circles) and chPD1 γδ T cells (squares) were used as effector cells with tumor or healthy cell targets at the indicated E:T ratios (1:1, 5:1, 25:1) and cell lysis was measured using an LDH assay. ChPD1 T cells had significantly higher specific lysis of tumor cell lines at all E:T ratios compared to non-transduced T cells (* p<0.001). Data are presented as mean+SD and are representative of one experiment.

FIG. 30 depicts the secretion of proinflammatory cytokines by human gamma delta chPD1-expressing T cells, in response to tumor cells. Human gamma delta chPD1-expressing T cells were found to secrete proinflammatory cytokines in response to tumor cells. Tumor and healthy cells were cultured with media (open), non-transduced (black), or chPD1 γδ T cells (grey). After 24 hours, secretion of cytokines was measured in cell-free supernatants by ELISA or LEGENDPlex analysis. chPD1 T cells produced higher levels of proinflammatory cytokines compared to non-transduced T cells when cultured with tumor cells (*p<0.0001). Data are presented as mean+SD and are representative of one experiment.

FIG. 31 depicts the phenotype of human gamma delta chPD1-expressing T cells. Human gamma delta chPD1 T cells were found to express central memory differentiation markers. Nontransduced (open) and chPD1 γδ T cells (black) were cultured with SKOV3 pretreated with TNFα or NCI-H226 cells. After 24 hr, expression of T cell differentiation markers were measured by flow cytometry. ChPD1 T cells produced higher levels of proinflammatory cytokines compared to non-transduced T cells when cultured with tumor cells (*p<0.0001). Data are presented as mean+SD and are representative of one experiment.

FIG. 32 depicts the purity and transduction efficiency of gdT cells transduced with a chPD1-DAP10 receptor. Human γδ T cells were transduced to express the chPD1 receptor, and the transduced cells were found to express the chPD1 receptor. Left) Purity of chPD1 γδ T cells and right) cell surface expression of PD1 was measured by flow cytometry. Cells were stained with anti-PD-1 antibodies (black) or isotype control (grey) and were analyzed using flow cytometry. Non-transduced γδ T cells (blue) were used as a control. Data are representative of one experiment.

FIG. 33 depicts the expression of PD-L1 in various target tumor cells, as measured by flow cytometry. Expression of PD-L1 was measured on NCI-H226 tumor cells. Expression of PD-L1 was determined using anti-PD-L1 (black) or isotype control (grey) antibodies. Cells were analyzed using flow cytometry. Data are representative of one experiment.

FIG. 34 depicts the effect of gdT cells transduced with a chPD1-DAP10 receptor on tumor growth in vivo. In vivo efficacy of chPD1 gdT cells is shown. The dotted vertical line indicates the day when the gdT cells were administered (+15). Graphs show the average values out of 10 mice (Saline, gdT cells, chPD1 gdT cells), or 5 mice (tumor-free), +/−95% C.I.

FIG. 35 depicts the pharmacokinetics of chPD1-expressing human gamma delta T cells in blood. In vivo pharmacokinetics of chPD1-expressing human gamma delta T cells is shown. NCI-H226 human mesothelioma cells (10⁶ cells) were mixed with 50% Matrigel and injected subcutaneously into 8-week-old female athymic Balb/c Nude mice. When the tumor size reached ˜150 mm³, the mice were treated intravenously with chPD1-expressing human gamma delta T cells (5×10⁶ cells), (n=10). (35A) ChPD1-expressing human gamma delta T cell numbers and (35B) cell surface expression of the chPD1 receptor on gamma delta T cells were monitored in the blood by flow cytometry 3, 7, 12, and 19 days after T cell injection. (35C) Upon sacrifice (19 days after T cell injection), the number of chPD1-expressing human gamma delta T cells in the spleen, lymph nodes, and bone marrow were analyzed. Data are shown as the average number of gamma delta T cells+1 standard deviation.

### DETAILED DESCRIPTION

### Mesothelins

Provided herein are binding molecules, such as antibodies and chimeric antigen receptors (CARs), that bind to an isoform of mesothelin. Mesothelin (MSLN) is a differentiation antigen whose expression in normal human tissues is limited to mesothelial cells lining the pleura, pericardium and peritoneum. However, mesothelin is highly expressed in several human cancers, including virtually all mesotheliomas and pancreatic adenocarcinomas, and approximately 70% of ovarian cancers and 50% of lung adenocarcinomas. It is a GPI-anchored cell surface glycoprotein that is overexpressed in about 30% of solid tumors.

The mesothelin gene encodes a precursor protein of 71 kDa that is processed to a 31 kDa shed protein called megakaryocyte potentiating factor (MPF) and a 40 kDa fragment, mesothelin, that is attached to the cell membrane by a glycosyl-phosphatidylinositol (GPI) anchor. MPF was isolated from the culture supernatant of a pancreatic cancer cell line and was so named because it stimulated the megakaryocyte colony-forming activity of interleukin-3 in mouse bone marrow cultures. The biologic function of mesothelin, however, is not known.

The human MSLN transcript has at least three isoforms. Isoform 1 encoding 622 amino acids is the predominant transcript detected in normal and tumor tissues. Isoform 2 is the minor transcript using alternatively spliced exons, producing an additional 8-amino acid insertion compared to Isoform 1. Isoform 3 produces a truncated and soluble MSLN.

Using proprietary SpliceDiff™ software, which is part of an integrated bioinformatic and artificial intelligence (A.I.) system such as that described in PCT application PCT/US20/35183, filed on May 29, 2020, it was found that Isoform 2 of MSLN (IsoMSLN; SEQ ID NO:129) is specifically expressed in cancers such as mesothelioma, ovarian cancers and pancreatic cancer and is more selective than Isoform 1, which often is also expressed and upregulated in normal (healthy) tissues. Furthermore, it was found that this alternatively spliced isoform (IsoMSLN) created unique epitopes (SEQ ID NOS:131 and 132) that are not present in MSLN polypeptides produced by translation of other MSLN transcripts.

### Binding Molecules

Provided herein are binding molecules that specifically bind to a polypeptide having the sequence set forth in SEQ ID NO:129, or that includes the sequence set forth in SEQ ID NO:129. Also provided herein are binding molecules that specifically bind to a polypeptide that includes the sequence set forth in SEQ ID NO:131. Also provided herein are binding molecules that specifically bind to a polypeptide that includes the sequence set forth in SEQ ID NO:132. In aspects, the binding molecules provided herein bind to a polypeptide that includes the sequence set forth in SEQ ID NO:131 and the polypeptide further shares 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more sequence identity with SEQ ID NO:129, e.g., sharing between about 70% to about 99%, or between about 75% to about 80%, 85%, 90%, 95%, 96%, 97%, 98%, 99%, 99.5% or more sequence identity with SEQ ID NO:129. In aspects, the binding molecules provided herein bind to a polypeptide that includes the sequence set forth in SEQ ID NO:132 and the polypeptide further shares 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or more sequence identity with SEQ ID NO:129, e.g., sharing between about 70% to about 99%, or between about 75% to about 80%, 85%, 90%, 95%, 96%, 97%, 98%, 99%, 99.5% or more sequence identity with SEQ ID NO:129.

In certain aspects, the binding molecules provided herein are antibodies (e.g., monoclonal antibodies), or antigen-binding fragments thereof. In aspects, the VH and VL domains of the antibodies provided herein are humanized and/or deimmunized so as to exhibit a reduced immunogenicity upon administration to recipient subjects. In aspects, the binding molecules provided herein can include, but are not limited to, bispecific, trispecific or multispecific IsoMSLN-binding molecules, including bispecific diabodies, BiTEs, bispecific antibodies, trivalent binding molecules and the like that include: (i) IsoMSLN binding Variable Domains (VH and VL) and (ii) a domain capable of binding to an epitope of a molecule present on the surface of an effector cell. A binding molecule sometimes includes one or more of the foregoing binding molecules, including a chimeric antigen receptor (CAR). Also provided herein are pharmaceutical compositions that contain any of the IsoMSLN-binding molecules provided herein, and methods involving the use of any of such IsoMSLN-binding molecules in the treatment of a cancer. In aspects, the cancer is ovarian cancer (OV).

In aspects, binding molecules (e.g., antibodies) provided herein are capable of specific binding to IsoMSLN or a fragment thereof that contains at least one antigenic determinant portion. In aspects, the binding molecules provided herein contain the VH sequence set forth in SEQ ID NO:2 and the VL sequence set forth in SEQ ID NO:11. In certain aspects, the binding molecules provided herein contain 1, or any combination of, 2, 3, 4 or 5, or all 6 of the CDR sequences set forth in SEQ ID NOS: 3-5 and 12-14. In aspects, the binding molecules provided herein contain the VH sequence set forth in SEQ ID NO:38 and the VL sequence set forth in SEQ ID NO:47. In certain aspects, the binding molecules provided herein contain 1, or any combination of, 2, 3, 4 or 5, or all 6 of the CDR sequences set forth in SEQ ID NOS:39-41 and 48-50. In aspects, the binding molecule is the 1B6 antibody having the component sequences set forth in SEQ ID NOS:2-9 and 11-18, or a variant thereof that binds to IsoMSLN. In aspects, the binding molecule is the 11C11 antibody having the component sequences set forth in SEQ ID NOS:38-45 and 47-54, or a variant thereof that binds to IsoMSLN.

As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single-chain antibodies, Fab fragments, F(ab′) fragments, disulfide-linked bispecific Fvs (sdFv), intrabodies, and epitope-binding fragments of any of the above. The term “antibody” includes immunoglobulin molecules and immunologically active fragments of immunoglobulin molecules, i.e., molecules that contain an epitope-binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass. Antibodies are capable of “immunospecifically binding” to a polypeptide or protein or a non-protein molecule (or of binding to such molecule in an “immunospecific manner”) due to the presence on such molecule of a particular domain or moiety or conformation (an “epitope”). In the context of antibodies or antigen-binding fragments thereof, or CAR molecules, the terms “immunospecific” or “immunospecifically binding” are used interchangeably herein with “specific” or “specifically binding,” respectively.

An epitope-containing molecule can have immunogenic activity, such that it elicits an antibody production response in an animal; such molecules are termed “antigens”. Examples of epitopes in the IsoMSLN polypeptide include those having the sequences set forth in SEQ ID NO:131 and SEQ ID NO:132. As used herein, an antibody, diabody or other epitope-binding molecule is said to “immunospecifically” bind a region of another molecule (i.e., an epitope) if it reacts or associates more frequently, more rapidly, with greater duration and/or with greater affinity with that epitope relative to alternate epitopes. For example, an antibody that immunospecifically binds to IsoMSLN is an antibody that binds to IsoMSLN with greater affinity, avidity, more readily, and/or with greater duration than it binds to other MSLN isoforms or other polypeptides. It also is understood by reading this definition that, for example, an antibody (or moiety or epitope) that immunospecifically binds to a first target may or may not bind to a second target. As such, “immunospecific binding” does not necessarily require (although it can include) exclusive binding. Generally, but not necessarily, reference to antibody (or CAR molecule) binding means “immunospecific” binding.

The term “monoclonal antibody,” as used herein, refers to a homogeneous antibody population wherein the monoclonal antibody contains amino acids (naturally occurring or non-naturally occurring) that are involved in the selective binding of an antigen. Monoclonal antibodies are specific, being directed against a single epitope (or antigenic site or determinant). The terms “antibody” or “monoclonal antibody,” as used herein, encompass not only intact antibodies/monoclonal antibodies and full-length antibodies/monoclonal antibodies, but also fragments thereof (such as Fab, Fab′, F(ab′)₂, Fv, etc.), single-chain (scFv) binding molecules, mutants thereof, fusion proteins comprising an antibody portion, humanized monoclonal antibodies, chimeric monoclonal antibodies, and any other modified configuration of the immunoglobulin molecule that contains an antigen recognition site of the required specificity and the ability to bind to an antigen. It is not intended to be limited as regards to the source of the antibody or the manner in which it is made (e.g., by hybridoma, phage selection, recombinant expression, transgenic animals, etc.). The term also includes whole immunoglobulins as well as the fragments etc. described above under the definition of “antibody.”

Antibodies, such as polyclonal antibodies and monoclonal antibodies, can be prepared using standard methods (see, e.g., Kohler et al., Nature 256:495-497 (1975); Kohler et al., Eur. J. Immunol. 6:511-519 (1976); and WO 02/46455). For example, to generate polyclonal antibodies, an immune response is elicited in a host animal, to an antigen of interest. Blood from the host animal is then collected and the serum fraction containing the secreted antibodies is separated from the cellular fraction, using methods known to those of skill in the art. To generate monoclonal antibodies, an animal is immunized by standard methods to produce antibody-secreting somatic cells. These cells then are removed from the immunized animal for fusion to myeloma cells. Somatic cells that can produce antibodies, particularly B cells, can be used for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511-519 (1976); Shulman et al., Nature, 276:269-282 (1978); Volk et al., J. Virol., 42:220-227 (1982)). These cell lines have three useful properties. The first is they facilitate the selection of fused hybridomas from unfused and similarly indefinitely self-propagating myeloma cells by having enzyme deficiencies that render them incapable of growing in selective medium that support the growth of hybridomas. The second is they have the ability to produce antibodies and are incapable of producing endogenous light or heavy immunoglobulin chains. A third property is they efficiently fuse with other cells. Other methods for producing hybridomas and monoclonal antibodies are well known to those of skill in the art. It is routine to produce antibodies against any polypeptide, e.g., antigenic marker on an immune cell population.

Typically, monoclonal antibodies are developed in mice, rats or rabbits. The antibodies can be produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization can be cultured for a period of time (e.g., at least 24 hours) prior to their use as an immunogen. Cells can be used as immunogens by themselves or in combination with a non-denaturing adjuvant, such as Ribi (see, e.g., Jennings, V. M. (1995) “Review of Selected Adjuvants Used in Antibody Production,” ILAR J. 37(3):119-125). In general, cells should be kept intact and preferably viable when used as immunogens. Intact cells can allow antigens to be better detected than ruptured cells by the immunized animal. Use of denaturing or harsh adjuvants, e.g., Freud's adjuvant, can rupture cells. The immunogen can be administered multiple times at periodic intervals such as, bi-weekly, or weekly, or can be administered in such a way as to maintain viability in the animal (e.g., in a tissue recombinant). Alternately, existing monoclonal antibodies and any other equivalent antibodies that are immunospecific for a desired pathogenic epitope can be sequenced and produced recombinantly by any means known in the art.

In aspects, an antibody can be sequenced, and the component polynucleotide sequences (or single sequence, in the case of ScFv) can then be cloned into a vector for expression or propagation. The polynucleotide sequence(s) encoding the antibody of interest can be maintained in a vector in a host cell and the host cell can then be expanded and frozen for future use. The polynucleotide sequence(s) of such antibodies can also be used for genetic manipulation to generate multispecific (e.g., bispecific, trispecific and tetraspecific) binding molecules as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody such as 1, 2, 3, 4, 5 or all 6 of the CDR sequences, while swapping the non-human remainder of the antibody with human antibody sequences.

Natural antibodies (such as IgG antibodies) contain two “Light Chains” complexed with two “Heavy Chains.” Each Light Chain contains a Variable Domain (“VL”) and a Constant Domain (“CL”). Each Heavy Chain contains a Variable Domain (“VH”), three Constant Domains (“CH1,” “CH2” and “CH3”), and a “Hinge” Region (“H”) located between the CH1 and CH2 Domains. The basic structural unit of naturally occurring immunoglobulins (e.g., IgG) is thus a tetramer having two light chains and two heavy chains, usually expressed as a glycoprotein of about 150,000 Da. The amino-terminal (“N-terminal”) portion of each chain includes a Variable Domain of about 100 to 110 or more amino acids primarily responsible for antigen recognition. The carboxy-terminal (“C-terminal”) portion of each chain defines a constant region, with light chains having a single Constant Domain and heavy chains usually having three Constant Domains and a Hinge Domain. Thus, the structure of the light chains of an IgG molecule is n-VL-CL-c and the structure of the IgG heavy chains is n-VH-CH1-H-CH2-CH3-c (where n and c represent, respectively, the N-terminus and the C-terminus of the polypeptide).

The Variable Domains of an IgG molecule include complementarity determining regions (“CDR”), which contain the residues in contact with epitope, and non-CDR segments, referred to as framework segments (“FR”), which in general maintain the structure and determine the positioning of the CDR loops so as to permit such contacting (although certain framework residues may also contact antigen). Thus, the VL and VH Domains have the structure n-FR1-CDR1-FR2-CDR2-FR3-CDR3-FR4-c. Polypeptides that are (or may serve as) the first, second and third CDR of the Light Chain of an antibody are herein respectively designated as: CDR_{L}1 Domain, CDR_{L}2 Domain, and CDR_{L}3 Domain. Similarly, polypeptides that are (or may serve as) the first, second and third CDR of the Heavy Chain of an antibody are herein respectively designated as: CDR_{H}1 Domain, CDR_{H}2 Domain, and CDR_{H}3 Domain. Thus, the terms CDR_{L}1 Domain, CDR_{L}2 Domain, CDR_{L}3 Domain, CDR_{H}1 Domain, CDR_{H}2 Domain, and CDR_{H}3 Domain are directed to polypeptides that when incorporated into a protein cause that protein to be able to bind to a specific epitope regardless of whether such protein is an antibody having light and heavy chains or is a diabody or a single-chain binding molecule (e.g., an scFv, a BiTe, etc.), or is another type of protein. Accordingly, as used herein, the term “epitope-binding fragment” denotes a fragment of a molecule capable of immunospecifically binding to an epitope. An epitope-binding fragment can contain any 1, 2, 3, 4, or 5 the CDR Domains of an antibody, or can contain all 6 of the CDR Domains of an antibody and, although capable of immunospecifically binding to such epitope, can in certain aspects exhibit an immunospecificity, affinity or selectivity toward such epitope that differs from that of such antibody. An epitope-binding fragment of an antibody may be a single polypeptide chain (e.g., an scFv), or can include two or more polypeptide chains, each having an amino terminus and a carboxy terminus (e.g., a diabody, a Fab fragment, an Fab₂ fragment, etc.). Unless specifically noted, the order of domains of the binding molecules provided herein is in the “N-terminal to C-Terminal” direction.

Also provided herein are single-chain Variable Domain fragments (“scFv”) containing a humanized or non-humanized IsoMSLN-VL and/or VH Domain. Single-chain Variable Domain (svFv) fragments contain VL and VH Domains that are linked together using a short “Linker” peptide. Such Linkers can be modified to provide additional functions, such as to permit the attachment of a drug or to permit attachment to a solid support. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In aspects, provided herein are the CDR_{H}1, CDR_{H}2, CDR_{H}3, CODR_{L}1, CDR_{L}2, CDR_{L}3, or the VL Domain and/or the VH Domain of humanized variants of the IsoMSLN antibodies provided herein, as well as multispecific-binding molecules that include the same. The term “humanized” antibody refers to a chimeric molecule, generally prepared using recombinant techniques, having an epitope-binding site of an immunoglobulin from a non-human species and a remaining immunoglobulin structure of the molecule that is based upon the structure and/or sequence of a human immunoglobulin. The anti-IsoMSLN antibodies provided herein can, in certain aspects, include humanized, chimeric or caninized variants of the antibodies 1B6, 11C11, 1B1E or 8D4. The polynucleotide sequence of the variable domains of such antibodies (e.g., SEQ ID NOS: 20, 29, 56 and 65) can be used for genetic manipulation to generate such derivatives and to improve the affinity, or other characteristics of such antibodies.

The general principle in humanizing an antibody involves retaining the basic sequence of the epitope-binding portion of the antibody, while swapping the non-human remainder of the antibody with human antibody sequences. There are four general steps to humanize a monoclonal antibody.

These are: (1) determining the nucleotide and predicted amino acid sequence of the starting antibody light and heavy variable domains (2) designing the humanized antibody or caninized antibody, i.e., deciding which antibody framework region to use during the humanizing or canonizing process (3) the actual humanizing or caninizing methodologies/techniques and (4) the transfection and expression of the humanized antibody. See, for example, U.S. Pat. Nos. 4,816,567; 5,807,715; 5,866,692; and 6,331,415.

The epitope-binding site can include either a complete Variable Domain fused onto Constant Domains or only the complementarity determining regions (CDRs) of such Variable Domain grafted to appropriate framework regions. Epitope-binding domains can be wild-type or modified by one or more amino acid substitutions. Such modification eliminates the constant region as an immunogen in human individuals, but the possibility of an immune response to the foreign variable domain remains. Another approach focuses not only on providing human-derived constant regions but to modify the variable domains as well so as to reshape them as closely as possible to human form. It is known that the variable domains of both heavy and light chains contain three complementarity determining regions (CDRs), which vary in response to the antigens in question and determine binding capability, flanked by four framework regions (FRs) which are relatively conserved in a given species and which putatively provide a scaffolding for the CDRs. When non-human antibodies are prepared with respect to a particular antigen, the variable domains can be “reshaped” or “humanized” by grafting CDRs derived from non-human antibody on the FRs present in the human antibody to be modified. In aspects, the humanized antibodies preserve all CDR sequences (for example, a humanized mouse antibody that contains all six CDRs from the mouse antibodies). In certain aspects, humanized antibodies can have one or more CDRs (one, two, three, four, five, or six) that differ in sequence relative to the original antibody.

Polymorphisms have been observed at a number of different positions within antibody constant regions) and, thus, such variants of the binding molecules provided herein are included, in certain aspects. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5). It is contemplated that the antibodies provided herein can incorporate any allotype, isoallotype, or haplotype of any immunoglobulin gene, and are not limited to the allotype, isoallotype or haplotype of the sequences provided herein. Furthermore, in some expression systems, the C-terminal amino acid residue of the CH3 Domain can be post-translationally removed. Accordingly, the C-terminal residue of the CH3 Domain is an optional amino acid residue in the IsoMSLN-binding molecules provided herein. In certain aspects, a binding molecule includes a CH3 Domain or CH3 Domains, does not include a CH3 Domain, does not include CH3 Domains, includes a CH2-CH3 Domain, includes CH2-CH3 Domains (i.e., a Fc Domain), does not include a CH2-CH3 Domain or does not include CH2-CH3 Domains.

In traditional immune function, the interaction of antibody-antigen complexes with cells of the immune system results in a wide array of responses, ranging from effector functions such as antibody dependent cytotoxicity, mast cell degranulation, and phagocytosis to immunomodulatory signals such as regulating lymphocyte proliferation and antibody secretion. These interactions normally are initiated through the binding of the Fc Domain of antibodies or immune complexes to specialized cell surface receptors on hematopoietic cells, and particularly to receptors (singularly referred to as an “Fc gamma receptor” “FcγR,” and collectively as “FcγRs”) found on the surfaces of multiple types of immune system cells (e.g., B lymphocytes, follicular dendritic cells, natural killer cells, macrophages, neutrophils, eosinophils, basophils and mast cells). Such receptors have an “extracellular” portion (which is thus capable of ligating to an Fc Domain), a “transmembrane” portion (which extends through the cellular membrane, and a “cytoplasmic” portion (positioned inside the cell). The diversity of cellular responses triggered by antibodies and immune complexes results from the structural heterogeneity of the three Fc receptors: FcγRI (CD64), CD32A (FcγRIIA), FcγRIIB (CD32B), CD16A (FcγRIIIA) and CD16B (FcγRIIIB). FcγRI (CD64), FcγRIIA (CD32A) and FcγRIII (CD16) are activating receptors such that their ligation to an Fc Domain activates the immune system or enhances the immune response. In contrast, FcγRIIB (CD32B) is an inhibiting receptor; ligation to an Fc Domain inhibits an immune response or dampens an existing immune response. In addition, interaction of an Fc Domain with with the neonatal Fc Receptor (FcRn) mediates the recycling of IgG molecules from the endosome to the cell surface and release into the blood.

CD16 is a generic name for the activating Fc receptors, FcγRIIIA (CD16A) and FcγRIIIB (CD16B). CD16 is expressed by neutrophils, eosinophils, natural killer (NK) cells, and tissue macrophages that bind aggregated but not monomeric human IgG. These receptors bind to the Fc portion of IgG antibodies, thereby triggering the release of cytokines. If such antibodies are bound to the antigen of foreign cells (e.g., tumor cells), then such release mediates the killing of the tumor cell. Since such killing is antibody-dependent, it is termed antibody-dependent cell-mediated cytotoxicity (ADCC).

CD32A (FcγRIIA) are activating Fc receptors that are expressed on macrophages, neutrophils, eosinophils and dendritic cells (and for CD32A, also on platelets and Langerhan cells). In contrast, CD32B (FcγRIIB) is an inhibiting Fc receptor on B lymphocytes (macrophages, neutrophils, and eosinophils).

The ability of the different FcγRs to mediate diametrically opposing functions reflects their structural differences, and in particular whether the FcγR possesses an immunoreceptor tyrosine-based activation motif (“ITAM”) or an immunoreceptor tyrosine-based inhibitory motif (“ITIM”). The recruitment of different cytoplasmic enzymes to these structures dictates the outcome of the FcγR-mediated cellular responses. ITAM-containing FcγRs include FcγRI, FcγRIIA, FcγRIIIA, and activate the immune system when bound to Fc Domains (e.g., aggregated Fc Domains present in an immune complex). FcγRIIB is the only currently known natural ITIM-containing FcγR; it acts to dampen or inhibit the immune system when bound to aggregated Fc Domains. Human neutrophils express the FcγRIIA gene. FcγRIIA clustering via immune complexes or specific antibody cross-linking serves to aggregate ITAMs with receptor-associated kinases which facilitate ITAM phosphorylation. ITAM phosphorylation serves as a docking site for Syk kinase, the activation of which results in the activation of downstream substrates (e.g., PI₃K). Cellular activation leads to release of pro-inflammatory mediators. The FcγRIIB gene is expressed on B lymphocytes; its extracellular domain is 96% identical to FcγRIIA and binds IgG complexes in an indistinguishable manner. The presence of an ITIM in the cytoplasmic domain of FcγRIIB defines this inhibitory subclass of FcγR. Recently, the molecular basis of this inhibition was established. When co-ligated along with an activating FcγR, the ITIM in FcγRIIB becomes phosphorylated and attracts the SH2 domain of the inositol polyphosphate 5′-phosphatase (SHIP), which hydrolyzes phosphoinositol messengers released as a consequence of ITAM-containing FcγR-mediated tyrosine kinase activation, consequently preventing the influx of intracellular Ca⁺⁺. Thus, cross-linking of FcγRIIB dampens the activating response to FcγR ligation and inhibits cellular responsiveness and aborts B-cell activation, B-cell proliferation and antibody secretion is thus aborted.

The functionality of antibodies can be enhanced by generating bispecific antibodies, multispecific antibodies or diabodies, all of which are contemplated in aspects of the binding molecules provided herein. Multispecific antibody-based molecules that can simultaneously bind two separate and distinct antigens (or different epitopes of the same antigen) and/or by generating antibody-based molecule having higher valency (i.e., more than two binding sites) for the same epitope and/or antigen, can haven enhanced functionality compared to the antibodies alone. A wide variety of recombinant multivalent antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (WO 2005/070966, WO 2006/107786A WO 2006/107617A, WO 2007/046893). PCT Publications Nos. WO 2013/174873, WO 2011/133886 and WO 2010/136172 disclose a trispecific antibody in which the CL and CH1 Domains are switched from their respective natural positions and the VL and VH Domains have been diversified (WO 2008/027236; WO 2010/108127) to allow them to bind to more than one antigen. PCT Publications Nos. WO 2013/163427 and WO 2013/119903 disclose modifying the CH2 Domain to contain a fusion protein adduct comprising a binding domain. PCT Publications Nos. WO 2010/028797, WO2010028796 and WO 2010/028795 disclose recombinant antibodies whose Fc Domains have been replaced with additional VL and VH Domains, so as to form trivalent binding molecules. PCT Publications Nos. WO 2003/025018 and WO2003012069 disclose recombinant diabodies whose individual chains contain scFv Domains. PCT Publication Nos. WO 2013/006544 discloses multivalent Fab molecules that are synthesized as a single polypeptide chain and then subjected to proteolysis to yield heterodimeric structures.

PCT Publications Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another).

Additionally, the capability to produce diabodies that differ from natural antibodies in being capable of binding two or more different epitope species is known and understood by those of skill in the art (i.e., exhibiting bispecificity or multispecificity in addition to bivalency or multivalency). The design of a diabody is based on the antibody derivative known as a single-chain Variable Domain fragment (scFv). Such molecules are made by linking Light and/or Heavy Chain Variable Domains by using a short linking peptide. Bird et al. (1988) (“Single-Chain Antigen-Binding Proteins,” Science 242:423-426) describes example of linking peptides which bridge approximately 3.5 nm between the carboxy terminus of one Variable Domain and the amino terminus of the other Variable Domain. Linkers of other sequences have been designed and used (Bird et al. (1988) “Single-Chain Antigen-Binding Proteins,” Science 242:423-426). Linkers can in turn be modified for additional functions, such as attachment of drugs or attachment to solid supports. The single-chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli.

Polynucleotides encoding the scFv of interest can be made by routine manipulations such as ligation of polynucleotides. The resultant scFv can be isolated using standard protein purification techniques known in the art.

In certain aspects, the serum half-life of binding molecules containing Fc Domains can be increased by increasing the binding affinity of the Fc Domain for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered.

In aspects, the binding molecules provided herein contain a variant Fc Domain. In certain aspects, the variant Fc Domain contains at least one amino acid modification relative to a wild-type Fc Domain, such that said molecule has an increased half-life (relative to a molecule containing a wild-type Fc Domain). In aspects, the IsoMSLN-binding molecules provided herein contain a variant IgG Fc Domain, where the variant Fc Domain includes a half-life extending amino acid substitution at one or more positions selected from among 238, 250, 252, 254, 256, 257, 256, 265, 272, 286, 288, 303, 305, 307, 308, 309, 311, 312, 317, 340, 356, 360, 362, 376, 378, 380, 382, 413, 424, 428, 433, 434, 435, and 436. Many mutations capable of increasing the half-life of an Fc Domain-containing molecule are known in the art and include, for example M252Y, S254T, T256E, and combinations thereof. For example, see the mutations described in U.S. Pat. Nos. 6,277,375, 7,083,784; 7,217,797, 8,088,376; U.S. Publication Nos. 2002/0147311; 2007/0148164; and PCT Publication Nos. WO 98/23289; WO 2009/058492; and WO 2010/033279, which are herein incorporated by reference in their entireties.

In certain aspects, the binding molecules provided herein contain a variant Fc Domain that includes one or more amino acid modifications that reduces the affinity of the variant Fc Domain for an FcγR and/or enhances the serum half-life of the binding molecule. In aspects, the variant Fc Domain that exhibits reduced ADCC effector function. In certain aspects, such binding molecules include any 1, 2, 3, or 4 of the substitutions: L234A, L235A, D265A, N297Q, and N297G. In certain aspects, the modifications include at least one substitution selected from the group consisting of: (a) L234A; (b) L235A; (c) L234A and L235A; (d) M252Y; M252Y and S254T; (e) M252Y and T256E; (f) M252Y, S254T and T256E; and (g) K288D and H435K. The amino acid numbering denoted in each of the foregoing variants is that of the EU index as in Kabat.

In certain aspects, the IsoMSLN-binding molecules provided herein are characterized by any one of two, three, four or five of the following criteria:
(1) the ability to immunospecifically bind human IsoMSLN as endogenously expressed on the surface of a cancer cell;
(2) specifically bind human IsoMSLN with an equilibrium binding constant (K_{D}) of 1 nM or less;
(3) specifically bind human IsoMSLN with an on rate (ka) of 1×10⁶ M⁻¹ min⁻¹ or more;
(4) specifically bind human IsoMSLN with an off rate (kd) of 15×10⁻⁴ min⁻¹ or less;
(5) ability to mediate redirected cell killing (e.g., killing of cancer cells expressing IsoMSLN).

### Binding Molecule Assays

The binding molecules provided herein can be assayed for the ability to bind to IsoMSLN by any method known to those of skill in the art. Binding assays can be performed in solution, suspension or on a solid support. For example, IsoMSLN (SEQ ID NO:129) or a fragment thereof that includes an epitope (antigenic determinant, e.g., having the sequence set forth in SEQ ID NO:131 or 132) can be immobilized to a solid support (e.g., a carbon or plastic surface, a tissue culture dish or chip) and contacted with a binding molecule, such as an antibody or a CAR molecule, provided herein. Unbound antibody or target protein can be washed away, and bound complexes can then be detected. Binding assays can be performed under conditions to reduce nonspecific binding, such as by using buffers with a high ionic strength (e.g., 0.3-0.4 M NaCl) and/or with nonionic detergent (e.g., 0.1% Triton X-100 or Tween 20) and/or blocking proteins (e.g., bovine serum albumin or gelatin). Negative controls also can be included in such assays as a measure of background binding. Binding affinities can be determined using quantitative ELISA, Scatchard analysis (Munson et al., (1980) Anal. Biochem., 107:220), surface plasmon resonance, isothermal calorimetry, or other methods known to one of skill in the art (e.g., Liliom et al. (1991) J. Immunol Methods. 143(1):119-25).

Such assays also can be performed, for example, in solution (e.g., Houghten (1992) Bio/Techniques 13:412-421), on beads (Lam (1991) Nature 354:82-84), on chips (Fodor (1993) Nature 364:555-556), on bacteria (U.S. Pat. No. 5,223,409), on spores (U.S. Pat. Nos. 5,571,698; 5,403,484; and 5,223,409), on plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

The binding can be detected using a method that is capable of being quantified such that the level of activity can be assessed. For example, methods of quantitation include, but are not limited to, spectrophotometric, fluorescent and radioactive methods. Such methods measure, for example, colorimetric signals, chemiluminescent signals, chemifluorescent signals or radioactive signals. In aspects, the binding molecules provided herein can be labeled with a detectable moiety or tag to facilitate detection and determination of IsoMSLN binding activity. The skilled artisan can select an appropriate detectable moiety or tag for use in the assays described or known in the art. Any detectable moiety (i.e., tag or other moiety known to one of skill in the art) that is capable of being detected or identified can be linked to the IsoMSLN-binding molecule or fragment thereof to be tested, directly or indirectly, for example using a linker. Linkage can be at the N- or C-terminus of the therapeutic antibody. Examples of tags and moieties are provided in the Table below:

Examples of tags and moieties

| | | # of | Size | SEQ ID |
|---|---|---|---|---|
| Name | Sequence | Residues | (Da) | NO |
| c-Myc | EQKLISEEDL | 10 | 1200 | 201 |
| FLAG | DYKDDDDK | 8 | 1012 | 202 |
| His | HHHHHH | 6 | | 203 |
| HA | YPYDVPDYA | 9 | 1102 | 204 |
| VSV-G | YTDIEMNRLGK | 11 | 1339 | 205 |
| HSV | QPELAPEDPED | 11 | 1239 | 206 |
| V5 | GKPIPNPLLGLDST | 14 | 1421 | 207 |
| Poly Arg | RRRRR | 5-6 | 800 | 208 |
| Strep-tag-II | WSHPQFEK | 8 | 1200 | 209 |
| S | KETAAAKFERQHMDS | 15 | 1750 | 210 |
| 3x FLAG | DYKDHDGDYKDHDIDYKDDDDK | 22 | 2730 | 211 |
| HAT | KDHLIHNVHKEFHAHAHNK | 19 | 2310 | 212 |
| SBP | MDEKTTGWRGGHVVEGLAGELEQLRARL | 38 | 4306 | 213 |
| | EHHPQGQREP | | | |

Binding assays can be performed in solution, by affixing the binding molecules to a solid support, or by affixing IsoMSLN to a solid support. Any solid support binding assay known to the skilled artisan is contemplated for testing the activities of the antibodies provided herein, including, but not limited to, surface plasmon resonance, bio-layer interferometry, immunoassays, binding to tissues using immunofluorescence or immunohistochemistry, solution binding assays, and cell based binding assays using cells that express IsoMSLN (e.g., IsoMSLN-eGFP expressing HeLa cells).

Solution binding assays, including any solution binding assay known to the skilled artisan, can be used to assess binding activity including equilibrium dialysis, competitive binding assays (e.g., Myers et al., (1975) Proc. Nat!. Acad. Sci. USA), radiolabeled binding assays (e.g., Feau et al., (2009) J. Biomol. Screen. 14(1):43-48), calorimetry, including isothermal titration calorimetry (ITO) and differential scanning calorimetry (e.g., Alvarenga et al. (2012) Anal. Biochem 421(1):138-151, Perozzo et al., (2004) J. Recept Signal. Transduct Res. 24(1-2):1-52; Holdgate (2001) Biotechniques 31(1):164-166, 168, 170, Celej et al. (2006) Anal. Biochem. 350(2):277-284), and spectroscopic fluorescence assays, including fluorescence resonance energy transfer (FRET) assays (Wu et al. (2007), J. Pharm. Biomed. Anal. 44(3):796-801). The conditions for binding assays in can be adapted from conditions discussed above for binding assays performed on a solid support.

Immunoassays include competitive and non-competitive assay systems using techniques such as, but not limited to, western blots or immunoblots, such as quantitative western blots; radioimmunoassays; ELISA (enzyme linked immunosorbent assay); Meso Scale Discovery (MSD, Gaithersburg, Maryland); “sandwich” immunoassays; immunoprecipitation assays; ELISPOT; precipitin reactions; gel diffusion precipitin reactions; immunodiffusion assays; agglutination assays; complement-fixation assays; immunoradiometric assays; fluorescent immunoassays; protein A immunoassays; immunohistochemistry; immuno-electron microscopy or liposome immunoassays (LIA). Such assays are routine and well-known in the art (see, e.g., Ausubel et al., Eds, 1994, Current Protocols in Molecular Biology, Vol. 1, John Wiley & Sons, Inc., New York).

In some examples, immunohistochemistry and/or immunofluorescence can be used to assess IsoMSLN binding in animal models. For example, antibody binding to xenograft tumors in a rodent or other animal model can be analyzed. In other examples, immunohistochemistry can be used to assess antibody binding to skin, such as primate skin. In other examples, immunohistochemistry can be used to assess binding to xenograft tumors and primate skin grafts, ex vivo, for example to visually or quantitatively compare binding preferences of the antibody and to determine if the tested antibody exhibits selective or specific binding.

In other examples, an animal model containing a xenograft tumor or skin graft, such as an animal model described herein, can be administered a binding molecule, such as an antibody, provided herein, such as by systemic administration, to assess in vivo binding of the antibody. In such examples, the tissue can be harvested at particular time(s) to assess binding ex vivo by immunohistochemistry or immunofluorescence as described above. In other examples, the administered binding molecule is conjugated to a fluorophore, such as an infrared fluorophore (e.g., DyLight⁷⁵⁵), which is capable of transmitting fluorescence through the skin. In such examples, antibody binding can be visualized in vivo using a fluorescent imaging system such as the IVIS Caliper imaging system, and antibody binding to xenograft tumors and/or primate skin grafts can be assessed. Tissue can subsequently be harvested for ex vivo confirmational immunohistochemical analysis.

Depending on the quantitative assay selected to measure antibody binding, absolute binding can be represented, for example, in terms of optical density (OD), such as from densitometry or spectrophotometry measurements; arbitrary fluorescent units (AFU), such as from fluorescence measurements; or lumens, such as from chemiluminescence measurements. In some examples, the specific activity is calculated by dividing the absolute binding signal by the antibody protein concentration. In some examples, the specific activity is normalized to give a normalized specific activity (NSA) for each antibody by dividing the specific activity of the antibody by the specific activity of a reference antibody, such as an antibody that is not specific for IsoMSLN, or that binds to both IsoMSLn and Isoform 1 of MSLN or is a parental antibody from which the antibody of interest is derived.

Binding activity also can be measured in terms of binding affinity, which can be determined in terms of binding kinetics, such as measuring rates of association (kₐ or kₒₙ) and/or dissociation (k_{d} or k_{off}), half maximal effective concentration (EC₅₀) values, and/or thermodynamic data (e.g., Gibbs free energy, enthalpy, entropy, and/or calculating association (K_{A}) or dissociation (K_{D}) constants. Typically, determination of binding kinetics requires known antibody and IsoMSLN protein concentrations. Rates of association (kₐ) and association constants (K_{A}) are positively correlated with binding affinity. In contrast, rates of dissociation (k_{d}), dissociation constants (K_{D}) and EC₅₀ values are negatively correlated with binding affinity. Thus, higher binding affinity is represented by lower k_{d}, K_{D} and EC₅₀ values.

### Chimeric PD1 Receptor Molecules

In aspects, provided herein are chimeric PD1 receptor (chPD1) molecules. In aspects, the chPD1 molecules provided herein can be used for adoptive cell therapy in cancers, such as hematological cancers and solid malignancies or tumors. In certain aspects, the chPD1 molecules provided herein can be used alone for the treatment of cancers, and in aspects, the chPD1 molecules provided herein can be used in combination with other binding molecules provided herein, such as the IsoMSLN binding molecules provided herein.

At the beginning of 1990s, PD-1 (programmed cell death protein 1 receptor) was first identified as a membrane protein expressed by a T-cell hybridoma undergoing apoptosis. Since then, numerous experimental works have clarified its function: after engaging its ligand, programmed death ligand 1 (PD-L1), PD-1 negatively shuts down the T-cell response. A few years later (early 2000s), it was suggested that the PD-1/PD-L1 signaling could make tumors capable of evading an antigen-specific T-cell response through an upregulation of PD-L1.

Initial phase I clinical studies evaluated humanized monoclonal antibodies (IgG4) capable of binding to PD-1 and PD-L1 as therapies for advanced solid malignancies and identified the first FDA-approved PD-1 inhibitors, nivolumab and pembrolizumab. Since then, these monoclonal antibodies have been approved for the treatment of several tumors, from Hodgkin lymphoma (HL) to head and neck squamous cell carcinoma (HNSCC). The FDA has approved PD-1/PD-L1 inhibitors for renal cell carcinoma (RCC), HNSCC, non-small cell lung cancer (NSCLC), gastric cancer, HL, urothelial carcinoma, and colorectal cancer. Anti-PD-1 antibodies pembrolizumab and nivolumab and anti-PD-L1 antibodies atelizumab, avelumab and durvalumab have been approved for treating multiple tumor types, such as melanoma, NSCLC, RCC, HNSCC, urothelial and hepatocellular carcinoma, CRC and gastric cancer, Merkel cell carcinoma and Hodgkin's lymphoma.

Anti-PD1/PDL1/2 monoclonal antibody therapies however suffer from certain drawbacks: 1) the persistence of the response is directly dependent on the duration of treatment, so repeated infusions are required for a sustained benefit; 2) despite the re-activation of tumor-infiltrating lymphocyte is possible, the occurrence of a memory response is rare; 3) in most cases the clinical benefit is hampered by the lack of an activator response, indicating that suppressing an inhibitory mechanism is not sufficient, and that anti-tumor T-cells require an activator stimulus as well. This observation is supported by the finding that monoclonal antibodies, such as antiCTLA-4, anti-PD-1/PD-L1, show positive outcomes when trialed in difficult-to-treat malignancies, albeit in a minority of patients, independently of the PDL expression status. Accordingly, a combination of PD1/PDLs blockade with T-cell agonists such as the anti-41BB antibody, or the anti-CD40 activating antibody, has been shown to improve the potency of the treatment and significantly benefit its outcome.

One of the relevant mechanisms of resistance to immune checkpoint inhibitors is the inadequate T cell infiltration due to the lack of tumor immunogenicity. The inability of host CD8+ T cells to localize to a tumor can be most simply attributed to an absence of sufficiently immunogenic tumor antigens for T cell recognition. This may be the case in tumors that are either not significantly dedifferentiated from their tissue of origin or possess insufficient mutational burden to express tumor antigens which are able to produce a focused CD8+ T cell response. The resulting absence of T cells that can differentially recognize unique tumor antigens renders such tumors non-responsive to PD-1/PD-L1 blockade therapy, despite they may express high levels of PD ligands. Indeed, tumors with high mutational burden and increased tumor neoantigen expression, such as melanoma, head and neck, NSCLC, bladder, and microsatellite unstable cancers are generally more responsive to anti-PD-1/PD-L1 therapy.

On top of tolerance/resistance to therapy, many patients show relevant side effects, although immune checkpoint inhibitors can result in fewer adverse events than conventional treatments. Immune-related adverse events (irAEs) are typical of immune checkpoint inhibitor treatment and differ from the adverse events from conventional chemotherapy. irAEs include rash, itching, diarrhea, enteritis, hepatitis, thyroiditis, pneumonitis, diabetes, myositis, neuritis, and myasthenia gravis.

A drawback of current antibody-based approaches to overcome the PD-1/PD-L1 immune suppression is the lack of long-lasting protection, due to the progressive reduction of the therapy potency once the administration of the antibody is discontinued, which is the reason for the need of infusions repeated periodically to prevent disease progression.

To overcome the limitations of monoclonal antibodies blocking the PD-1/PD-L1 axis, recent efforts have been directed in engineering the responder T-cells in such a way that they would turn the otherwise inhibitory PD-1 intracellular signaling into an activator one, in response to the same ligands (PD-L1/PD-L2). This strategy can leverage the use of chimeric antigen receptor (CAR) modified T cells. In fact, solid tumor-induced suppression of CAR T-cells function can be largely accounted for by PD-1 upregulation on tumor-infiltrating CAR T-cells. A polypeptide containing the truncated extracellular domain of PD1 and the transmembrane and cytoplasmic signaling domains of CD28 was constructed; in the presence of PD-L1, T-cells showed increased ERK phosphorylation, cytokine secretion, proliferation, and granzyme B release. It has been shown that PD1-CD28 modified T-cells had enhanced anti-tumor efficacy. It also has been shown that the PD1-CD28 chimeric receptor enhanced the secretion of IL2 by T-cells carrying an anti-Mesothelin and an anti-CD19 CAR in a PDL1-dependent manner. Mesothelin CAR-PD1-CD28 T-cells secreted greater than 30-fold more IL2 than Mesothelin CAR T-cells when co-cultured with target cells expressing Mesothelin and PD-L, while CD19 CAR-PD1-CD28 T-cells secreted greater than 10-fold more IL2 than CD19 CAR T-cells when co-cultured with target cells expressing CD19 and Mesothelin. Other similar approaches have also been described, including a PD1-CD28-41 BB switch receptor. Some different approaches include, using CAR T-cells engineered to secrete anti-PD-L1 or anti-PD1 antibodies, or anti-PD1 single-chain antibody fragment (scFv), to block the PD-1/PD-L1 axis selectively on engineered cells.

A drawback of the above strategies is that the cells armored with a PD-1 switch receptor or with the anti-PD-1/PD-L1 antibodies need to co-express a “classical” CAR molecule, since the PD-1 switch receptor is dependent on the activation of the CAR due to the presence of a co-stimulatory domain only (e.g., CD28) and the lack of an activation domain (such as that of CD3z). In other words, the above-described newer approaches are still dependent on tumor antigen expression and recognition by the modified T-cells.

To enhance the anticancer activity of certain therapeutic cells, provided herein are cells that express a PD1 receptor that when bound to the PD ligands activates the T-cell through a CD3z signal boosted by a DAP10-mediated co-stimulation. The chimeric PD-1 receptor (chPD1) provided herein includes both the activation (CD3z) and co-stimulatory signal (Dap10) within the same receptor. These two features overcome the need of combined anti-PD1/PDLs with anti-41 BB/CD40 antibody therapy, and the resistance to such therapies due to the lack of tumor immunoreactive antigens. In certain aspects, therapeutic cells express (i) a chimeric PD1 receptor, and (ii) a binding molecule that specifically binds to a cancer-associated isoform (e.g., a binding molecule that specifically binds to an IsoMeso-2 isoform), which sometimes are referred to as “ALEXIS” or “ALEXIS 1” therapeutic cells.

In aspects, the chimeric PD1 molecules (chPD1) provided herein can include the formula:

PD1 region-transmembrane region-DAP10 region-CD3z region

In certain aspects, the transmembrane region comprises a CD28 transmembrane domain.

In aspects, the CD3z region is of Isoform 1. In aspects, the chPD1 molecules provided herein do not contain a polypeptide linker sequence between the DAP10 region and the CD3z region. In certain aspects, the chPD1 molecules provided herein do not contain a polypeptide linker sequence of 7 amino acids between the DAP10 region and the CD3z region. In aspects, the chPD1 molecules provided herein do not contain the polypeptide linker sequence GVILTAL (SEQ ID NO:215) between the DAP10 region and the CD3z region. In aspects, the chPD1 molecules provided herein include a CD34 tag. In aspects, the CD34 tag precedes the PD1 region, e.g., a formula that includes:

CD34 tag-PD1 region-transmembrane region-DAP10 region-CD3z region

Without being bound by theory, the CD34 tag can facilitate detection of the chimeric PD1 molecules, e.g., by flow cytometry, and/or facilitate purification of cells transduced with the chimeric PD1 molecules. The choice of the co-stimulatory domains to include in CARs of PD1 chimeric receptors affects the T-cell functions, differentiation, and persistence. Among the co-stimulatory domains are CD28, 4-1BE, or other T-cell co-stimulatory domains. Each co-stimulatory domain is unique for its outcome on effector functions and differentiation: 4-1BE promotes the differentiation into a central memory phenotype with prolonged persistence in vivo, whereas the CD28 domain do not persist as long in vivo. In certain aspects, the co-stimulatory domain used in the chPD1 molecules provided herein is DAP10. In aspects, DAP10 signaling has been shown to enhance T cell effector response, induce activation, and trigger differentiation into memory precursor cells. In certain instances, DAP10-containing chPD1 CAR T cells shows prolonged persistence, development of a central memory phenotype and enhanced anti-tumor activity in vivo compared with the same chPD1-CAR cells containing a CD28 co-stimulation domain. Mechanistically, the stimulation of NKG2D/DAP10, unlike that of CD28, induces the activation of mTOR and supports the development of a central memory phenotype.

Comparing the cytokine profile of different CAR cells, secreted cytokines usually include pro-inflammatory IFN-γ, TNF, IL-2, GM-CSF, IL-17, and IL-21, as well as anti-inflammatory IL-10. Unlike most other signaling domains, DAP10 was shown not to induce IL-10 secretion, but to strongly enhance T cell effector functions via pro-inflammatory cytokines (see Example 8).

In aspects, the chPD1 molecules provided herein can be expressed in immune cells, such as gdT cells (γδT cells, used interchangeably herein) or iNKT cells. The use of unconventional T cells such as gdT cells, which do not respond to HLA-peptide complexes can, in certain aspects, allow for allogeneic, “off-the-shelf” therapies. This can simplify the manufacturing procedure, because the harvesting of T cells from the patient can be avoided and they pose a reduced risk of cytokine release syndrome (CRS). Patients with advanced disease undergoing CAR T cell therapy typically are heavily pre-treated, having previously undergone numerous rounds of chemotherapy, which can result in low T cell counts and/or T cells that may not be healthy enough to expand well making it very difficult to manufacture an efficacious CAR T cell product. Additionally, given that many of these patients have advanced disease, a patient may experience disease progression, co-morbidities, or even death in the time it takes to manufacture autologous CAR T cells. An alternative to autologous CAR T cell manufacturing is the use of allogeneic T cells as the cell source. In order to make this approach feasible, expression of the endogenous αβTCR in allogeneic CAR T cells must be blocked as it would likely result in GvHD, unless the donor is a human leukocyte antigen (HLA) match. While αβ T cells function as a part of the adaptive immune system, γδ T cells play roles in both the innate and the adaptive immune systems. γδ T cells are the only innate immune cells expressing a TCR. However, their target recognition is independent of MHC recognition. Lack of MHCI- and MHCII-restriction make γδ T cells attractive candidates for allogeneic cell therapy. To date, numerous preclinical studies have evaluated CAR-modified γδ T cells targeting neuroblastoma, melanoma, B cell malignancies, and epithelial cell adhesion molecule (epCAM)-positive adenocarcinomas. Additionally, expression of a CAR targeting melanoma-associated chondroitin sulfate proteoglycan (MCSP) was established in γδ T cells, and comparable anti-tumor cytotoxicity, lower cytokine secretion was observed in MCSP-CAR-modified yb T cells compared to that from conventional CAR-modified αβ T cells. Reduced pro-inflammatory cytokine secretion is favorable due to anticipated reduced severity of CRS. Lastly, epCAM CAR-modified γδ T cells demonstrated high levels of in vitro cytotoxicity of tumor cell lines when γδ T cells were both fresh and cryopreserved. These studies demonstrate that engineering of γδ T cells is feasible and results in enhanced in vitro and in vivo cytotoxicity upon CAR expression.

In aspects, the chPD1 molecules provided herein can be used to treat cancers, including hematological malignancies and solid tumors. Previous anti-CD19 CAR T cell therapies have shown difficulty in replicating comparable results in patients with solid tumors. The obstacles can be due to many factors, including: a) the complex and immune-suppressive tumor microenvironment, b) the lack of optimal tumor targets that are not also expressed on normal cells and c) the use of autologous, patient-derived cells, which are frequently sub-optimal due to chemotherapy. Provided herein are chPD1 molecules that can overcome these barriers by using, in certain aspects, unconventional, MHC-independent gamma delta T cells and a chimeric PD-1 protein. It was found, in certain aspects, that the chPD1 molecules provided herein could specifically target cancer cells without a CAR for a specific tumor-associated antigen by turning PD-1 immune suppression into T-cell activation and using donor-derived, “off-the-shelf” effector cells (see Example 8). The chimeric PD-1 receptor molecules provided herein can include, in certain aspects, the extracellular portion of PD-1 fused to the intracellular domains of DAP10 and CD3z.

In aspects, the chimeric PD1 molecules provided herein can include the following structural components, e.g.,

PD1 region-CD28 transmembrane region-DAP10 region-CD3z region; and

Signal polypeptide-linker-PD1 region-transmembrane region-DAP10 region-CD3z region,

depicted by one of the following formulae:

Nterm-(PD1 region (extracellular))-(truncated CD28 region (extracellular))-(CD28 transmembrane region)-(DAP10 region (cytoplasmic))-(CD3-zeta region (cytoplasmic))-Cterm, Formula G:

wherein “Nterm” is the N-terminus of the binding molecule and “Cterm” is the C-terminus of the binding molecule.

Nterm-(PD1 signal)-(PD1 region (extracellular))-(truncated CD28 region (extracellular))-(CD28 transmembrane region)-(DAP10 region (cytoplasmic))-(CD3-zeta region (cytoplasmic))-Cterm, Formula H:

wherein “Nterm” is the N-terminus of the binding molecule and “Cterm” is the C-terminus of the binding molecule.

Nterm-(linker 1)-(CD34 tag)-(linker 2)-(PD1 region (extracellular))-(truncated CD28 region (extracellular))-(CD28 transmembrane region)-(DAP10 region (cytoplasmic))-(CD3-zeta region (cytoplasmic))-Cterm, Formula I:

wherein “Nterm” is the N-terminus of the binding molecule and “Cterm” is the C-terminus of the binding molecule.

Nterm-(CD8 signal)-(linker 1)-(CD34 tag)-(linker 2)-(PD1 region (extracellular))-(truncated CD28 region (extracellular))-(CD28 transmembrane region)-(DAP10 region (cytoplasmic))-(CD3-zeta region (cytoplasmic))-Cterm, Formula J:

wherein “Nterm” is the N-terminus of the binding molecule and “Cterm” is the C-terminus of the binding molecule.

In aspects, a chimeric PD1 molecule having a structure of any one of Formula G-J can include one or more of the following polypeptide regions independently chosen from:
a PD1 signal polypeptide of SEQ ID NO:135;
a CD8 signal polypeptide of SEQ ID NO:149;
a linker 1 polypeptide of SEQ ID NO:151;
a CD34 tag polypeptide of SEQ ID NO:153;
a linker 2 polypeptide of SEQ ID NO:155;
a PD1 region (extracellular) polypeptide of SEQ ID NO:137 or SEQ ID NO:157;
a truncated CD28 region (extracellular) polypeptide of SEQ ID NO:139 or SEQ ID NO:159;
a CD28 transmembrane region polypeptide of SEQ ID NO:141 or SEQ ID NO:161;
a DAP10 region (cytoplasmic) polypeptide of SEQ ID NO:143 or SEQ ID NO:163;
a CD3-zeta region (cytoplasmic) polypeptide of SEQ ID NO:99, SEQ ID NO:127, SEQ ID NO:145, SEQ ID NO:165 or SEQ ID NO:194; and
a combination of the foregoing.

### Chimeric Antigen Receptor Binding Molecules

In aspects, the IsoMSLN-binding molecules and PD ligand binding molecules (chimeric PD1 receptors) provided herein are chimeric antigen receptors (CARs). T cells engineered with chimeric antigen receptors (CARs) have emerged as a potent new class of therapeutics for cancer. Since the first clinical reports of their efficacy emerged a few years ago, investigators have focused on the mechanisms and properties that make CARs effective or toxic, and their effects on T cell biology. Novel CAR designs, coupled with improvements in gene transfer technology, incorporating advances in gene editing, have the potential to increase access to engineered cell therapies, as well as improve their potency in hematologic malignancies as well as solid tumors. The receptors are chimeric because they combine both antigen-binding (i.e., IsoMSLN-binding or PD ligand binding) and T-cell activating functions into a single receptor.

The IsoMSLN-binding molecules provided herein can, in aspects, be monospecific single-chain molecules, such as single-chain variable fragments (“anti-IsoMsLN-scFvs”), as discussed above and elsewhere herein or, in certain aspects, the IsoMSLN-binding molecules provided herein can be Chimeric Antigen Receptors (“anti-IsoMSLN-CARs”). CARs are designed in a modular fashion that typically consists of an extracellular target-binding domain, a hinge region, a transmembrane domain that anchors the CAR to the cell membrane, and one or more intracellular domains that transmit activation signals. The extracellular domain has an scFv domain for the recognition of tumor-associated antigens with specificity and affinity. The intracellular domain is derived from the immunoreceptor tyrosine-based activation motif (ITAM) of the TCR complex CD3ζ chain (also referred to herein as “CD3z” chain”), which activates the costimulatory signal. Depending on the number of costimulatory domains, CARs can be classified into first (CD3z only), second (one costimulatory domain+CD3z), or third generation CARs (more than one costimulatory domain+CD3z). Introduction of CAR molecules into a T cell successfully redirects the T cell with additional antigen specificity and provides the necessary signals to drive full T cell activation. Because antigen recognition by CAR T cells is based on the binding of the target-binding single-chain variable fragment (scFv) to intact surface antigens, targeting of tumor cells is not MHC restricted, co-receptor dependent, or dependent on processing and effective presentation of target epitopes.

As discussed above, scFvs can be made by linking Light and Heavy Chain Variable Domains together via a short linking peptide. First-generation CARs typically have the intracellular domain from the CD3ζ-chain, which is the primary transmitter of signals from endogenous TCRs. Second-generation CARs possess additional intracellular signaling domains from various costimulatory protein receptors (e.g., CD28, 41BB, ICOS, etc.) to the cytoplasmic tail of the CAR in order to provide additional signals to the T-cell. Third-generation CARs combine multiple signaling domains, such as CD3z-CD28-41BB or CD3z-CD28-OX40, in order to further augment potency.

Provided herein are anti-IsoMSLN CAR molecules that specifically bind to a polypeptide having the sequence set forth in SEQ ID NO:129, or that includes the sequence set forth in SEQ ID NO:129. Also provided herein are binding molecules that specifically bind to a polypeptide that includes the sequence set forth in SEQ ID NO:131. Also provided herein are binding molecules that specifically bind to a polypeptide that includes the sequence set forth in SEQ ID NO:132. In aspects, the CAR molecules provided herein bind to a polypeptide that includes the sequence set forth in SEQ ID NO:131 and the polypeptide further shares 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:129, e.g., sharing between about 70% to about 99%, or between about 75% to about 80%, 85%, 90%, 95%, 96%, 97%, 98%, 99%, 99.5%, or more, sequence identity with SEQ ID NO:129. In aspects, the CAR molecules provided herein bind to a polypeptide that includes the sequence set forth in SEQ ID NO:132 and the polypeptide further shares 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:129, e.g., sharing between about 70% to about 99%, or between about 75% to about 80%, 85%, 90%, 95%, 96%, 97%, 98%, 99%, 99.5%, or more, sequence identity with SEQ ID NO:129.

In aspects, the CAR molecules provided herein contain the VH sequence set forth in SEQ ID NO:2 and the VL sequence set forth in SEQ ID NO: 11. In certain aspects, the CAR molecules provided herein contain 1, or any combination of, 2, 3, 4 or 5, or all 6 of the CDR sequences set forth in SEQ ID NOS: 3-5 and 12-14. In aspects, the CAR molecules provided herein contain the VH sequence set forth in SEQ ID NO:38 and the VL sequence set forth in SEQ ID NO:47. In certain aspects, the CAR molecules provided herein contain 1, or any combination of, 2, 3, 4 or 5, or all 6 of the CDR sequences set forth in SEQ ID NOS:39-41 and 48-50. In aspects, the CAR molecule has or includes the sequence of amino acids set forth in SEQ ID NO:73 or SEQ ID NO:196. In certain aspects, the CAR molecule has or includes the sequence of amino acids set forth in SEQ ID NO:101 or SEQ ID NO:197. In certain aspects, the CAR molecule has or includes the sequence of amino acids set forth in SEQ ID NO:168 or SEQ ID NO:198.

In certain aspects, the CAR molecules share 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198, e.g., sharing between about 70% to about 99%, or between about 75% to about 80%, 85%, 90%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198. In aspects, the CAR molecules provided herein contain the VH sequence set forth in SEQ ID NO:2 and the VL sequence set forth in SEQ ID NO: 11 and share 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198, e.g., sharing between about 70% to about 99%, or between about 75% to about 80%, 85%, 90%, 95%, 96%, 97%, 98%, 99%, 99.5% or more sequence identity with SEQ ID NO:73, SEQ ID NO:101, SEQ ID NO:168, SEQ ID NO:196, SEQ ID NO:197 or SEQ ID NO:198. In certain aspects, the CAR molecules provided herein contain 1, or any combination of, 2, 3, 4 or 5, or all 6 of the CDR sequences set forth in SEQ ID NOS: 3-5 and 12-14 and share 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198, e.g., sharing between about 70% to about 99%, or between about 75% to about 80%, 85%, 90%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198. In aspects, the CAR molecules provided herein contain the VH sequence set forth in SEQ ID NO:38 and the VL sequence set forth in SEQ ID NO:47 and share 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198, e.g., sharing between about 70% to about 99%, or between about 75% to about 80%, 85%, 90%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198. In certain aspects, the CAR molecules provided herein contain 1, or any combination of, 2, 3, 4 or 5, or all 6 of the CDR sequences set forth in SEQ ID NOS:39-41 and 48-50 and share 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198, e.g., sharing between about 70% to about 99%, or between about 75% to about 80%, 85%, 90%, 95%, 96%, 97%, 98%, 99%, 99.5%, or more, sequence identity with SEQ ID NO:73, SEQ ID NO:101, SEQ ID NO:168, SEQ ID NO:196, SEQ ID NO:197 or SEQ ID NO:198.

### Cells Containing Binding Molecules

Provided herein, in certain aspects, are cells that contain a binding molecule described herein, and optionally, a polynucleotide encoding a binding molecule described herein. Any type of immune cells can express a CAR molecule provided herein including, but not limited to, αβ-T cells, γδ-T cells, natural killer cells (NK cells), natural killer T cells, iNKT cells and macrophages. In aspects, the cells are γδ-T cells. Provided herein, in aspects, are immune cells that express a CAR molecule provided herein.

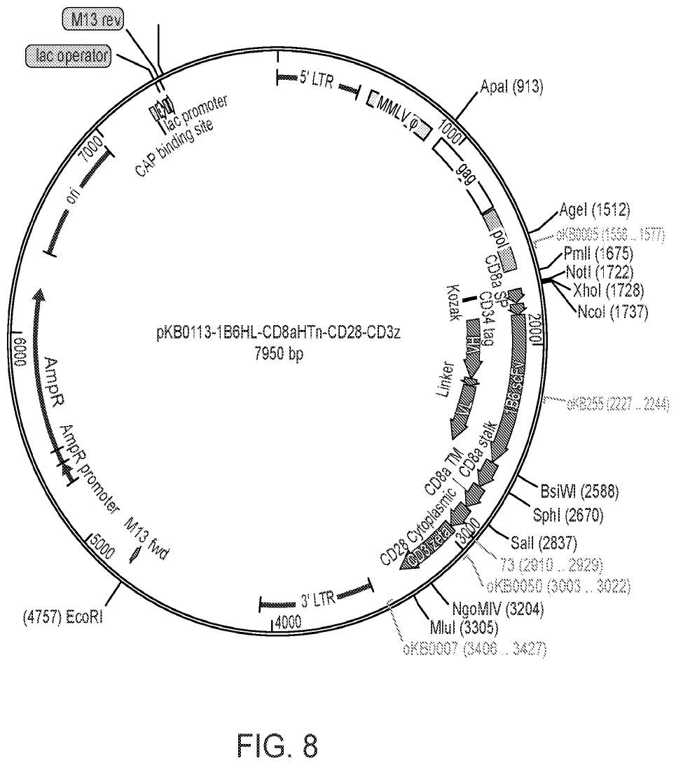

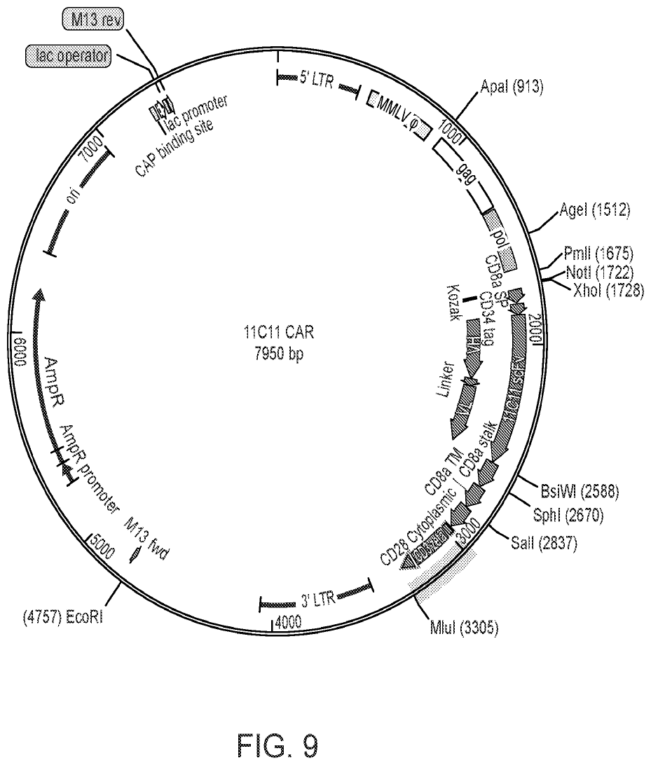

CAR-T cells can be manufactured by generating a single-chain variable fragment (scFv) that recognizes tumor-associated antigen (TAA) recombinants and an intracellular, recombinant “immunoreceptor tyrosine activation motif” (ITAM) region, which are incorporated into a recombinant plasmid in vitro. Examples of polynucleotide sequences encoding CAR molecules are set forth in SEQ ID NOS:74 and 102. Examples of plasmid constructs expressing CAR molecules provided herein are shown in FIGS. 8 and 9. Subsequently, the recombinant plasmid can be transduced into T cells, allowing T cells, such as γδ-T cells, to express the appropriate tumor surface antigen receptors (i.e., anti-IsoMSLN binding molecule), and T cells are expanded after transfection. An example of a protocol is overviewed below:

The first step in the production of CAR-T cells is the isolation of T cells, such as γδ-T cells, from human blood. The CAR-T cells can be manufactured either from the subject's own blood (subject to be treated, i.e., patient), for autologous treatment, or from the blood of a healthy donor, for allogeneic treatment.

Leukocytes can be isolated using a blood cell separator in a process such as, for example, leukocyte apheresis. Peripheral blood mononuclear cells (PBMC) can then be separated and collected. The products of leukocyte apheresis can then be transferred to a cell-processing center. In the cell processing center, specific T cells can be stimulated so that they will actively proliferate and expand to large numbers. To drive their expansion, T cells typically are treated with the cytokine interleukin 2 (IL-2) and anti-CD3 antibodies. In aspects, the T cells can be treated with the cytokine interleukin 2 (IL-2) and the cytokine 7 (IL-7), to drive their expansion. In aspects, the expansion conditions can include bisphosphonates including, but not limited to, zoledronic acid/zoledronate, pamidronate and risedronate. In aspects, the expansion conditions include zoledronic acid or zoledronate.

The expanded T cells can be purified and then transduced with a gene encoding the engineered CAR via a retroviral vector, typically either an integrating gammaretrovirus (RV) or a lentiviral (LV) vector, which generally are rendered safe by partial deletion of the U3 region. Alternately, CRISPR gene editing tools, such as CRISPR/Cas9, can be used instead of retroviral vectors to integrate the CAR gene into specific sites in the genome.

An example of an overview of the manufacture of cells containing binding molecules, such as γδ-T cells or iNKT cells, is as described below. It is understood by those of skill in the art that modifications can be made to the method described below and to any of the methods provided herein, such as adjusting the concentration of one or more components or reagents, changing the order in which the steps are performed, etc., while achieving the same or similar desired end result. In any of the methods of isolation/expansion of a cell population as described and/or provided herein, enrichment of a cell population of interest can periodically be monitored during the steps of the method by flow cytometry to detect subpopulations including, but not limited to, lymphoid cells, myeloid cells, subpopulations of lymphoid cells such as T cells, B cells and NK cells, subpopulations of myeloid cells such as monocytes and granulocytes (can be subjected to Ficoll gradient separation if granulocytes are detected at >1%), and subpopulations of T cells such as αβ-T cells, iNKT cells and γδ-T cells, using methods/markers for detection known to those of skill in the art.

A source of PBMCs, such as whole blood, buffy coat or a Leukopak (product obtained by leukapheresis of whole blood that contains a high concentration of one or more cell types including, but not limited to, mononuclear cells, B cells, T cells, stem/progenitor cells, dendtitic cells and other cell types) is used to prepare an enriched cell population, e.g., of γδ-T cells.

On Day 0, the source of PBMCs, such as fresh Leukopak, is subjected to Ficoll gradient separation according to standard methods known to those of skill in the art to obtain mononuclear cells (PBMCs), followed by αβ-T cell depletion using a CliniMACS® Plus device (Miltenyi Biotec, Germany) and following the manufacturer's protocols.

On Days 1-7, the remaining cell population (after depletion of the αβ-T cells) is subjected to primary cell expansion in the presence of IL-2, IL-7 and zoledronic acid.

On Days 7-10, the expanded cell population is subjected to retroviral transduction to introduce one or more binding molecules selected from among those provided herein.

On Days 10-13, the transduced cells are subjected to a second expansion.

On Day 14, the expanded cells can be used, or are cryopreserved for future use.

If frozen Leukopak is used, the protocol can be amended as follows:

On Days 0-7, the frozen Leukopak is thawed and subjected to primary cell expansion in the presence of IL-2, IL-7 and zoledronic acid.

On Day 7, the expanded cell population undergoes αβ-T cell depletion using a CliniMACS® Plus device (Miltenyi Biotec, Germany), followed by retroviral transduction to introduce one or more binding molecules selected from among those provided herein.

On Days 10-14, the transduced cells are subjected to a second expansion.

On Day 14, the expanded cells can be used, or are cryopreserved for future use.

For example, on Day 0, if frozen Leukopak is used, (a) a 10 μL of the Leukopak sample is diluted to 2 mL in thawing medium (10% HSA (human serum albumin) in PBS) and analyzed for granulocyte content using flow cytometry. If the granulocyte content is >1%, the Leukopak is subjected to a Ficoll gradient separation according to standard methods known to those of skill in the art (if fresh Leukopak is used, the sample is subjected to Ficoll gradient separation).

(b) After removal of the 10 μL aliquot of frozen Leukopak, the remaining frozen Leukopak is diluted to a total volume of about 150 mL by injecting thawing medium. The resulting thawed sample is diluted 1:1 in a sterile bottle using CTS medium containing 2% human antibody serum (Valley Biomedical), for a final volume of about 300 mL.

(c) The resulting 300 mL sample is divided into six 50 mL aliquots and centrifuged at 4° C., 300×g, for 10 minutes. 10 mL of the supernatant was stored at −20° C. for a sterility, mycoplasma, endotoxin quality control (QC) check.

(d) The six cell pellets obtained from (c) are combined in one 50 mL tube and resuspended in 40 mL CTS medium containing 2% human antibody serum by mixing gently, about ten times.

(e) 2 mL of the resuspended cells from (d) are placed in a separate tube. Three 50 μL aliquots are diluted 1:1 with AOPI (acridine orange propidium iodide) staining solution for a cell counting analysis. The total number of cells are counted, and the density and viability calculated. The cell count generally is in the range of about 60×10⁶ cells/mL

(f) About 4×10⁶ cells (about 66.6 μL sample) is subjected to flow cytometry analysis. If the granulocyte content is ≤1%, Ficoll gradient separation is not necessary.

(g) If the granulocyte content is >1%, the resuspended cells are subjected to Ficoll gradient separation. To 30 mL resuspended cells is added 15 mL Ficoll, followed by Ficoll gradient separation.

(h) Following Ficoll gradient separation, the MNCs (mononuclear cells) at the interface are collected and washed with CTS medium supplemented with 2% human antibody serum. A cell count is performed as described in (e). If Ficoll gradient separation is not performed, the resuspended cells from (e) are directly processed according to the next steps.

(i) An aliquot of 2×10⁶ cells is subjected to flow cytometry analysis.

(j) The cell suspension is divided into 2 sterile 250 mL tubes. The volume of each tube is adjusted to 200 mL using Running Buffer (CliniMACs PBS/EDTA buffer supplemented with 0.5% HSA; formulated to a final concentration of 0.5% HSA by adding 20 mL of 25% HSA to each liter of CliniMACs PBS/EDTA buffer). The samples are centrifuged at 4-10° C. for 15 minutes at 400×g.

(k) The supernatant is saved for sterility, mycoplasma, endotoxin testing. The cell pellets are resuspended in 45 mL Running Buffer.

(l) To each tube is added 3.75 ml/tube of CliniMACS TCRα/β Biotin Reagent (Miltenyi Biotec) to label up to 26×10⁹ total cells. The tubes are incubated at room temperature on a rotating shaker at 2 rpm for 30 min.

(m) The volume of each tube is adjusted to 200 ml using Running Buffer, the tubes are centrifuged at 4-10° C. for 15 minutes at 400×g, and the supernatants discarded.

(n) the cell pellets are resuspended to a volume of 45 mL, using Running Buffer, and 7.5 mL CliniMACS Anti-Biotin Reagent is added to each tube. The tubes are incubated at room temperature on a rotating shaker at 2 rpm for 30 min.

(o) The volume of each tube is adjusted to 200 mL, using Running Buffer. The samples are centrifuged at 4-10° C. for 15 minutes at 400×g.

(p) The cell pellets are resuspended and pooled in 150 mL of Running Buffer, or to a volume that results in a cell concentration of between 20×10⁶/mL to 400×10⁶/mL, with a sample loading volume of between 40 mL to 300 mL.

(q) A 0.5 mL aliquot is removed for a cell count and flow analysis (2.2.×10⁶ cells). The cell count and viability is recorded.

(r) The cell suspension is transferred to a 600 mL transfer bag using a spike connector and 50 mL syringes, and the cells are subjected to TCRα/β cell depletion using a CliniMACs plus device and following the operator's manual.

γδT Cell Expansion after αβ TCR T Cell Depletion

(a) CTS™ OpTmizer™ T-Cell Expansion SFM was prepared (add 25 mL CTS OpTmizer Expansion Supplement+2% heat-inactivated human serum+1% Glutamax). 1 Liter of complete medium is prepared to culture 1 billion cells.

(b) Zoledronic acid (ZA) solution can be obtained or prepared by dissolving 4 mg Zoledronic acid powder in 5 ml 0.1 N NaOH (0.8 mg/mL stock solution). Aliquots can be stored at −20° C. for long term storage (up to 1 year). To prepare a 5 μM solution, 51 μl of ZA solution is added to 30 ml of culture medium.

(c) Vials of IL-2 are resuspended with 1 mL of optimizer medium and frozen in aliquots if not used immediately. Human IL-2 (IL-2) and Zoledronic Acid (ZA) are added to complete medium for a final concentration of 300 IU/ml and 5 μM, respectively.

### Cell Culture at Day 0

(d) The cell concentration is adjusted to 1×10⁶ cells/mL in complete medium, with 300 IU/mL IL-2 and 5 μM zoledronic acid added.

(e) The cells are placed in the appropriate vessel, depending on total volume:

| | |
|---|---|
| T25 Flask | 6 mL |
| T75 | 17 mL |
| T175 | 41 mL |

Biofactory (single Layer): 150 mL
Biofactory (double Layer): 300 mL total (150 mL/layer).

(f) The vessels are incubated at 37° C., 5% CO₂

### γδT Cell Expansion in Culture at Day 3

(g) Cell counts and flow cytometry staining are performed as needed on a representative sample of the γδT cell culture.

(h) To the cells of (e) are added human IL-2 (IL-2), human IL-7 (IL-7) and Zoledronic Acid (ZA) to the culture vessels at a final concentration of 300 IU/ml, 500 IU/mL IL7 and 5 μM, respectively.

(i) To coat 6 well plates, 2 mL of RN 20 ug/mL are added per well using a 5 mL serological pipette. To coat 72-AC bags, 40.6 ml of RN 20 mg/ml in PBS1× solution are added.

(j) Retronectin (RN) in PBS1× (20 mg/ml) is prepared. A syringe (20-60 mL) is used to transfer the retronectin solution to the bag.

(k) The bag is placed at 4-8° C. overnight, without flipping.

(l) IL-7 is added to the cell culture at 500 IU/mL.

### Cell Collection at Day 5

(m) The cells are harvested from the culture and placed in appropriately sized tubes (50 mL or 250 mL conical tubes). Cell count and flow cytometry staining analyses are performed as needed on a representative sample of the culture.

γδT Cell CAR Transduction with Retrovirus and Expansion at Day 5.

(n) The RN solution was removed and a retrovirus volume corresponding to 4×10⁶ TU was added per plate (2 mL per plate for an MOI of 2). For plate transduction, the plates were spun for 2 hours at 2000 rpm at 32° C. For transduction in bags, the bags were placed for 4 hours at 4-8° C., after virus addition.

(o) 2×10⁶ gdT cells/well are added at the density of 10⁶/mL in complete medium, along with 500 IU/mL IL-7.

(p) 2-3 days after transduction, the cells are fed with complete medium without IL-7, cell numbers are counted, 2.2×10⁶ cells are taken for cell staining to assess CAR transduction efficiency. The remaining cells are resuspended in the complete culture medium with hIL-2 300 IU/ml (cell concentration adjusted to 10⁶/ml), transferred to G-rex, and cultured at 37° C. in 5% CO₂.

(q) The cells are fed with fresh medium and 300 IU/ml hIL-2 every 3 days. The cell density is maintained at no more than 1×10⁶/ml.

(r) Anti-IFNg antibody (10 ug/mL) is added to PD1 CAR γδT cells.

(s) 2.2×10⁶ cells from the cell suspension is collected as needed during the process to assess and control for cell purity and transduction efficiency (expression of CD34).

(t) On day 8-10, the cells are harvested and placed in 250 and 50 mL conical tubes, as needed. The tubes are spun at 300×g for 10 minutes at RT (room temperature), resuspended in Optimizer complete medium and samples collected for cell counting and flow cytometry analyses. If the percentage of γδT cells is less that 99%, depletion of αβ TCR T cells is repeated.

(u) The resulting product is used, or is frozen for future use.

### Switch Polypeptides

A cell expressing a binding molecule described and provided herein can be configured to further express one or more types of switch polypeptides. In certain implementations, a switch polypeptide serves as a safety switch by facilitating cell elimination. Cell elimination sometimes is desirable should cells expressing a binding molecule described herein induce an adverse event in a subject. An adverse event can be an undesirable immune activity, non-limiting examples of which include undesirably high cytokine activity (e.g., a cytokine storm) and/or graft-versus-host disease (GvHD). In certain implementations, a switch polypeptide serves as an activation switch that facilitates stimulation (e.g., proliferation and/or activation) of cells expressing a binding molecule described herein. In certain implementations, a cell contains a safety switch polypeptide and an activation switch polypeptide. A switch polypeptide may be inactive or exhibit low baseline activity, and activity of a switch polypeptide can be induced and/or significantly increased by induced multimerization of two or more molecules of the switch polypeptide in a cell. Multimerization of a switch polypeptide in a cell can be facilitated by contacting the cell with a multimeric agent. A cell expressing a switch polypeptide may be contacted by a multimeric agent by administering a multimeric agent to the cell (e.g., administering a multimeric agent to a plurality of cells containing one or more cells expressing a switch polypeptide) or a subject containing the cell.

In certain implementations, a cell can express a switch polypeptide that induces cell elimination (e.g., cell death (e.g., apoptosis) and/or cell clearance) after the cell is contacted with a multimeric agent capable of binding to the switch polypeptide. In certain implementations, a cell can be configured to express a switch polypeptide that includes (i) a first polypeptide capable of binding to a multimeric agent, and (ii) a second polypeptide capable of facilitating elimination of the cell upon multimeric agent-induced multimerization of the switch polypeptide. In certain implementations, a switch polypeptide optionally further includes a third polypeptide capable of binding to the multimeric agent to which the first polypeptide is capable of binding, or a third polypeptide capable of binding to a multimeric agent different than the multimeric agent to which the first polypeptide is capable of binding. In certain implementations, a cell can be configured to express (a) a first switch polypeptide that includes (i) a first polypeptide capable of binding to a multimeric agent, and (ii) a second polypeptide capable of facilitating elimination of the cell upon multimeric agent-induced multimerization of the switch polypeptide; and (b) a second switch polypeptide that includes (1) a third polypeptide capable of binding to the multimeric agent to which the first polypeptide is capable of binding, and (2) the second polypeptide capable of facilitating elimination of the cell upon multimeric agent-induced multimerization of the switch polypeptide.

In certain implementations, a polypeptide capable of facilitating cell elimination is a native polypeptide or functional fragment thereof. A polypeptide capable of facilitating cell elimination is an apoptosis-facilitating polypeptide in certain implementations. Non-limiting examples of apoptosis-facilitating polypeptides include Fas, Fas-associated death domain-containing protein (FADD), caspase-1, caspase-3, caspase-8 and caspase-9. An apoptosis-facilitating polypeptide can be a caspase-9 polypeptide, and in certain implementations, can be a caspase-9 polypeptide fragment lacking a CARD domain. An apoptosis-facilitating polypeptide can be a FADD or can be a death effector domain (DED) of FADD in certain implementations. Non-limiting examples of polypeptides capable of facilitating cell elimination are described in Savrou et al., Molecular Therapy 26(5), 1266-1276 (2018); Duong et al., Molecular Therapy: Oncolytics 12, 124-137 (2019); and U.S. Patent Application Publication No. US20160166613A1.

In certain implementations, a cell can express a switch polypeptide that induces cell stimulation (e.g., cell proliferation and/or cell activation) after the cell is contacted with a multimeric agent capable of binding to the switch polypeptide. In certain implementations, a cell can be configured to express a switch polypeptide that includes (i) a first polypeptide capable of binding to a multimeric agent, and (ii) a second polypeptide capable of stimulating the cell upon multimeric agent-induced multimerization of the switch polypeptide. A switch polypeptide optionally can include a third polypeptide capable of binding to the multimeric agent or a third polypeptide capable of binding to a multimeric agent different than the multimeric agent to which the first polypeptide binds. In certain implementations, a cell can be configured to express a first switch polypeptide that includes (i) a first polypeptide capable of binding to a multimeric agent, and (ii) a second polypeptide capable of stimulating the cell upon multimeric agent-induced multimerization of the switch polypeptide; and (b) a second switch polypeptide that includes (1) a third polypeptide capable of binding to the multimeric agent, and (2) the second polypeptide capable of stimulating the cell upon multimeric agent-induced multimerization of the switch polypeptide.

A stimulatory switch polypeptide can include one or more polypeptides capable of stimulating a cell. In certain implementations, a switch polypeptide can include multiple copies of a stimulatory polypeptide. In certain implementations, a switch polypeptide can include one or more copies of one type of stimulatory polypeptide and one or more copies of another type of stimulatory polypeptide. Any suitable polypeptide capable of simulating a cell upon multimeric agent-induced multimerization of the switch polypeptide can be utilized. Sometimes a cell is an immune cell, non-limiting examples of which include T-cells (e.g., gamma.delta T-cells, CD4+ T-cells, CD8+ T-cells), NK cells, invariant natural killer T cells (iNKT), mucosal-associated innate T (MAIT) cells and the like. Non-limiting examples of polypeptides capable of stimulating an immune cell include CD27, CD28, ICOS, 4-1BB, CD40, RANK/TRANCE-R, CD3 zeta chain, OX40, a pattern recognition receptor (e.g., MyD88 (e.g., MyD88 lacking a TIR region), TRIF, NOD-like receptor (e.g., NOD1, NOD2), RIG-like helicase (e.g., RIG-1 or Mda-5)) or functional fragment of the foregoing. A functional fragment sometimes is a cytoplasmic region (e.g., cytoplasmic domain) of a native polypeptide (e.g., cytoplasmic domain of CD40). A stimulatory polypeptide of a switch polypeptide may be considered a co-stimulatory polypeptide in instances where (i) the switch polypeptide includes another type of stimulatory polypeptide, and/or a binding molecule comprises another type of stimulatory molecule, for example. Non-limiting examples of stimulatory polypeptides are described in PCT Application Publication No. WO2014/151960 and PCT Application Publication No. WO2010/033949.

In certain implementations, a polypeptide capable of binding to a multimeric agent is a native polypeptide receptor, or functional fragment thereof, or modified counterpart thereof (e.g., containing one or more point mutations), capable of binding to a small molecule multimeric agent. A polypeptide capable of binding to a multimeric agent sometimes is about 50 amino acids to about 500 amino acids in length (e.g., about 50 amino acids to about 350 amino acids; about 50 amino acids to about 250 amino acids). Non-limiting examples of a polypeptide capable of binding to a multimeric agent include (i) a FKBP polypeptide (i.e., mTOR polypeptide), (ii) a modified FKBP polypeptide (e.g., FKBP(F36V)), (iii) a FRB polypeptide, (iv) a modified FRB polypeptide, (v) a cyclophilin receptor polypeptide, (vi) a modified cyclophilin receptor polypeptide, (vii) a steroid receptor polypeptide, (viii) a modified steroid receptor polypeptide, (ix) a tetracycline receptor polypeptide, (x) a modified tetracycline receptor polypeptide, and (xi) a polypeptide containing complementarity determining regions (CDRs) of an antibody capable of immunospecifically binding to a multimeric agent. Non-limiting examples of a polypeptide containing CDRs of an antibody capable of immunospecifically binding to a multimeric agent include a polypeptide containing a light chain CDR3 and a heavy chain CDR3, optionally containing a light chain CDR1 and a heavy chain CDR1, optionally containing a light chain CDR2 and a heavy chain CDR2, optionally containing one or more light chain framework regions and one or more heavy chain framework regions, and optionally containing a light chain variable domain and a heavy chain variable domain, of an antibody. In certain implementations, a polypeptide capable of binding to a multimeric agent binds to the multimeric agent with an affinity of 500 nM or less, 100 nM or less, 50 nM or less, 5 nM or less, or 1 nM or less, as determined in a suitable in vitro binding assay containing the switch polypeptide and the multimeric agent. Non-limiting examples FKBP, modified FKBP, FRB, modified FRB polypeptides, and combinations of such polypeptides, are described in Clackson et al., PNAS 95, 10437-10442 (1998); Bayle et al., Chemistry & Biology 13, 99-107 (2006); Savrou et al., Molecular Therapy 26(5), 1266-1276 (2018); Duong et al., Molecular Therapy: Oncolytics 12, 124-137 (2019); and U.S. Patent Application Publication No. US20160166613A1.

A switch polypeptide molecule can include one or more polypeptide sub-portions capable of binding to a multimeric agent (e.g., 1, 2, 3, 4, 5, 6, 7, 8, 9 or 10 polypeptides capable of binding to a multimeric agent). In certain implementations, switch polypeptide contains multiple sub-portions of the same type of polypeptide capable of binding to a multimeric agent (e.g., two or three FKBP(F36V) polypeptide sub-portions in each switch polypeptide molecule). In certain implementations, a switch polypeptide contains one or more copies of one type of agent-binding polypeptide (e.g., one or more native FKBP polypeptide sub-portions in each switch polypeptide molecule) and one or more copies of a different type of agent-binding polypeptide (e.g., one FRB or modified FRB polypeptide sub-portions in each switch polypeptide molecule).

A switch polypeptide can contain one or more membrane-association components in certain implementations. A membrane-association component can be a native portion of a polypeptide sub-portion contained in a switch polypeptide. A membrane-association component can be exogenous to other components in a switch polypeptide. Any suitable component can be incorporated into a switch polypeptide that can associate a switch polypeptide with a cell membrane when the switch polypeptide is expressed in the cell. A membrane-association component can be a fatty acid-containing component or lipid-containing component (e.g., a myristoyl-containing region of a polypeptide). A membrane-association component can be a membrane-association region of a transmembrane protein. A switch polypeptide can contain no membrane-association component in certain implementations.

A multimeric agent administered to induce a switch polypeptide activity can be selected according to the agent-binding polypeptide(s) incorporated in the switch polypeptide. For example, (i) a FK506 agent can be administered when a FKBP polypeptide is incorporated into a switch polypeptide, (ii) a FK506 analog agent (e.g., rimiducid (AP1903)) can be administered when a modified FKBP polypeptide (e.g., FKBP(F36V)) is incorporated into a switch polypeptide, and/or (iii) a rapamycin (i.e., sirolimus) or a rapamycin analog (i.e. a rapalog, e.g., temsirolimus, everolimus, ridaforolimus (i.e., defrolimus)) can be administered when a FKBP polypeptide, a FRB polypeptide, a modified FRB polypeptide, or combination of such polypeptides, is incorporated into a switch polypeptide. Non-limiting examples of FK506, FK506 analog, rapamycin, and rapalog multimeric agents are described in Clackson et al., PNAS 95, 10437-10442 (1998); Bayle et al., Chemistry & Biology 13, 99-107 (2006); Savrou et al., Molecular Therapy 26(5), 1266-1276 (2018); Duong et al., Molecular Therapy: Oncolytics 12, 124-137 (2019); and U.S. Patent Application Publication No. US20160166613A1.

A cell can be configured to contain a polynucleotide that encodes a switch polypeptide. A switch polypeptide can be expressed in a cell by induced expression, non-induced expression or a combination of non-induced expression and induced expression, from a polynucleotide that encodes the switch polypeptide. A polynucleotide encoding a switch polypeptide sometimes is incorporated into a circular nucleic acid or non-circular (e.g., linear) nucleic acid prior to incorporation into a cell for expression of the switch polypeptide (e.g., expression plasmid, DNA vector, RNA vector). A polynucleotide encoding a switch polypeptide can be incorporated into a genome of a cell (e.g., by employing a gene editing technology (e.g., CRISPR), for example. In certain implementations, a polynucleotide encoding a switch polypeptide can be incorporated in a cell and not incorporated into a genome of a cell (e.g., incorporation of an expression plasmid in a cell). A polynucleotide encoding a switch polypeptide can be incorporated into a cell using a known technique (e.g., electroporation of nucleic acid; incorporation of naked nucleic acid).

A polynucleotide encoding a switch polypeptide may be present in a nucleic acid in one or more copies. A polynucleotide encoding a switch polypeptide can be present in a cell with one or more exogenous polynucleotides encoding one or more other polypeptide(s) (e.g., an exogenous polynucleotide encoding a binding molecule described herein, an exogenous polynucleotide encoding another type of switch polypeptide). A polynucleotide encoding a switch polypeptide can be present on one nucleic acid containing one or more exogenous polynucleotides encoding the other polypeptide(s), for example. In certain implementations, a polynucleotide encoding a switch polypeptide can be present in one nucleic acid and the one or more exogenous polynucleotides encoding the other polypeptide(s) can be present on one or more other nucleic acids.

### Triple Switch Systems

A cell expressing a binding molecule described and provided herein can be configured to further express a triple switch system comprising switch polypeptides. Chimeric antigen receptor (CAR)-based cellular therapies, such as CAR-T cell therapies, can be very effective in treating cancers and other diseases by their ability to target a specific antigen, for example, a cancer antigen. There however is the danger of toxicity, including immunological toxicity, caused by sustained intense activation of the CAR containing cells, resulting in a macrophage activation syndrome (MAS) and “on-target off-tumor” toxicity that includes unwanted CAR recognition of a target antigen on normal tissues. MAS can be caused by persistent antigen-driven activation and proliferation of T-cells, which in turn releases inflammatory cytokines leading to hyper-activation of macrophages and a feed-forward cycle of immune activation, for example, including a large spike in serum IL-6, resulting in a severe systemic illness. CAR containing cells, such as CAR-T cells, do not have a half-life, so it is not possible to simply cease administration and wait for the cells to breakdown or be excreted. The cells are autonomous and can engraft and proliferate, resulting in a toxicity that can be progressive and fulminant.

Accordingly, there is a need to regulate CAR-mediated therapies in a manner that maximizes their therapeutic efficacy while minimizing toxic side effects. A triple-switch system for use in the cells expressing the binding molecules as described and provided herein can include: (1) a switch comprising an inhibitory polypeptide for reversible inhibition of CAR activity (Switch 1); (2) a switch comprising an activating polypeptide for reversible activation of CAR activity (Switch 2); and (3) a switch comprising a polypeptide that triggers apoptosis of the cell (Switch 3).

A Switch 1 (attenuator switch) can include, for example, parts (a) and (b) as described below:
(a) a polypeptide comprising (i) an FRB domain (FKBP and rapamycin binding domain, i.e., a domain that binds to FKBP (FK506 binding protein) and rapamycin) fused to (ii) a CAR-inhibitory peptide, e.g., an inhibitory tyrosine phosphatase activity such as SHP1; and
(b) a cognate FKBP polypeptide as a domain (e.g., intracellular domain) of a chimeric antigen receptor (CAR), whereby
   administration of a rapamycin or rapamycin analog recruits or joins the inhibitory peptide to the CAR, resulting in reversible attenuation of CAR activity and/or the activity of the CAR-comprising cell.

A Switch 2 (activation switch) can include, for example, parts (a) and (b) as described below:
(a) a Lck tyrosine kinase (lymphocyte specific protein tyrosine kinase) for activating CAR, wherein the Lck tyrosine kinase is truncated at the N-terminus to eliminate ligand-independent membrane association and the N-terminus is replaced by a ligand-dependent membrane association domain. In aspects, the ligand-dependent membrane association domain can be a modified FRB domain, FRB* (e.g., FRBT2098L/FRB_{L}) that can bind to a non-immunosuppressive (NIS) rapamycin analog, wherein the rapamycin analog binds to the modified FRB domain of the activation switch and the rapamycin analog substantially does not bind to the wild-type FRB domain (e.g., mTOR-derived wild-type FRB domain).
   Thus, the NIS rapamycin analog is selective for the activation switch and does not (or minimally) triggers the attenuation switch. While rapamycin and some analogs can bind to both wild-type FRB and the modified FRB* domain (thereby potentially turning on both the attenuation and activation switches), a high ratio of tyrosine phosphatase:tyrosine kinase activity (e.g., a high inducible SHP1 (iSHP1) to inducible Lck (iLck) ratio) should still favor attenuation when rapamycin or other analogs that bind both FRB and FRB* is used. Alternatively, a second orthotopic non-immunosuppressive (NIS) rapalog that binds to a distinct FRB mutant fused to Lck on one side and the FKBP12-fused CAR on the other end can be used.
   In aspects, the truncated Lck tyrosine kinase further comprises a mutation of the tyrosine at position 505 (Y505 mutation, e.g., Y505F), which further increases the activation potential of the Lck tyrosine kinase; and
(b) a cognate FKBP polypeptide as a domain (e.g., intracellular domain) of a chimeric antigen receptor (CAR), whereby administration of the NIS rapamycin analog recruits or joins the Lck tyrosine kinase to the CAR, resulting in reversible induction of CAR activity and/or the activity of the CAR-comprising cell.

While Switch 1 addresses possible deleterious effects of CAR-based treatment, such as cytokine overproduction, it does not address tepid efficacy concerns, or the persistence of therapeutic cells when target ligand is limiting. In response, an inducible activation switch (Switch 2) is used, to provide optimal therapeutic efficacy while minimizing the deleterious effects.

Examples of rapamycin/rapamycin analogs, for use in Switch 1 or Switch 2, or as dimerizer ligands in Switch 3: (1) Sirolimus/Rapamycin (CAS No: 53123-88-9), or (3S,6R,7E,9R,10R,12R,14S,15E,17E,19E,21S,23S,26R,27R,34aS)-9,10,12,13,14, 21,22,23,24,25,26,27,32,33,34,34a-Hexadecahydro-9,27-dihydroxy-3-[(1R)-2-[(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4] oxaazacyclo-hentriacontine-1,5,11,28,29(4H,6H,31H)-pentone; (2) Temsirolimus/CCI-779 (CAS No: 162635-04-3), or 42-[3-Hydroxy-2-(hydroxymethyl)-2-methylpropanoate]-rapamycin; (3) a non-immunosuppressive NIS) rapalog (having a C7 substitution or substitutions) as described, for example, in U.S. Pat. No. 6,187,757; and (4)S-o,p-dimethoxyphenyl (DMOP)-Rapamycin (substituted C7 position).

In the event that an unanticipated high-grade, acute toxicity occurs, the Triple-Switch system includes a polypeptide that can initiate apoptosis to rapidly kill the most activated, toxic cells. A Switch 3 (kill switch) can include, for example, caspase-9, which is activated and initiates apoptosis by dimerization, fused to a polypeptide that binds to a chemical inducer of dimerization (CID). For example, an FKBP12V36-fused caspase-9 (inducible caspase-9, or icaspase9) is homodimerized (activated and initiates apoptosis) when rimiducid (Rimiducid/AP1903 (CAS No: 195514-63-7), or [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl] oxypropyl]phenoxy]acetyl]amino]ethylamino]-2-oxoethoxy] phenyl]propyl] (2S)-1-[(2S)-2-(3,4,5-trimethoxyphenyl)butanoyl]piperidine-2-carboxylate) or a rimiducid analog binds to FKBP12V36, wherein the rimiducid or rimiducid analog cannot bind to a wild type (wt) FKBP12 domain or FRB variant (i.e., cannot trigger Switch 1 or Switch 2).

In aspects, alternative or equivalent chemical inducers of dimerization and binding domains can be used. For example, a CID and CID-binding domain can be any combination of molecules or peptides or domains that enables the selective co-localization and dimerization of a receptor component and a signaling component in the presence of the CID. The CID can be any pharmaceutically acceptable molecule which can simultaneously be bound by at least two binding domains, wherein the CID is capable being delivered to the cytoplasm of a target cell, for example, a T cell or natural killer (NK) cell. Any small molecule dimerization system that can facilitate co-localization of peptides can be used (see, e.g., Corson et al.; 2008; ACS Chemical Biology; 3(11); 667).

The binding moieties of the CID may interact with identical binding domains present on the receptor component and the signaling component, or the CID may comprise two identical binding moieties such that it can simultaneously interact with a binding domain on the receptor component and an identical binding domain on the signaling component. For example, the CID and CID-binding domains can be the FK506 binding protein (FKBP) ligand dimerization system (see e.g., Clackson et al. PNAS; 1998; 95; 10437-10442); dimerization system comprises two FKBP-like binding domains with a F36V mutation in the FKBP binding domain and a dimerization agent (AP1903) with complementary amino acid substitutions. Exposing cells engineered to express FKBP-like binding domain fusion proteins to AP103 results in the dimerization of the proteins comprising the FKBP-like binding domains but no interactions involving endogenous FKBP.

In aspects, a dimerization system as described by Farrar et al., Methods Enzymol., (2000) 327: 421-419 and Nature, (1996) 383:178-181 can be used, which utilizes bacterial DNA gyrase B (GyrB) binding domains and the antibiotic coumermycin as the CID. The binding moieties of the CID may interact with different binding domains on the receptor component and the signaling component, or the CID may comprise two different binding moieties which can simultaneously interact with a binding domain on the receptor component and a different binding domain on the signaling component.

In aspects, a CID and CID-binding domain may comprise the dimerization system described by Belshaw et al. Proc. Natl. Acad. Sci. USA, (1996) 93:4604-4607, which utilizes a FK506 (Tacrolimus)/cyclosporin fusion molecule as the CID agent with FK-binding protein 12 (FKBP12) and cyclophilin A as the binding domains. In certain aspects, a CID/CID-binding domain pairing may also be the rapamycin and FKBP12/FKBP12-Rapamycin Binding (FRB) domain of mTOR system described by Rivera et al., Nature Med., (1996) 2:1028-1032, or the non-immunosuppressive rapamycin analogs (rapalogs) and FKBP12/FRB system described by Bayle et al., Chem. Bio., (2006) 13:99-107. For example, the CID may be C-20-methyllyrlrapamycin (MaRap) or C16(S)-Butylsulfonamidorapamycin (C16-BS-Rap), as described by Bayle et al. in combination with the corresponding binding domains. The CID may be C16-(S)-3-methylindolerapamycin (C16-iRap) or C16-(S)-7-methylindolerapamycin (AP21976/C16-AiRap) as described by Bayle et al., in combination with the respective complementary binding domains for each.

Other dimerization systems that can be used also comprise an estrone/biotin CID in combination with an estrogen-binding domain (EBD) and a streptavidin binding domain, see for example, Muddana & Peterson, Org. Lett., (2004) 6:1409-1412; Hussey et al., J. Am. Chem. Soc., (2003) 125: 3692-3693; a dexamethasone/methotrexate CID in combination with a glucocorticoid-binding domain (GBD) and a dihydrofolate reductase (DHFR) binding domain as described by Lin et al., J.

Am. Chem. Soc., (2000) 122:4247-4248; a system in which the methotrexate portion of the CID is replaced with the bacterial specific DHFR inhibitor trimethoprim as described by Gallagher et al., Anal. Biochem., (2007) 363:160-162; or, an 0⁶-benzylguanine derivative/methotrexate CID in combination with an 0⁶-alkylguanine-DNA alkyltransferase (AGT) binding domain and a DHFR binding domain, as described by Gendreizig et al., J. Am. Chem. Soc., (2003) 125:14970-14971.

Because Switch 3 is an independent safety switch, other safety switches also can be used, such as for example, an HSV-tk or bacterial cytosine deaminase.

In aspects, nucleic acids encoding the polypeptide components of the triple switch systems can be inserted and expressed intracellularly using any expression vehicle, for example, using a vector such as a viral vector, such as a retroviral vector or a lentiviral vector, a plasmid, or a transposon-based vector or synthetic nucleic acid such as a synthetic mRNA. For example, a 2-vector retroviral system or a single lentivirus or equivalent can be used. In aspects, the vector is capable of transfecting or transducing any desired cell, for example, a T cell, a natural killer (NK) cell or other immune cell or somatic cell. The T cell can be a helper T cell, a cytotoxic T cell, a regulatory T cell (Treg cell), a gamma delta T cell, a iNKT cell, or a memory T cell. In alternative embodiments, the cell can be a B cell, a macrophage or a hematopoietic stem cell. Examples of nucleic acids in vectors encoding polypeptide components of the triple switch systems are as follows:

### Reporter Systems

In aspects, the triple switch systems can include assayable reporter proteins that are inducible by transcription factors, such as NF-AT or NF-_{K}B, following cell activation, such as T or NK cell activation. These signaling reporters can be stably integrated into T and NK cell lines (e.g., TALL-104 (T) and NK-92 (NK)) to facilitate selection for clones with the highest S:N (signal:noise), following mitogenic activation.

### Pharmaceutical Compositions, Articles of Manufacture, Kits

Provided herein are pharmaceutical compositions that include any of the anti-IsoMSLN binding molecules, including antibodies or antigen-binding fragments thereof, or a CAR, provided herein, and a pharmaceutically acceptable carrier or excipient. A pharmaceutical composition sometimes includes a chimeric PD1 molecule described herein, optionally in combination with an anti-IsoMSLN binding molecule described herein. In certain aspects, a pharmaceutical composition includes a cell that expresses, or can be induced to express, an anti-IsoMSLN binding molecule described herein, a chimeric PD1 molecule described herein, or combination of such a binding molecule and chimeric molecule. A pharmaceutical composition provided herein can be formulated as a gel, ointment, liquid, suspension, aerosol, tablet, pill, powder or lyophile, and/or can be formulated for systemic, parenteral, topical, oral, mucosal, intranasal, subcutaneous, aerosolized, intravenous, bronchial, pulmonary, vaginal, vulvovaginal, esophageal, or oroesophageal administration. A pharmaceutical composition provided herein can be formulated for single dosage administration or for multiple dosage administration. In certain aspects, a pharmaceutical composition provided herein can be a sustained release formulation.

The pharmaceutical compositions provided herein can be packaged as articles of manufacture containing packaging material, a pharmaceutical composition that is effective for treating a disease, such as a cancer, by administration of an anti-IsoMSLN binding molecule, such as the diseases and conditions described herein or known in the art, and a label that indicates that the binding molecule is to be used for treating the infection, disease or disorder. The pharmaceutical compositions can be packaged in unit dosage forms containing an amount of the pharmaceutical composition for a single dose or multiple doses. In aspects, the packaged compositions can contain a lyophilized powder of the pharmaceutical compositions, which can be reconstituted (e.g., with water or saline) prior to administration.

The pharmaceutical compositions provided herein also can be included in kits. Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. For example, the kits can include an anti-IsoMSLN antibody as provided herein, and can optionally include instructions for use, a device for administering the antibody to a subject, a device for detecting the antibody in a subject, a device for detecting the antibody in samples obtained from a subject, and a device for administering an additional therapeutic agent to a subject. The kit, optionally, can include instructions. Instructions typically include a tangible expression describing the anti-IsoMSLN binding molecules and, optionally, other components included in the kit, and methods for administration, including methods for determining the proper state of the subject, the proper dosage amount, dosing regimens, and the proper administration method for administering the anti-IsoMSLN binding molecules. Instructions also can include guidance for monitoring the subject over the duration of the treatment time.

In aspects, the anti-IsoMSLN binding molecules provided herein can be used as a companion diagnostic, e.g., to detect IsoMSLN in cancer tissue and then treat the cancer with a therapeutic agent such as a chemotherapeutic agent or CAR-T cells. In such aspects, the therapeutic agent can be included in the articles of manufacture and kits provided herein.

For treatment of a disease or condition, such as cancer, the dosage of the anti-IsoMSLN binding molecules, and the frequency of administration, can vary depending on the type and severity of the disease. The binding molecules can be administered in a single dose, in multiple separate administrations, or by continuous infusion. For repeated administrations over several days or longer, depending on the condition, the treatment can be repeated until a desired suppression of disease symptoms occurs or the desired improvement in the patient's condition is achieved.

Repeated administrations can include increased or decreased amounts of the anti-IsoMSLN binding molecule, depending on the progress. For example, anti-IsoMSLN antibodies can be administered at a dosage of about or 0.1 mg/kg to about or 100 mg/kg, such as, for example, about or 0.5 mg/kg to about or 50 mg/kg, about or 5 mg/kg to about or 50 mg/kg, about or 1 mg/kg to about or 20 mg/kg, about or 1 mg/kg to about or 100 mg/kg, about or 10 mg/kg to about or 80 mg/kg, or about or 50 mg/kg to about or 100 mg/kg or more; or at a dosage of about or 0.01 mg/m2 to about or 800 mg/m² or more, such as for example, about or 0.01 mg/m², about or 0.1 mg/m2, about or 0.5 mg/m², about or 1 mg/m², about or 5 mg/m², about or 10 mg/m², about or 15 mg/m2, about or 20 mg/m², about or 25 mg/m², about or 30 mg/m², about or 35 mg/m², about or 40 mg/m2, about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² about or 700 mg/m².

Cells that express a binding molecule (e.g., CAR-T cells provided herein) and/or chimeric PD1 molecule as described herein (e.g., gdT cells or iNKT cells) also can be formulated as a pharmaceutical composition in conjunction with a pharmaceutically acceptable carrier (i.e., pharmaceutical compositions that contain therapeutic cells). The pharmaceutical compositions provided herein can be used for treating diseases such as cancers. Also provided herein are kits containing the pharmaceutical compositions provided herein, including pharmaceutical compositions that contain therapeutic cells, and, optionally, instructions for use. A pharmaceutical composition or kit sometimes includes specific dosage of therapeutic cells, and sometimes the pharmaceutical composition or kit provides a unit dosage of therapeutic cells. The pharmaceutical compositions or kits provided herein can be stored at refrigeration temperatures e.g., 10 degrees Celsius or less, for example, 9, 8, 7, 6, 5, 4, 3, 2, 1 up to negative 4 degrees Celsius or less) or freezing temperatures (e.g., negative 15, 20, 25, 30, 35, 40, 45, 50, 55, 60, 65, 70, 75, 80, 85 degrees or less) as necessary for storage and/or transportation. In certain aspects, the kits contain between about 1×10⁵ cells to about 1×10¹² cells, for example about 1×10⁶, 1×10⁷, 1×10⁸, 1×10⁹ or 1×10¹⁰ cells. In certain aspects, in the pharmaceutical compositions provided herein, or in the kits provided herein that contain the pharmaceutical compositions provided herein, the cells are present in a unit dosage form. In certain aspects, a unit dosage is about 10⁴ to about 10¹⁰ cells per kilogram of weight of an intended subject, or between about 10⁶ to about 10¹² cells per subject (e.g., about 10¹⁰ cells per subject or about 10⁸ cells per kilogram of weight of the intended subject).

Any of the pharmaceutical compositions or kits provided herein can include a pharmaceutically acceptable carrier. The term “pharmaceutically acceptable” means approved by a regulatory agency of a Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A pharmaceutical composition sometimes is provided as a pharmaceutical pack or kit containing one or more containers filled with a therapeutic composition of cells prepared by a method described herein, alone or with such pharmaceutically acceptable carrier. Additionally, one or more other prophylactic or therapeutic agents useful for the treatment of a disease can also be included in the pharmaceutical pack or kit. A pharmaceutical pack or kit may include one or more containers filled with one or more of the ingredients of the pharmaceutical compositions described herein. Optionally associated with such container(s) can be a notice in the form prescribed by a governmental agency regulating the manufacture, use or sale of pharmaceuticals or biological products, which notice reflects approval by the agency of manufacture, use or sale for human administration. A pharmaceutical pack or kit sometimes includes one or more other prophylactic and/or therapeutic agents useful for the treatment of a disease, in one or more containers.

### Methods of Manufacturing Immune Cells, and the Resulting Immune Cell Compositions

Also provided herein, in aspects, are methods of manufacturing enriched compositions containing gamma delta T cells (gdT cells), and the resulting compositions. In certain aspects, the population of cells enriched in gdT cells, which are obtained by the methods provided herein, contains 80% or more gdT cells. In some aspects, between about 80% to about 100%, or at least about 80%, 81%, 82%, 83%, 84%, 85%, 86%, 87%, 88%, 89%, 90%, 91%, 92%, 93%, 94%, 95%, 96%, 97%, 98%, 99% or more, up to 100% of the cells are gdT cells.

The resulting compositions can be used per se in immunotherapy, e.g., for the treatment of cancers as provided herein, or can be modified and used in therapies, such as cancer therapies. For example, the cells in the enriched population can further include a genetic modification containing an exogenous polynucleotide, a mutated polynucleotide, a deleted polynucleotide or combinations thereof. In aspects, the genetic modification includes an exogenous polynucleotide. In aspects, the exogenous polynucleotide expresses the binding molecules provided herein. The exogenous polynucleotide sometimes is in a retroviral vector or a lentiviral vector and, sometimes, the exogenous polynucleotide is integrated into genomes of one or more cells of the modified cell population.

The exogenous polynucleotide can, in certain aspects, encode an exogenous or heterologous T-cell receptor, a tumor necrosis factor receptor, a chimeric antigen receptor (CAR), a myeloid differentiation primary response protein, an innate immune signal transduction adaptor or other protein or polypeptide of interest and can, in some aspects, include a promoter or other regulator of gene expression. In aspects, the exogenous polynucleotide is a regulatory sequence, such as a promoter or enhancer.

In certain aspects, the exogenous polynucleotide encodes a chimeric antigen receptor (CAR) and the cells in the composition comprise a CAR. CARs are recombinant receptors that provide both antigen-binding and T cell activating functions (see, e.g., Sadelain et al., Cancer Discov., 3(4):388-398 (2013)).

The methods of manufacturing enriched gdT cell compositions, as provided herein, include conditions in which the cells are exposed to one or more cytokines whose activity is mediated by all or a portion of the IL-7 receptor. Any source of immune cells can be used in the method provided herein. In certain aspects, the conditions include exposure to IL-7. Without being bound by theory, it is believed that IL-7 or other cytokines whose activity is mediated by all or a portion of the IL-7 receptor can preserve the potential of the gdT cells by reducing exhaustion of the cells. In aspects, exposure of the gdT cells to IL-7 or other cytokines whose activity is mediated by all or a portion of the IL-7 receptor can increase expression of the receptor to which a protein expressed by a transducing retroviral vector, such as RD114, can bind. In aspects, exposure to IL-7 or other cytokines whose activity is mediated by all or a portion of the IL-7 receptor can increase transduction efficiency. In aspects, the methods of manufacturing enriched gdT cell compositions, as provided herein, include conditions in which the cells are exposed to II-7 and/or one or more cytokines whose activity is mediated by all or a portion of the IL-7 receptor, in the absence of IL-15.

In aspects, the source of cells used to prepare a composition enriched in gdT cells is exposed to conditions that include a bisphosphonate in the activation or expansion conditions, such as, but not limited to, clodronate, etidronate, alendronate, pamidronate, zoledronate (zoledronic acid), neridronate and the like. In aspects, the methods provided herein do not include the use of feeder cells.

An example of a method of preparing a composition enriched in gdT cells, as provided herein, includes exposing a sample containing a mixed population of immune cells, such as white blood cells, to a bisphosphonate such as zoledronic acid, IL-2 and IL-7, thereby obtaining an expanded population of gdT cells. In aspects, the expanded population can further be treated to deplete alpha beta T cells in the population, thereby further enriching for the gdT cells. The resulting gdT cell composition can be used in immunotherapy or can be transduced to obtain a genetical modified gdT cell as described elsewhere herein.

Also provided herein, in aspects, are methods of manufacturing enriched compositions containing iNKT cells, and the resulting compositions. In certain aspects, the population of cells enriched in iNKT cells, which are obtained by the methods provided herein, contains 80% or more iNKT cells. In some aspects, between about 80% to about 100%, or at least about 80%, 81%, 82%, 83%, 84%, 85%, 86%, 87%, 88%, 89%, 90%, 91%, 92%, 93%, 94%, 95%, 96%, 97%, 98%, 99% or more, up to 100% of the cells are iNKT cells.

The resulting compositions can be used per se in immunotherapy, e.g., for the treatment of cancers as provided herein, or all or a fraction of the cells can be modified and used in therapies, such as cancer therapies. For example, the cells in the enriched population can further include a genetic modification containing an exogenous polynucleotide, a mutated polynucleotide, a deleted polynucleotide or combinations thereof. In aspects, the genetic modification includes an exogenous polynucleotide. In aspects, the exogenous polynucleotide expresses the binding molecules provided herein. The exogenous polynucleotide sometimes is in a retroviral vector or a lentiviral vector and, sometimes, the exogenous polynucleotide is integrated into genomes of one or more cells of the modified cell population.

The exogenous polynucleotide can, in certain aspects, encode an exogenous or heterologous T-cell receptor, a tumor necrosis factor receptor, a chimeric antigen receptor (CAR), a myeloid differentiation primary response protein, an innate immune signal transduction adaptor or other protein or polypeptide of interest and can, in some aspects, include a promoter or other regulator of gene expression. In some aspects, the exogenous polynucleotide is a regulatory sequence, such as a promoter or enhancer.

In certain aspects, the exogenous polynucleotide encodes a chimeric antigen receptor (CAR) and the cells in the composition comprise a CAR. CARs are recombinant receptors that provide both antigen-binding and T cell activating functions.

Effective chimeric antigen receptor (CAR) T-cell therapy targeting B-cell malignancies has paved the way for alternate strategies for targeting cancer. Current active research is directed towards development of safe, allogeneic off the shelf cell (OTS) therapy products. This could be potentially a step forward in the targeted cancer immunotherapy field. Invariant NKT cells (iNKT) are deemed as one of the unconventional T-cell populations with semi invariantly re-arranged TCR. They recognize lipid antigens via CD1d, an MHC Class 1 like molecule. Recognition of CD1d expressed on various hematopoietic cells is important for targeted tumor specific iNKT cytotoxicity in various leukemia, lymphoma malignancies. The relative percentage of iNKTs is very low in peripheral blood (˜0.01% of T-lymphocytes). Provided herein is an efficient method of enriching and expanding a large and pure population of iNKTs which, in aspects, can be genetically modified for targeting cancers, including haematological malignancies, solid tumors and other cancers known in the art and provided herein. As shown herein (e.g., Example 7), the CAR transduced iNKTs are highly cytotoxic and suggests a central memory phenotype that could potentially persist longer in vivo.

In the methods provided herein, in certain aspects, a highly pure population of of iNKT cells, with over 99% purity for CD3+ iNKT+ cells, can be obtained. The methods provided herein have the potential to produce sufficient iNKT cells for clinical use. In aspects, genetic modification of expanded iNKT cells obtained by the methods provided herein show high cytotoxic potential against Isomesothelin, as measured by in vitro killing and Granzyme B staining. The central memory phenotype of CARiNKTs suggests that, in aspects, they show better persistence in vivo.

In the methods provided herein, in certain aspects, donor screening is performed to select donors whose immune cell samples are more likely to result in a purified, enriched population of iNKT cells (see, e.g., Example 6). In aspects, the methods provided herein, use GMP reagents. Without being bound by theory, it is believed that the use of certain GMP reagents, such as CTS media, in combination with RPMI, can result in a more effective enrichment for iNKT cells. In aspects, the enriched iNKT cell compositions obtained by the methods provided herein are expanded in a co-culture with monocytes, obtained from the same donor, resulting in a significantly pure population of iNKT cells. Without being bound by theory, it is believed that the monocytes can function as APCs, presenting the stimulating molecules (e.g., a-GC, IL-2, IL-21) in a manner that is more effective at expanding the iNKT cell population. In aspects, the methods provided herein avoid the use of tumor feeder cells, such as irradiated K562 cells, thereby reducing the risk of introducing live tumor cells into the resulting iNKT cell composition when used in immunotherapy.

In certain aspects of the methods of enriching for gdT cells or iNKT cells provided herein, a sample obtained from a donor (e.g., a tissue, organ or blood sample from a healthy subject or from a subject who is a patient to be treated with the population of cells). Any source of immune cells can be used as a sample, in the methods provided herein. In certain aspects, the sample is selected from among bone marrow, peripheral blood, liver tissue, epithelial tissue and cord blood. In some aspects of the methods provided herein, the sample is not derived from an embryonic source. In certain aspects of the methods provided herein, the sample is peripheral blood and in some aspects, the peripheral blood sample is a processed sample. For example, the peripheral blood sample can be processed by density gradient centrifugation to separate and/or isolate a buffy coat containing white blood cells, platelets, granulocytes and the like, which then can be subjected to the gdT cell or iNKT cell enrichment methods provided herein. In certain aspects, the buffy coat can further undergo a Ficoll gradient separation to obtain mononuclear cells (PBMCs), which then can be subjected to the gdT cell or iNKT cell enrichment methods provided herein. In some aspects, the peripheral blood sample can undergo apheresis to separate the plasma from the cells, and sometimes the cells then are subjected to the gdT cell or iNKT cell enrichment methods provided herein. In certain aspects of the methods provided herein, the sample is cord blood and sometimes the cord blood is processed cord blood that is processed prior to being subjected to the gdT cell or iNKT cell enrichment methods provided herein.

The term “enriched,” as used herein in reference to the enriched populations of gdT cells and iNKT cells, means that the following two ratios: (i) gdT cells to alpha.beta T cells, and (ii) iNKT cells to alpha.beta T cells in the compositions provided herein are higher than these ratios in nature, e.g., in biological samples such as peripheral blood. In general, as used herein, “enriched” means that the ratio of gdT cells or iNKT cells to alpha.beta T cells in the compositions provided herein is increased by at least 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, or 1000-fold or higher, relative to the ratio in a biological sample, such as a tissue, cord blood or peripheral blood. In certain aspects, the term “enriched” means that the compositions provided herein have a ratio of gdT cells or iNKT cells to alpha.beta T cells of greater than 1 (this ratio generally being less than 1 in nature).

### Methods of Use

The binding molecules provided herein, including the chimeric chPD1 receptor molecules and the IsoMSLN binding molecules and cells transduced with the binding molecules, can be used to treat hematological malignancies, solid tumors and other cancers as provided herein.

The anti-IsoMSLN binding molecules and therapeutic cells (e.g., CAR-T cells) provided herein can be used to diagnose or treat any condition associated with selective expression, specific expression and/or upregulation of expression of IsoMSLN, compared to the corresponding or adjacent normal (healthy) tissues. In certain aspects, the anti-IsoMSLN binding molecules provided herein can be used as a companion diagnostic, e.g., to detect expression of IsoMSLN associated with a disease or condition and then to treat the condition with a second agent, such as a chemotherapeutic agent, immunotherapy, including CAR-T cell therapy, or radiation therapy. Provided herein are methods of treatment by administering, to a subject in need thereof, a therapeutically effective amount of the anti-IsoMSLN binding molecules and/or CAR-T cells provided herein. In certain aspects, provided herein are methods that include screening a subject to detect the selective, specific or upregulated expression of IsoMSLN that is associated with a disease or condition using the anti-IsoMSLN binding molecules provided herein and, if selective, specific or upregulated expression of IsoMSLN is detected, administering a therapeutic agent that treats or ameliorates the disease or condition in the subject.

In any of the methods provided herein, in certain aspects, the disease or condition is cancer. Any cancers that are characterized by selective, specific and/or upregulated expression of IsoMSLN can be diagnosed and/or treated using the anti-IsoMSLN binding molecules and CAR-T cells provided herein. Such cancers can include carcinomas, gliomas, sarcomas (including liposarcoma), adenocarcinomas, adenosarcomas, and adenomas and can occur in virtually all parts of the body, including, for example, breast, heart, lung, small intestine, colon, spleen, kidney, bladder, head and neck, ovary, prostate, brain, pancreas, skin, bone, bone marrow, blood, thymus, uterus, testicles, cervix or liver. Other types of cancers include, but are not limited to, colorectal and head and neck tumors, especially squamous cell carcinoma of the head and neck, brain tumors such as glioblastomas, tumors of the lung, breast, pancreas, esophagus, bladder, kidney, ovary, cervix, and prostate, Kaposi's sarcoma, CNS neoplasms, neuroblastomas, capillary hemangioblastomas, meningiomas and cerebral metastases, melanoma, gastrointestinal and renal carcinomas and sarcomas, rhabdomyosarcoma, glioblastoma (such as glioblastoma multiforme), leiomyosarcoma, lymphoma, blastoma, neuroendocrine tumors, mesothelioma, schwannoma, meningioma, melanoma, leukemia or lymphoid malignancies, hematologic malignancies, such as Hodgkin's lymphoma; non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells, including B-cell acute lymphoblastic leukemia/lymphoma, and T-cell acute lymphoblastic leukemia/lymphoma, thymoma, tumors of the mature T and NK cells, including peripheral T-cell leukemias, adult T-cell leukemia/T-cell lymphomas and large granular lymphocytic leukemia, Langerhans cell histocytosis, myeloid neoplasias such as acute myelogenous leukemias, including AML with maturation, AML without differentiation, acute promyelocytic leukemia, acute myelomonocytic leukemia, and acute monocytic leukemias, myelodysplastic syndromes, and chronic myeloproliferative disorders, including chronic myelogenous leukemia; tumors of the central nervous system such as glioma, glioblastoma, neuroblastoma, astrocytoma, medulloblastoma, ependymoma, and retinoblastoma; solid tumors of the head and neck (e.g., nasopharyngeal cancer, salivary gland carcinoma, and esophageal cancer), lung (e.g., small-cell lung cancer, non-small cell lung cancer, adenocarcinoma of the lung and squamous carcinoma of the lung), digestive system (e.g., gastric or stomach cancer including gastrointestinal cancer, cancer of the bile duct or biliary tract, colon cancer, rectal cancer, colorectal cancer, and anal carcinoma), reproductive system (e.g., testicular, penile, or prostate cancer, uterine, vaginal, vulval, cervical, ovarian, and endometrial cancer), skin (e.g., melanoma, basal cell carcinoma, squamous cell cancer, actinic keratosis), liver (e.g., liver cancer, hepatic carcinoma, hepatocellular cancer, and hepatoma), bone (e.g., osteoclastoma, and osteolytic bone cancers) additional tissues and organs (e.g., pancreatic cancer, bladder cancer, kidney or renal cancer, thyroid cancer, breast cancer, cancer of the peritoneum, and Kaposi's sarcoma), and tumors of the vascular system (e.g., angiosarcoma and hemangiopericytoma). In certain aspects, the cancer is selected from among mesothelioma, ovarian cancer, cervical squamous cell carcinoma, endocervical adenocarcinoma, lung adenocarcinoma, pancreatic adenocarcinoma and/or stomach adenocarcinoma. In aspects, the cancer is an ovarian cancer.

### Certain Implementations

Following are non-limiting examples of certain implementations of the technology.

A1. A binding molecule that specifically binds to a polypeptide of SEQ ID NO:129, comprising the six CDRs of SEQ ID NO:2 and SEQ ID NO:11.

A2. A binding molecule that specifically binds to a polypeptide epitope that includes SEQ ID NO:131 or SEQ ID NO:132, comprising the CDR3 of SEQ ID NO:2 and the CDR3 of SEQ ID NO:11.

A3. The binding molecule of embodiment A2, comprising the CDR1 and CDR2 of SEQ ID NO:2 and the CDR1 and CDR2 of SEQ ID NO:11.

A4. The binding molecule of any one of embodiments A1-A3, comprising a heavy chain variable domain about 70% or more identical to the heavy chain variable domain of SEQ ID NO:2.

A5. The binding molecule of embodiment A4, comprising a heavy chain variable domain about 80% or more identical to the heavy chain variable domain of SEQ ID NO:2.

A6. The binding molecule of embodiment A5, comprising a heavy chain variable domain about 90% or more identical to the heavy chain variable domain of SEQ ID NO:2.

A7. The binding molecule of embodiment A6, comprising a heavy chain variable domain about 95% or more identical to the heavy chain variable domain of SEQ ID NO:2.

A8. The binding molecule of embodiment A7, comprising the heavy chain variable domain of SEQ ID NO:2.

A9. The binding molecule of any one of embodiments A1-A8, comprising a light chain variable domain about 70% or more identical to the light chain variable domain of SEQ ID NO:11.

A10. The binding molecule of embodiment A9, comprising a light chain variable domain about 80% or more identical to the light chain variable domain of SEQ ID NO:11.

A11. The binding molecule of embodiment A10, comprising a light chain variable domain about 90% or more identical to the light chain variable domain of SEQ ID NO:11.

A12. The binding molecule of embodiment A11, comprising a light chain variable domain about 95% or more identical to the light chain variable domain of SEQ ID NO:11.

A13. The binding molecule of embodiment A12, comprising the light chain variable domain of SEQ ID NO:11.

A14. The binding molecule of any one of embodiments A1-A13, comprising the heavy chain variable domain of SEQ ID NO:2 and the light chain variable domain of SEQ ID NO:11.

A15. The binding molecule of any one of embodiments A1-A14, comprising a CDR3 of SEQ ID NO:5 and a CDR3 of SEQ ID NO:14.

A16. The binding molecule of any one of embodiments A1-A15, comprising a CDR1 of SEQ ID NO:3 and a CDR1 of SEQ ID NO:12.

A17. The binding molecule of any one of embodiments A1-A16, comprising a CDR2 of SEQ ID NO:4 and a CDR2 of SEQ ID NO:13.

A18. The binding molecule of any one of embodiments A1-A17, comprising an antibody, antibody fragment, single-chain antibody, diabody, or BiTe.

A19. The binding molecule of embodiment A18, wherein the antibody is chosen from a monoclonal antibody, a polyclonal antibody, a recombinant antibody, an IgE antibody, an IgD antibody, an IgM antibody, an IgG antibody, an antibody comprising at least one amino acid substitution, an antibody comprising at least one non-naturally occurring amino acid, or combination of the foregoing.

A20. The binding molecule of embodiments A19, wherein the antibody is an IgG antibody.

A21. The binding molecule of embodiment A18, wherein the antibody fragment is chosen from an scFv, a Fab, a Fab′, a Fv, a F(ab′)2.

A22. The binding molecule of any one of embodiments A1-A24, which specifically binds to a polypeptide of SEQ ID NO:129 with a binding affinity of 100 nM or less.

A23. The binding molecule of embodiment A22, which specifically binds to a polypeptide of SEQ ID NO:129 with a binding affinity of 10 nM or less.

A24. The binding molecule of embodiment A22, which specifically binds to a polypeptide of SEQ ID NO:129 with a binding affinity of 1 nM or less.

B1. A binding molecule that specifically binds to a polypeptide of SEQ ID NO:129, comprising the six CDRs of SEQ ID NO:38 and SEQ ID NO:47.

B2. A binding molecule that specifically binds to a polypeptide epitope that includes SEQ ID NO:131 or SEQ ID NO:132, comprising the CDR3 of SEQ ID NO:38 and the CDR3 of SEQ ID NO:47.

B3. The binding molecule of embodiment B2, comprising the CDR1 and CDR2 of SEQ ID NO:38 and the CDR1 and CDR2 of SEQ ID NO:47.

B4. The binding molecule of any one of embodiments B1-B3, comprising a heavy chain variable domain about 70% or more identical to the heavy chain variable domain of SEQ ID NO:38.

B5. The binding molecule of embodiment B4, comprising a heavy chain variable domain about 80% or more identical to the heavy chain variable domain of SEQ ID NO:38.

B6. The binding molecule of embodiment B5, comprising a heavy chain variable domain about 90% or more identical to the heavy chain variable domain of SEQ ID NO:38.

B7. The binding molecule of embodiment B6, comprising a heavy chain variable domain about 95% or more identical to the heavy chain variable domain of SEQ ID NO:38.

B8. The binding molecule of embodiment B7, comprising the heavy chain variable domain of SEQ ID NO:38.

B9. The binding molecule of any one of embodiments B1-B8, comprising a light chain variable domain about 70% or more identical to the light chain variable domain of SEQ ID NO:47.

B10. The binding molecule of embodiment B9, comprising a light chain variable domain about 80% or more identical to the light chain variable domain of SEQ ID NO:47.

B11. The binding molecule of embodiment 10, comprising a light chain variable domain about 90% or more identical to the light chain variable domain of SEQ ID NO:47.

B12. The binding molecule of embodiment B11, comprising a light chain variable domain about 95% or more identical to the light chain variable domain of SEQ ID NO:47.

V13. The binding molecule of embodiment B12, comprising the light chain variable domain of SEQ ID NO:47.

B14. The binding molecule of any one of embodiments B1-13, comprising the heavy chain variable domain of SEQ ID NO:38 and the light chain variable domain of SEQ ID NO:47.

B15. The binding molecule of any one of embodiments B1-B14, comprising a CDR3 of SEQ ID NO:41 and a CDR3 of SEQ ID NO:50.

B16. The binding molecule of any one of embodiments B1-15, comprising a CDR1 of SEQ ID NO:39 and a CDR1 of SEQ ID NO:48.

B17. The binding molecule of any one of embodiments B1-B16, comprising a CDR2 of SEQ ID NO:40 and a CDR2 of SEQ ID NO:49.

B18. The binding molecule of any one of embodiments B1-17, comprising an antibody, antibody fragment, single-chain antibody, diabody, or BiTe.

B19. The binding molecule of embodiment B18, wherein the antibody is chosen from a monoclonal antibody, a polyclonal antibody, a recombinant antibody, an IgE antibody, an IgD antibody, an IgM antibody, an IgG antibody, an antibody comprising at least one amino acid substitution, an antibody comprising at least one non-naturally occurring amino acid, or combination of the foregoing.

B20. The binding molecule of embodiments B19, wherein the antibody is an IgG antibody.

B21. The binding molecule of embodiment B18, wherein the antibody fragment is chosen from an scFv, a Fab, a Fab′, a Fv, a F(ab′)2.

B22. The binding molecule of any one of embodiments B1-B21, which specifically binds to a polypeptide of SEQ ID NO:129 with a binding affinity of 100 nM or less.

B23. The binding molecule of embodiment B22, which specifically binds to a polypeptide of SEQ ID NO:129 with a binding affinity of 10 nM or less.

B24. The binding molecule of embodiment B22, which specifically binds to a polypeptide of SEQ ID NO:129 with a binding affinity of 1 nM or less.

B25. The binding molecule of any one of embodiments B1-B24, wherein X in SEQ ID NO:47 or SEQ ID NO:48 is isoleucine (I).

C1. The binding molecule of any one of embodiments A1-A24, B1-B25 and G0-G3.2, which is a chimeric antigen receptor molecule.

C2. The binding molecule of embodiment C1, comprising the scFv of embodiment A21 or B21.

C3. The binding molecule of embodiment C1 or C2, comprising a membrane association polypeptide.

C4. The binding molecule of embodiment C3, wherein the membrane association polypeptide is a region of a native transmembrane polypeptide.

C5. The binding molecule of embodiment C4, wherein the membrane association polypeptide is a stalk region polypeptide.

C6. The binding molecule of embodiment C5, wherein the stalk region polypeptide is a CD8 stalk region polypeptide comprising SEQ ID NO:91.

C7. The binding molecule of embodiment C4, wherein the membrane association polypeptide is a transmembrane region polypeptide.

C8. The binding molecule of embodiment C7, wherein the transmembrane region polypeptide is a CD8 transmembrane region polypeptide comprising SEQ ID NO:93 or a CD28 transmembrane region polypeptide comprising SEQ ID NO:140.

C9. The binding molecule of any one of embodiments C3-C8, comprising a stalk region polypeptide and a transmembrane region polypeptide.

C10. The binding molecule of any one of embodiments C1-C9, comprising a signal polypeptide.

C11. The binding molecule of embodiment C10, wherein the signal polypeptide is a region of a transmembrane polypeptide.

C12. The binding molecule of embodiment C11, wherein the signal polypeptide is a signal region polypeptide of CD8 comprising SEQ ID NO:75.

C13. The binding molecule of any one of embodiments C1-C12, comprising a tag polypeptide.

C14. The binding molecule of embodiment C13, wherein the tag polypeptide is a portion of an extracellular region of a cell membrane associated polypeptide.

C15. The binding molecule of embodiment C14, wherein the tag polypeptide is a portion of the extracellular region of a CD34 polypeptide.

C16. The binding molecule of embodiment C15, wherein the tag polypeptide comprises SEQ ID NO:79.

C17. The binding molecule of any one of embodiments C1-C16, comprising one or more stimulatory polypeptides.

C18. The binding molecule of embodiment C17, comprising a cytoplasmic region or portion thereof of a native stimulatory polypeptide.

C19. The binding molecule of embodiment C17 or C18, wherein the stimulatory polypeptide is capable of stimulating an immune cell.

C20. The binding molecule of embodiment C19, wherein the immune cell is chosen from one or more of a T-cell, NK cell, invariant natural killer T cell (iNKT) and mucosal-associated innate T (MAIT) cell.

C21. The binding molecule of embodiment C20, wherein the T-cell is chosen from one or more of a gamma.delta T-cell, CD4+ T-cell and CD8+ T-cell.

C22. The binding molecule of embodiment C21, wherein the stimulatory polypeptide independently is chosen from CD27, CD28, ICOS, 4-1BB, CD40, RANK/TRANCE-R, CD3-zeta chain, OX40, a pattern recognition receptor, TRIF, DNAX activating protein, NOD-like receptor and RIG-like helicase.

C23. The binding molecule of embodiment C22, wherein the stimulatory polypeptide comprises a cytoplasmic region of the CD3-zeta chain.

C24. The binding molecule of embodiment C22 or C23, wherein the stimulatory polypeptide comprises a cytoplasmic region of CD28.

C24.1. The binding molecule of embodiment C22 or C23, wherein the stimulatory polypeptide comprises a cytoplasmic region of DAP10.

C25. The binding molecule of any one of embodiments C17-C24, comprising two stimulatory polypeptides.

C26. The binding molecule of embodiment C25, comprising a cytoplasmic region of the CD3-zeta chain and a cytoplasmic region of CD28.

C26.1. The binding molecule of embodiment C25, comprising a cytoplasmic region of the CD3-zeta chain and a cytoplasmic region of DAP10.

C27. The binding molecule of embodiment C23, C26 or C26.1, wherein the cytoplasmic region of the CD3-zeta chain comprises SEQ ID NO:99 or SEQ ID NO:145.

C28. The binding molecule of embodiment C24 or C26, wherein the cytoplasmic region of CD28 comprises SEQ ID NO:97.

C28.1. The binding molecule of embodiment C24.1 or C26.1, wherein the cytoplasmic region of DAP10 comprises SEQ ID NO:143.

C29. The binding molecule of any one of embodiments C1-C28, comprising a signal polypeptide and a tag polypeptide and a linker between the signal polypeptide and the tag polypeptide.

C30. The binding molecule of embodiment C29, wherein the linker between the signal polypeptide and the tag polypeptide is about 1 amino acid to about 10 consecutive amino acids in length.

C31. The binding molecule of embodiment C30, wherein the linker between the signal polypeptide and the tag polypeptide comprises SEQ ID NO:77.

C32. The binding molecule of any one of embodiments C1-C31, comprising a tag polypeptide and a heavy chain variable (VH) domain polypeptide and a linker between the tag polypeptide and the VH domain polypeptide.

C33. The binding molecule of embodiment C32, wherein the linker between the tag polypeptide and the VH domain polypeptide is about 1 amino acid to about 10 consecutive amino acids in length.

C34. The binding molecule of embodiment C33, wherein linker between the tag polypeptide and the VH domain polypeptide comprises SEQ ID NO:81.

C35. The binding molecule of any one of embodiments C1-C34, comprising a heavy chain variable (VH) domain polypeptide and a light chain variable (VL) domain polypeptide and a linker between the VH domain polypeptide and the VL domain polypeptide.

C36. The binding molecule of embodiment C35, wherein the linker between the VH domain polypeptide and the VL domain polypeptide is about 5 to about 25 consecutive amino acids in length.

C37. The binding molecule of embodiment C35 or C36, wherein the linker between the VH domain polypeptide and the VL domain polypeptide comprises two more consecutive glycine amino acids, and optionally comprises one or more serine amino acids.

C38. The binding molecule of embodiment C37, wherein the linker between the VH domain polypeptide and the VL domain polypeptide comprises ((G)ₘS)ₙ, wherein m is an integer between 2 and 10 and n independently is an integer between 2 and 10.

C39. The binding molecule of embodiment C38, wherein linker between the VH domain polypeptide and the VL domain polypeptide comprises SEQ ID NO:85.

C40. The binding molecule of any one of embodiments C1-C39, comprising a light chain variable (VL) domain polypeptide and a stalk region polypeptide and a linker between the VL domain polypeptide and the stalk region polypeptide.

C41. The binding molecule of embodiment C40, wherein the linker between the VL domain polypeptide and the stalk region polypeptide is about 1 amino acid to about 10 consecutive amino acids in length.

C42. The binding molecule of embodiment C41, wherein linker between the VL domain polypeptide and the stalk region polypeptide comprises SEQ ID NO:89.

C43. The binding molecule of any one of embodiments C1-C42, comprising a transmembrane region polypeptide and a stimulatory polypeptide and a linker between the transmembrane region polypeptide and the stimulatory polypeptide.

C44. The binding molecule of embodiment C43, wherein the linker between the transmembrane region polypeptide and the stimulatory polypeptide is about 1 amino acid to about 10 consecutive amino acids in length.

C45. The binding molecule of embodiment C44, wherein linker between the transmembrane region polypeptide and the stimulatory polypeptide comprises SEQ ID NO:95.

C46. The binding molecule of any one of embodiments C1-C45, wherein the binding molecule comprises the structure of Formula A:

Nterm-(VH Domain)-(VL Domain)-(transmembrane region)-(first stimulatory molecule cytoplasmic region)-(second stimulatory molecule cytoplasmic region)-Cterm Formula A

wherein “Nterm” is the N-terminus of the binding molecule and “Cterm” is the C-terminus of the binding molecule.

C47. The binding molecule of any one of embodiments C1-C46, wherein the binding molecule comprises the structure of Formula B:

Nterm-(VH Domain)-(VL Domain)-(transmembrane region)-(CD28 cytoplasmic region)-(CD3-zeta cytoplasmic region)-Cterm Formula B

wherein “Nterm” is the N-terminus of the binding molecule and “Cterm” is the C-terminus of the binding molecule.

C48. The binding molecule of any one of embodiments C1-C47, wherein the binding molecule comprises the structure of Formula C:

Nterm-(VH Domain)-(VL Domain)-(CD8 transmembrane region)-(CD28 cytoplasmic region)-(CD3-zeta cytoplasmic region)-Cterm Formula C

wherein “Nterm” is the N-terminus of the binding molecule and “Cterm” is the C-terminus of the binding molecule.

C49. The binding molecule of any one of embodiments C1-C48, wherein the binding molecule comprises the structure of Formula D:

Nterm-(VH Domain)-(VL Domain)-(CD8 stalk region)-(CD8 transmembrane region)-(CD28 cytoplasmic region)-(CD3-zeta cytoplasmic region)-Cterm Formula D

wherein “Nterm” is the N-terminus of the binding molecule and “Cterm” is the C-terminus of the binding molecule.

C50. The binding molecule of any one of embodiments C1-C49, wherein the binding molecule comprises the structure of Formula E:

Nterm-(CD34 tag)-(VH Domain)-(VL Domain)-(CD8 stalk region)-(CD8 transmembrane region)-(CD28 cytoplasmic region)-(CD3-zeta cytoplasmic region)-Cterm Formula E

wherein “Nterm” is the N-terminus of the binding molecule and “Cterm” is the C-terminus of the binding molecule.

C51. The binding molecule of any one of embodiments C1-C50, wherein the binding molecule comprises the structure of Formula F:

Nterm-(CD8 signal)-(Linker 1)-(CD34 tag)-(Linker 2)-(VH Domain)-(Linker 3)-(VL Domain)-(Linker 4)-(CD8 stalk region)-(CD8 transmembrane region)-(Linker 5)-(CD28 cytoplasmic region)-(CD3-zeta cytoplasmic region)-Cterm Formula F

wherein “Nterm” is the N-terminus of the binding molecule and “Cterm” is the C-terminus of the binding molecule.

C52. The binding molecule of any one of embodiments C1-C51, comprising a VH Domain that comprises SEQ ID NO:83.

C53. The binding molecule of any one of embodiments C1-C52, comprising a VL Domain that comprises SEQ ID NO:87.

C54. The binding molecule of any one of embodiments C1-C53, comprising SEQ ID NO:73.

C54.1. The binding molecule of any one of embodiments C1-C53, comprising SEQ ID NO:196.

C55. The binding molecule of any one of embodiments C1-C51, comprising a VH Domain that comprises SEQ ID NO:111.

C56. The binding molecule of any one of embodiments C1-C51 and C55, comprising a VL Domain that comprises SEQ ID NO:115.

C57. The binding molecule of embodiment C56, wherein X in SEQ ID NO:115 is valine (V).

C58. The binding molecule of any one of embodiments C1-C51 and C55-C57, comprising SEQ ID NO:101.

C58.1. The binding molecule of any one of embodiments C1-C51 and C55-C57, comprising SEQ ID NO:197.

D1. The binding molecule of any one of embodiments A1-A24, B1-B25, C1-C58.1 and G0-G3.2, which is isolated.

D2. A nucleic acid comprising a polynucleotide that encodes a binding molecule of any one of embodiments A1-A24, B1-B25, C1-C58 and G0-G3.2.

D2.1. The nucleic acid of embodiment D2, which is an isolated nucleic acid.

D3. A cell, comprising:
one or more binding molecules of any one of embodiments A1-A24, B1-B25, C1-C58 and G0-G3.2; and/or
one or more nucleic acids each encoding one or more binding molecules of any one of embodiments A1-A24, B1-B25, C1-C58 and G0-G3.2.

D4. A cell comprising a nucleic acid of embodiment D2.

D5. The cell of embodiment D3 or D4, which is an immune cell in a population of cells.

D6. The cell of embodiment D5, wherein the immune cell is chosen from one or more of a T-cell, NK cell, invariant natural killer T cell (iNKT) and mucosal-associated innate T (MAIT) cell.

D6.1. The cell of embodiment D6, wherein the immune cell is an iNKT cell.

D6.2. The cell of embodiment D6.1, wherein the iNKT cell is prepared by the method of any one of embodiments J1-J143.

D7. The cell of embodiment D6, wherein the T-cell is chosen from one or more of a gamma.delta T-cell, CD4+ T-cell and CD8+ T-cell.

D7.1. The cell of embodiment D7, wherein the T-cell is a gamma.delta T cell.

D7.2. The cell of embodiment D7.1, wherein the gamma.delta T cell is prepared by the method of any one of embodiments H1-H95.

D8. The cell of any one of embodiments D3-D7, wherein the cell is isolated and/or a population of cells that includes the cell is isolated.

D9. The cell of any one of embodiments D3-D8, which is in vitro or ex vivo.

D10. The cell of any one of embodiments D3-D7, which is in vivo.

D11. The cell of any one of embodiments D3-D10, comprising a switch polypeptide and/or a polynucleotide encoding a switch polypeptide.

D12. The cell of embodiment D11, wherein the switch polypeptide is capable of inducing cell elimination after the cell is contacted with a multimeric agent capable of binding to the switch polypeptide.

D13. The cell of embodiment D12, wherein the switch polypeptide comprises, and/or comprises one or more nucleic acids that encode, (i) a first polypeptide capable of binding to a multimeric agent, and (ii) a second polypeptide capable of facilitating elimination of the cell upon multimeric agent-induced multimerization of the switch polypeptide.

D14. The cell of embodiment D13, wherein the switch polypeptide comprises, and/or comprises one or more nucleic acids that encode, a third polypeptide capable of binding to the multimeric agent to which the first polypeptide is capable of binding, or a third polypeptide capable of binding to a multimeric agent different than the multimeric agent to which the first polypeptide is capable of binding.

D15. The cell of embodiment D12, wherein the cell comprises, and/or comprises one or more nucleic acids that encode, (a) a first switch polypeptide comprising (i) a first polypeptide capable of binding to a multimeric agent, and (ii) a second polypeptide capable of facilitating elimination of the cell upon multimeric agent-induced multimerization of the switch polypeptide; and (b) a second switch polypeptide comprising (1) a third polypeptide capable of binding to the multimeric agent to which the first polypeptide is capable of binding, and (2) the second polypeptide capable of facilitating elimination of the cell upon multimeric agent-induced multimerization of the switch polypeptide.

D16. The cell of any one of embodiments D12-D15, wherein the polypeptide capable of facilitating cell elimination is a native polypeptide or functional fragment thereof.

D17. The cell of any one of embodiments D12-D16, wherein the polypeptide capable of facilitating cell elimination is an apoptosis-facilitating polypeptide.

D18. The cell of embodiment D17, wherein the apoptosis-facilitating polypeptide is chosen from Fas, Fas-associated death domain-containing protein (FADD), caspase-1, caspase-3, caspase-8 and caspase-9.

D19. The cell of embodiment D18, wherein the apoptosis-facilitating polypeptide is a caspase-9 polypeptide, or a functional fragment thereof.

D19.1. The cell of embodiment D19, wherein the apoptosis-facilitating polypeptide is a caspase-9 polypeptide fragment lacking a CARD domain.

D20. The cell of any one of embodiments D3-D19.1, wherein the switch polypeptide is capable of inducing cell stimulation after the cell is contacted with a multimeric agent capable of binding to the switch polypeptide.

D21. The cell of embodiment D20, wherein the switch polypeptide comprises, and/or comprises one or more nucleic acids that encode, (i) a first polypeptide capable of binding to a multimeric agent, and (ii) a second polypeptide capable of stimulating the cell upon multimeric agent-induced multimerization of the switch polypeptide.

D22. The cell of embodiment D21, wherein the switch polypeptide comprises, and/or comprises one or more nucleic acids that encode, a third polypeptide capable of binding to the multimeric agent or a third polypeptide capable of binding to a multimeric agent different than the multimeric agent to which the first polypeptide binds.

D23. The cell of embodiment D20, wherein the cell comprises, and/or comprises one or more nucleic acids that encode, (a) a first switch polypeptide comprising (i) a first polypeptide capable of binding to a multimeric agent, and (ii) a second polypeptide capable of stimulating the cell upon multimeric agent-induced multimerization of the switch polypeptide; and (b) a second switch polypeptide comprising (1) a third polypeptide capable of binding to the multimeric agent, and (2) the second polypeptide capable of stimulating the cell upon multimeric agent-induced multimerization of the switch polypeptide.

D24. The cell of any one of embodiments D20-D23, wherein the switch polypeptide capable of inducing cell stimulation comprises one or more polypeptides capable of stimulating a cell.

D25. The cell of embodiment D24, wherein the switch polypeptide comprises (i) multiple copies of one type of stimulatory polypeptide, or (ii) one or more copies of one type of stimulatory polypeptide and one or more copies of another type of stimulatory polypeptide.

D26. The cell of any one of embodiments D20-D25, wherein the polypeptide capable of simulating a cell upon multimeric agent-induced multimerization of the switch polypeptide is chosen independently from CD27, CD28, ICOS, 4-1BB, CD40, RANK/TRANCE-R, CD3 zeta chain, OX40, a pattern recognition receptor, TRIF, NOD-like receptor, RIG-like helicase, or functional fragment of the foregoing.

D27. The cell of embodiment D26, wherein the functional fragment is a cytoplasmic region of a native polypeptide.

D28. The cell of embodiment D26 or D27, wherein the pattern recognition receptor is a native MyD88 or a MyD88 fragment lacking a TIR region.

D29. The cell of any one of embodiments D13-D28, wherein the polypeptide capable of binding to a multimeric agent is chosen from (i) a FKBP polypeptide, (ii) a modified FKBP polypeptide (e.g., FKBP(F36V)), (iii) a FRB polypeptide, (iv) a modified FRB polypeptide, (v) a cyclophilin receptor polypeptide, (vi) a modified cyclophilin receptor polypeptide, (vii) a steroid receptor polypeptide, (viii) a modified steroid receptor polypeptide, (ix) a tetracycline receptor polypeptide, (x) a modified tetracycline receptor polypeptide, and (xi) a polypeptide containing complementarity determining regions (CDRs) of an antibody capable of immunospecifically binding to a multimeric agent.

D30. The cell of embodiment D29, wherein the modified FKBP polypeptide comprises a F36V amino acid substitution.

D31. The cell of any one of embodiments D13-D30, wherein the polypeptide capable of binding to a multimeric agent binds to the multimeric agent with an affinity of 100 nM or less.

D32. The cell of embodiment D31, wherein the polypeptide capable of binding to a multimeric agent binds to the multimeric agent with an affinity of 10 nM or less.

D33. The cell of embodiment D32, wherein the polypeptide capable of binding to a multimeric agent binds to the multimeric agent with an affinity of 1 nM or less.

D34. The cell of any one of embodiments D11-D33, wherein the switch polypeptide comprises one or more membrane-association components.

D35. The cell of any one of embodiments D11-D34, wherein the binding molecule is a chimeric antigen receptor molecule, and the switch polypeptide is in a triple-switch system comprising: (1) a switch comprising an inhibitory polypeptide for inhibition of chimeric antigen receptor activity; (2) a switch comprising an activating polypeptide for activation of chimeric antigen receptor activity; and (3) a switch comprising a polypeptide that triggers apoptosis of the cell.

D36. The cell of embodiment D35, wherein in (1), the inhibition of chimeric antigen receptor activity is reversible.

D37. The cell of embodiment D35 or D36, wherein in (2), the activation of chimeric antigen receptor activity is reversible.

D38. The cell of any one of embodiments D35-D37, wherein (1) comprises:
(a) a polypeptide comprising an FRB domain fused to an inhibitory polypeptide for inhibition of chimeric antigen receptor activity; and
(b) a cognate FKBP polypeptide associated with the chimeric antigen receptor, wherein, when the FRB domain is exposed to a chemical inducer of dimerization, the FRB domain binds to the cognate FKBP polypeptide, thereby recruiting the inhibitory polypeptide to the chimeric antigen receptor.

D39. The cell of embodiment any one of embodiments D35-D38, wherein the inhibitory polypeptide comprises tyrosine phosphatase activity.

D40. The cell of embodiment D39, wherein the tyrosine phosphatase is SHP1.

D41. The cell of any one of embodiments D38-D40, wherein the chemical inducer of dimerization comprises rapamycin or an analog thereof.

D41. The cell of any one of embodiments D35-D40, wherein (2) comprises:
(a) a polypeptide comprising (i) a second FRB domain fused to an activating polypeptide for activation of chimeric antigen receptor activity, wherein the second FRB domain is different than the FRB domain in (1); and
(b) a second cognate FKBP polypeptide associated with the chimeric antigen receptor, wherein the second cognate FKBP polypeptide is different than the cognate FKBP polypeptide in (1) and wherein, when the FRB domain is exposed to a second chemical inducer of dimerization, wherein the second chemical inducer of dimerization is different than the chemical inducer of dimerization in (1), the FRB domain binds to the cognate FKBP polypeptide, thereby recruiting the activating polypeptide to the chimeric antigen receptor.

D42. The cell of embodiment any one of embodiments D35-D41, wherein the activating polypeptide comprises tyrosine kinase activity.

D43. The cell of embodiment D42, wherein the tyrosine kinase comprises a modified Lck kinase.

D44. The cell of any one of embodiments D41-D43, wherein the second FRB domain comprises FRBT2098L/FRB_{L}.

D45. The cell of any one of embodiments D41-D44, wherein the second chemical inducer of dimerization comprises a non-immunosuppressive rapamycin analog.

D46. The cell of embodiment D45, wherein the non-immunosuppressive rapamycin analog binds to the second FRB domain and substantially does not bind to the FRB domain in (1).

D47. The cell of any one of embodiments D43-D46, wherein the modified Lck kinase comprises a truncated myristoylation domain, a truncated SH3 domain, or a truncated myristoylation domain and a truncated SH3 domain.

D48. The cell of any one of embodiments D43-D47, wherein the modified Lck kinase comprises a Y505 mutation.

D49. The cell of embodiment D48, wherein the modified Lck kinase comprises a Y505F mutation.

D50. The cell of any one of embodiments D35-D49, wherein the polypeptide that triggers apoptosis of the cell in (3) comprises a caspase-9 polypeptide fused to a polypeptide that binds to a third chemical inducer of dimerization, wherein the third chemical inducer of dimerization is different than the second chemical inducer of dimerization in (2) and the chemical inducer of dimerization in (1), and wherein the third chemical inducer of dimerization activates the caspase-9, thereby initiating apoptosis.

D51. The cell of embodiment D50, wherein the third chemical inducer of dimerization comprises rimiducid.

E1. A composition comprising a binding molecule of any one of embodiments A1-A24, B1-B25, C1-C58, D1 and G0-G3.2, a nucleic acid of embodiment D2 or D2.1, and/or a cell of any one of embodiments D3-D51.

E2. The composition of embodiment E2, comprising a pharmaceutically acceptable carrier, excipient or diluent.

E3. A binding molecule of any one of embodiments A1-A24, B1-B25, C1-C58, D1 and G0-G3.2, a cell of any one of embodiments D3-D51, or a composition of embodiment E1 or E2, for use as a medicament.

E4. A binding molecule of any one of embodiments A1-A24, B1-B25, C1-C58, D1 and G0-G3.2, a cell of any one of embodiments D3-D51, or a composition of embodiment E1 or E2, for treatment of a cancer.

E5. Use of a binding molecule of any one of embodiments A1-A24, B1-B25, C1-C58, D1 and G0-G3.2, a cell of any one of embodiments D3-D51, or a composition of embodiment E1 or E2, for treatment of a cancer.

E6. Use of a binding molecule of any one of embodiments A1-A24, B1-B25, C1-C58, D1 and G0-G3.2, a cell of any one of embodiments D3-D51, or a composition of embodiment E1 or E2, in the manufacture of a medicament for treating a cancer.

E7. A method for treating a cancer in a subject, comprising administering to a subject in need thereof a binding molecule of any one of embodiments A1-A24, B1-B25, C1-C58, D1 and G0-G3.2, a cell of any one of embodiments D3-D51, or a composition of embodiment E1 or E2, in a therapeutically effective amount to treat the cancer.

E8. The method of embodiment E7, wherein the cancer is chosen from a cancer of the ovary, cervix, lung, abdomen, heart, pancreas and/or stomach.

E9. The method of embodiment E7, wherein the cancer is chosen from mesothelioma, ovarian cancer, cervical squamous cell carcinoma, endocervical adenocarcinoma, lung adenocarcinoma, pancreatic adenocarcinoma and/or stomach adenocarcinoma.

E10. An agent that reduces a level of a mesothelin isoform-2 polypeptide in cells of a subject, for treatment of a cancer, wherein the mesothelin isoform-2 polypeptide comprises SEQ ID NO:129.

E11. Use of an agent that reduces a level of a mesothelin isoform-2 polypeptide in cells of a subject, for treatment of a cancer, wherein the mesothelin isoform-2 polypeptide comprises SEQ ID NO:129.

E12. A method for treating a cancer in a subject, comprising administering to a subject in need thereof an agent that reduces a level of mesothelin isoform-2 polypeptide in cells of a subject, in an amount effective to reduce the level of the mesothelin isoform-2 polypeptide in the cells, wherein the mesothelin isoform-2 polypeptide comprises SEQ ID NO:129.

E13. The agent, use or method of any one of embodiments E10-E12, wherein the agent is a binding molecule of any one of embodiments A1-A24, B1-B25, C1-C58 and D1, a cell of any one of embodiments D3-D51, or a composition of embodiment E1 or E2.

E14. The agent, use or method of any one of embodiments E10-E12, wherein the agent (i) deletes or disrupts one or more copies of a gene in DNA of the cells that encodes the mesothelin isoform-2 polypeptide, and/or (ii) reduces a level of a RNA transcript of a gene in the cells that encodes the mesothelin isoform-2 polypeptide.

E15. The binding molecule, cell, composition or method of any one of embodiments E4-E14, wherein the cancer is chosen from a cancer of the ovary, cervix, lung, abdomen, heart, pancreas and/or stomach.

E16. The binding molecule, cell or composition of embodiment E14, wherein the cancer is chosen from mesothelioma, ovarian cancer, cervical squamous cell carcinoma, endocervical adenocarcinoma, lung adenocarcinoma, pancreatic adenocarcinoma and/or stomach adenocarcinoma.

E17. The agent, use or method of any one of embodiments E10-E16, wherein the agent reduces the level of the mesothelin isoform-2 polypeptide to a greater extent than another mesothelin isoform polypeptide in the cells.

F1. A method for determining presence, absence or amount of a mesothelin isoform-2 polypeptide comprising SEQ ID NO:129, or a polynucleotide encoding the polypeptide.

F2. The method of embodiment F1, comprising contacting a biological sample or biological preparation with (i) a binding molecule that specifically binds to the mesothelin isoform-2 polypeptide, and/or (ii) a polynucleotide complementary to the polynucleotide encoding the mesothelin isoform-2 polypeptide or complement thereof.

F3. The method of embodiment F2, wherein the binding molecule is a binding molecule of any one of embodiments A1-A24, B1-B25, C1-C58 and D1.

F4. The method of embodiment F2 or F3, comprising contacting the biological sample or biological preparation with two different binding molecules, wherein each of the binding molecules specifically binds to the mesothelin isoform-2 polypeptide.

F5. The method of any one of embodiments F1-F4, comprising administering a therapy to a subject for treating a cancer.

F6. The method of embodiment F5, wherein the therapy comprises administering an agent to the subject that (i) specifically binds to the mesothelin isoform-2 polypeptide, (ii) deletes or disrupts one or more copies of a polynucleotide of the cells that encodes the mesothelin isoform-2 polypeptide, and/or (iii) reduces a level of a RNA polynucleotide in the cells that encodes the mesothelin isoform-2 polypeptide

F7. The method of embodiment F6, wherein the agent is a binding molecule of any one of embodiments A1-A24, B1-B25, C1-C58 and D1, a cell of any one of embodiments D3-D34, or a composition of embodiment E1 or E2.

F8. The method of any one of embodiments, F5-F7, wherein the cancer is chosen from a cancer of the ovary, cervix, lung, abdomen, heart, pancreas and/or stomach.

F9. The binding molecule, cell or composition of embodiment F8, wherein the cancer is chosen from mesothelioma, ovarian cancer, cervical squamous cell carcinoma, endocervical adenocarcinoma, lung adenocarcinoma, pancreatic adenocarcinoma, stomach adenocarcinoma, mesothelin epithelial ovarian cancer, and/or mesothelin malignant pleural mesothelioma.

G0. A binding molecule that is a chimeric PD1 molecule (chPD1) comprising a polypeptide according to Formula A:

PD1 region-transmembrane region-DAP10 region-CD3z region Formula A

wherein:
the polypeptide of Formula A is presented in the N-terminal to C-terminal direction; and
the transmembrane region comprises a CD28 transmembrane domain.

G0.1. The binding molecule of embodiment G0, wherein the CD3z region is of Isoform 1.

G0.2. The binding molecule of embodiment G0 or G0.1 that does not comprise a polypeptide linker between the DAP10 region and the CD3z region.

G0.3. The binding molecule of embodiment G0 or G0.1 that does not comprise a amino acid or polypeptide linker of 1, 2, 3, 4, 5, 6 or 7 or more amino acids between the DAP10 region and the CD3z region.

G0.4. The binding molecule of any one of embodiments G0-G0.3 that does not comprise the polypeptide linker sequence GVILTAL between the DAP10 region and the CD3z region.

G1. The binding molecule of any one of embodiments G0-G0.4, wherein:
the PD1 region comprises SEQ ID NO:137; and
the DAP10 region comprises SEQ ID NO:143; and
the CD3z region comprises SEQ ID NO:145.

G1.1. The binding molecule of any one of embodiments G0-G1, wherein the transmembrane region comprises SEQ ID NO:141.

G2. The binding molecule of any one of embodiments G0-G1.1, comprising a polypeptide according to Formula B:

PD1membrane signal-PD1 region-connector-transmembrane region-DAP10 region-CD3z region Formula B

wherein:
the polypeptide of Formula B is presented in the N-terminal to C-terminal direction; and
the polypeptide optionally comprises one or more of:
   the PD1 membrane signal comprising SEQ ID NO:135; and
   the connector comprising SEQ ID NO:139.

G3. The binding molecule of embodiment G2, comprising the polypeptide of SEQ ID NO:147.

G3.1. The binding molecule of any one of embodiments G0-G3, wherein one or more regions of the polypeptide are of human origin.

G3.2. The binding molecule of any one of embodiments G0-G3, wherein all the regions of the polypeptide are of human origin.

G4. A nucleic acid comprising a polynucleotide encoding a binding molecule of any one of embodiments G0-G3.

G5. The nucleic acid of embodiment G4, wherein the polynucleotide comprises one or more of:
a polynucleotide encoding the PD1 region comprising SEQ ID NO:136;
a polynucleotide encoding the connector comprising SEQ ID NO:138;
a polynucleotide encoding the CD28 transmembrane region comprising SEQ ID NO:140;
a polynucleotide encoding the DAP10 region comprising SEQ ID NO:142;
a polynucleotide encoding the CD3z region comprising SEQ ID NO:144; and
a polynucleotide encoding the PD1 membrane signal comprising SEQ ID NO:134.

G6. The nucleic acid of embodiment G4 or G5, wherein the polynucleotide comprises SEQ ID NO:146.

G7. The nucleic acid of any one of embodiments G4-G6, which is a plasmid.

G8. The nucleic acid of any one of embodiments G4-G6, which is a viral vector.

G9. The nucleic acid of embodiment G8, wherein the viral vector is a retroviral vector or lentiviral vector.

G10. A virus particle comprising the nucleic acid of embodiment G8 or G9.

G11. The virus particle of embodiment G10, which is a retrovirus or lentivirus.

G12. A cell comprising a binding molecule of any one of embodiments G0-G3, a nucleic acid of any one of embodiments G4-G9, or the virus particle of embodiment G10 or G11.

G13. The cell of embodiment G12, which is an immune cell.

G14. The cell of embodiment G13, wherein the immune cell is chosen from a T-cell, NK cell, invariant natural killer T cell (iNKT) and mucosal-associated innate T (MAIT) cell.

G15. The cell of embodiment G14, wherein the T-cell is chosen from a gamma.delta (γδ) T-cell, CD4− T-cell, CD8− T-cell, CD4+ T-cell and CD8+ T-cell.

G16. The cell of any one of embodiments G12-G15, comprising a binding molecule, a chimeric antigen receptor protein and/or a polynucleotide encoding the binding molecule or chimeric antigen receptor protein.

G16.1. The cell of any one of embodiments G12-G15, comprising a binding molecule of any one of embodiments A1-A24, B1-B25, C1-C58, D1 and G0-G3.2.

G17. A pharmaceutical composition comprising a cell of any one of embodiments G12-G16 and a pharmaceutically acceptable carrier.

G18. A cell of any one of embodiments G12-G16 or a pharmaceutical composition of embodiment G17 for treatment of a cancer.

G19. Use of a cell of any one of embodiments G12-G16 or a pharmaceutical composition of embodiment G17 for treatment of a cancer.

G20. Use of a cell of any one of embodiments G12-G16 in the manufacture of a medicament for treating a cancer.

G21. A method for treating a cancer, comprising administering a composition comprising a cell of any one of embodiments G12-G16 to a subject in need thereof in an amount effective for treating a cancer.

G22. The cell of embodiment G18, the use of embodiment G19 or G20 or the method of embodiment G21, wherein the cancer is chosen from a cancer of the ovary, cervix, lung, abdomen, heart, pancreas and/or stomach.

G23. The cell of embodiment G18, the use of embodiment G19 or G20 or the method of embodiment G21, wherein the cancer is chosen from mesothelioma, ovarian cancer, cervical squamous cell carcinoma, endocervical adenocarcinoma, lung adenocarcinoma, pancreatic adenocarcinoma, stomach adenocarcinoma, mesothelin epithelial ovarian cancer, and mesothelin malignant pleural mesothelioma.

H1. A method for preparing a cell composition enriched for gamma.delta T-cells, comprising: exposing a cell composition comprising gamma.delta T-cells (gdT-cells) to cell culture conditions comprising isolated interleukin-7 (IL-7) sufficient to enrich the gdT-cells in the cell composition.

H2. The method of embodiment H1, wherein the IL-7 is human IL-7.

H3. The method of embodiment H1 or H2, wherein the IL-7 is recombinant IL-7.

H4. The method of any one of embodiments H1-H3, wherein the cell composition comprising gdT-cells originates from peripheral blood cells.

H5. The method of embodiment H4, wherein the peripheral blood cells were preserved or were not preserved prior to exposing the cell composition to the cell culture conditions.

H6. The method of embodiment H5, wherein the peripheral blood cells were cryopreserved or were not cryopreserved prior to exposing the cell composition to the cell culture conditions.

H7. The method of any one of embodiments H3-H6, wherein the peripheral blood cells comprise peripheral blood mononuclear cells (PBMCs).

H8. The method of embodiment H7, wherein the peripheral blood cells comprise lymphocytes.

H9. The method of embodiment H8, wherein the peripheral blood cells comprise monocytes.

H10. The method of any one of embodiments H3-H9, wherein the peripheral blood cells comprise human cells.

H11. The method of embodiment H10, wherein the peripheral blood cells consist of, or consist essentially of, human cells.

H12. The method of any one of embodiments H1-H11, wherein the cell culture conditions comprise isolated interleukin-2 (IL-2).

H13. The method of embodiment H12, wherein the IL-2 is human IL-2.

H14. The method of embodiment H12 or H13, wherein the IL-2 is recombinant IL-2.

H15. The method of any one of embodiments H1-H14, wherein the cell culture conditions comprise zoledronic acid (ZA).

H16. The method of any one of embodiments H1-H15, wherein the cell culture conditions include no added extracts from non-human cells.

H17. The method of embodiment H16, wherein the cell culture conditions include no added non-human cells.

H17.1. The method of any one of embodiments H1-H17, wherein the cell culture conditions comprise added human antigen presenting cells and/or non-human antigen-presenting cells.

H18. The method of any one of embodiments H1-H17.1, wherein the cell culture conditions comprise no added irradiated cells and/or tumor cells.

H19. The method of any one of embodiments H1-H18, wherein the cell culture conditions comprise human serum.

H19.1. The method of any one of embodiments H1-H18, wherein the cell culture conditions comprise human platelet lysate.

H20. The method any one of embodiments H1-H19.1, wherein the cell composition comprises alpha.beta T-cells (abT-cells).

H21. The method of embodiment H20, comprising exposing the cell composition to depletion conditions that selectively remove abT-cells, thereby generating an abT-cell depleted cell composition.

H22. The method of embodiment H21, wherein the depletion conditions comprise exposing the cell composition to a binding molecule that immunospecifically binds to the abT-cells.

H23. The method of embodiment H21, wherein the binding molecule binds to a T-cell receptor (TCR) of abT-cells.

H23.1. The method of embodiment H22 or H23, wherein the binding molecule that immunospecifically binds to the abT-cells is an antibody or fragment thereof.

H24. The method of any one of embodiments H22-H23.1, wherein the binding molecule comprises a magnetic particle.

H25. The method of embodiment H24, comprising: contacting the binding molecule with a first binding partner linked to a magnetic particle, wherein:
the binding molecule comprises a second binding partner,
the binding molecule is contacted with the first binding partner under conditions in which the first binding partner binds to the second partner, and
the binding molecule is linked to the magnetic particle.

H26. The method of embodiment H24 or H25, comprising separating cells bound to the binding molecule with a magnet from cells not bound to the binding molecule in the cell composition.

H27. The method of any one of embodiments H21-H26, comprising exposing the cell composition to culture conditions for about 3 days to about 15 days prior to exposing the cell composition to the abT-cell depletion conditions.

H28. The method of embodiment H27, comprising exposing the cell composition to culture conditions for about 7 days prior to exposing the cell composition to the abT-cell depletion conditions.

H29. The method of any one of embodiments H21-H28, wherein the culture conditions comprise about 5 ng/mL to about 15 ng/mL of the IL-7 prior to exposing the cell composition to the abT-cell depletion conditions.

H30. The method of embodiment H29, wherein the culture conditions comprise about 10 ng/mL of the IL-7 prior to exposing the cell composition to the abT-cell depletion conditions.

H31. The method of any one of embodiments H21-H30, wherein the culture conditions comprise about 100 IU/mL to about 500 IU/mL of IL-2 prior to exposing the cell composition to the abT-cell depletion conditions.

H32. The method of embodiment H31, wherein the culture conditions comprise about 300 IU/mL of the IL-2 prior to exposing the cell composition to the abT-cell depletion conditions.

H33. The method of any one of embodiments H21-H32, wherein the culture conditions comprise about 1 micromolar to about 10 micromolar zoledronic acid (ZA) prior to exposing the cell composition to the abT-cell depletion conditions.

H34. The method of embodiment H31, wherein the culture conditions comprise about 5 micromolar zoledronic acid (ZA) prior to exposing the cell composition to the abT-cell depletion conditions.

H34.1. The method of any one of embodiments H1-H34, wherein about 40% to about 90% of cells in the cell composition are CD3 positive, V.gamma.9 positive and V.delta.2 positive prior to exposing the cell composition to the abT-cell depletion conditions.

H34.2. The method of any one of embodiments H1-H34.1, wherein about 50% to about 80% of cells in the cell composition are CD3 positive, V.gamma.9 positive and V.delta.2 positive prior to exposing the cell composition to the abT-cell depletion conditions.

H35. The method of any one of embodiments H21-H34.2, comprising exposing the abT-cell depleted cell composition to cell culture conditions.

H36. The method of embodiment H35, wherein the cell culture conditions to which the abT-cell depleted cell composition is exposed comprise IL-7.

H37. The method of embodiment H36, wherein the IL-7 is human IL-7.

H38. The method of embodiment H36 or H37, wherein the IL-7 is recombinant IL-7.

H39. The method of any one of embodiments H35-H38, wherein the cell culture conditions to which the abT-cell depleted cell composition is exposed comprise IL-2.

H40. The method of embodiment H39, wherein the IL-2 is human IL-2.

H41. The method of embodiment H39 or H40, wherein the IL-2 is recombinant IL-2.

H42. The method of any one of embodiments H25-H41, wherein the cell culture conditions to which the abT-cell depleted cell composition is exposed contains no added zoledronic acid (ZA).

H43. The method of any one of embodiments H25-H42, wherein the abT-cell depleted cell composition is exposed to the cell culture conditions for about 3 days to about 20 days.

H44. The method of embodiment H43, wherein the abT-cell depleted cell composition is exposed to the cell culture conditions for about 7 days to about 14 days.

H45. The method of any one of embodiments H25-H44, wherein the culture conditions to which the abT-cell depleted cell composition is exposed comprises about 5 ng/mL to about 15 ng/mL of the IL-7.

H46. The method of embodiment H45, wherein the culture conditions to which the abT-cell depleted cell composition is exposed comprises about 10 ng/mL of the IL-7.

H47. The method of any one of embodiments H25-H46, wherein the culture conditions to which the abT-cell depleted cell composition is exposed comprises about 100 IU/mL to about 500 IU/mL of IL-2.

H48. The method of embodiment H31, wherein the culture conditions to which the abT-cell depleted cell composition is exposed comprises about 300 IU/mL of the IL-2.

H48.1. The method of any one of embodiments H1-H48, wherein:
about 90% to about 99.9% of cells in the cell composition are CD3 positive,
about 90% to about 99.9% of cells in the cell composition are V.gamma.9 positive and/or V.delta.2 positive, and
about 5% or fewer cells in the cell composition are abT-cells,
after exposing the abT-cell depleted cell composition to the cell culture conditions.

H48.2. The method of any one of embodiments H1-H48.1, wherein:
about 97% to about 99.9% of cells in the cell composition are CD3 positive,
about 94% to about 99.9% of cells in the cell composition are V.gamma.9 positive and/or V.delta.2 positive, and
about 1% or fewer cells in the cell composition are abT-cells,
after exposing the abT-cell depleted cell composition to the cell culture conditions.

H48.3. The method of any one of embodiments H1-H48.2, wherein total expansion of cells in the cell population is about 1,000-fold to about 20,000-fold.

H48.4. The method of any one of embodiments H1-H48.3, wherein total expansion of cells in the cell population is about 2,500-fold to about 12,000-fold.

H48.5. The method of any one of embodiments H21-H48.4, wherein expansion of gdT-cells is about 50-fold to about 200-fold prior to exposing the cell composition to the abT-cell depletion conditions.

H48.6. The method of any one of embodiments H21-H48.5, wherein expansion of gdT-cells is about 5-fold to about 10-fold after the abT-cell depleted cell population is exposed to the cell culture conditions.

H48.7. The method of any one of embodiments H1-H48.6, wherein total expansion of gdT-cells is about 250-fold to about 2,000-fold.

H49. The method any one of embodiments H1-H48.7, comprising introducing a prepared nucleic acid into cells of the cell composition.

H50. The method of embodiment H49, wherein the prepared nucleic acid is introduced to the abT-cell depleted cell composition.

H51. The method of embodiment H50, wherein the prepared nucleic acid is introduced to the abT-cell depleted cell composition prior to exposing the abT-cell depleted cell composition to cell culture conditions, or within 1 day of exposing the abT-cell depleted cell composition to cell culture conditions.

H52. The method of any one of embodiments H49-H51, comprising introducing viral particles containing the prepared nucleic acid to the cell composition under conditions in which the viral particles enter cells of the cell composition.

H53. The method of any one of embodiments H49-H51, comprising introducing the prepared nucleic acid to the cell composition under conditions in which a polynucleotide of the nucleic acid integrates into cellular DNA of cells of the cell composition.

H54. The method of any one of embodiments H49-H51, comprising introducing the prepared nucleic acid to the cell composition under electroporation conditions in which the nucleic acid enters cells of the cell composition.

H55. The method of any one of embodiments H49-H54, wherein the nucleic acid comprises a polynucleotide encoding a protein.

H56. The method of embodiment H55, wherein the nucleic acid comprises a polynucleotide encoding a chimeric antigen receptor.

H56.1. The method of embodiment H55 or H56, wherein the polynucleotide encodes a binding molecule of any one of embodiments A1-A24, B1-B24 or C1-C58.

H56.2. The method of any one of embodiments H55-H56.1, wherein the polynucleotide encodes a chimeric protein of any one of embodiments G0-G3.

H56.3. The method of any one of embodiments H49-H56.2, wherein the nucleic acid and/or the polynucleotide encoding the protein is in about 20% to about 95% of gdT-cells in the cell population.

H56.4. The method of embodiment H49-H56.3, wherein the nucleic acid and/or the polynucleotide encoding the protein is in about 60% to about 95% of gdT-cells in the cell population.

H56.5. The method of embodiment H49-H56.2, wherein the nucleic acid and/or the polynucleotide encoding the protein is in about 65% or more of gdT-cells in the cell population.

H56.6. The method of embodiment H49-H56.2, wherein the nucleic acid and/or the polynucleotide encoding the protein is in about 70% or more of gdT-cells in the cell population.

H56.7. The method of embodiment H49-H56.2, wherein the nucleic acid and/or the polynucleotide encoding the protein is in about 75% or more of gdT-cells in the cell population.

H56.8. The method of embodiment H49-H56.2, wherein the nucleic acid and/or the polynucleotide encoding the protein is in about 80% or more of gdT-cells in the cell population.

H56.9. The method of embodiment H49-H56.2, wherein the nucleic acid and/or the polynucleotide encoding the protein is in about 85% or more of gdT-cells in the cell population.

H56.10. The method of any one of embodiments H1-H56.9, comprising selecting a cell composition prior to exposure to the IL-7 based on a threshold amount of cells in the cell composition that are positive for cell surface proteins.

H56.11. The method of embodiment H56.10, wherein the cell surface proteins are one or more of CD3, a V.gamma protein and a V.delta protein.

H56.12. The method of embodiment H56.11, wherein the V.gamma protein is a V.gamma.9 protein and/or the V.delta protein is a V.delta.2 protein.

H56.13. The method of embodiment H56.12, wherein a cell composition, in which (i) an amount of cells that are CD3 positive, V.gamma.9 positive and V.delta.2 positive in the cell composition, is greater than about 2% of (ii) an amount of cells that are CD3 positive in the cell composition, is selected for exposure to the IL-7.

H57. The method of any one of embodiments H1-H56.13, comprising:
(a) exposing a cell composition comprising human peripheral blood mononuclear cells (PBMCs) to first cell culture conditions comprising IL-7, wherein the PBMCs comprises abT-cells and gdT-cells;
(b) exposing the cell composition after (a) to depletion conditions that selectively remove the abT-cells, thereby generating an abT-cell depleted cell composition;
(c) exposing the abT-cell depleted cell composition to second cell culture conditions comprising IL-7, thereby generating a cell composition enriched for the gdT-cells relative to the amount of gdT-cells present in the PBMCs.

H58. The method of embodiment H57, wherein the first cell culture conditions and the second cell culture conditions comprise IL-2.

H59. The method of embodiment H57 and H58, wherein the first cell culture conditions comprise zoledronic acid (ZA).

H60. The method of any one of embodiments H57-H59, wherein the second cell culture conditions include no added zoledronic acid (ZA).

H61. The method of any one of embodiments H57-H60, wherein part (a) is performed for about 3 days to about 15 days.

H62. The method of embodiment H61, wherein part (a) is performed for about 7 days.

H63. The method of any one of embodiments H57-H62, wherein part (c) is performed for about 3 days to about 20 days.

H64. The method of embodiment H63, wherein part (c) is performed for about 7 days to about 14 days.

H65. The method of any one of embodiments H57-H64, wherein the first culture conditions and the second culture conditions independently comprise about 5 ng/mL to about 15 ng/mL of the IL-7.

H66. The method of embodiment H65, wherein the first culture conditions and the second culture conditions independently comprise about 10 ng/mL of the IL-7.

H67. The method of any one of embodiments H57-H66, wherein the first culture conditions and the second culture conditions independently comprise about 100 IU/mL to about 500 IU/mL of IL-2.

H68. The method of embodiment H67, wherein the first culture conditions and the second culture conditions independently comprise about 300 IU/mL of the IL-2.

H69. The method of any one of embodiments H57-H68, wherein the first culture conditions comprise about 1 micromolar to about 10 micromolar zoledronic acid (ZA).

H70. The method of embodiment H69, wherein the first culture conditions comprise about 5 micromolar zoledronic acid (ZA).

H71. The method any one of embodiments H57-H70, comprising introducing a prepared nucleic acid into cells of the cell composition after part (b).

H72. The method any one of embodiments H57-H71, comprising introducing a prepared nucleic acid into cells of the cell composition prior to part (c).

H73. The method embodiment H71 or H72, wherein the prepared nucleic acid is introduced to the abT-cell depleted cell composition prior to exposing the abT-cell depleted cell composition to cell culture conditions, or within 1 day of exposing the abT-cell depleted cell composition to cell culture conditions.

H74. The method of any one of embodiments H71-H73, comprising introducing viral particles containing the prepared nucleic acid to the cell composition under conditions in which the viral particles enter cells of the cell composition.

H75. The method of any one of embodiments H71-H73, comprising introducing the prepared nucleic acid to the cell composition under conditions in which a polynucleotide of the nucleic acid integrates into cellular DNA of cells of the cell composition.

H76. The method of any one of embodiments H71-H73, comprising introducing the prepared nucleic acid to the cell composition under electroporation conditions in which the nucleic acid enters cells of the cell composition.

H77. The method of any one of embodiments H71-H76, wherein the nucleic acid comprises a polynucleotide encoding a protein.

H78. The method of embodiment H77, wherein the nucleic acid comprises a polynucleotide encoding a chimeric antigen receptor.

H79. The method of embodiment H77 or H78, wherein the polynucleotide encodes a binding molecule of any one of embodiments A1-A24, B1-B24 or C1-C58.

H80. The method of any one of embodiments H77-H79, wherein the polynucleotide encodes a chimeric protein of any one of embodiments G1-G3.

H81. The method of any one of embodiments H71-H80, wherein the nucleic acid and/or the polynucleotide encoding the protein is in about 20% to about 95% of cells in the cell population.

H82. The method of embodiment H71-H81, wherein the nucleic acid and/or the polynucleotide encoding the protein is in about 60% to about 95% of cells in the cell population.

H82.1. The method of embodiment H57-H80, wherein the nucleic acid and/or the polynucleotide encoding the protein is in about 65% or more of gdT-cells in the cell population.

H82.2. The method of embodiment H57-H80, wherein the nucleic acid and/or the polynucleotide encoding the protein is in about 70% or more of gdT-cells in the cell population.

H82.3. The method of embodiment H57-H80, wherein the nucleic acid and/or the polynucleotide encoding the protein is in about 75% or more of gdT-cells in the cell population.

H82.4. The method of embodiment H57-H80, wherein the nucleic acid and/or the polynucleotide encoding the protein is in about 80% or more of gdT-cells in the cell population.

H82.5. The method of embodiment H57-H80, wherein the nucleic acid and/or the polynucleotide encoding the protein is in about 85% or more of gdT-cells in the cell population.

H83. The method of any one of embodiments H57-H82.5, wherein about 40% to about 90% of cells in the cell composition are CD3 positive, V.gamma.9 positive and V.delta.2 positive prior to exposing the cell composition to the abT-cell depletion conditions.

H84. The method of any one of embodiments H57-H83, wherein about 50% to about 80% of cells in the cell composition are CD3 positive, V.gamma.9 positive and V.delta.2 positive prior to exposing the cell composition to the abT-cell depletion conditions.

H85. The method of any one of embodiments H57-H84, wherein:
about 90% to about 99.9% of cells in the cell composition are CD3 positive,
about 90% to about 99.9% of cells in the cell composition are V.gamma.9 positive and/or V.delta.2 positive, and
about 5% or fewer cells in the cell composition are abT-cells,
after exposing the abT-cell depleted cell composition to the cell culture conditions.

H86. The method of any one of embodiments H57-H85, wherein:
about 97% to about 99.9% of cells in the cell composition are CD3 positive,
about 94% to about 99.9% of cells in the cell composition are V.gamma.9 positive and/or V.delta.2 positive, and
about 1% or fewer cells in the cell composition are abT-cells,
after exposing the abT-cell depleted cell composition to the cell culture conditions.

H87. The method of any one of embodiments H57-H86, wherein total expansion of cells in the cell population is about 1,000-fold to about 20,000-fold.

H88. The method of any one of embodiments H57-H87, wherein total expansion of cells in the cell population is about 2,500-fold to about 12,000-fold.

H88.1. The method of any one of embodiments H57-H88, wherein expansion of gdT-cells is about 50-fold to about 200-fold prior to part (b).

H88.2. The method of any one of embodiments H57-H88.1, wherein expansion of gdT-cells is about 5-fold to about 10-fold after part (b).

H88.3. The method of any one of embodiments H57-H88.2, wherein total expansion of gdT-cells is about 250-fold to about 2,000-fold.

H88.4. The method of any one of embodiments H57-H88.3, comprising selecting a cell composition prior to exposure to the IL-7 based on a threshold amount of cells in the cell composition that are positive for one or more cell surface proteins.

H88.5. The method of embodiment H88.4, wherein the cell surface proteins are chosen from one or more of CD3, a V.gamma protein and a V.delta protein.

H88.6. The method of embodiment H88.4, wherein the gamma protein is a V.gamma.9 protein and/or the delta protein is a V.delta.2 protein.

H88.7. The method of embodiment H88.6, wherein a cell composition, in which (i) an amount of cells that are CD3 positive, V.gamma.9 positive and V.delta.2 positive in the cell composition, is greater than about 2% of (ii) an amount of cells that are CD3 positive in the cell composition, is selected for exposure to the IL-7.

H89. The method of any one of embodiments H57-H88.7, comprising preserving cells after (a), after (b) and/or after (c).

H90. The method of embodiment H89, wherein cells are preserved after (c).

H91. The method of embodiment H89 or H90, wherein cells are cryopreserved.

H92. The method of embodiment H91, wherein the cells are in a composition comprising a cryopreservation medium.

H93. The method of embodiment H91 or H92, wherein the cells are in cryopreservation conditions.

H94. The method of any one of embodiments H89-H93, wherein the cells are in a container.

H95. The method of any one of embodiments H1-G94, wherein cells of the cell population comprise a switch polypeptide of any one of embodiments D11-D34.

H95.1 The method of any one of embodiments H1-H95, wherein the culture conditions do not comprise IL-15.

H96. A cell composition prepared by a method of any one of embodiments H1-H95.

H97. A pharmaceutical composition comprising a cell composition of embodiment H96 and a pharmaceutically acceptable carrier.

I1 (reserved).

J1. A method for preparing a cell composition enriched for invariant natural killer T-cells, comprising:
exposing an input cell composition comprising invariant natural killer T-cells (iNKT-cells) and other cells to separation conditions that separate the iNKT-cells from the other cells, thereby generating a separated cell composition comprising the iNKT-cells, and
exposing the separated cell composition to cell culture conditions comprising a prepared cell composition, wherein the prepared cell composition comprises non-irradiated peripheral blood cells.

J2. The method of embodiment J1, wherein the input cell composition comprises peripheral blood cells.

J3. The method of embodiment J2, wherein the peripheral blood cells of the input cell composition were preserved or were not preserved prior to exposing the input cell composition to the separation conditions.

J4. The method of embodiment J3, wherein the peripheral blood cells of the input cell composition were cryopreserved or were not cryopreserved prior to exposing the input cell composition to the separation conditions.

J5. The method of any one of embodiments J1-J4, wherein the peripheral blood cells of the prepared cell composition were preserved or were not preserved prior to exposing the separated cell composition to the cell culture conditions.

J6. The method of embodiment J5, wherein the peripheral blood cells of the prepared cell composition were cryopreserved or were not cryopreserved prior to exposing the separated cell composition to the cell culture conditions.

J7. The method of any one of embodiments J1-J6, wherein the peripheral blood cells of the input cell composition and/or the prepared cell composition independently comprise peripheral blood mononuclear cells (PBMCs).

J8. The method of embodiment J7, wherein the peripheral blood cells of the input cell composition and/or the prepared cell composition independently comprise lymphocytes.

J9. The method of embodiment J8, wherein the peripheral blood cells of the input cell composition and/or the prepared cell composition independently comprise monocytes.

J10. The method of any one of embodiments J3-J9, wherein the peripheral blood cells of the input cell composition and/or the prepared cell composition independently comprise human cells.

J11. The method of embodiment J10, wherein the peripheral blood cells of the input cell composition and/or the prepared cell composition independently consist of, or independently consist essentially of, human cells.

J12. The method of any one of embodiments J1-J11, wherein the input cell composition and the prepared cell composition comprise cells from the same human subject.

J13. The method of embodiment J12, wherein the input cell composition and the prepared cell composition comprise cells prepared from one cell sample obtained from one human subject.

J14. The method of embodiment J12 or J13, comprising apportioning a cell composition obtained from one human subject into a first cell composition and a second cell composition, wherein:
the first cell composition is the input cell composition or the input cell composition is prepared from the first cell composition, and
the prepared cell composition is prepared from the second cell composition.

J15. The method of any one of embodiments J1-J14, wherein the prepared cell composition is prepared by a process comprising exposing non-irradiated peripheral blood cells to cell culture conditions in which the non-irradiated peripheral blood cells adhere to a surface of a container.

J15.1. The method of embodiment J15, wherein about 5 million to about 20 million of the non-irradiated peripheral blood cells are exposed to the cell culture conditions.

J15.2. The method of embodiment J15, wherein about 10 million to about 15 million of the non-irradiated peripheral blood cells are exposed to the cell culture conditions.

J16. The method of any one of embodiments J15-J15.2, wherein the cell culture conditions to which the non-irradiated peripheral blood cells are exposed are serum-free cell culture conditions.

J17. The method of embodiment J16, wherein the cell culture conditions to which the non-irradiated peripheral blood cells are exposed comprise glutathione and vitamins.

J18. The method of embodiment J17, wherein the vitamins comprise one or more of biotin, vitamin B12, and para-amino benzoic acid.

J19. The method of any one of embodiments J1-J18, wherein the cell culture conditions to which the separated cell composition is exposed comprise isolated interleukin-2 (IL-2).

J20. The method of embodiment J19, wherein the IL-2 is human IL-2.

J21. The method of embodiment J19 or J20, wherein the IL-2 is recombinant IL-2.

J22. The method of any one of embodiments J19-J21, wherein the cell culture conditions comprises about 50 U/mL to about 500 U/mL of IL-2.

J23. The method of any one of embodiments J19-J22, wherein the cell culture conditions comprises about 100 U/mL to about 300 U/mL of IL-2.

J24. The method of any one of embodiments J1-J23, wherein the cell culture conditions to which the separated cell composition is exposed comprise isolated interleukin-21 (IL-21).

J25. The method of embodiment J24, wherein the IL-21 is human IL-21.

J26. The method of embodiment J24 or J25, wherein the IL-21 is recombinant IL-21.

J27. The method of any one of embodiments J24-J26, wherein the cell culture conditions comprises about 1 ng/mL to about 100 ng/mL of IL-21.

J28. The method of any one of embodiments J24-J27, wherein the cell culture conditions comprises about 5 ng/mL to about 20 ng/mL of IL-21.

J29. The method of any one of embodiments J1-J28, wherein the cell culture conditions to which the separated cell composition is exposed comprise an isolated component comprising a lipid.

J30. The method of embodiment J29, wherein the isolated component is a glycolipid.

J31. The method of embodiment J29 or J30, wherein the isolated component is alpha-galactosylceramide.

J32. The method of any one of embodiments J29-J31, wherein the cell culture conditions comprises about 10 ng/mL to about 1,000 ng/mL of the isolated component comprising the lipid.

J33. The method of any one of embodiments J29-J31, wherein the cell culture conditions comprises about 50 ng/mL to about 200 ng/mL of the isolated component comprising the lipid.

J34. The method of any one of embodiments J1-J33, wherein the cell culture conditions include no added extract from a non-human cells.

J35. The method of embodiment J34, wherein the cell culture conditions include no added non-human cells.

J36. The method of any one of embodiments J1-J35, wherein the cell culture conditions comprise no added irradiated cells and/or tumor cells.

J37. The method of any one of embodiments J1-J36, wherein the cell culture conditions to which the separated cell composition is exposed comprise human serum.

J38. The method of any one of embodiments J1-J37, comprising exposing the separated cell composition to the culture conditions for about 3 days to about 25 days.

J39. The method of embodiment J38, comprising exposing the separated cell composition to the culture conditions for about 5 days to about 15 days.

J40. The method of embodiment J39, comprising exposing the separated cell composition to the culture conditions for about 10 days.

J41. The method any one of embodiments J1-J40, wherein the separation conditions to which the input cell composition is exposed comprise a component that immunospecifically binds to a T-cell receptor (TCR) expressed on the iNKT-cells.

J42. The method of embodiment J41, wherein the component immunospecifically binds to an alpha chain of the TCR.

J43. The method of embodiment J42, wherein the alpha chain is a V.alpha.24-J.alpha.18 chain.

J43.1. The method of any one of embodiments J41-J43, wherein the component that immunospecifically binds to the TCR is an antibody or fragment thereof.

J44. The method of any one of embodiments J41-J43.1, wherein the component comprises a magnetic particle.

J45. The method of embodiment J44, wherein the separation conditions comprise separating cells bound to the component with a magnet from cells not bound to the component in the input cell composition.

J46. The method of any one of embodiments J1-J45, comprising, prior to exposing the input cell composition to the separation conditions:
contacting a cell composition with one or more agents that immunospecifically bind to iNKT-cells, and
determining the amount of cells in the cell composition bound to the one or more agents.

J47. The method of embodiment J46, comprising selecting a cell composition as the input cell composition based on the amount of cells bound to the one or more agents.

J48. The method of embodiment J47, comprising selecting a cell composition for which the amount of cells bound to the one or more agents is greater than about 0.05% of cells in the cell composition as the input cell composition.

J49. The method of embodiment J46, comprising not selecting as the input cell composition based on the amount of cells bound to the one or more agents.

J50. The method of embodiment J49, comprising not selecting a cell composition for which the amount of cells bound to the one or more agents is less than about 0.05% of cells in the cell composition as the input cell composition.

J51. The method of any one of embodiments J46-J50, wherein the one or more agents are chosen independently from one or more of: an antibody or fragment thereof that immunospecifically binds to CD3 expressed on iNKT-cells and an antibody or fragment thereof that immunospecifically binds to a TCR expressed on iNKT-cells.

J52. The method of any one of embodiments J46-J51, comprising:
contacting a cell composition with a first agent and a second agent, wherein the first agent immunospecifically binds to CD3 expressed on iNKT-cells and the second agent immunospecifically binds to a TCR expressed on iNKT-cells;
determining the amount of cells in the cell composition bound to the first agent and the second agent; and
selecting a cell composition as the input cell composition based on the amount of cells bound to the first agent and the second agent.

J53. The method of embodiment J52, comprising selecting a cell composition for which the amount of cells bound to the first agent and the second agent is greater than about 0.05% of cells in the cell composition as the input cell composition.

J54. The method of any one of embodiments J46-J51, comprising:
contacting a cell composition with a first agent and a second agent, wherein the first agent immunospecifically binds to CD3 expressed on iNKT-cells and the second agent immunospecifically binds to a TCR expressed on iNKT-cells;
determining the amount of cells in the cell composition bound to the first agent and the second agent; and
not selecting a cell composition as the input cell composition based on the amount of cells bound to the first agent and the second agent.

J55. The method of embodiment J54, comprising not selecting a cell composition for which the amount of cells bound to the first agent and the second agent is less than about 0.05% of cells in the cell composition as the input cell composition.

J56. The method of any one of embodiments J1-J55, wherein:
about 70% to about 99.9% of cells in the cell composition are CD3 positive and iNKT-TCR positive, and/or
about 20% or fewer cells in the cell composition are CD3 positive and iNKT-TCR negative,
after exposing the separated cell composition to the cell culture conditions for about 5 days or more.

J57. The method of any one of embodiments J1-J56, wherein:
about 80% to about 90% of cells in the cell composition are CD3 positive and iNKT-TCR positive, and/or
about 15% or fewer cells in the cell composition are CD3 positive and iNKT-TCR negative,
after exposing the separated cell composition to the cell culture conditions for about 7 days or more.

J58. The method of any one of embodiments J1-J57, wherein:
about 90% or more of cells in the cell composition are CD3 positive and iNKT-TCR positive, and/or
about 5% or fewer cells in the cell composition are CD3 positive and iNKT-TCR negative,
after exposing the separated cell composition to the cell culture conditions for about 14 days or more.

J59. The method of any one of embodiments J1-J57, wherein:
about 97% or more of cells in the cell composition are CD3 positive and iNKT-TCR positive, and/or
about 0.5% or fewer cells in the cell composition are CD3 positive and iNKT-TCR negative,
after exposing the separated cell composition to the cell culture conditions for about 14 days or more.

J60. The method of any one of embodiments J1-J59, wherein total expansion of cells in the cell population is about 100-fold to about 10,000-fold after exposing the separated cell composition to the cell culture conditions.

J61. The method of any one of embodiments J1-J60, wherein total expansion of cells in the cell population is about 300-fold to about 1,000-fold after exposing the separated cell composition to the cell culture conditions.

J62. The method any one of embodiments J1-J61, comprising introducing a prepared nucleic acid into cells of the cell composition.

J63. The method of embodiment J62, wherein the prepared nucleic acid is introduced to the separated cell composition.

J64. The method of embodiment J62, wherein the prepared nucleic acid is introduced to the separated cell composition prior to exposing the separated cell composition to cell culture conditions, or within 1 day of exposing the separated cell composition to cell culture conditions.

J65. The method of any one of embodiments J62-J64, comprising introducing viral particles containing the prepared nucleic acid to the cell composition under conditions in which the viral particles enter cells of the cell composition.

J66. The method of any one of embodiments J62-J64, comprising introducing the prepared nucleic acid to the cell composition under conditions in which a polynucleotide of the nucleic acid integrates into cellular DNA of cells of the cell composition.

J67. The method of any one of embodiments J62-J64, comprising introducing the prepared nucleic acid to the cell composition under electroporation conditions in which the nucleic acid enters cells of the cell composition.

J68. The method of any one of embodiments J62-J67, wherein the nucleic acid comprises a polynucleotide encoding a protein.

J69. The method of embodiment J68, wherein the nucleic acid comprises a polynucleotide encoding a chimeric antigen receptor.

J70. The method of embodiment J68 or J70, wherein the polynucleotide encodes a binding molecule of any one of embodiments A1-A24, B1-B24 or C1-C58.

J71. The method of any one of embodiments J68-J70, wherein the polynucleotide encodes a chimeric protein of any one of embodiments G1-G3.

J72. The method of any one of embodiments J62-J71, wherein the nucleic acid and/or the polynucleotide encoding the protein is in about 20% to about 70% of cells in the cell population.

J73. The method of embodiment J62-J72, wherein the nucleic acid and/or the polynucleotide encoding the protein is in about 30% to about 60% of cells in the cell population.

J73.1. The method of embodiment J62-J72, wherein the nucleic acid and/or the polynucleotide encoding the protein is in about 65% or more of iNKT-cells in the cell population.

J73.2. The method of embodiment J62-J72, wherein the nucleic acid and/or the polynucleotide encoding the protein is in about 70% or more of iNKT-cells in the cell population.

J73.3. The method of embodiment J62-J72, wherein the nucleic acid and/or the polynucleotide encoding the protein is in about 75% or more of iNKT-cells in the cell population.

J73.4. The method of embodiment J62-J72, wherein the nucleic acid and/or the polynucleotide encoding the protein is in about 80% or more of iNKT-cells in the cell population.

J73.5. The method of embodiment J62-J72, wherein the nucleic acid and/or the polynucleotide encoding the protein is in about 85% or more of iNKT-cells in the cell population.

J74. The method of any one of embodiments J1-J56.4, comprising:
(a) identifying an amount of invariant natural killer T-cells (iNKT-cells) in a cell composition comprising human peripheral blood mononuclear cells (PBMCs);
(b) if the amount of the iNKT-cells identified in the cell composition exceeds a threshold amount of the iNKT-cells, apportioning the cell composition into a first portion and a second portion;
(c) exposing the first portion to cell culture conditions in which cells of the first portion adhere to a surface of a container, thereby generating a prepared cell composition;
(d) exposing the second portion to separation conditions that separate the iNKT-cells from other cells, thereby generating a separated cell composition comprising the iNKT-cells; and
(e) exposing the separated cell composition to cell culture conditions comprising the prepared cell composition of part (c).

J75. The method of embodiment J74, wherein cells of the cell composition are contacted in (a) with one or more agents that bind to iNKT-cells and the amount of cells bound to the one or more agents is identified as the amount of the iNKT-cells.

J76. The method of embodiment J75, wherein the one or more agents are chosen from an agent that binds to CD3 and/or an agent that binds to a T-cell receptor expressed on iNKT-cells.

J77. The method of embodiment J76, wherein the one or more agents are chosen from an antibody or fragment thereof that immunospecifically binds to CD3 and/or an antibody or fragment thereof that immunospecifically to a T-cell receptor expressed on iNKT-cells.

J78. The method of any one of embodiments J74-J77, wherein the threshold is about 0.01%.

J79. The method of embodiment J78, wherein the threshold is about 0.05%.

J80. The method of any one of embodiments J74-J79, wherein in (b) about 5 million to about 20 million cells in the cell composition are apportioned into the first portion.

J81. The method of embodiment J80, wherein about 10 million to about 15 million cells in the cell composition are apportioned into the first portion.

J82. The method of any one of embodiments J74-J81, wherein the cell culture conditions in part (c) are serum-free cell culture conditions.

J83. The method of any one of embodiments J74-J81, wherein the cell culture conditions in part (c) comprise glutathione and vitamins.

J84. The method of embodiment J83, wherein the vitamins comprise one or more of biotin, vitamin B12, and para-amino benzoic acid.

J85. The method any one of embodiments J74-J84, wherein the separation conditions in part (d) comprise a component that binds to a T-cell receptor (TCR) expressed on the iNKT-cells.

J86. The method of embodiment J41, wherein the component binds to an alpha chain of the TCR.

J87. The method of embodiment J86, wherein the alpha chain is a V.alpha.24-J.alpha.18 chain.

J88. The method of any one of embodiments J85-J87, wherein the component that binds to the TCR is an antibody or fragment thereof that immunospecifically binds to the TCR.

J89. The method of any one of embodiments J85-J88, wherein the component comprises a magnetic particle.

J90. The method of embodiment J89, wherein the separation conditions of part (d) comprise separating cells bound to the component with a magnet from cells not bound to the component in the input cell composition.

J91. The method of any one of embodiments J74-J90, wherein the cell population in part (a) comprises lymphocytes.

J92. The method of any one of embodiments J74-J91, wherein the cell population in part (a) comprises monocytes.

J93. The method of any one of embodiments J74-J92, wherein the cell population in part (a) comprises human cells.

J94. The method of embodiment J93, wherein the cell population in part (a) consists of, or consists essentially of, human cells.

J95. The method of any one of embodiments J74-J94, wherein the cell population in part (a) is from the same human subject.

J96. The method of embodiment J95, wherein the cell population in part (a) is prepared from a cell sample obtained from one human subject.

J97. The method of any one of embodiments J74-J96, wherein the cell culture conditions of part (e) comprise isolated interleukin-2 (IL-2).

J98. The method of embodiment J97, wherein the IL-2 is human IL-2.

J99. The method of embodiment J97 or J98, wherein the IL-2 is recombinant IL-2.

J100. The method of any one of embodiments J74-J99, wherein the cell culture conditions of part (e) comprise about 50 U/mL to about 500 U/mL of IL-2.

J101. The method of any one of embodiments J74-J100, wherein the cell culture conditions of part (e) comprise about 100 U/mL to about 300 U/mL of IL-2.

J102. The method of any one of embodiments J74-J101, wherein the cell culture conditions of part (e) comprise isolated interleukin-21 (IL-21).

J103. The method of embodiment J102, wherein the IL-21 is human IL-21.

J104. The method of embodiment J102 or J103, wherein the IL-21 is recombinant IL-21.

J105. The method of any one of embodiments J74-J105, wherein the cell culture conditions of part (e) comprise about 1 ng/mL to about 100 ng/mL of IL-21.

J106. The method of any one of embodiments J74-J106, wherein the cell culture conditions of part (e) comprise about 5 ng/mL to about 20 ng/mL of IL-21.

J107. The method of any one of embodiments J74-J106, wherein the cell culture conditions of part (e) comprise an isolated component comprising a lipid.

J108. The method of embodiment J107, wherein the isolated component is a glycolipid.

J109. The method of embodiment J107 or J108, wherein the isolated component is alpha-galactosylceramide.

J110. The method of any one of embodiments J74-J109, wherein the cell culture conditions of part (e) comprise about 10 ng/mL to about 1,000 ng/mL of the isolated component comprising the lipid.

J111. The method of any one of embodiments J74-J109, wherein the cell culture conditions of part (e) comprise about 50 ng/mL to about 200 ng/mL of the isolated component comprising the lipid.

J112. The method of any one of embodiments J74-J111, wherein the cell culture conditions include no added extract from a non-human cells.

J113. The method of embodiment J112, wherein the cell culture conditions include no added non-human cells.

J114. The method of any one of embodiments J74-J113, wherein the cell culture conditions comprise no added irradiated cells and/or tumor cells.

J115. The method of any one of embodiments J74-J114, wherein the cell culture conditions of part (e) comprise human serum.

J116. The method of any one of embodiments J74-J115, wherein the cell culture conditions of part (e) are for about 3 days to about 25 days.

J117. The method of embodiment J116, wherein the cell culture conditions of part (e) are for about 5 days to about 15 days.

J118. The method of embodiment J117, wherein the cell culture conditions of part (e) are for about 10 days.

J119. The method of any one of embodiments J74-J118, wherein:
about 70% to about 99.9% of cells in the cell composition are CD3 positive and iNKT-TCR positive, and/or
about 20% or fewer cells in the cell composition are CD3 positive and iNKT-TCR negative,
after (e) is performed for about 5 days or more.

J120. The method of any one of embodiments J74-J119, wherein:
about 80% to about 90% of cells in the cell composition are CD3 positive and iNKT-TCR positive, and/or
about 15% or fewer cells in the cell composition are CD3 positive and iNKT-TCR negative,
after (e) is performed for about 7 days or more.

J121. The method of any one of embodiments J74-J120, wherein:
about 90% or more of cells in the cell composition are CD3 positive and iNKT-TCR positive, and/or
about 5% or fewer cells in the cell composition are CD3 positive and iNKT-TCR negative,
after (e) is performed for about 14 days or more.

J122. The method of any one of embodiments J74-J121, wherein:
about 97% or more of cells in the cell composition are CD3 positive and iNKT-TCR positive, and/or
about 0.5% or fewer cells in the cell composition are CD3 positive and iNKT-TCR negative,
after (e) is performed for about 14 days or more.

J123. The method of any one of embodiments J74-J122, wherein total expansion of cells in the cell population is about 100-fold to about 10,000-fold after part (e).

J124. The method of any one of embodiments J74-J123, wherein total expansion of cells in the cell population is about 300-fold to about 1,000-fold after part (e).

J125. The method any one of embodiments J74-J124, comprising introducing a prepared nucleic acid into cells of the cell composition after part (d).

J126. The method of embodiment J125, wherein the prepared nucleic acid is introduced to the cell composition after part (d) and before part (e).

J127. The method of embodiment J125, wherein the prepared nucleic acid is introduced to the cell composition before part (e), or within 1 day after exposing the separated cell composition to cell culture conditions.

J128. The method of any one of embodiments J125-J127, comprising introducing viral particles containing the prepared nucleic acid to the cell composition under conditions in which the viral particles enter cells of the cell composition.

J129. The method of any one of embodiments J125-J127, comprising introducing the prepared nucleic acid to the cell composition under conditions in which a polynucleotide of the nucleic acid integrates into cellular DNA of cells of the cell composition.

J130. The method of any one of embodiments J125-J127, comprising introducing the prepared nucleic acid to the cell composition under electroporation conditions in which the nucleic acid enters cells of the cell composition.

J131. The method of any one of embodiments J125-J130, wherein the nucleic acid comprises a polynucleotide encoding a protein.

J132. The method of embodiment J131, wherein the nucleic acid comprises a polynucleotide encoding a chimeric antigen receptor.

J133. The method of embodiment J131 or J132, wherein the polynucleotide encodes a binding molecule of any one of embodiments A1-A24, B1-B24 or C1-C58.

J134. The method of any one of embodiments J131-J133, wherein the polynucleotide encodes a chimeric protein of any one of embodiments G1-G3.

J135. The method of any one of embodiments J125-J134, wherein the nucleic acid and/or the polynucleotide encoding the protein is in about 20% to about 70% of cells in the cell population.

J136. The method of embodiment J125-J135, wherein the nucleic acid and/or the polynucleotide encoding the protein is in about 30% to about 60% of cells in the cell population.

J136.1. The method of embodiment J125-J134, wherein the nucleic acid and/or the polynucleotide encoding the protein is in about 65% or more of iNKT-cells in the cell population.

J136.2. The method of embodiment J125-J134, wherein the nucleic acid and/or the polynucleotide encoding the protein is in about 70% or more of iNKT-cells in the cell population.

J136.3. The method of embodiment J125-J134, wherein the nucleic acid and/or the polynucleotide encoding the protein is in about 75% or more of iNKT-cells in the cell population.

J136.4. The method of embodiment J125-J134, wherein the nucleic acid and/or the polynucleotide encoding the protein is in about 80% or more of iNKT-cells in the cell population.

J136.5. The method of embodiment J125-J134, wherein the nucleic acid and/or the polynucleotide encoding the protein is in about 85% or more of iNKT-cells in the cell population.

J137. The method of any one of embodiments J74-J136.5, comprising preserving cells after part (a), after part (b), after part (c), after part (d) and/or after part (e).

J138. The method of embodiment J137, wherein cells are preserved after (e).

J139. The method of embodiment J136 or J137, wherein cells are cryopreserved.

J140. The method of embodiment J139, wherein the cells are in a composition comprising a cryopreservation medium.

J141. The method of embodiment J139 or J140, wherein the cells are in cryopreservation conditions.

J142. The method of any one of embodiments J137-J141, wherein the cells are in a container.

J143. The method of any one of embodiments J1-J142, wherein cells of the cell population comprise a switch polypeptide of any one of embodiments D11-D34.

J144. A cell composition prepared by a method of any one of embodiments J1-J143.

J145. A pharmaceutical composition comprising a cell composition of embodiment J144 and a pharmaceutically acceptable carrier.

### Certain Polypeptides and Polynucleotides

Provided hereafter are non-limiting examples of polypeptides and polynucleotides described herein.

| | | |
|---|---|---|
| | SEQ ID | |
| Element | NO | Sequence and applicable structural formula |

Binding Molecule A

| | | |
|---|---|---|
| Heavy Chain Variable | 1 | MNFGFSLIFLVLVLKGVQCEVKLVESGGGLVKPGGSLKLSCEASGFTFSRYAMSWVRHTPEKRLAWVASITFGGDPYY |
| (VH) Domain with optional | | PDGLKGRFTISRDNTRNILYLQMNSLRSEDTAMYYCVRHESWFAYWGQGTLVTVSA |
| leader sequence | | (leader sequence in italic text; CDRs in bold text) |
| polypeptide | | Structural formula: Nterm-(leader sequence)-FR1-CDR1-FR2-CDR2-FR3-CDR3-FR4- |
| | | Cterm |
| Heavy Chain Variable | 2 | EVKLVESGGGLVKPGGSLKLSCEASGFTFSRYAMSWVRHTPEKRLAWVASITFGGDPYYPDGLKGRFTISRDNTRNIL |
| (VH) Domain | | YLQMNSLRSEDTAMYYCVRHESWFAYWGQGTLVTVSA |
| polypeptide | | (CDRs in bold text) |
| | | Structural formula: Nterm-FR1-CDR1-FR2-CDR2-FR3-CDR3-FR4-Cterm |
| VH Domain CDR1 | 3 | RYAMS |
| polypeptide | | |
| VH Domain CDR2 | 4 | SITFGGDPYYPDGLKG |
| polypeptide | | |
| VH Domain CDR3 | 5 | HESWFAY |
| polypeptide | | |
| VH Domain FR1 | 6 | EVKLVESGGGLVKPGGSLKLSCEASGFTFS |
| polypeptide | | |
| VH Domain FR2 | 7 | WVRHTPEKRLAWVA |
| polypeptide | | |
| VH Domain FR3 | 8 | RFTISRDNTRNILYLQMNSLRSEDTAMYYCVR |
| polypeptide | | |
| VH Domain FR4 | 9 | WGQGTLVTVSA |
| polypeptide | | |
| Light Chain Variable (VL) | 10 | MKLPVRLLVLMFWIPASSSDVLMTQTPLSLPVSLGEQVSISCRSSQTIVHTDGNIYLEWYLQNPGQSPRLLIYKISNR |
| Domain with optional | | FSGVPDRFSGSGSGTDFTLKISRVEAEDLGIYYCFQASHVPYTFGGGTKLEIK |
| leader sequence | | (leader sequence in italic text; CDRs in bold text) |
| polypeptide | | Structural formula: Nterm-(leader sequence)-FR1-CDR1-FR2-CDR2-FR3-CDR3-FR4- |
| | | Cterm |
| Light Chain Variable (VL) | 11 | DVLMTQTPLSLPVSLGEQVSISCRSSQTIVHTDGNIYLEWYLQNPGQSPRLLIYKISNRFSGVPDRFSGSGSGTDFTL |
| Domain | | KISRVEAEDLGIYYCFQASHVPYTFGGGTKLEIK |
| polypeptide | | (CDRs in bold text) |
| | | Structural formula: Nterm-FR1-CDR1-FR2-CDR2-FR3-CDR3-FR4-Cterm |
| VL Domain CDR1 | 12 | RSSQTIVHTDGNIYLE |
| polypeptide | | |
| VL Domain CDR2 | 13 | KISNRFS |
| polypeptide | | |
| VL Domain CDR3 | 14 | FQASHVPYT |
| polypeptide | | |
| VL Domain FR1 | 15 | DVLMTQTPLSLPVSLGEQVSISC |
| polypeptide | | |
| VL Domain FR2 | 16 | WYLQNPGQSPRLLIY |
| polypeptide | | |
| VL Domain FR3 | 17 | GVPDRFSGSGSGTDFTLKISRVEAEDLGIYYC |
| polypeptide | | |
| VL Domain FR4 | 18 | FGGGTKLEIK |
| polypeptide | | |
| Heavy Chain Variable | 19 | ATGAACTTCGGGTTCAGCTTGATTTTCCTTGTCCTTGTTTTAAAAGGTGTCCAGTGTGAAGTGAAGCTGGTGGAGTCT |
| (VH) Domain with optional | | GGGGGAGGCTTAGTGAAGCCTGGGGGGTCCCTGAAACTCTCCTGTGAGGCCTCTGGGTTCACTTTCAGTCGCTATGCC |
| leader sequence in italic | | ATGTCTTGGGTTCGCCATACTCCAGAGAAGAGACTGGCCTGGGTCGCATCCATAACTTTTGGTGGTGACCCGTATTAT |
| text; CDRs in bold text | | CCCGACGGTTTGAAGGGCCGATTCACCATCTCCAGAGATAATACCAGGAACATCCTGTACCTGCAAATGAACAGTCTG |
| polynucleotide | | AGGTCTGAAGACACGGCCATGTATTATTGTGTACGACACGAGTCATGGTTTGCTTACTGGGGCCAAGGGACTCTGGTC |
| | | ACTGTCTCTGCA |
| | | Structural formula: 5′-(leader sequence)-FR1-CDR1-FR2-CDR2-FR3-CDR3-FR4-3′ |
| Heavy Chain Variable | 20 | GAAGTGAAGCTGGTGGAGTCTGGGGGAGGCTTAGTGAAGCCTGGGGGGTCCCTGAAACTCTCCTGTGAGGCCTCTGGG |
| (VH) Domain (CDRs in | | TTCACTTTCAGTCGCTATGCCATGTCTTGGGTTCGCCATACTCCAGAGAAGAGACTGGCCTGGGTCGCATCCATAACT |
| bold text) | | TTTGGTGGTGACCCGTATTATCCCGACGGTTTGAAGGGCCGATTCACCATCTCCAGAGATAATACCAGGAACATCCTG |
| polynucleotide | | TACCTGCAAATGAACAGTCTGAGGTCTGAAGACACGGCCATGTATTATTGTGTACGACACGAGTCATGGTTTGCTTAC |
| | | TGGGGCCAAGGGACTCTGGTCACTGTCTCTGCA |
| | | Structural formula: 5′-FR1-CDR1-FR2-CDR2-FR3-CDR3-FR4-3′ |
| VH Domain CDR1 | 21 | CGCTATGCCATGTCT |
| polynucleotide | | |
| VH Domain CDR2 | 22 | TCCATAACTTTTGGTGGTGACCCGTATTATCCCGACGGTTTGAAGGGC |
| polynucleotide | | |
| VH Domain CDR3 | 23 | CACGAGTCATGGTTTGCTTAC |
| polynucleotide | | |
| VH Domain FR1 | 24 | GAAGTGAAGCTGGTGGAGTCTGGGGGAGGCTTAGTGAAGCCTGGGGGGTCCCTGAAACTCTCCTGTGAGGCCTCTGGG |
| polynucleotide | | TTCACTTTCAGT |
| VH Domain FR2 | 25 | TGGGTTCGCCATACTCCAGAGAAGAGACTGGCCTGGGTCGCA |
| polynucleotide | | |
| VH Domain FR3 | 26 | CGATTCACCATCTCCAGAGATAATACCAGGAACATCCTGTACCTGCAAATGAACAGTCTGAGGTCTGAAGACACGGCC |
| polynucleotide | | ATGTATTATTGTGTACGA |
| VH Domain FR4 | 27 | TGGGGCCAAGGGACTCTGGTCACTGTCTCTGCA |
| polynucleotide | | |
| Light Chain Variable (VL) | 28 | ATGAAGTTGCCTGTTAGGCTGTTGGTGCTGATGTTCTGGATTCCTGCTTCCAGCAGTGATGTTTTGATGACCCAAACT |
| Domain with optional | | CCACTCTCCCTGCCTGTCAGTCTTGGAGAACAAGTCTCCATCTCTTGCAGATCTAGTCAGACCATTGTACATACTGAT |
| leader sequence in italic | | GGCAACATCTATTTAGAATGGTACCTCCAGAACCCAGGCCAGTCTCCTAGGCTCCTGATCTACAAAATTTCCAACCGA |
| text; CDRs in bold text | | TTTTCTGGGGTCCCTGACAGGTTCAGTGGCAGTGGATCAGGGACAGATTTCACACTCAAGATCAGCAGAGTGGAGGCT |
| polynucleotide | | GAGGATCTGGGAATTTATTACTGCTTTCAAGCTTCACATGTTCCGTACACGTTCGGAGGGGGGACCAAGCTGGAAATA |
| | | AAA |
| | | Structural formula: 5′-(leader sequence)-FR1-CDR1-FR2-CDR2-FR3-CDR3-FR4-3′ |
| Light Chain Variable (VL) | 29 | GATGTTTTGATGACCCAAACTCCACTCTCCCTGCCTGTCAGTCTTGGAGAACAAGTCTCCATCTCTTGCAGATCTAGT |
| Domain (CDRs in bold | | CAGACCATTGTACATACTGATGGCAACATCTATTTAGAATGGTACCTCCAGAACCCAGGCCAGTCTCCTAGGCTCCTG |
| text) | | ATCTACAAAATTTCCAACCGATTTTCTGGGGTCCCTGACAGGTTCAGTGGCAGTGGATCAGGGACAGATTTCACACTC |
| polynucleotide | | AAGATCAGCAGAGTGGAGGCTGAGGATCTGGGAATTTATTACTGCTTTCAAGCTTCACATGTTCCGTACACGTTCGGA |
| | | GGGGGGACCAAGCTGGAAATAAAA |
| | | Structural formula: 5′-FR1-CDR1-FR2-CDR2-FR3-CDR3-FR4-3′ |
| VL Domain CDR1 | 30 | AGATCTAGTCAGACCATTGTACATACTGATGGCAACATCTATTTAGAA |
| polynucleotide | | |
| VL Domain CDR2 | 31 | AAAATTTCCAACCGATTTTCT |
| polynucleotide | | |
| VL Domain CDR3 | 32 | TTTCAAGCTTCACATGTTCCGTACACG |
| polynucleotide | | |
| VL Domain FR1 | 33 | GATGTTTTGATGACCCAAACTCCACTCTCCCTGCCTGTCAGTCTTGGAGAACAAGTCTCCATCTCTTGC |
| polynucleotide | | |
| VL Domain FR2 | 34 | TGGTACCTCCAGAACCCAGGCCAGTCTCCTAGGCTCCTGATCTAC |
| polynucleotide | | |
| VL Domain FR3 | 35 | GGGGTCCCTGACAGGTTCAGTGGCAGTGGATCAGGGACAGATTTCACACTCAAGATCAGCAGAGTGGAGGCTGAGGAT |
| polynucleotide | | CTGGGAATTTATTACTGC |
| VL Domain FR4 | 36 | TTCGGAGGGGGGACCAAGCTGGAAATAAAA |
| polynucleotide | | |

Binding Molecule B

| | | |
|---|---|---|
| Heavy Chain Variable | 37 | MNFGFSLIFLVLVLKGVQCEVKLVESGGGLVKPGGSLKVSCTASGFTFSRYAMSWVRQTPERRLEWVASITFGGSAYY |
| (VH) Domain with optional | | LDSVKGRFTISRDNAQNILYLQMNSLVSEDTAIYNCARHQPWFDYWGQGTLVTVSA |
| leader sequence | | (leader sequence in italic text; CDRs in bold text) |
| polypeptide | | Structural formula: Nterm-(leader sequence)-FR1-CDR1-FR2-CDR2-FR3-CDR3-FR4- |
| | | Cterm |
| Heavy Chain Variable | 38 | EVKLVESGGGLVKPGGSLKVSCTASGFTFSRYAMSWVRQTPERRLEWVASITFGGSAYYLDSVKGRFTISRDNAQNIL |
| (VH) Domain | | YLQMNSLVSEDTAIYNCARHQPWFDYWGQGTLVTVSA |
| polypeptide | | (CDRs in bold text) |
| | | Structural formula: Nterm-FR1-CDR1-FR2-CDR2-FR3-CDR3-FR4-Cterm |
| VH Domain CDR1 | 39 | RYAMS |
| polypeptide | | |
| VH Domain CDR2 | 40 | SITFGGSAYYLDSVKG |
| polypeptide | | |
| VH Domain CDR3 | 41 | HQPWFDY |
| polypeptide | | |
| VH Domain FR1 | 42 | EVKLVESGGGLVKPGGSLKVSCTASGFTFS |
| polypeptide | | |
| VH Domain FR2 | 43 | WVRQTPERRLEWVA |
| polypeptide | | |
| VH Domain FR3 | 44 | RFTISRDNAQNILYLQMNSLVSEDTAIYNCAR |
| polypeptide | | |
| VH Domain FR4 | 45 | WGQGTLVTVSA |
| polypeptide | | |
| Light Chain Variable (VL) | 46 | MNLPIRLLVLMFWIPASSSDVLLTQTPLSLPVSLGDQASISCRSSQNIXHSDGDTYLDWFLQKPGQSPNLLIYKVSNR |
| Domain with optional | | FSGVPDRFSGSGSGTDFTLKISRVEADDLGVYYCFQASHVPYTFGGGTKLEVK |
| leader sequence | | (leader sequence in italic text; CDRs in bold text) |
| polypeptide | | Structural formula: Nterm-(leader sequence)-FR1-CDR1-FR2-CDR2-FR3-CDR3-FR4- |
| | | Cterm leader sequence-FR1-CDR1-FR2-CDR2-FR3-CDR3-FR4) |
| | | X is I or V |
| Light Chain Variable (VL) | 47 | DVLLTQTPLSLPVSLGDQASISCRSSQNIXHSDGDTYLDWFLQKPGQSPNLLIYKVSNRFSGVPDRFSGSGSGTDFTL |
| Domain | | KISRVEADDLGVYYCFQASHVPYTFGGGTKLEVK |
| polypeptide | | (CDRs in bold text) |
| | | Structural formula: Nterm-FR1-CDR1-FR2-CDR2-FR3-CDR3-FR4-Cterm |
| | | X is I or V |
| VL Domain CDR1 | 48 | RSSQNIXHSDGDTYLD |
| polypeptide | | X is I or V |
| VL Domain CDR2 | 49 | KVSNRFS |
| polypeptide | | |
| VL Domain CDR3 | 50 | FQASHVPYT |
| polypeptide | | |
| VL Domain FR1 | 51 | DVLLTQTPLSLPVSLGDQASISC |
| polypeptide | | |
| VL Domain FR2 | 52 | WFLQKPGQSPNLLIY |
| polypeptide | | |
| VL Domain FR3 | 53 | GVPDRFSGSGSGTDFTLKISRVEADDLGVYYC |
| polypeptide | | |
| VL Domain FR4 | 54 | FGGGTKLEVK |
| polypeptide | | |
| Heavy Chain Variable | 55 | ATGAACTTCGGGTTCAGCTTGATTTTCCTTGTCCTTGTTTTAAAAGGTGTCCAGTGCGAAGTGAAGCTGGTGGAGTCT |
| (VH) Domain with optional | | GGGGGAGGCTTAGTGAAGCCTGGAGGGTCCCTGAAAGTCTCCTGTACAGCCTCTGGATTCACTTTCAGTCGTTATGCC |
| leader sequence in italic | | ATGTCTTGGGTTCGCCAGACTCCAGAGAGGAGGCTGGAGTGGGTCGCATCCATTACTTTTGGTGGTAGCGCCTACTAT |
| text; CDRs in bold text | | TTAGACAGTGTGAAGGGCCGATTCACCATCTCCAGAGATAATGCCCAGAACATCCTGTACCTGCAAATGAACAGTCTG |
| polynucleotide | | GTGTCTGAGGACACGGCCATTTATAACTGTGCAAGACACCAGCCCTGGTTTGATTACTGGGGCCAAGGGACTCTGGTC |
| | | ACTGTCTCTGCA |
| | | Structural formula: 5′-(leader sequence)-FR1-CDR1-FR2-CDR2-FR3-CDR3-FR4-3′ |
| Heavy Chain Variable | 56 | GAAGTGAAGCTGGTGGAGTCTGGGGGAGGCTTAGTGAAGCCTGGAGGGTCCCTGAAAGTCTCCTGTACAGCCTCTGGA |
| (VH) Domain (CDRs in | | TTCACTTTCAGTCGTTATGCCATGTCTTGGGTTCGCCAGACTCCAGAGAGGAGGCTGGAGTGGGTCGCATCCATTACT |
| bold text) | | TTTGGTGGTAGCGCCTACTATTTAGACAGTGTGAAGGGCCGATTCACCATCTCCAGAGATAATGCCCAGAACATCCTG |
| polynucleotide | | TACCTGCAAATGAACAGTCTGGTGTCTGAGGACACGGCCATTTATAACTGTGCAAGACACCAGCCCTGGTTTGATTAC |
| | | TGGGGCCAAGGGACTCTGGTCACTGTCTCTGCA |
| | | Structural formula: 5′-FR1-CDR1-FR2-CDR2-FR3-CDR3-FR4-3′ |
| VH Domain CDR1 | 57 | CGTTATGCCATGTCT |
| polynucleotide | | |
| VH Domain CDR2 | 58 | TCCATTACTTTTGGTGGTAGCGCCTACTATTTAGACAGTGTGAAGGGC |
| polynucleotide | | |
| VH Domain CDR3 | 59 | CACCAGCCCTGGTTTGATTAC |
| polynucleotide | | |
| VH Domain FR1 | 60 | GAAGTGAAGCTGGTGGAGTCTGGGGGAGGCTTAGTGAAGCCTGGAGGGTCCCTGAAAGTCTCCTGTACAGCCTCTGGA |
| polynucleotide | | TTCACTTTCAGT |
| VH Domain FR2 | 61 | TGGGTTCGCCAGACTCCAGAGAGGAGGCTGGAGTGGGTCGCA |
| polynucleotide | | |
| VH Domain FR3 | 62 | CGATTCACCATCTCCAGAGATAATGCCCAGAACATCCTGTACCTGCAAATGAACAGTCTGGTGTCTGAGGACACGGCC |
| polynucleotide | | ATTTATAACTGTGCAAGA |
| VH Domain FR4 | 63 | TGGGGCCAAGGGACTCTGGTCACTGTCTCTGCA |
| polynucleotide | | |
| Light Chain Variable (VL) | 64 | ATGAACTTGCCTATTAGGCTGTTGGTGCTGATGTTCTGGATTCCTGCTTCCAGCAGTGATGTCTTGCTGACCCAGACT |
| Domain with optional | | CCACTCTCCCTGCCTGTCAGTCTTGGAGATCAGGCCTCCATCTCTTGCAGATCTAGTCAGAACATTZTYCATAGTGAT |
| leader sequence in italic | | GGAGACACCTATTTAGACTGGTTCCTGCAGAAACCAGGCCAGTCTCCAAACCTCCTAATCTACAAAGTTTCCAACCGA |
| text; CDRs in bold text | | TTTTCTGGGGTCCCAGACAGGTTCAGTGGCAGTGGATCAGGGACAGATTTCACCCTCAAGATCAGCAGAGTGGAGGCT |
| polynucleotide | | GACGATCTGGGAGTTTATTACTGCTTTCAAGCTTCACATGTTCCGTACACGTTCGGAGGGGGGACCAAGCTGGAAGTA |
| | | AAA |
| | | Structural formula: Nterm-(leader sequence)-FR1-CDR1-FR2-CDR2-FR3-CDR3-FR4- |
| | | Cterm |
| | | Z is A or G; Y is A or T |
| Light Chain Variable (VL) | 65 | GATGTCTTGCTGACCCAGACTCCACTCTCCCTGCCTGTCAGTCTTGGAGATCAGGCCTCCATCTCTTGCAGATCTAGT |
| Domain (CDRs in bold | | CAGAACATTZTYCATAGTGATGGAGACACCTATTTAGACTGGTTCCTGCAGAAACCAGGCCAGTCTCCAAACCTCCTA |
| text) | | ATCTACAAAGTTTCCAACCGATTTTCTGGGGTCCCAGACAGGTTCAGTGGCAGTGGATCAGGGACAGATTTCACCCTC |
| polynucleotide | | AAGATCAGCAGAGTGGAGGCTGACGATCTGGGAGTTTATTACTGCTTTCAAGCTTCACATGTTCCGTACACGTTCGGA |
| | | GGGGGGACCAAGCTGGAAGTAAAA |
| | | Structural formula: 5′-FR1-CDR1-FR2-CDR2-FR3-CDR3-FR4-3′ |
| | | Z is A or G; Y is A or T |
| VL Domain CDR1 | 66 | AGATCTAGTCAGAACATTZTYCATAGTGATGGAGACACCTATTTAGAC |
| polynucleotide | | Z is A or G; Y is A or T |
| VL Domain CDR2 | 67 | AAAGTTTCCAACCGATTTTCT |
| polynucleotide | | |
| VL Domain CDR3 | 68 | TTTCAAGCTTCACATGTTCCGTACACG |
| polynucleotide | | |
| VL Domain FR1 | 69 | GATGTCTTGCTGACCCAGACTCCACTCTCCCTGCCTGTCAGTCTTGGAGATCAGGCCTCCATCTCTTGC |
| polynucleotide | | |
| VL Domain FR2 | 70 | TGGTTCCTGCAGAAACCAGGCCAGTCTCCAAACCTCCTAATCTAC |
| polynucleotide | | |
| VL Domain FR3 | 71 | GGGGTCCCAGACAGGTTCAGTGGCAGTGGATCAGGGACAGATTTCACCCTCAAGATCAGCAGAGTGGAGGCTGACGAT |
| polynucleotide | | CTGGGAGTTTATTACTGC |
| VL Domain FR4 | 72 | TTCGGAGGGGGGACCAAGCTGGAAGTAAAA |
| polynucleotide | | |

Binding Molecule C

| | | |
|---|---|---|
| CAR polypeptide | 73 | MALPVTALLLPLALLLHAARPGSELPTQGTFSNVSTNVSSPEVKLVESGGGLVKPGGSLKLSCEASGFTFSRYAMSWV |
| | | RHTPEKRLAWVASITFGGDPYYPDGLKGRFTISRDNTRNILYLQMNSLRSEDTAMYYCVRHESWFAYWGQGTLVTVSA |
| | | GGGGSGGGGSGGGGSDVLMTQTPLSLPVSLGEQVSISCRSSQTIVHTDGNIYLEWYLQNPGQSPRLLIYKISNRFSGV |
| | | PDRFSGSGSGTDFTLKISRVEAEDLGIYYCFQASHVPYTFGGGTKLEIKRTTTPAPRPPTPAPTIASQPLSLRPEACR |
| | | PAAGGAVHTRGLDFACDIYIWAPLAGTCGVLLLSLVITLYCNHRNRRRVCKCPRVDRSKRSRLLHSDYMNMTPRRPGP |
| | | TRKHYQPYAPPRDFAAYRSRVKESRSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| | | Structural formula: Nterm-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-Cterm |
| CAR polynucleotide | 74 | ATGGCTCTTCCGGTAACCGCTCTGCTGCTTCCTTTGGCTCTGCTTCTTCACGCGGCTAGGCCAGGGAGCGAATTGCCT |
| | | ACGCAGGGGACTTTTTCAAATGTAAGCACGAATGTgagCTCTCCGGAAGTGAAATTGGTAGAGTCTGGTGGGGGACTG |
| | | GTTAAACCTGGAGGCAGTCTGAAGCTCTCCTGCGAAGCCAGCGGCTTTACGTTCAGTCGGTACGCTATGTCCTGGGTG |
| | | CGACACACACCAGAAAAGCGACTCGCTTGGGTAGCGAGCATAACGTTTGGGGGCGATCCTTATTACCCGGATGGTTTG |
| | | AAAGGTCGCTTCACTATCAGTCGAGATAATACGCGAAACATTCTTTATCTTCAAATGAATAGCCTGCGCTCTGAAGAC |
| | | ACCGCGATGTATTATTGTGTTAGGCATGAGTCTTGGTTTGCATACTGGGGTCAGGGTACTCTTGTCACTGTATCTGCC |
| | | GGAGGTGGGGGTAGCGGCGGCGGAGGTAGTGGGGGAGGCGGCAGCGACGTGTTGATGACTCAAACTCCGCTTAGCTTG |
| | | CCAGTGTCTCTTGGAGAACAGGTCTCCATATCATGTCGATCCAGTCAAACTATCGTTCATACCGATGGGAATATCTAT |
| | | CTCGAATGGTATCTGCAAAATCCAGGGCAGAGTCCCCGGCTTCTGATaTATAAGATTAGCAATAGATTTAGTGGTGTT |
| | | CCCGACaGGTTTTCAGGTTCTGGGTCCGGAACGGATTTTACCCTGAAGATAAGTCGAGTTGAAGCGGAAGACCTGGGC |
| | | ATATACTATTGCTTCCAGGCGTCCCACGTCCCCTATACATTTGGGGGTGGGACTAAACTGGAAATTAAACGtACgACG |
| | | ACaCCAGCCCCAAGACCCCCAACTCCCGCTCCTACCATAGCTTCCCAACCCCTGTCACTGAGGCCAGAGGCATGCAGG |
| | | CCCGCTGCGGGTGGCGCGGTACATACGCGGGGACTGGACTTTGCATGTGACATTTACATATGGGCGCCACTGGCGGGA |
| | | ACATGTGGAGTTTTGTTGCTTAGCCTGGTCATAACACTGTATTGCAATCATCGCAACAGACGCCGCGTCTGCAAGTGT |
| | | CCCAGGGTCGACAGATCTAAGAGAAGTAGACTTCTTCACAGTGATTATATGAATATGACGCCTCGAAGACCCGGCCCG |
| | | ACACGCAAACACTATCAGCCGTATGCCCCCCCTCGGGATTTTGCTGCCTACCGCAGCCGCGTTAAGTTCTCTAGGTCC |
| | | GCTGACGCCCCTGCCTACCAGCAGGGTCAAAACCAACTGTACAATGAATTGAATCTTGGGAGACGGGAGGAGTATGAC |
| | | GTACTCGACAAGCGGAGGGGGAGAGATCCTGAGATGGGTGGAAAGCCTCGACGAAAAAACCCACAAGAAGGGTTGTAT |
| | | AATGAACTGCAGAAAGACAAGATGGCTGAGGCATATAGTGAAATCGGGATGAAGGGAGAACGCCGGCGCGGGAAAGGG |
| | | CACGATGGTCTGTATCAAGGGCTTAGTACGGCTACGAAGGACACATATGACGCTTTGCACATGCAGGCCCTTCCTCCT |
| | | AGG |
| | | Structural formula: 5′-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-3′ |
| CD8 signal polypeptide | 75 | MALPVTALLLPLALLLHAARP |
| CD8 signal polynucleotide | 76 | ATGGCTCTTCCGGTAACCGCTCTGCTGCTTCCTTTGGCTCTGCTTCTTCACGCGGCTAGGCCA |
| Linker 1 polypeptide | 77 | GS |
| Linker 1 polynucleotide | 78 | GGGAGC |
| CD34 tag polypeptide | 79 | ELPTQGTFSNVSTNVS |
| CD34 tag polynucleotide | 80 | GAATTGCCTACGCAGGGGACTTTTTCAAATGTAAGCACGAATGTGAGC |
| Linker 2 polypeptide | 81 | SP |
| Linker 2 polynucleotide | 82 | TCTCCG |
| VH Domain polypeptide | 83 | EVKLVESGGGLVKPGGSLKLSCEASGFTFSRYAMSWVRHTPEKRLAWVASITFGGDPYYPDGLKGRFTISRDNTRNIL |
| | | YLQMNSLRSEDTAMYYCVRHESWFAYWGQGTLVTVSA |
| VH Domain polynucleotide | 84 | GAAGTGAAATTGGTAGAGTCTGGTGGGGGACTGGTTAAACCTGGAGGCAGTCTGAAGCTCTCCTGCGAAGCCAGCGGC |
| | | TTTACGTTCAGTCGGTACGCTATGTCCTGGGTGCGACACACACCAGAAAAGCGACTCGCTTGGGTAGCGAGCATAACG |
| | | TTTGGGGGCGATCCTTATTACCCGGATGGTTTGAAAGGTCGCTTCACTATCAGTCGAGATAATACGCGAAACATTCTT |
| | | TATCTTCAAATGAATAGCCTGCGCTCTGAAGACACCGCGATGTATTATTGTGTTAGGCATGAGTCTTGGTTTGCATAC |
| | | TGGGGTCAGGGTACTCTTGTCACTGTATCTGCC |
| Linker 3 polypeptide | 85 | GGGGSGGGGSGGGGS |
| Linker 3 polynucleotide | 86 | GGAGGTGGGGGTAGCGGCGGCGGAGGTAGTGGGGGAGGCGGCAGC |
| VL Domain polypeptide | 87 | DVLMTQTPLSLPVSLGEQVSISCRSSQTIVHTDGNIYLEWYLQNPGQSPRLLIYKISNRFSGVPDRFSGSGSGTDFTL |
| | | KISRVEAEDLGIYYCFQASHVPYTFGGGTKLEIK |
| VL Domain polynucleotide | 88 | GACGTGTTGATGACTCAAACTCCGCTTAGCTTGCCAGTGTCTCTTGGAGAACAGGTCTCCATATCATGTCGATCCAGT |
| | | CAAACTATCGTTCATACCGATGGGAATATCTATCTCGAATGGTATCTGCAAAATCCAGGGCAGAGTCCCCGGCTTCTG |
| | | ATATATAAGATTAGCAATAGATTTAGTGGTGTTCCCGACAGGTTTTCAGGTTCTGGGTCCGGAACGGATTTTACCCTG |
| | | AAGATAAGTCGAGTTGAAGCGGAAGACCTGGGCATATACTATTGCTTCCAGGCGTCCCACGTCCCCTATACATTTGGG |
| | | GGTGGGACTAAACTGGAAATTAAA |
| Linker 4 polypeptide | 89 | R |
| Linker 4 polynucleotide | 90 | CGT |
| CD8 stalk polypeptide | 91 | TTTPAPRPPTPAPTIASQPLSLRPEACRPAAGGAVHTRGLDFACD |
| CD8 stalk polynucleotide | 92 | ACGACGACACCAGCCCCAAGACCCCCAACTCCCGCTCCTACCATAGCTTCCCAACCCCTGTCACTGAGGCCAGAGGCA |
| | | TGCAGGCCCGCTGCGGGTGGCGCGGTACATACGCGGGGACTGGACTTTGCATGTGAC |
| CD8 transmembrane | 93 | IYIWAPLAGTCGVLLLSLVITLYCNHRNRRRVCKCPR |
| polypeptide | | |
| CD8 transmembrane | 94 | ATTTACATATGGGCGCCACTGGCGGGAACATGTGGAGTTTTGTTGCTTAGCCTGGTCATAACACTGTATTGCAATCAT |
| polynucleotide | | CGCAACAGACGCCGCGTCTGCAAGTGTCCCAGG |
| Linker 5 polypeptide | 95 | VD |
| Linker 5 polynucleotide | 96 | GTCGAC |
| CD28 cytoplasmic | 97 | RSKRSRLLHSDYMNMTPRRPGPTRKHYQPYAPPRDFAAYRS |
| polypeptide | | |
| CD28 cytoplasmic | 98 | AGATCTAAGAGAAGTAGACTTCTTCACAGTGATTATATGAATATGACGCCTCGAAGACCCGGCCCGACACGCAAACAC |
| polynucleotide | | TATCAGCCGTATGCCCCCCCTCGGGATTTTGCTGCCTACCGCAGC |
| CD3-zeta cytoplasmic | 99 | RVKFSRSADAPAYQQGQNQLYNELNLGRREEYDVLDKRRGRDPEMGGKPRRKNPQEGLYNELQKDKMAEAYSEIGMKG |
| polypeptide | | ERRRGKGHDGLYQGLSTATKDTYDALHMQALPPR |
| CD3-zeta cytoplasmic | 100 | CGCGTTAAGTTCTCTAGGTCCGCTGACGCCCCTGCCTACCAGCAGGGTCAAAACCAACTGTACAATGAATTGAATCTT |
| polynucleotide | | GGGAGACGGGAGGAGTATGACGTACTCGACAAGCGGAGGGGGAGAGATCCTGAGATGGGTGGAAAGCCTCGACGAAAA |
| | | AACCCACAAGAAGGGTTGTATAATGAACTGCAGAAAGACAAGATGGCTGAGGCATATAGTGAAATCGGGATGAAGGGA |
| | | GAACGCCGGCGCGGGAAAGGGCACGATGGTCTGTATCAAGGGCTTAGTACGGCTACGAAGGACACATATGACGCTTTG |
| | | CACATGCAGGCCCTTCCTCCTAGG |

Binding Molecule D

| | | |
|---|---|---|
| CAR polypeptide | 101 | MALPVTALLLPLALLLHAARPGSELPTQGTFSNVSTNVSSPEVKLVESGGGLVKPGGSLKVSCTASGFTFSRYAMSWV |
| | | RQTPERRLEWVASITFGGSAYYLDSVKGRFTISRDNAQNILYLQMNSLVSEDTAIYNCARHQPWFDYWGQGTLVTVSA |
| | | GGGGSGGGGSGGGGSDVLLTQTPLSLPVSLGDQASISCRSSQNIXHSDGDTYLDWFLQKPGQSPNLLIYKVSNRFSGV |
| | | PDRFSGSGSGTDFTLKISRVEADDLGVYYCFQASHVPYTFGGGTKLEVKRTTTPAPRPPTPAPTIASQPLSLRPEACR |
| | | PAAGGAVHTRGLDFACDIYIWAPLAGTCGVLLLSLVITLYCNHRNRRRVCKCPRVDRSKRSRLLHSDYMNMTPRRPGP |
| | | TRKHYQPYAPPRDFAAYRSRVKFSRSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| | | Structural formula: Nterm-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-Cterm |
| | | X is I or V |
| CAR polynucleotide | 102 | ATGGCTCTTCCGGTAACCGCTCTGCTGCTTCCTTTGGCTCTGCTTCTTCACGCGGCTAGGCCAGGGAGCGAATTGCCT |
| | | ACGCAGGGGACTTTTTCAAATGTAAGCACGAATGTGAGCTCTCCGAAGTCAAATTGGTGGAGAGTGGCGGTGGGCTCG |
| | | TAAAACCAGGAGGCAGCTTGAAAGTAAGTTGCACAGCGTCTGGGTTCACTTTCAGCCGATACGCTATGAGTTGGGTGC |
| | | GGCAAACGCCCGAAAGACGATTGGAGTGGGTAGCTAGTATTACTTTTGGGGGTTCCGCGTATTACTTGGATAGCGTTA |
| | | AAGGGAGATTCACCATATCACGAGATAATGCCCAAAACATACTCTACCTCCAGATGAACTCATTGGTGTCTGAAGATA |
| | | CTGCAATTTACAACTGCGCCAGGCACCAGCCATGGTTTGACTATTGGGGTCAAGGTACGTTGGTCACAGTCTCAGCCG |
| | | GCGGTGGCGGATCTGGAGGGGGAGGCAGTGGAGGAGGTGGTTCAGATGTTCTCCTGACGCAAACTCCTTTGTCATTGC |
| | | CGGTAAGTCTGGGCGACCAAGCGAGCATTTCCTGTCGCAGCAGCCAGAACATAZTYCATAGTGATGGCGACACTTACT |
| | | TGGATTGGTTCCTCCAGAAACCGGGGCAATCTCCGAACCTCTTGATATATAAGGTCTCAAATCGCTTTAGCGGGGTTC |
| | | CCGACCGCTTTTCAGGTTCTGGCTCAGGTACCGATTTCACGCTGAAAATTTCTAGAGTAGAGGCTGACGATCTGGGTG |
| | | TTTACTATTGTTTTCAAGCTTCACACGTTCCTTATACGTTCGGAGGGGGGACTAAACTGGAGGTTAAACGTACGACGA |
| | | CACCAGCCCCAAGACCCCCAACTCCCGCTCCTACCATAGCTTCCCAACCCCTGTCACTGAGGCCAGAGGCATGCAGGC |
| | | CCGCTGCGGGTGGCGCGGTACATACGCGGGGACTGGACTTTGCATGTGACATTTACATATGGGCGCCACTGGCGGGAA |
| | | CATGTGGAGTTTTGTTGCTTAGCCTGGTCATAACACTGTATTGCAATCATCGCAACAGACGCCGCGTCTGCAAGTGTC |
| | | CCAGGGTCGACAGATCTAAGAGAAGTAGACTTCTTCACAGTGATTATATGAATATGACGCCTCGAAGACCCGGCCCGA |
| | | CACGCAAACACTATCAGCCGTATGCCCCCCCTCGGGATTTTGCTGCCTACCGCAGCCGCGTTAAGTTCTCTAGGTCCG |
| | | CTGACGCCCCTGCCTACCAGCAGGGTCAAAACCAACTGTACAATGAATTGAATCTTGGGAGACGGGAGGAGTATGACG |
| | | TACTCGACAAGCGGAGGGGGAGAGATCCTGAGATGGGTGGAAAGCCTCGACGAAAAAACCCACAAGAAGGGTTGTATA |
| | | ATGAACTGCAGAAAGACAAGATGGCTGAGGCATATAGTGAAATCGGGATGAAGGGAGAACGCCGGCGCGGGAAAGGGC |
| | | ACGATGGTCTGTATCAAGGGCTTAGTACGGCTACGAAGGACACATATGACGCTTTGCACATGCAGGCCCTTCCTCCTA |
| | | GG |
| | | Structural formula: 5′-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-3′ |
| | | Z is A or G; Y is A or T |
| CD8 signal polypeptide | 103 | MALPVTALLLPLALLLHAARP |
| CD8 signal polynucleotide | 104 | ATGGCTCTTCCGGTAACCGCTCTGCTGCTTCCTTTGGCTCTGCTTCTTCACGCGGCTAGGCCA |
| Linker 1 polypeptide | 105 | GS |
| Linker 1 polynucleotide | 106 | GGGAGC |
| CD34 tag polypeptide | 107 | ELPTQGTFSNVSTNVS |
| CD34 tag polynucleotide | 108 | GAATTGCCTACGCAGGGGACTTTTTCAAATGTAAGCACGAATGTGAGC |
| Linker 2 polypeptide | 109 | SP |
| Linker 2 polynucleotide | 110 | TCTCCG |
| VH Domain polypeptide | 111 | EVKLVESGGGLVKPGGSLKVSCTASGFTFSRYAMSWVRQTPERRLEWVASITFGGSAYYLDSVKGRFTISRDNAQNIL |
| | | YLQMNSLVSEDTAIYNCARHQPWFDYWGQGTLVTVSA |
| VH Domain polynucleotide | 112 | GAAGTCAAATTGGTGGAGAGTGGCGGTGGGCTCGTAAAACCAGGAGGCAGCTTGAAAGTAAGTTGCACAGCGTCTGGG |
| | | TTCACTTTCAGCCGATACGCTATGAGTTGGGTGCGGCAAACGCCCGAAAGACGATTGGAGTGGGTAGCTAGTATTACT |
| | | TTTGGGGGTTCCGCGTATTACTTGGATAGCGTTAAAGGGAGATTCACCATATCACGAGATAATGCCCAAAACATACTC |
| | | TACCTCCAGATGAACTCATTGGTGTCTGAAGATACTGCAATTTACAACTGCGCCAGGCACCAGCCATGGTTTGACTAT |
| | | TGGGGTCAAGGTACGTTGGTCACAGTCTCAGCC |
| Linker 3 polypeptide | 113 | GGGGSGGGGSGGGGS |
| Linker 3 polynucleotide | 114 | GGCGGTGGCGGATCTGGAGGGGGAGGCAGTGGAGGAGGTGGTTCA |
| VL Domain polypeptide | 115 | DVLLTQTPLSLPVSLGDQASISCRSSQNIXHSDGDTYLDWFLQKPGQSPNLLIYKVSNRFSGVPDRFSGSGSGTDFTL |
| | | KISRVEADDLGVYYCFQASHVPYTFGGGTKLEVK |
| | | X is I or V |
| VL Domain polynucleotide | 116 | GATGTTCTCCTGACGCAAACTCCTTTGTCATTGCCGGTAAGTCTGGGCGACCAAGCGAGCATTTCCTGTCGCAGCAGC |
| | | CAGAACATAZTYCATAGTGATGGCGACACTTACTTGGATTGGTTCCTCCAGAAACCGGGGCAATCTCCGAACCTCTTG |
| | | ATATATAAGGTCTCAAATCGCTTTAGCGGGGTTCCCGACCGCTTTTCAGGTTCTGGCTCAGGTACCGATTTCACGCTG |
| | | AAAATTTCTAGAGTAGAGGCTGACGATCTGGGTGTTTACTATTGTTTTCAAGCTTCACACGTTCCTTATACGTTCGGA |
| | | GGGGGGACTAAACTGGAGGTTAAA |
| | | Z is A or G; Y is A or T |
| Linker 4 polypeptide | 117 | R |
| Linker 4 polynucleotide | 118 | CGT |
| CD8 stalk polypeptide | 119 | TTTPAPRPPTPAPTIASQPLSLRPEACRPAAGGAVHTRGLDFACD |
| CD8 stalk polynucleotide | 120 | ACGACGACACCAGCCCCAAGACCCCCAACTCCCGCTCCTACCATAGCTTCCCAACCCCTGTCACTGAGGCCAGAGGCA |
| | | TGCAGGCCCGCTGCGGGTGGCGCGGTACATACGCGGGGACTGGACTTTGCATGTGAC |
| CD8 transmembrane | 121 | IYIWAPLAGTCGVLLLSLVITLYCNHRNRRRVCKCPR |
| polypeptide | | |
| CD8 transmembrane | 122 | ATTTACATATGGGCGCCACTGGCGGGAACATGTGGAGTTTTGTTGCTTAGCCTGGTCATAACACTGTATTGCAATCAT |
| polynucleotide | | CGCAACAGACGCCGCGTCTGCAAGTGTCCCAGG |
| Linker 5 polypeptide | 123 | VD |
| Linker 5 polynucleotide | 124 | GTCGAC |
| CD28 cytoplasmic | 125 | RSKRSRLLHSDYMNMTPRRPGPTRKHYQPYAPPRDFAAYRS |
| polypeptide | | |
| CD28 cytoplasmic | 126 | AGATCTAAGAGAAGTAGACTTCTTCACAGTGATTATATGAATATGACGCCTCGAAGACCCGGCCCGACACGCAAACAC |
| polynucleotide | | TATCAGCCGTATGCCCCCCCTCGGGATTTTGCTGCCTACCGCAGC |
| CD3-zeta cytoplasmic | 127 | RVKFSRSADAPAYQQGQNQLYNELNLGRREEYDVLDKRRGRDPEMGGKPRRKNPQEGLYNELQKDKMAEAYSEIGMKG |
| polypeptide | | ERRRGKGHDGLYQGLSTATKDTYDALHMQALPPR |
| CD3-zeta cytoplasmic | 128 | CGCGTTAAGTTCTCTAGGTCCGCTGACGCCCCTGCCTACCAGCAGGGTCAAAACCAACTGTACAATGAATTGAATCTT |
| polynucleotide | | GGGAGACGGGAGGAGTATGACGTACTCGACAAGCGGAGGGGGAGAGATCCTGAGATGGGTGGAAAGCCTCGACGAAAA |
| | | AACCCACAAGAAGGGTTGTATAATGAACTGCAGAAAGACAAGATGGCTGAGGCATATAGTGAAATCGGGATGAAGGGA |
| | | GAACGCCGGCGCGGGAAAGGGCACGATGGTCTGTATCAAGGGCTTAGTACGGCTACGAAGGACACATATGACGCTTTG |
| | | CACATGCAGGCCCTTCCTCCTAGG |
| Mesothelin isoform 2 | 129 | MALPTARPLLGSCGTPALGSLLFLLFSLGWVQPSRTLAGETGQEAAPLDGVLANPPNISSLSPRQLLGFPCAEVSGLS |
| polypeptide (unique | | TERVRELAVALAQKNVKLSTEQLRCLAHRLSEPPEDLDALPLDLLLFLNPDAFSGPQACTRFFSRITKANVDLLPRGA |
| sequence in bold text) | | PERQRLLPAALACWGVRGSLLSEADVRALGGLACDLPGRFVAESAEVLLPRLVSCPGPLDQDQQEAARAALQGGGPPY |
| | | GPPSTWSVSTMDALRGLLPVLGQPIIRSIPQGIVAAWRQRSSRDPSWRQPERTILRPRFRREVEKTACPSGKKAREID |
| | | ESLIFYKKWELEACVDAALLATQMDRVNAIPFTYEQLDVLKHKLDELYPQGYPESVIQHLGYLFLKMSPEDIRKWNVT |
| | | SLETLKALLEVNKGHEMSPQAPRRPLPQVATLIDRFVKGRGQLDKDTLDTLTAFYPGYLCSLSPEELSSVPPSSIWAV |
| | | RPQDLDTCDPRQLDVLYPKARLAFQNMNGSEYFVKIQSFLGGAPTEDLKALSQQNVSMDLATFMKLRTDAVLPLTVAE |
| | | VQKLLGPHVEGLKAEERHRPVRDWILRQRQDDLDTLGLGLQGGIPNGYLVLDLSMQEALSGTPCLLGPGPVLTVLALL |
| | | LASTLA |
| Mesothelin canonical | 130 | MALPTARPLLGSCGTPALGSLLFLLFSLGWVQPSRTLAGETGQEAAPLDGVLANPPNISSLSPRQLLGFPCAEVSGLS |
| polypeptide | | TERVRELAVALAQKNVKLSTEQLRCLAHRLSEPPEDLDALPLDLLLFLNPDAFSGPQACTRFFSRITKANVDLLPRGA |
| | | PERQRLLPAALACWGVRGSLLSEADVRALGGLACDLPGRFVAESAEVLLPRLVSCPGPLDQDQQEAARAALQGGGPPY |
| | | GPPSTWSVSTMDALRGLLPVLGQPIIRSIPQGIVAAWRQRSSRDPSWRQPERTILRPRFRREVEKTACPSGKKAREID |
| | | ESLIFYKKWELEACVDAALLATQMDRVNAIPFTYEQLDVLKHKLDELYPQGYPESVIQHLGYLFLKMSPEDIRKWNVT |
| | | SLETLKALLEVNKGHEMSPQVATLIDRFVKGRGQLDKDTLDTLTAFYPGYLCSLSPEELSSVPPSSIWAVRPQDLDTC |
| | | DPRQLDVLYPKARLAFQNMNGSEYFVKIQSFLGGAPTEDLKALSQQNVSMDLATEMKLRTDAVLPLTVAEVQKLLGPH |
| | | VEGLKAEERHRPVRDWILRQRQDDLDTLGLGLQGGIPNGYLVLDLSMQEALSGTPCLLGPGPVLTVLALLLASTLA |
| Mesothelin isoform 2 | 131 | PQAPRRPL |
| polypeptide subsequence | | |
| Mesothelin isoform 2 | 132 | RPLPQVATLIDR |
| polypeptide subsequence | | |
| Mesothelin canonical | 133 | GHEMSPQVATLIDR |
| polypeptide subsequence | | |
| PD1 membrane signal | 134 | ATGCAGATCCCCCAGGCCCCCTGGCCCGTGGTGTGGGCCGTGCTGCAGCTGGGCTGGAGGCCCGGCTGG |
| polynucleotide | | |

Chimeric PD1 Molecule A

| | | |
|---|---|---|
| PD1 membrane signal | 135 | MQIPQAPWPVVWAVLQLGWRPGW |
| polypeptide | | |
| PD1 region polynucleotide | 136 | TTCCTGGACAGCCCCGACAGGCCCTGGAACCCCCCCACCTTCAGCCCCGCCCTGCTGGTGGTGACCGAGGGCGACAAC |
| | | GCCACCTTCACCTGCAGCTTCAGCAACACCAGCGAGAGCTTCGTGCTGAACTGGTACAGGATGAGCCCCAGCAACCAG |
| | | ACCGACAAGCTGGCCGCCTTCCCCGAGGACAGGAGCCAGCCCGGCCAGGACTGCAGGTTCAGGGTGACCCAGCTGCCC |
| | | AACGGCAGGGACTTCCACATGAGCGTGGTGAGGGCCAGGAGGAACGACAGCGGCACCTACCTGTGCGGCGCCATCAGC |
| | | CTGGCCCCCAAGGCCCAGATCAAGGAGAGCCTGAGGGCCGAGCTGAGGGTGACCGAGAGGAGGGCCGAGGTGCCCACC |
| | | GCCCACCCCAGCCCCAGCCCCAGGCCCGCCGGCCAGTTCCAGACCCTGGTG |
| PD1 region polypeptide | 137 | FLDSPDRPWNPPTFSPALLVVTEGDNATFTCSFSNTSESFVLNWYRMSPSNQTDKLAAFPEDRSQPGQDCRFRVTQLP |
| | | NGRDFHMSVVRARRNDSGTYLCGAISLAPKAQIKESLRAELRVTERRAEVPTAHPSPSPRPAGQFQTLV |
| truncated CD28 | 138 | TGCCCCAGCCCCCTGTTCCCCGGCCCCAGCAAGCCC |
| region polynucleotide | | |
| truncated CD28 | 139 | CPSPLFPGPSKP |
| region polypeptide | | |
| CD28 transmembrane | 140 | TTCTGGGTGCTGGTGGTGGTGGGCGGCGTGCTGGCCTGCTACAGCCTGCTGGTGACCGTGGCCTTCATCATCTTCTGG |
| region polynucleotide | | GTG |
| CD28 transmembrane | 141 | FWVLVVVGGVLACYSLLVTVAFIIFWV |
| region polypeptide | | |
| DAP10 region | 142 | CTGTGCGCCAGGCCCAGGAGGAGCCCCGCCCAGGAGGACGGCAAGGTGTACATCAACATGCCCGGCAGGGGC |
| polynucleotide | | |
| DAP10 region polypeptide | 143 | LCARPRRSPAQEDGKVYINMPGRG |
| CD3z region | 144 | AGGGTGAAGTTCAGCAGGAGCGCCGACGCCCCCGCCTACCAGCAGGGCCAGAACCAGCTGTACAACGAGCTGAACCTG |
| polynucleotide | | GGCAGGAGGGAGGAGTACGACGTGCTGGACAAGAGGAGGGGCAGGGACCCCGAGATGGGCGGCAAGCCCCAGAGGAGG |
| | | AAGAACCCCCAGGAGGGCCTGTACAACGAGCTGCAGAAGGACAAGATGGCCGAGGCCTACAGCGAGATCGGCATGAAG |
| | | GGCGAGAGGAGGAGGGGCAAGGGCCACGACGGCCTGTACCAGGGCCTGAGCACCGCCACCAAGGACACCTACGACGCC |
| | | CTGCACATGCAGGCCCTGCCCCCCAGG |
| CD3z region polypeptide | 145 | RVKFSRSADAPAYQQGQNQLYNELNLGRREEYDVLDKRRGRDPEMGGKPQRRKNPQEGLYNELQKDKMAEAYSEIGMK |
| | | GERRRGKGHDGLYQGLSTATKDTYDALHMQALPPR |
| chimeric PD1-A | 146 | ATGCAGATCCCCCAGGCCCCCTGGCCCGTGGTGTGGGCCGTGCTGCAGCTGGGCTGGAGGCCCGGCTGGTTCCTGGAC |
| polynucleotide | | AGCCCCGACAGGCCCTGGAACCCCCCCACCTTCAGCCCCGCCCTGCTGGTGGTGACCGAGGGCGACAACGCCACCTTC |
| | | ACCTGCAGCTTCAGCAACACCAGCGAGAGCTTCGTGCTGAACTGGTACAGGATGAGCCCCAGCAACCAGACCGACAAG |
| | | CTGGCCGCCTTCCCCGAGGACAGGAGCCAGCCCGGCCAGGACTGCAGGTTCAGGGTGACCCAGCTGCCCAACGGCAGG |
| | | GACTTCCACATGAGCGTGGTGAGGGCCAGGAGGAACGACAGCGGCACCTACCTGTGCGGCGCCATCAGCCTGGCCCCC |
| | | AAGGCCCAGATCAAGGAGAGCCTGAGGGCCGAGCTGAGGGTGACCGAGAGGAGGGCCGAGGTGCCCACCGCCCACCCC |
| | | AGCCCCAGCCCCAGGCCCGCCGGCCAGTTCCAGACCCTGGTGTGCCCCAGCCCCCTGTTCCCCGGCCCCAGCAAGCCC |
| | | TTCTGGGTGCTGGTGGTGGTGGGCGGCGTGCTGGCCTGCTACAGCCTGCTGGTGACCGTGGCCTTCATCATCTTCTGG |
| | | GTGCTGTGCGCCAGGCCCAGGAGGAGCCCCGCCCAGGAGGACGGCAAGGTGTACATCAACATGCCCGGCAGGGGCAGG |
| | | GTGAAGTTCAGCAGGAGCGCCGACGCCCCCGCCTACCAGCAGGGCCAGAACCAGCTGTACAACGAGCTGAACCTGGGC |
| | | AGGAGGGAGGAGTACGACGTGCTGGACAAGAGGAGGGGCAGGGACCCCGAGATGGGCGGCAAGCCCCAGAGGAGGAAG |
| | | AACCCCCAGGAGGGCCTGTACAACGAGCTGCAGAAGGACAAGATGGCCGAGGCCTACAGCGAGATCGGCATGAAGGGC |
| | | GAGAGGAGGAGGGGCAAGGGCCACGACGGCCTGTACCAGGGCCTGAGCACCGCCACCAAGGACACCTACGACGCCCTG |
| | | CACATGCAGGCCCTGCCCCCCAGG |
| | | Structural formula: 5′-(PD1 signal)-(PD1 region (extracellular))-(truncated |
| | | CD28 region (extracellular))-(CD28 transmembrane region)-(DAP10 region |
| | | (cytoplasmic))-(CD3-zeta region (cytoplasmic))-3′ |
| chimeric PD1-A | 147 | MQIPQAPWPVVWAVLQLGWRPGWFLDSPDRPWNPPTESPALLVVTEGDNATFTCSESNTSESFVLNWYRMSPSNQTDK |
| polypeptide | | LAAFPEDRSQPGQDCRFRVTQLPNGRDFHMSVVRARRNDSGTYLCGAISLAPKAQIKESLRAELRVTERRAEVPTAHP |
| | | SPSPRPAGQFQTLVCPSPLFPGPSKPFWVLVVVGGVLACYSLLVTVAFIIFWVLCARPRRSPAQEDGKVYINMPGRGR |
| | | VKFSRSADAPAYQQGQNQLYNELNLGRREEYDVLDKRRGRDPEMGGKPQRRKNPQEGLYNELQKDKMAEAYSEIGMKG |
| | | ERRRGKGHDGLYQGLSTATKDTYDALHMQALPPR |
| | | Structural formula: Nterm-(PD1 signal)-(PD1 region (extracellular))-(truncated |
| | | CD28 region (extracellular))-(CD28 transmembrane region)-(DAP10 region |
| | | (cytoplasmic))-(CD3-zeta region (cytoplasmic))-Cterm |

Chimeric PD1 Molecule B

| | | |
|---|---|---|
| CD8 signal polynucleotide | 148 | ATGGCTCTTCCGGTAACCGCTCTGCTGCTTCCTTTGGCTCTGCTTCTTCACGCGGCTAGGCCA |
| CD8 signal polypeptide | 149 | MALPVTALLLPLALLLHAARP |
| Linker 1 polynucleotide | 150 | GGGAGC |
| Linker 1 polypeptide | 151 | GS |
| CD34 tag polynucleotide | 152 | GAATTGCCTACGCAGGGGACTTTTTCAAATGTAAGCACGAATGTGAGC |
| CD34 tag polypeptide | 153 | ELPTQGTFSNVSTNVS |
| Linker 2 polynucleotide | 154 | TCTCCGGGATCCCCAGGATGG |
| Linker 2 polypeptide | 155 | SPGSPGW |
| PD1 region polynucleotide | 156 | TTCCTGGACAGCCCAGATCGGCCCTGGAATCCCCCTACCTTTTCCCCTGCCCTGCTGGTGGTGACAGAGGGCGACAAC |
| | | GCCACCTTCACATGCAGCTTTTCCAACACCTCTGAGAGCTTCGTGCTGAATTGGTACAGAATGTCCCCATCTAACCAG |
| | | ACAGATAAGCTGGCCGCATTTCCAGAGGACAGGTCCCAGCCTGGACAGGATTGCCGCTTCCGGGTGACCCAGCTGCCC |
| | | AATGGCAGAGACTTTCACATGAGCGTGGTGAGGGCCCGGAGAAACGATTCCGGCACATACCTGTGCGGAGCCATCTCT |
| | | CTGGCCCCAAAGGCACAGATCAAGGAGAGCCTGAGGGCAGAGCTGAGGGTGACCGAGAGGAGGGCAGAGGTG |
| PD1 region polypeptide | 157 | FLDSPDRPWNPPTFSPALLVVTEGDNATFTCSFSNTSESFVLNWYRMSPSNQTDKLAAFPEDRSQPGQDCRFRVTQLP |
| | | NGRDFHMSVVRARRNDSGTYLCGAISLAPKAQIKESLRAELRVTERRAEVPTAHPSPSPRPAGQFQTLV |
| truncated CD28 | 158 | TGCCCTTCCCCACTGTTCCCAGGACCTTCTAAGCCC |
| region polynucleotide | | |
| truncated CD28 | 159 | FLDSPDRPWNPPTFSPALLVVTEGDNATFTCSFSNTSESFVLNWYRMSPSNQTDKLAAFPEDRSQPGQDCRFRVTQLP |
| region polypeptide | | NGRDFHMSVVRARRNDSGTYLCGAISLAPKAQIKESLRAELRVTERRAEVPTAHPSPSPRPAGQFQTLV |
| CD28 transmembrane | 160 | TTTTGGGTGCTGGTGGTGGTGGGAGGCGTGCTGGCCTGCTACTCTCTGCTGGTGACAGTGGCCTTCATCATCTTTTGG |
| region polynucleotide | | GTG |
| CD28 transmembrane | 161 | FWVLVVVGGVLACYSLLVTVAFIIFWV |
| region polypeptide | | |
| DAP10 region | 162 | CTGTGCGCCCGCCCTCGGAGAAGCCCAGCACAGGAGGACGGCAAGGTGTATATCAATATGCCAGGCAGAGGC |
| polynucleotide | | |
| DAP10 region polypeptide | 163 | LCARPRRSPAQEDGKVYINMPGRG |
| CD3z region | 164 | AGGGTGAAGTTTTCTCGGAGCGCCGATGCACCAGCATACCAGCAGGGACAGAACCAGCTGTATAACGAGCTGAATCTG |
| polynucleotide | | GGCAGGCGCGAGGAGTACGACGTGCTGGATAAGCGGAGAGGCAGAGACCCAGAGATGGGAGGCAAGCCACAGAGGAGG |
| | | AAGAACCCTCAGGAGGGCCTGTACAATGAGCTGCAGAAGGACAAGATGGCCGAGGCCTATTCCGAGATCGGCATGAAG |
| | | GGAGAGCGGAGAAGGGGCAAGGGACACGATGGCCTGTACCAGGGCCTGTCTACCGCCACAAAGGACACCTATGATGCC |
| | | CTGCACATGCAGGCCCTGCCACCCAGG |
| CD3z region polypeptide | 165 | RVKFSRSADAPAYQQGQNQLYNELNLGRREEYDVLDKRRGRDPEMGGKPQRRKNPQEGLYNELQKDKMAEAYSEIGMK |
| | | GERRRGKGHDGLYQGLSTATKDTYDALHMQALPPR |
| chimeric PD1-B | 166 | ATGGCTCTTCCGGTAACCGCTCTGCTGCTTCCTTTGGCTCTGCTTCTTCACGCGGCTAGGCCAGGGAGCGAATTGCCT |
| polynucleotide | | ACGCAGGGGACTTTTTCAAATGTAAGCACGAATGTgagCTCTCCGGGATCCCCAGGATGGTTCCTGGACAGCCCAGAT |
| | | CGGCCCTGGAATCCCCCTACCTTTTCCCCTGCCCTGCTGGTGGTGACAGAGGGCGACAACGCCACCTTCACATGCAGC |
| | | TTTTCCAACACCTCTGAGAGCTTCGTGCTGAATTGGTACAGAATGTCCCCATCTAACCAGACAGATAAGCTGGCCGCA |
| | | TTTCCAGAGGACAGGTCCCAGCCTGGACAGGATTGCCGCTTCCGGGTGACCCAGCTGCCCAATGGCAGAGACTTTCAC |
| | | ATGAGCGTGGTGAGGGCCCGGAGAAACGATTCCGGCACATACCTGTGCGGAGCCATCTCTCTGGCCCCAAAGGCACAG |
| | | ATCAAGGAGAGCCTGAGGGCAGAGCTGAGGGTGACCGAGAGGAGGGCAGAGGTGCCTACAGCACACCCAAGCCCTTCC |
| | | CCAAGACCAGCAGGACAGTTCCAGACCCTGGTGTGCCCTTCCCCACTGTTCCCAGGACCTTCTAAGCCCTTTTGGGTG |
| | | CTGGTGGTGGTGGGAGGCGTGCTGGCCTGCTACTCTCTGCTGGTGACAGTGGCCTTCATCATCTTTTGGGTGCTGTGC |
| | | GCCCGCCCTCGGAGAAGCCCAGCACAGGAGGACGGCAAGGTGTATATCAATATGCCAGGCAGAGGCAGGGTGAAGTTT |
| | | TCTCGGAGCGCCGATGCACCAGCATACCAGCAGGGACAGAACCAGCTGTATAACGAGCTGAATCTGGGCAGGCGCGAG |
| | | GAGTACGACGTGCTGGATAAGCGGAGAGGCAGAGACCCAGAGATGGGAGGCAAGCCACAGAGGAGGAAGAACCCTCAG |
| | | GAGGGCCTGTACAATGAGCTGCAGAAGGACAAGATGGCCGAGGCCTATTCCGAGATCGGCATGAAGGGAGAGCGGAGA |
| | | AGGGGCAAGGGACACGATGGCCTGTACCAGGGCCTGTCTACCGCCACAAAGGACACCTATGATGCCCTGCACATGCAG |
| | | GCCCTGCCACCCAGG |
| | | Structural formula: 5′-(CD8 signal)-(linker 1)-(CD34 tag)-(linker 2)-(PD1 |
| | | region (extracellular))-(truncated CD28 region (extracellular))-(CD28 |
| | | transmembrane region)-(DAP10 region (cytoplasmic))-(CD3-zeta region |
| | | (cytoplasmic))-3′ |
| chimeric PD1-B | 167 | MALPVTALLLPLALLLHAARPGSELPTQGTFSNVSTNVSSPGSPGWFLDSPDRPWNPPTFSPALLVVTEGDNATFTCS |
| polypeptide | | FSNTSESFVLNWYRMSPSNQTDKLAAFPEDRSQPGQDCRFRVTQLPNGRDFHMSVVRARRNDSGTYLCGAISLAPKAQ |
| | | IKESLRAELRVTERRAEVPTAHPSPSPRPAGQFQTLVCPSPLFPGPSKPFWVLVVVGGVLACYSLLVTVAFIIFWVLC |
| | | ARPRRSPAQEDGKVYINMPGRGRVKFSRSADAPAYQQGQNQLYNELNLGRREEYDVLDKRRGRDPEMGGKPQRRKNPQ |
| | | EGLYNELQKDKMAEAYSEIGMKGERRRGKGHDGLYQGLSTATKDTYDALHMQALPPR |
| | | Nterm-(CD8 signal)-(linker 1)-(CD34 tag)-(linker 2)-(PD1 region |
| | | (extracellular))-(truncated CD28 region (extracellular))-(CD28 transmembrane |
| | | region)-(DAP10 region (cytoplasmic))-(CD3-zeta region (cytoplasmic))-Cterm |
| Binding Molecule E | | |
| CAR polypeptide | 168 | MALPVTALLLPLALLLHAARPGSELPTQGTFSNVSTNVSSPEVKLVESGGGLVKPGGSLKVSCTASGFTFSRYAMSWV |
| | | RQTPERRLEWVASITFGGSAYYLDSVKGRFTISRDNAQNILYLQMNSLVSEDTAIYNCARHQPWFDYWGQGTLVTVSA |
| | | GGGGSGGGGSGGGGSDVLLTQTPLSLPVSLGDQASISCRSSQNIXHSDGDTYLDWFLQKPGQSPNLLIYKVSNRFSGV |
| | | PDRFSGSGSGTDFTLKISRVEADDLGVYYCFQASHVPYTFGGGTKLEVKRTTTPAPRPPTPAPTIASQPLSLRPEACR |
| | | PAAGGAVHTRGLDFACDIYIWAPLAGTCGVLLLSLVITLYCNHRNRRRVCKCPRVDRSKRSRLLHSDYMNMTPRRPGP |
| | | TRKHYQPYAPPRDFAAYRSRVKFSRSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| | | Structural formula: Nterm-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-Cterm |
| | | X is I or V |
| CAR polynucleotide | 169 | Structural formula: 5′-(CD8 signal)-(Linker 1)-(CD34 tag)-(Linker 2)-(VH |
| | | Domain)-(Linker 3)-(VL Domain)-(Linker 4)-(CD8 stalk region)-(CD8 |
| | | transmembrane region)-(Linker 5)-(CD28 cytoplasmic region)-(CD3-zeta |
| | | cytoplasmic region)-3′ |
| | | Z is A or G; Y is A or T |
| CD8 signal polypeptide | 170 | MALPVTALLLPLALLLHAARP |
| CD8 signal polynucleotide | 171 | ATGGCTCTTCCGGTAACCGCTCTGCTGCTTCCTTTGGCTCTGCTTCTTCACGCGGCTAGGCCA |
| Linker 1 polypeptide | 172 | GS |
| Linker 1 polynucleotide | 173 | GGGAGC |
| CD34 tag polypeptide | 174 | ELPTQGTFSNVSTNVS |
| CD34 tag polynucleotide | 175 | GAATTGCCTACGCAGGGGACTTTTTCAAATGTAAGCACGAATGTGAGC |
| Linker 2 polypeptide | 176 | SP |
| Linker 2 polynucleotide | 177 | TCTCCG |
| VH Domain polypeptide | 178 | EVKLVESGGGLVKPGGSLKVSCTASGFTFSRYAMSWVRQTPERRLEWVASITFGGSAYYLDSVKGRFTISRDNAQNIL |
| | | YLQMNSLVSEDTAIYNCARHQPWFDYWGQGTLVTVSA |
| VH Domain polynucleotide | 179 | GAAGTCAAATTGGTGGAGAGTGGCGGTGGGCTCGTAAAACCAGGAGGCAGCTTGAAAGTAAGTTGCACAGCGTCTGGG |
| | | TTCACTTTCAGCCGATACGCTATGAGTTGGGTGCGGCAAACGCCCGAAAGACGATTGGAGTGGGTAGCTAGTATTACT |
| | | TTTGGGGGTTCCGCGTATTACTTGGATAGCGTTAAAGGGAGATTCACCATATCACGAGATAATGCCCAAAACATACTC |
| | | TACCTCCAGATGAACTCATTGGTGTCTGAAGATACTGCAATTTACAACTGCGCCAGGCACCAGCCATGGTTTGACTAT |
| | | TGGGGTCAAGGTACGTTGGTCACAGTCTCAGCC |
| Linker 3 polypeptide | 180 | GGGGSGGGGSGGGGS |
| Linker 3 polynucleotide | 181 | GGCGGTGGCGGATCTGGAGGGGGAGGCAGTGGAGGAGGTGGTTCA |
| VL Domain polypeptide | 182 | DVLLTQTPLSLPVSLGDQASISCRSSQNIXHSDGDTYLDWFLQKPGQSPNLLIYKVSNRFSGVPDRFSGSGSGTDFTL |
| | | KISRVEADDLGVYYCFQASHVPYTFGGGTKLEVK |
| | | X is I or V |
| VL Domain polynucleotide | 183 | GATGTTCTCCTGACGCAAACTCCTTTGTCATTGCCGGTAAGTCTGGGCGACCAAGCGAGCATTTCCTGTCGCAGCAGC |
| | | CAGAACATAYTZCATAGTGATGGCGACACTTACTTGGATTGGTTCCTCCAGAAACCGGGGCAATCTCCGAACCTCTTG |
| | | ATATATAAGGTCTCAAATCGCTTTAGCGGGGTTCCCGACCGCTTTTCAGGTTCTGGCTCAGGTACCGATTTCACGCTG |
| | | AAAATTTCTAGAGTAGAGGCTGACGATCTGGGTGTTTACTATTGTTTTCAAGCTTCACACGTTCCTTATACGTTCGGA |
| | | GGGGGGACTAAACTGGAGGTTAAA |
| | | Y is G or A; Z is T when Y is G and Z is T, C or A when Y is A |
| Linker 4 polypeptide | 184 | R |
| Linker 4 polynucleotide | 185 | CGT |
| CD8 stalk polypeptide | 186 | TTTPAPRPPTPAPTIASQPLSLRPEACRPAAGGAVHTRGLDFACD |
| CD8 stalk polynucleotide | 187 | ACGACGACACCAGCCCCAAGACCCCCAACTCCCGCTCCTACCATAGCTTCCCAACCCCTGTCACTGAGGCCAGAGGCA |
| | | TGCAGGCCCGCTGCGGGTGGCGCGGTACATACGCGGGGACTGGACTTTGCATGTGAC |
| CD8 transmembrane | 188 | IYIWAPLAGTCGVLLLSLVITLYCNHRNRRRVCKCPR |
| polypeptide | | |
| CD8 transmembrane | 189 | ATTTACATATGGGCGCCACTGGCGGGAACATGTGGAGTTTTGTTGCTTAGCCTGGTCATAACACTGTATTGCAATCAT |
| polynucleotide | | CGCAACAGACGCCGCGTCTGCAAGTGTCCCAGG |
| Linker 5 polypeptide | 190 | VD |
| Linker 5 polynucleotide | 191 | GTCGAC |
| CD28 cytoplasmic | 192 | RSKRSRLLHSDYMNMTPRRPGPTRKHYQPYAPPRDFAAYRS |
| polypeptide | | |
| CD28 cytoplasmic | 193 | AGATCTAAGAGAAGTAGACTTCTTCACAGTGATTATATGAATATGACGCCTCGAAGACCCGGCCCGACACGCAAACAC |
| polynucleotide | | TATCAGCCGTATGCCCCCCCTCGGGATTTTGCTGCCTACCGCAGC |
| CD3-zeta cytoplasmic | 194 | RVKFSRSADAPAYQQGQNQLYNELNLGRREEYDVLDKRRGRDPEMGGKPRRKNPQEGLYNELQKDKMAEAYSEIGMKG |
| polypeptide | | ERRRGKGHDGLYQGLSTATKDTYDALHMQALPPR |
| CD3-zeta cytoplasmic | 195 | CGCGTTAAGTTCTCTAGGTCCGCTGACGCCCCTGCCTACCAGCAGGGTCAAAACCAACTGTACAATGAATTGAATCTT |
| polynucleotide | | GGGAGACGGGAGGAGTATGACGTACTCGACAAGCGGAGGGGGAGAGATCCTGAGATGGGTGGAAAGCCTCGACGAAAA |
| | | AACCCACAAGAAGGGTTGTATAATGAACTGCAGAAAGACAAGATGGCTGAGGCATATAGTGAAATCGGGATGAAGGGA |
| | | GAACGCCGGCGCGGGAAAGGGCACGATGGTCTGTATCAAGGGCTTAGTACGGCTACGAAGGACACATATGACGCTTTG |
| | | CACATGCAGGCCCTTCCTCCTAGG |
| Binding Molecule C | 196 | GSELPTQGTFSNVSTNVSSPEVKLVESGGGLVKPGGSLKLSCEASGFTFSRYAMSWVRHTPEKRLAWVASITFGGDPY |
| CAR polypeptide (without | | YPDGLKGRFTISRDNTRNILYLQMNSLRSEDTAMYYCVRHESWFAYWGQGTLVTVSAGGGGSGGGGSGGGGSDVLMTQ |
| signal region) | | TPLSLPVSLGEQVSISCRSSQTIVHTDGNIYLEWYLQNPGQSPRLLIYKISNRFSGVPDRESGSGSGTDFTLKISRVE |
| | | AEDLGIYYCFQASHVPYTFGGGTKLEIKRTTTPAPRPPTPAPTIASQPLSLRPEACRPAAGGAVHTRGLDFACDIYIW |
| | | APLAGTCGVLLLSLVITLYCNHRNRRRVCKCPRVDRSKRSRLLHSDYMNMTPRRPGPTRKHYQPYAPPRDFAAYRSRV |
| | | KFSRSADAPAYQQGQNQLYNELNLGRREEYDVLDKRRGRDPEMGGKPRRKNPQEGLYNELQKDKMAEAYSEIGMKGER |
| | | RRGKGHDGLYQGLSTATKDTYDALHMQALPPR |
| | | Structural formula: Nterm-(Linker 1)-(CD34 tag)-(Linker 2)-(VH Domain)-(Linker |
| | | 3)-(VL Domain)-(Linker 4)-(CD8 stalk region)-(CD8 transmembrane region) - |
| | | (Linker 5)-(CD28 cytoplasmic region)-(CD3-zeta cytoplasmic region)-Cterm |
| Binding Molecule D | 197 | GSELPTQGTFSNVSTNVSSPEVKLVESGGGLVKPGGSLKVSCTASGFTFSRYAMSWVRQTPERRLEWVASITFGGSAY |
| CAR polypeptide (without | | YLDSVKGRFTISRDNAQNILYLQMNSLVSEDTAIYNCARHQPWFDYWGQGTLVTVSAGGGGSGGGGSGGGGSDVLLTQ |
| signal region) | | TPLSLPVSLGDQASISCRSSQNIXHSDGDTYLDWFLQKPGQSPNLLIYKVSNRFSGVPDRESGSGSGTDFTLKISRVE |
| | | ADDLGVYYCFQASHVPYTFGGGTKLEVKRTTTPAPRPPTPAPTIASQPLSLRPEACRPAAGGAVHTRGLDFACDIYIW |
| | | APLAGTCGVLLLSLVITLYCNHRNRRRVCKCPRVDRSKRSRLLHSDYMNMTPRRPGPTRKHYQPYAPPRDFAAYRSRV |
| | | KFSRSADAPAYQQGQNQLYNELNLGRREEYDVLDKRRGRDPEMGGKPRRKNPQEGLYNELQKDKMAEAYSEIGMKGER |
| | | RRGKGHDGLYQGLSTATKDTYDALHMQALPPR |
| | | Structural formula: Nterm-(Linker 1)-(CD34 tag)-(Linker 2)-(VH Domain)-(Linker |
| | | 3)-(VL Domain)-(Linker 4)-(CD8 stalk region)-(CD8 transmembrane region)- |
| | | (Linker 5)-(CD28 cytoplasmic region)-(CD3-zeta cytoplasmic region)-Cterm |
| | | X is I or V |
| Binding Molecule E | 198 | GSELPTQGTFSNVSTNVSSPEVKLVESGGGLVKPGGSLKVSCTASGFTFSRYAMSWVRQTPERRLEWVASITFGGSAY |
| CAR polypeptide (without | | YLDSVKGRFTISRDNAQNILYLQMNSLVSEDTAIYNCARHQPWFDYWGQGTLVTVSAGGGGSGGGGSGGGGSDVLLTQ |
| signal region) | | TPLSLPVSLGDQASISCRSSQNIXHSDGDTYLDWFLQKPGQSPNLLIYKVSNRFSGVPDRESGSGSGTDFTLKISRVE |
| | | ADDLGVYYCFQASHVPYTFGGGTKLEVKRTTTPAPRPPTPAPTIASQPLSLRPEACRPAAGGAVHTRGLDFACDIYIW |
| | | APLAGTCGVLLLSLVITLYCNHRNRRRVCKCPRVDRSKRSRLLHSDYMNMTPRRPGPTRKHYQPYAPPRDFAAYRSRV |
| | | KFSRSADAPAYQQGQNQLYNELNLGRREEYDVLDKRRGRDPEMGGKPRRKNPQEGLYNELQKDKMAEAYSEIGMKGER |
| | | RRGKGHDGLYQGLSTATKDTYDALHMQALPPR |
| | | Structural formula: Nterm-(Linker 1)-(CD34 tag)-(Linker 2)-(VH Domain)-(Linker |
| | | 3)-(VL Domain)-(Linker 4)-(CD8 stalk region)-(CD8 transmembrane region) - |
| | | (Linker 5)-(CD28 cytoplasmic region)-(CD3-zeta cytoplasmic region) -Cterm |
| | | X is I or V |
| chimeric PD1-A | 199 | FLDSPDRPWNPPTFSPALLVVTEGDNATFTCSFSNTSESFVLNWYRMSPSNQTDKLAAFPEDRSQPGQDCRFRVTQLP |
| polypeptide (without signal | | NGRDFHMSVVRARRNDSGTYLCGAISLAPKAQIKESLRAELRVTERRAEVPTAHPSPSPRPAGQFQTLVCPSPLFPGP |
| region) | | SKPFWVLVVVGGVLACYSLLVTVAFIIFWVLCARPRRSPAQEDGKVYINMPGRGRVKFSRSADAPAYQQGQNQLYNEL |
| | | NLGRREEYDVLDKRRGRDPEMGGKPQRRKNPQEGLYNELQKDKMAEAYSEIGMKGERRRGKGHDGLYQGLSTATKDTY |
| | | DALHMQALPPR |
| | | Structural formula: Nterm-(PD1 region (extracellular))-(truncated CD28 region |
| | | (extracellular))-(CD28 transmembrane region)-(DAP10 region (cytoplasmic))- |
| | | (CD3-zeta region (cytoplasmic))-Cterm |
| chimeric PD1-B | 200 | GSELPTQGTFSNVSTNVSSPGSPGWFLDSPDRPWNPPTFSPALLVVTEGDNATFTCSFSNTSESFVLNWYRMSPSNQT |
| polypeptide (without signal | | DKLAAFPEDRSQPGQDCRFRVTQLPNGRDFHMSVVRARRNDSGTYLCGAISLAPKAQIKESLRAELRVTERRAEVPTA |
| region) | | HPSPSPRPAGQFQTLVCPSPLFPGPSKPFWVLVVVGGVLACYSLLVTVAFIIFWVLCARPRRSPAQEDGKVYINMPGR |
| | | GRVKFSRSADAPAYQQGQNQLYNELNLGRREEYDVLDKRRGRDPEMGGKPQRRKNPQEGLYNELQKDKMAEAYSEIGM |
| | | KGERRRGKGHDGLYQGLSTATKDTYDALHMQALPPR |
| | | Structural formula: Nterm-(CD34 tag)-(linker)-(PD1 region (extracellular))- |
| | | (truncated CD28 region (extracellular))-(CD28 transmembrane region)-(DAP10 |
| | | region (cytoplasmic))-(CD3-zeta region (cytoplasmic)) -Cterm |

### EXAMPLES

The examples set forth below illustrate certain implementations and do not limit the technology.

### Example 1: Identification of an Isoform of Mesothelin Selectively Expressed in Cancer Tissues

Mesothelin (MSLN) is considered a cancer marker for cancers such as Ovarian Cancer (OV) and Mesothelioma. However, its expression in normal tissue can cause specificity problems that interfere with the ability to utilize it as a cancer immunotherapy target. The human MSLN transcript has at least three isoforms. Isoform 1 encoding 622 amino acids is the predominant transcript detected in normal and tumor tissues and has been a promising target for cancer immunotherapy. Isoform 2 (IsoMSLN) is the minor transcript using alternatively spliced exons, producing an additional 8-amino acid insertion compared to Isoform 1. Isoform 3 produces a truncated and soluble MSLN.

SpliceDiff™ is a proprietary software module for the identification of potential new immunotherapeutic cancer targets that originate from differentially expressed, alternatively spliced transcripts. The proprietary software SpliceDiff™ is part of an integrated bioinformatics and artificial intelligence (AI) system described in PCT Application No. PCT/US20/35183, filed on May 29, 2020, the contents of which are expressly incorporated by reference herein. In this example, SpliceDiff™ was used to predict the splice variants specifically upregulated in OV and other cancers. It was found MSLN Isoform 2 (“IsoMSLN”) is specifically expressed in certain cancers, such as mesothelioma, ovarian cancers and pancreatic cancer. Furthermore, this alternatively spliced isoform created a unique epitope predicted to be present on the cell surface.

### Transcriptomics

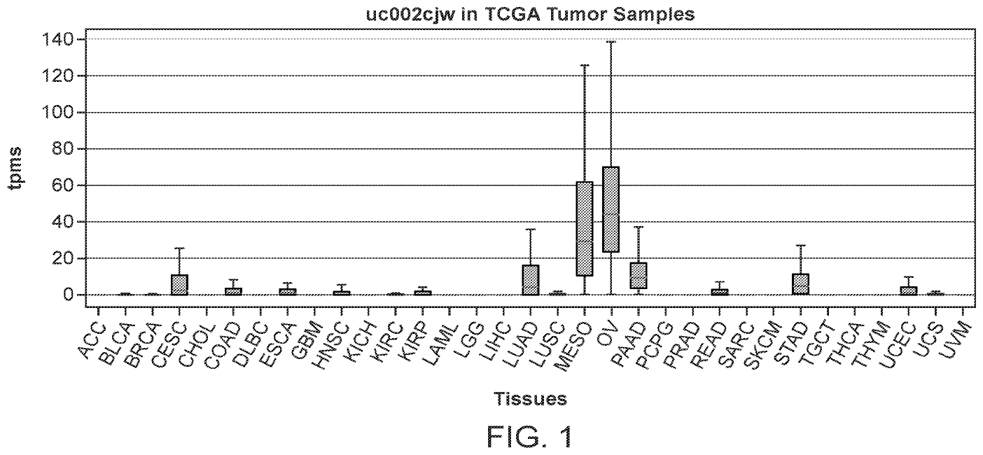

The SpliceDiff™ software was used to process input data from The Cancer Genome Atlas (TCGA), a public program for the genomic profile of cancer that includes transcriptomics data from 33 human cancer types, and from the Genotype-Tissue Expression (GTEX), a public program to study tissue-specific gene expression that includes data for 54 healthy tissues. A mesothelin splice variant transcript, uc002cjw, was predicted to be upregulated. FIG. 1 shows the SpliceDiff™ generated expression profile of the uc002cjw transcript in Transcripts per Million (TPM) in TCGA tumor tissues. The uc002cjw transcript was detectable in several cancer tissues, including cervical squamous cell carcinoma and endocervical carcinoma, lung adenocarcinoma, mesothelioma, ovarian cancer, pancreatic adenocarcinoma and stomach adenocarcinoma. Significant expression was seen in mesothelioma and ovarian cancer samples. In Ovarian Cancer (OV), a median Transcripts per Million (TPM) of 44.42 was observed, with TPM>23.68 (lower quartile) in 75% of OV specimens. In addition, the low expression of the mesothelin (MSLN) transcript uc002cjw in GTEX healthy tissues and in TCGA adjacent normal tissues (adjacent to cancer tissue) indicates selective upregulation in cancer tissues, such as OV and Mesothelioma.

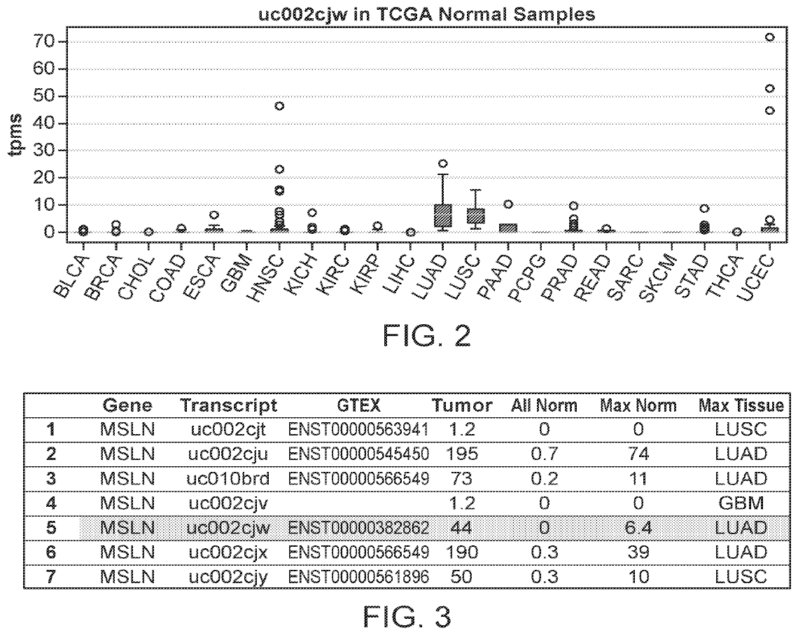

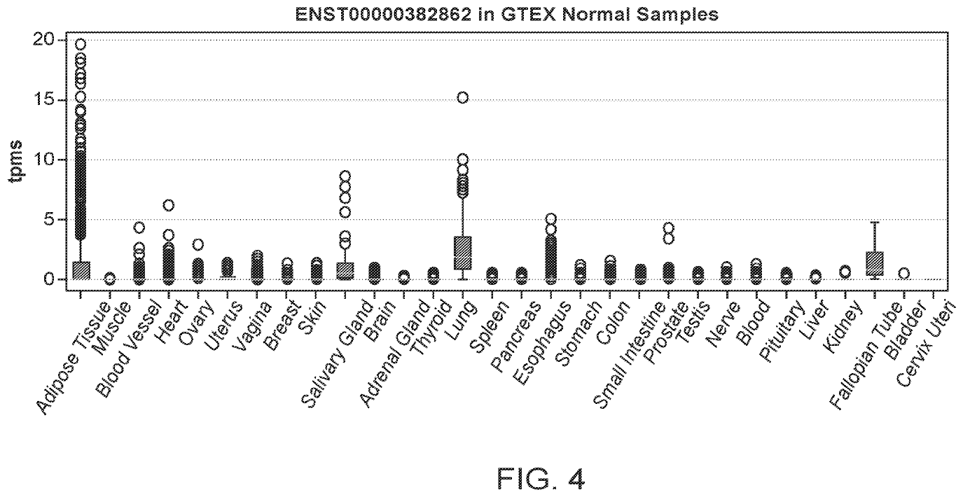

FIG. 2 depicts the SpliceDiff™ generated expression profile of the uc002cjw transcript in TCGA adjacent normal (healthy) tissues. The median of expression was found to be lower than the median of expression in several cancer tissues, indicating tumor specificity. In OV, the difference in median TPM of the uc002cjw transcript relative to the highest median TPM measured in adjacent healthy tissue (that of healthy tissue adjacent to LUAD) is significant and indicates selectivity as a marker of OV, as shown in FIG. 3. Expression of the uc002cjw transcript in the GTEX healthy tissues also was found to be low, as shown in FIG. 4. The results demonstrate that the mRNA transcript uc002cjw shows selectivity for cancers, such as OV, and is significantly upregulated in some tumor tissues, including OV.

### PCR Analysis

qPCR was performed on a human ovarian cancer cDNA array, to detect MSLN isoforms. The array (OriGene TissueScan™ Ovarian Cancer cDNA Array II) contains 7 non-cancerous ovarian tissue samples, 19 tissue samples of ovarian cancer Stages I to II, and 22 tissue samples of ovarian cancer Stages III-IV. The results, presented in the Table below, show that while Isoform 1 was detected and, in some cases, upregulated in at least a fraction of both normal and cancer samples, the Isoform 2 (IsoMSLN) was specifically detected and upregulated in the Stage III-IV ovarian cancer samples compared to the normal samples.

| | MSLN isoform 1 | | | | MSLN isoform 2 | | | |
|---|---|---|---|---|---|---|---|---|
| Ovarian tissues | Positive rate | | upregulation | | Positive rate | | upregulation | |
| non-cancerous | 4/7 | (57.1%) | 2/7 | (28.6%) | 0/7 | (0.0%) | 0/7 | (0.0%) |
| Stage I-II | 19/19 | (100%) | 10/19 | (52.6%) | 0/19 | (0.0%) | 0/19 | (0.0%) |
| Stage III-IV | 22/22 | (100%) | 19/22 | (86.4%) | 3/22 | (13.6%) | 3/22 | (13.6%) |

### Proteomics

The transcript uc002cjw is translated into a Mesothelin protein isoform (IsoMSLN) that was found to be distinguished by the presence of a unique peptide that is absent from the Mesothelin (MSLN) protein sequences originating from other transcripts, e.g., uc002cjt, uc002cju, uc010brd, uc002cjv, uc002cjx and uc002cjy shown in FIG. 3. The identification of cancer selective transcripts is important for the discovery of novel target candidates for immunotherapy. However, while upregulation of an mRNa transcript in cancers can indicate an increase in the corresponding protein in the tissue, it does not take in account factors such as posttranslational modifications and protein degradation. Therefore, the transcriptomics data presented above, based on RNA-seq data mining using the technology described herein, was confirmed at the protein level, by analyzing a mass spectrometry dataset containing ovarian cancer samples and adjacent non-tumoral tissues.

To validate the prediction and to confirm the presence of the IsoMSLN in OV specimens, publicly available proteomic datasets were analyzed to detect the presence of the unique peptide. Data from the clinical study “S038 Confirmatory Study” were downloaded from CPTAC (World Wide Web Uniform Resource Locator cptac-data-portal.georgetown.edu/study-summary/S038). Data originated from TMT10plex quantification for global proteomic profiling was acquired with Orbitrap Fusion Lumos mass spectrometer. This study analyzed the proteomics of OV tissue samples from a cohort of 109 OV cancer patients, with 100% Serous Adenocarcinoma histological subtype, 81% of tumors of grade 3, and 64% tumor stage IIIC and 15% stage IV. 13 datasets were analyzed, comprising 94 ovarian tumor and 23 ovarian normal tissue samples from the same group of ovarian cancer patients. Data parsing and data quality control data were processed by MS Biowork through the MaxQuant software v1.6.2.3 for recalibration of MS data, filtering of database search results at the 1% protein and peptide false discovery rate (FDR), calculation of reporter ion intensities (TMT), and isotopic correction of reporter ion intensities (TMT).

As shown in the Table below, the peptide that is present only in the isoform variant (IsoMSLN) was detected in 71% of tumor tissues and in 61% of normal tissues, while a peptide translated from other canonical forms of Mesothelin transcripts (MSLN) was detected in 100% of both tumors and normal samples, indicating that, as predicted by the technology described herein, the isoform (IsoMSLN) expression was more selective for cancer tissues.

| | | MSLN | % of Adjacent | % of Tumor |
|---|---|---|---|---|
| Protein | Peptide (SEQ ID NO) | Transcripts | Normal Tissues | Tissues |
| IsOMSLN | RPLPQVATLIDR (132) | uc002cjw | 61 | 71 |
| MSLN | GHEMSPQVATLIDR | uc002cjv; | 100 | 100 |
| | (133) | uc010brd; | | |
| | | uc002cju; | | |
| | | uc002cjy | | |

### Example 2: Generation of Antibodies that Specifically Target Mesothelin Isoform 2 (IsoMSLN)

Antibodies were manufactured by immunizing mice with the IsoMSLN-specific peptide, PQAPRRPL (SEQ ID NO: 131) conjugated to KLH. Antibody-producing hybridomas were obtained by fusing splenocytes with the murine Sp2/0 cell line, using standard fusion methods. The antibodies were screened for specific binding using an ELISA assay.

The Table below shows EC₅₀ values of various anti-MSLN monoclonal antibody (mAb) clones (1B1, 1B6, 11C11, 8D4) against different MSLN isoforms:

EC₅₀ to bind MSLN (μg/ml)

| Anti-MSLN mAb clone | Isoform 1 | Isoform 2 |
|---|---|---|
| *K1 | 0.15 | 0.11 |
| 1B1 | No binding | 2.31 |
| 1B6 | No binding | 0.073 |
| 11C11 | 6.88 | 0.046 |
| 8D4 | No binding | 0.24 |

*Clone K1 is a commercially available anti-MSLN antibody known to bind to both isoforms

The results demonstrate that the isolated clones specifically bind IsoMSLN, with clones IB6 and 11C11 showing a high degree of specificity for the unique IsoMSLN antigenic determinant peptide sequence (SEQ ID NO:131). Clone 11C11 showed a slight partial reaction with MSLN Isoform 1.

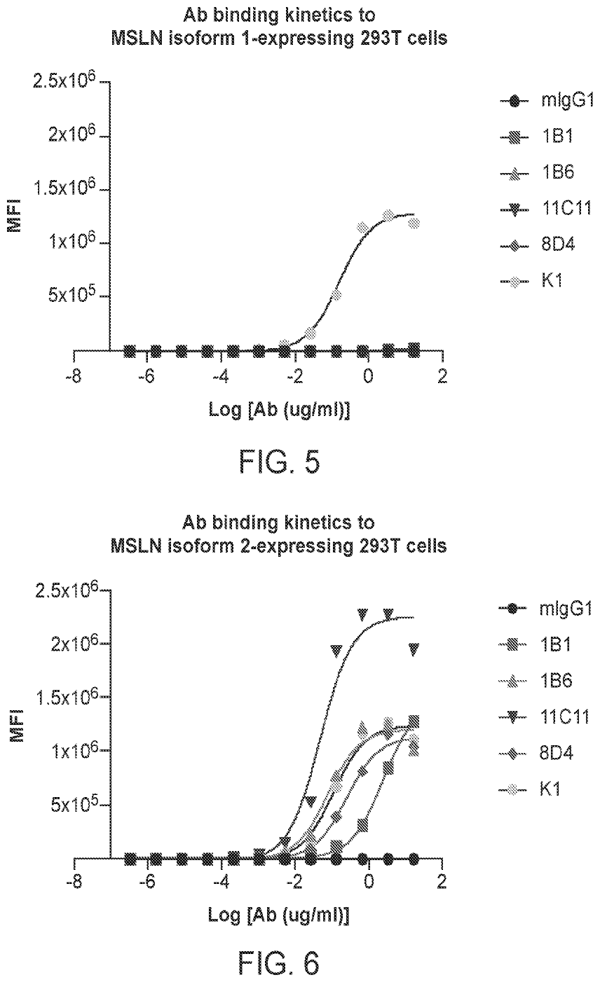

FIGS. 5 and 6 depict flow cytometry staining of anti-IsoMSLN-specific antibodies on 293T cells overexpressing either MSLN Isoform 1 (FIG. 5) or Isoform 2 (IsoMSLN) (FIG. 6). mlgG1 was included as a negative control for staining. Anti-MSLN (clone K1) is a known, commercially available MSLN-specific antibody that shows binding to both isoforms. The results depicted in FIGS. 5 and 6 demonstrate that the monoclonal antibodies 1B1, 1B6, 11C11, and 8D4 show specific binding to IsoMSLN.

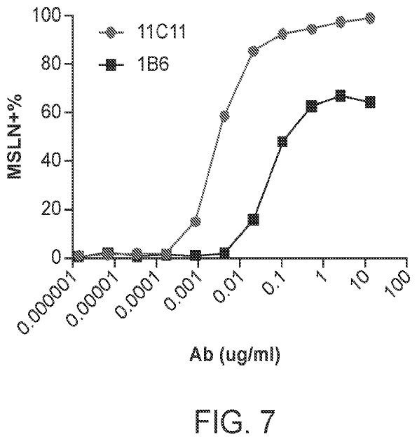

FIG. 7 shows the detection of IsoMSLN on a cell surface by anti-IsoMSLN-specific monoclonal antibodies. Clone 1B6 was found to specifically recognize IsoMSLN and demonstrated that ˜60% of NCI H226 cells express IsoMSLN on the cell surface. Clone 11C11, which has high affinity to IsoMSLN and low affinity to MSLN Isoform 1, was found to be able to bind to nearly all the NCI H226 cells.

### Example 3: CAR Molecule Constructs Containing Anti-IsoMSLN Antibodies, Expression in Gamma-Delta (γδ) T Cells and Cytotoxicity Assays

DNA encoding the scFv of the 1B6 and 11C11 clones were manufactured by gene synthesis and and sub-cloned into a pSFG gamma-retrovirus vector along with the CD8 stalk (hinge) sequence, the CD8 transmembrane domain, and the CD28/CD3-zeta cytoplasmatic (signaling) domains by restriction cloning via Notl and BsWI restriction sites. The resulting DNA molecule encoding CAR (SEQ ID NO:74 for 1B6; SEQ ID NO:102 for 11C11), and the translated CAR molecule, can be represented as follows:

5′-(CD8 signal)-(Linker 1)-(CD34 tag)-(Linker 2)-(VH Domain)-(Linker 3)-(VL Domain)-(Linker 4)-(CD8 stalk region)-(CD8 transmembrane region)-(Linker 5)-(CD28 cytoplasmic region)-(CD3-zeta cytoplasmic region)-3′.

The plasmid construct expressing the 1B6 scFv is depicted in FIG. 8, and the plasmid construct expressing the 11C11 scFv is depicted in FIG. 9. The resulting constructs were transduced and expressed in gamma-delta (γδ) T cells, as described below:

### Step 1: γδ-T Cell Expansion for 7 Days

Cryopreserved PBMCs were thawed. Alternately, PBMCs can be freshly prepared from a peripheral blood sample, the sample can be processed by density gradient centrifugation to separate and/or isolate a buffy coat containing white blood cells, platelets, granulocytes and the like. The buffy coat can further undergo a Ficoll gradient separation to obtain mononuclear cells (PBMCs). To culture medium (RPMI+10% FBS+P/S) was added adding human IL-2 (IL-2) and Zoledronic Acid (ZA) to final concentrations of 300 IU/ml and 5 μM, respectively. The PBMC cell pellet was suspended in culture medium and adjusted to 1×10⁶ cells/ml. The cells were cultured at 37° C. with 5% CO₂ for 7 days. Every 2 to 3 days, half of the volume of total culture medium was replaced with fresh culture medium containing human IL-2 300 IU/ml.

### Step 1b: γδ-T Cell Enrichment on Day 7

On day 7 after PBMC culture and stimulation, cells were harvested by centrifugation at 400×g for 5 minutes. The cell pellet was resuspended in RPMI+10% FBS+ P/S and the cells were counted. A small aliquot containing about 1 million PBMCs was saved for cell staining and flow analysis to determine γδ-T cell purity.

The γδ-T cells were isolated using MACS LS columns (Miltenyi Biotec #130-092-892) and following the manufacturer's protocol (previously filed with U.S. Provisional Patent Application Nos. 63/048,488 and 63/115,465 as Appendix 2, the contents of which are expressly incorporated by reference herein). The type of MACS column that is used depends on the number of cells to be isolated, as follows:

| | Maximum No. of | Maximum No. of | |
|---|---|---|---|
| Column | Labeled Cells | Total Cells | Separator |
| MS | 10⁷ | 2 × 10⁸ | MiniMACS, OctoMACS, |
| | | | VarioMACS, SuperMACS |
| LS | 10⁸ | 2 × 10⁹ | MidiMACS, QuadroMACS, |
| | | | VarioMACS, SuperMACS |
| XS | 10⁹ | 2 × 10¹⁰ | SuperMACS |
| autoMACS | 2 × 10⁸ | 4 × 10⁹ | autoMACS |

| | Maximum No. of | Maximum No. of |
|---|---|---|
| LD Column | Labeled Cells | Total Cells |
| Manual Use | 1 × 10⁸ | 5 × 10⁸ |
| Use with MultiMACS | 1 × 10⁸ | 5 × 10⁸ |
| Cell24 Separator Plus | | |

The cell suspension was centrifuged at 400×g for 5 minutes. The supernatant was aspirated, and the cell pellet was resuspended in 80 μL of MACS buffer per 10⁷ total cells. 20 μL of Biotin-Antibody Cocktail was added per 10⁷ total cells, mixed well and refrigerated for 10 minutes (4-8° C.). The cells were washed by adding 1-2 mL of MACS buffer per 10⁷ cells, centrifuged at 400×g for 5 minutes, and the supernatant aspirated. 80 μL of MACS buffer per 10⁷ total cells was then added, followed by the addition of 20 μL of anti-biotin microbeads per 10⁷ total cells. The resulting composition was mixed well and refrigerated for an additional 15 minutes (4-8° C.). The cells were washed by adding 1-2 mL of buffer per 10⁷ cells, centrifuged at 400×g for 5 minutes, and the supernatant aspirated. Up to 10⁸ cells were resuspended in 500 μL of buffer and subjected to magnetic separation with MACS Columns chosen according to the needed capacity as shown above.

The MACS column was prepared by rinsing with appropriate amount of buffer: LS: 3 mL. The cell suspension was applied onto the column, allowed to pass through the column and the effluent was collected as the fraction with unlabeled cells, representing the enriched γδ-T cell fraction. The column was washed two times with a column-appropriate amount of buffer: LS: 3×3 mL, and the total effluent representing the unlabeled cell fraction was collected. The effluent was centrifuged at 400×g for 5 minutes, and the supernatant aspirated. The cell pellet was resuspended in RPMI+10% FBS+ P/S, and the cells were counted. A small aliquot containing about 1 million purified γδ-T cells was saved for cell staining and flow analysis to determine γδ-T cell purity.

Step 2: γδ-T Cell Expansion from Day 7 to 14

After γδ-T cell enrichment, the cell pellet was resuspended in expansion culture medium (RPMI+10% FBS+P/S+hIL-2 (300 U/ml)) and the cell concentration was adjusted to 10⁶/ml. The cells were cultured at 37° C. in 5% CO₂ for 7 days. Half the volume of media was replaced with fresh culture medium (containing IL-2 (300 IU/ml) every 2 to 3 days. The cell density was maintained at no more than 2×10⁶/ml. On day 14 of the cell expansion, the cells were harvested for determining γδ-T purity and functional assay(s).

### Cell Staining and Flow Cytometry to Determine γδ-T Cell Purity.

The cells were stained (0.2 million cells per reaction) in 100 ul FACS staining buffer (DPBS+2% FBS) containing 1 ul human Fc block for 10-15 minutes at 4° C. Without washing, staining antibodies were added as follows:

| | Clone | | |
|---|---|---|---|
| Antibody | name | Isotype | Dilutions |
| PerCP anti-human TCR Vδ2 Antibody | B6 | Mouse IgG1, κ | 5 μl per million |
| PE anti-human TCR Vγ9 Antibody | B3 | Mouse IgG1, κ | cells in 100 μl |
| APC/Fire ™ 750 anti-human TCR α/β antibody | IP26 | Mouse IgG1, κ | staining volume |
| Brilliant Violet 510 ™ anti-human CD4 Antibody | A161A1 | Rat IgG2b, κ | |
| PE/Cy7 anti-human CD8 Antibody | SK1 | Mouse IgG1, κ | |
| FITC anti-human CD3 Antibody | UCHT1 | Mouse IgG1, κ | |

The resulting mixture was incubated at 4° C. for 30 minutes. The cells were then centrifuged and washed twice with FACS buffer, then resuspended in 100 ul FACS buffer. Samples were analyzed using a Novocyte 3000.

Transduction of γδ-T Cells with CAR Constructs

### (1) CAR Retrovirus Packaging

4-4.5 million Lenti-X 293T cells were plated in 10 ml DMEM+10% FBS+P/S one day before transfection. On the 2nd day, Optima® Medium (470 ul) plus Genejuice (30 ul) was added, mixed with the cells and the resulting mixture incubated at room temperature for 5 minutes.

A total of 10 ug DNA (3.75 ug pEQ-Pam3 package plasmid pKB0032+2.5 ug RD114 package plasmid pKB0033+3.75 ug SFG plus CAR plasmid construct (1B6 or 11C11, depicted above; 3:2:3 ratio) was mixed together and incubated at room temperature for 15 minutes. The final mixture (500 ul) was added dropwise to the Lenti-X 293T cells, gently rocking the plate for even mixing. The cells were cultured for about 4-6 h, then 5 ml more of prewarmed DMEM complete medium was added to the cells. The plates were kept incubated in culture for 2-3 days. The (CAR packaged retroviral) supernatant was harvested/filtered (48 h) and was ready for immediate transduction or for freezing at −80° C. for later use. 15 ml fresh DMEM+10% FBS+P/S was added to the cells and culture continued for another 24 h, following which the supernatant was harvested/filtered again (72 h).

### (2) γδ-T Cell Transduction

10 ug/ml retronectin in 2 ml PBS was coated per well of a 6-well non-TC treated plate overnight at 4° C. Day 6's PBMC gd (γδ)-T cells in culture were added with half volume of RPMI complete medium with 5 uM ZA plus 300 IU/ml hIL-2, stimulating overnight. gd-T cells purified on Day 7 according to Step 1b above were added, followed by the addition of retrovirus supernatant: 5 ml/well, spin 2000×g for 2 h at 32° C. The supernatant was removed, the wells were washed with RPMI culture medium, the medium was removed, and purified gd-T cells (2 million per well) were added in RPMI complete medium plus 300 IU/ml hIL-2, spin 1000×g for 10 minutes at 32° C. The cells were cultured in an incubator for 1 day and the transduction process was repeated on the second day. The cells were expanded by adding fresh medium with hIL-2 every 2-3 days. The transduction efficiency was measured on day 3-7 post transduction.

### (3) CAR γδ-T Cell Cytotoxicity Test

Green fluorescent protein (GFP)-expressing tumor cell lines (e.g., HeLa cells that express IsoMSLN and GFP, termed IsoMLSN-eGFP HeLa cells herein) were plated at 10,000 cells in 100 ul of RPMI+10% FBS+1% P/S per well in a 96-well TC-treated flat-bottom plate. The cells were cultured in an incubator overnight. On the second day, CAR γδ-T cells, prepared as described in (2) above, or non-transduced gd-T cells from different donors were resuspended in RPMI+10% FBS+1% P/S at different densities. Different numbers of CAR γδ-T cells in 100 ul of complete culture medium were added the tumor cells (resulting in different E (T cell):T (tumor cell) ratios, e.g., 5:1, 1:1, 1:5, 1:10, 1:30, etc.). The plate was incubated in incucyte S3, and live imaging for GFP detection (tumor cells) was performed over 3-7 days. The CAR-specific γδ-T cell cytotoxicity was measured by the decrease in GFP+ tumor cells compared to non-transduced (NT) γδ-T cells, or tumor cells alone.

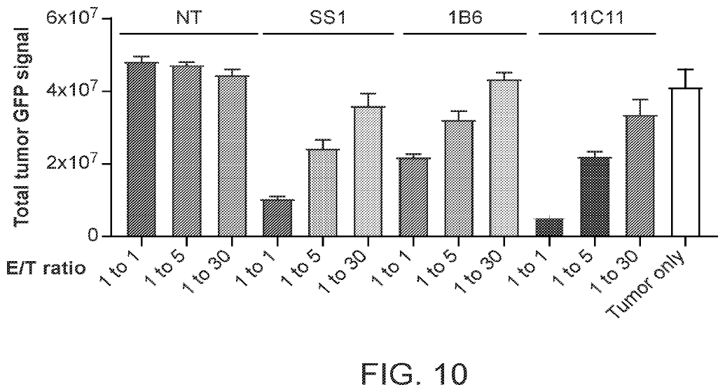

FIG. 10 shows the effects of treating IsoMLSN-eGFP HeLa cells with CAR γδ-T cells that express an anti-IsoMSLN scFv. In FIG. 10, the two types of controls are “NT” (non-transduced γδ-T cells) or tumor cells alone (“Tumor only”), “SS1” are CAR γδ-T cells that express an anti-Isoform 1/2 scFv, “1B6” are CAR γδ-T cells that express the 1B6 antibody anti-IsoMSLN scFv: 11CCAR γδ-T cells that express the 11C11 antibody anti-IsoMSLN scFv. The results demonstrate that both the 1B6 and the 11C11 CAR γδ-T cells are cytotoxic against the tumor cells, as seen by the dose-dependent decrease in the GFP signal. The results demonstrate that CAR γδ-T cells targeting IsoMSLN are cytotoxic against tumor cells and have the potential to control, regress or abolish tumor growth.

### Example 4: Method of Manufacturing a Composition Enriched in γδ-T Cells

Provided is an example of the methods provided herein for manufacturing a composition enriched in γδ-T cells (e.g., Vγ9Vδ2-T cell isolation and expansion). The resulting composition can be used per se as an immune cell composition, or can be genetically modified, e.g., by modifying one or more polypeptides expressed by the cell, by adding one or more exogenous polypeptides or genes expressing exogenous polypeptides, such as by retrovirus transduction.

### Abbreviations

| Abbreviation | Definition |
|---|---|
| ZA | Zoledronic Acid |
| P/S | penicillin and streptomycin |
| FBS | Fetal Bovine Serum |
| IL-2 | Interleukin-2 |
| MACS buffer | autoMACS Rinsing Solution + 1% BSA |

| Product | Company | Catalog # |
|---|---|---|
| Human IL-2 | Miltenyi Biotec | 130-097-748 |
| Human IL-7 | Miltenyi Biotec | 130-095-361 |
| Zoledronic acid | Sigma | 1724827 |
| TCRγ/δ⁺ T Cell Isolation Kit, human | Miltenyi Biotec | 130-092-892 |
| MACS LS column | Miltenyi Biotec | 130-042-401 |
| PerCP anti-human TCR Vδ2 Antibody | Biolegend | 331410 |
| PE anti-human TCR Vγ9 Antibody | Biolegend | 331308 |
| APC/Fire ™ 750 anti-human TCR α/β antibody | Biolegend | 306736 |
| FITC anti-human CD3 Antibody | Biolegend | 300440 |
| Human Fc block | Miltenyi Biotec | 130-059-901 |
| FITC anti-human CD3 Antibody | Biolegend | 300440 |
| Human Fc block | Miltenyi Biotec | 130-059-901 |
| CD34 Monoclonal Antibody (QBEND/10), PE | thermofisher | MA1-10205 |
| CTS ™ OpTmizer ™ T-Cell Expansion SFM | Gibco | A1048501 |
| Human AB Serum | Valley Biomedical | HP1022 |
| Fetal Bovine Serum, heat inactivated | Gibco | 10100147 |
| Penicillin and Streptomycin | Gibco | 15140122 |
| GlutaMAX ™ Supplement | Gibco | 35050079 |
| retronectin | Takara | T100B |
| Mirus transit lt1 | Mirus Bio | MIR 2305 |

### Abbreviations for Plasmids Provided Herein

| Plasmid ID | Note |
|---|---|
| pKB32 | pEQ-Pam3 (-e) package plasmid |
| pKB33 | RD114 package plasmid |
| pKB03 | SFG plus empty vector |
| pKB113 | Anti-IsoMSLN 1B6 CAR retrovirus plasmid |
| pKB115 | Anti-IsoMSLN 11C11 CAR retrovirus plasmid |

### Reagent Preparation

Zoledronic acid (ZA) solution: 5 mg Zoledronic acid powder in 4 ml 0.1N NaOH (0.8 mg/ml stock solution). A small aliquot was prepared and stored at −20° C. for long term storage. To prepare a 5 μM solution of ZA, 50 μl of the concentrated ZA solution was added to 30 ml of culture medium (see below).

Culture medium: The culture medium contained CTS™ OpTmizer™ T-Cell Expansion SFM (add CTS OpTmizer Expansion Supplement)+2% heat-inactivated human serum+1% GluMAX+ 1% P/S.

### METHOD

### A PBMC Isolation

Total PBMC are isolated from fresh blood according to standard methods known in the art and/or as described herein. A small aliquot of PBMC cells were saved for cell staining and flow analysis for γδT cell purity (see Part E and Table therein). 1 or 2 donors with >2% CD3+g9+d2+ T cells in the total CD3+ T cell population were selected for further processing.

### B Step 1: γδT Cell Expansion in the First 7 Days

(Below is the method starting with 20 million PBMCs, which can be scaled-up or down as needed.)

To the complete culture medium described above was added human IL-2 (IL-2), human IL-7 and Zoledronic Acid (ZA) to a final concentration of 300 IU/ml, 10 ng/mL and 5 μM, respectively. 20 million PBMC was resuspended in 20 ml culture medium (1×10⁶ cells/ml). The cells were cultured at 37° C. with 5% CO₂ (day 1). On day 3, the cells were fed with 10 ml complete culture medium containing human IL-2 (IL-2), human IL-7 and Zoledronic Acid (ZA) with concentration of 300 IU/ml, 10 ng/mL and 5 μM, respectively. On day 5 or 6, the cells were fed with 15 ml complete culture medium containing human IL-2 300 IU/ml plus human IL-7 10 ng/ml.

### C γδT Cell Enrichment on Day 7

On day 7 after PBMC culture and stimulation, the cells were harvested by centrifuging at 400×g for 5 minutes. The cell pellet was resuspended in culture medium and the cells were counted. A small aliquot containing 0.5 million PBMCs was set aside for cell staining and flow analysis for γδT cell purity.

αβ-T cells were depleted according to the manufacturer's protocol, as follows:
1) The cell suspension was centrifuged at 400×g for 5 min. The supernatant was completely aspirated.
2) The cell pellet was resuspended in 80 μL of MACS buffer per 10⁷ total cells.
3) 5 μL Biotin-anti-human TCR α/β Antibody (cat #130-113-529) was added per 10⁷ total cells, mixed well and refrigerated for 10 minutes (4-8° C.).
4) The cells were washed by adding 1 mL of MACS buffer per 10⁷ cells, then centrifuged at 400×g for 5 min. The supernatant was completely aspirated.
5) 80 μL of MACS buffer was added per 10⁷ total cells.
6) 5 μL of anti-biotin microbeads was added per 10⁷ total cells, mixed well and refrigerated for an additional 15 min (4-8° C.).
7) The cells were washed by adding 1 mL of buffer per 10⁷ cells, then centrifuged at 400×g for 5 min. The supernatant was completely aspirated.
8) Up to 10⁸ cells were resuspended in 500 μL of buffer and subjected to magnetic separation using a LS MACS Column. The MACS column was selected as described above, in a manner that ensured the cell number did not exceed its capacity.
9) The column was prepared by rinsing with an appropriate amount of buffer: e.g., LS=3 mL. The cell suspension was applied onto the column.
10) The cells were allowed to pass through column and the effluent was collected as the fraction with unlabeled cells, representing the enriched γδT cell fraction.
11) The column was washed with column-appropriate amount of buffer: e.g., LS=3 mL. The washing steps were performed by adding buffer two times. New buffer was added when the column reservoir became empty.
12) Total effluent was collected; this is the unlabeled cell fraction.
13) The cells were centrifuged at 400×g for 5 min. The supernatant was completely aspirated.
14) The cell pellet was resuspended in culture medium, and the cells were counted. A small aliquot containing 0.5 million purified γδT cells was set aside for cell staining and flow analysis for γδT cell purity as described in Part E.
   D Step 2: γδT Cell Expansion after Day 7

After γδT cell enrichment, the cells were either subjected to virus transduction (see Example 5) or expansion was continued in the complete culture medium with hIL-2 300 IU/ml plus hIL-7 10 ng/ml (cell concentration adjusted to 10⁶/ml). The cells were cultured at 37° C. in 5% CO₂ for another 7-14 days. A half volume of fresh culture medium (containing 300 IU/ml hIL-2 and hIL-7 10 ng/ml) was added every 2 to 3 days. Cell density was maintained at no more than 2×10⁶/ml. On day 14-21 of cell expansion, the cells were harvested for determining γδT cell purity and for performing functional assay(s).

### E Cell Staining and Flow Cytometry to Determine γδT Purity.

0.2 million cells per reaction were stained in 100 μl FACS staining buffer (DPBS+2% FBS) containing 1 μl human Fc block for 10-15 minutes at 4° C. Without washing, staining antibodies were added as shown in the Table below, and the mixture was incubated at 4° C. for 30 minutes. The cells were centrifuged and washed twice with FACS buffer. The cells were then resuspended in 100 μl FACS buffer. Samples were analyzed using a Novocyte 3000.

TABLE
Flow Cytometry staining panel to determine γδT cell purity.

| | Clone | | |
|---|---|---|---|
| Antibody | name | Isotype | Dilutions |
| PerCP anti-human TCR Vδ2 Antibody | B6 | Mouse IgG1, κ | 5 μl per million |
| PE anti-human TCR Vγ9 Antibody | B3 | Mouse IgG1, κ | cells in 100 μl |
| APC/Fire ™ 750 anti-human TCR α/β antibody | IP26 | Mouse IgG1, κ | staining volume |
| FITC anti-human CD3 Antibody | UCHT1 | Mouse IgG1, κ | |

### Example 5: Transduction of γδ-T Cells with Retrovirus

### CAR Retrovirus Packaging

This procedure produces 100 ml cell culture supernatant with either CAR pKB1115 retrovirus particles (construct depicted in FIG. 9) or CAR pKB113 retrovirus particles (construct depicted in FIG. 8) or any other CAR retrovirus construct known to those of skill in the art or provided herein.

### Procedure

Day 1: 30e6 293T cells were plated in 100 ml DMEM+10% FBS+P/S (no Plasmocin) in a Biofactory 1 Chamber, Wide Mouth (one-layer of total area 647 cm²)

Day 2: The following mixture was prepared:
1. Optima® Medium 10 ml
2. 100 ug total DNA was added into the Optima medium (37.5 ug pKB32+25 ug pKB33+37.5 ug SFG plus CAR pKB115 plasmid (3:2:3 ratio)) and mixed. The CAR pKB113 plasmid could also be used instead of the CAR pKB115 plasmid, or any other CAR retrovirus construct known to those of skill in the art or provided herein.
3. 300 ul Mirus TranslT-LT1 was added directly into the Optima/DNA mixture of 2., pipetted gently to mix completely, and incubated at room temperature for 15-30 minutes.
4. The final mixture was added dropwise to the one-layer of total area 647 cm² in the Biofactory chamber containing the 293T cells, gently rocking the plate for even mixing. The plate was kept in culture in incubation.

Day 5: The supernatant from the culture in 4 was harvested and filtered using a 0.45 uM filter. The supernatant was them divided into aliquots and frozen at −80° C. The cells were harvested with Trypsin+EDTA and stained with PE anti-CD34 antibody to determine the transfection efficiency.

### γδT Cell Transduction on Day 7

The day before transduction, a 6-well non-TC treated plate was coated with retronectin (10 μg/ml) in 2 ml PBS per well, at 4° C. overnight. Purified γδT cells obtained on day 7 as described in Part C of Example 4 above were used for the transduction. The purified cells were transduced with retrovirus (RV)—either pKB115 or pKB113, as follows:
(a) Retrovirus supernatant: pKB115 RV (or pKB113 RV; other CAR retrovirus also could be used) was added to the plate at 5 ml/well and spun at 2000×g for 2 hours at 32° C.
(b) The supernatant was removed, each well was washed with 2 ml culture medium and the medium was then aspirated.
(c) 2 million purified γδT cells were added per well in 2 ml complete culture medium plus 300 IU/ml hIL-2+10 ng/ml hIL-7, then spun at 1000×g for 10 minutes at 32° C.
(d) The transduction efficiency was determined on day 3-7 post-transduction.
(e) Half a volume of fresh culture medium (containing 300 IU/ml hIL-2 and hIL-7 10 ng/ml) was added every 2 to 3 days, and the cell density was maintained at no more than 2×10⁶/ml.
(f) On day 14-21 of cell expansion, the cells were harvested for determining γδT cell purity and for functional assay(s).

### CAR γδT Cell Cytotoxicity Test

(1) GFP-expressing tumor cell lines (e.g., IsoMLSN-eGFP Hela cells) were plated at 5,000 cells in 100 μl of complete culture medium per well in a 96-well TC-treated flat-bottom plate. The cells were cultured in an incubator overnight.
(2) On the following day (2^{nd} day), CAR γδT cells or non-transduced γδT cells from different donors were harvested and resuspended in complete culture medium at different cell concentrations. 100 μl complete culture medium containing different numbers of CAR γδT cells were added into the wells containing tumor cells, resulting in different E:T (effector to target) ratios (e.g., 5:1, 1:1, 1:10, 1:30, etc.).
(3) The plates were incubated in Incucyte S3 and live imaging for GFP (tumor cells) was initiated for a period of over 3-5 days.
(4) CAR-specific γδT cell cytotoxicity was measured by the decrease of GFP+ tumor cells compared to non-transduced γδT cells or tumor cells alone. The killing was found to be notable as early as 24 hours after coculture and peaked on day 3-5.

### Efficiency of γδT Cell Enrichment and γδT Cell Transduction:

On day 7 post ZA+IL-2 stimulation, CD3⁺Vγ9⁺Vδ2⁺ T cells are expected to be between 50-80% in cultured PBMCs, depending on the donors. The enrichment procedure further increases purity. After a 14-day expansion, cells should be ˜99% CD3⁺ and >95% Vγ9⁺ or Vδ2⁺ and <1% αβ⁺ T cells. Total expansion-fold is from 2500 to 12,000, depending on the donors.

The transduction efficiency of anti-MSLN CAR was found to be around 30-60%, depending on the CAR constructs and donors.

Non-transduced γδT cell may show some anti-tumor toxicity at high E to T (effector to target) ratios (e.g., 5:1). CAR γδT cells show target-specific cytotoxicity compared to non-transduced γδT cells even at lower E to T ratios (e.g., 1:1, 1:10).

### Example 6: Method of Manufacturing a Composition Enriched in iNKT Cells

Provided is an example of the methods provided herein for manufacturing a composition enriched in iNKT cells. The resulting composition can be used per se as an immune cell composition, or can be genetically modified, e.g., by modifying one or more polypeptides expressed by the cell, by adding one or more exogenous polypeptides or genes expressing exogenous polypeptides, such as by retrovirus transduction.

### Abbreviations

| Abbreviation | Definition |
|---|---|
| a-GC (α-GC) | Alpha galactoceramide |
| P/S | penicillin and streptomycin |
| HS | Human Serum |
| IL-2 | Interleukin-2 |
| IL-21 | Interleukin-21 |
| MACS buffer | autoMACS Rinsing Solution + 1% BSA |

### Materials and Equipment

| Product | Company | Catalog # |
|---|---|---|
| Human IL-2 | Miltenyi Biotec | 170-076-147 |
| alpha-galactoceramide | Avanti | 867000 |
| iNKT Cell Isolation Kit, human | Miltenyi Biotec | 130-092-892 |
| Human IL-21 | Miltenyi Biotec | 170-076-115 |
| MACS Columns | Miltenyi Biotec | 130-042-401 |
| PerCP anti-human CD3 Antibody | Biolegend | 300428 |
| FITC anti-human CD3 Antibody | BD Biosciences | 555332 |
| Brilliant Violet 421 anti-human iNKT Antibody | Biolegend | 342916 |
| FITC anti-human CD3 Antibody | Biolegend | 300440 |
| GlutaMAX | Gibco | 25030-081 |
| CTS media | Gibco | A37040-01 |
| Human Serum | Valley Biomedical | HP1022 |
| Penicillin and Streptomycin | HyClone | 15140-122 |
| Novocyte 3000 | ACEA BIO | |

### METHOD

### A PBMC Isolation

Total PBMC are isolated from fresh blood according to standard methods known in the art and/or as described herein. A small aliquot of PBMC cells were saved for cell staining and flow analysis for iNKT cell purity (see Part E and Table therein).

B Step 1: Screening of Donors for Initial iNKT Population

The purity of iNKT in the total PBMC sample was checked before proceeding with iNKT expansion and enrichment. Cell staining was performed as described in Part E and Table therein. Only donors with visible, distinct initial iNKT % (CD3+iNKT+)>0.05% were selected for further steps, i.e., iNKT expansion and enrichment.

### Step 2: Plate-Bound Generation of APCs (Antigen Presenting Cells)

(1) From the total PBMC sample, 10-15 million cells were counted and plated in 2 ml of serum free RPMI at 37° C. with 5% CO₂.
(2) Monocytes in the PBMC sample can adhere to the plate by 2 hours. Following a 2-hour incubation, non-adherent cells were washed 3 times with warm 1× PBS.
(3) Plate bound monocytes were supplemented with the culture medium.
   C iNKT Cell Enrichment on Day 0

The remaining PBMCs were used for iNKT enrichment, following the manufacture's protocol (Miltenyi Biotec #130-094-842) for iNKT cells isolation using MACS LS Columns. A small aliquot containing 1 million PBMCs was saved for cell staining and flow analysis for iNKT cell purity—see Part 5 and Table therein).
1) The cell suspension was centrifuged at 400×g for 5 minutes. The supernatant was aspirated completely.
2) The cell pellet was resuspended in 400 μL of buffer per 10⁸ total cells.
3) 100 μL of Anti-iNKT Microbeads was added per 10⁸ total cells, mixed well and incubated for 15 minutes in the refrigerator (2-8° C.). The cells were washed by adding 1-2 mL of MACS buffer per 10⁷ cells.
4) The cells were washed by adding 1-2 mL of buffer per 10⁸ cells and centrifuged at 300×g for 10 minutes. The supernatant was aspirated completely. 80 μL of MACS buffer was added per 10⁷ total cells.
5) Up to 10⁸ cells were resuspended in 500 μL of buffer, mixed well and refrigerated for an additional 15 min (4-8° C.).
6) The cells were washed by adding 1-2 mL of buffer per 10⁷ cells, then centrifuged at 400×g for 5 minutes. The supernatant was aspirated completely.
7) Up to 10⁸ cells were resuspended in 500 μL of buffer, then subjected to magnetic separation using MACS Columns. The column was selected to ensure that the cell number does not exceed the capacity of the column (see Table of column types and capacities in Example 3).
8) The selected MACS was prepared by rinsing with appropriate amount of buffer, e.g., LS=3 mL
9) The cell suspension was applied onto the column.
10) The cells were allowed to pass through the column, and effluent was collected as the fraction with unlabeled cells.
11) The column was washed with a column-appropriate amount of buffer, e.g., LS=3 mL Washing steps were performed by adding buffer two times, only adding new buffer when the column reservoir was empty.
12) The column was removed from the separator and placed on a suitable collection tube. The appropriate amount of buffer was pipetted, e.g., LS=5 ml, onto the column. The magnetic cells were immediately flushed out by firmly pushing the plunger into the column.
13) To increase the purity of iNKT cells, the eluted fraction was enriched over a second LS Column. The magnetic separation procedure as described in steps 8) to 12) was repeated using a new column.
14) The cell pellet was resuspended in CTS-Optimizer media+5% HS+P/S+Glutamax, and the cells were counted. A small aliquot containing purified iNKT cells was set aside for cell staining and flow analysis for iNKT cell purity, as described in Part E and the Table therein.
   D iNKT Cell Expansion from Day 0 to 10
1) After iNKT cell enrichment, the cell pellet was resuspended in 1.5 ml of expansion culture medium (CTS Optimizer media+5% HS+1% P/S+1% Glutamax+a-GC (100 ng/ml)+hIL-2 (200 U/ml)+hIL-21 (10 ng/ml). Cells were cultured at 37° C. in 5% CO₂ for 10 days.
2) The cultures were supplemented with fresh culture medium (containing IL-2 (200 IU/ml)) every other day. The cell density was maintained at no more than 2×10⁶/ml.
3) On days 7 and 10 of cell expansion, cells were harvested for determining iNKT purity and for functional assay(s).
   E Cell Staining and Flow Cytometry to Determine iNKT Purity.

0.2 million cells per reaction were stained in 100 μl FACS staining buffer (DPBS+2% FBS) containing 1 μl human Fc block for 10-15 minutes at 4° C. Without washing, staining antibodies were added as shown in the Table below, and the mixture was incubated at 4° C. for 30 minutes. The cells were centrifuged and washed twice with FACS buffer. The cells were then resuspended in 100 μl FACS buffer. Samples were analyzed using a Novocyte 3000.

TABLE
Flow Cytometry staining panel to determine iNKT cell purity

| | Clone | | |
|---|---|---|---|
| Antibody | name | Isotype | Dilutions |
| BV 421 anti-human iNKT TCR Antibody | 6B11 | Mouse IgG1, κ | 5 μl per million |
| PerCp anti-human CD3 Antibody | HIT3a | Mouse IgG1, κ | cells in 100 μl |
| | | | staining volume |

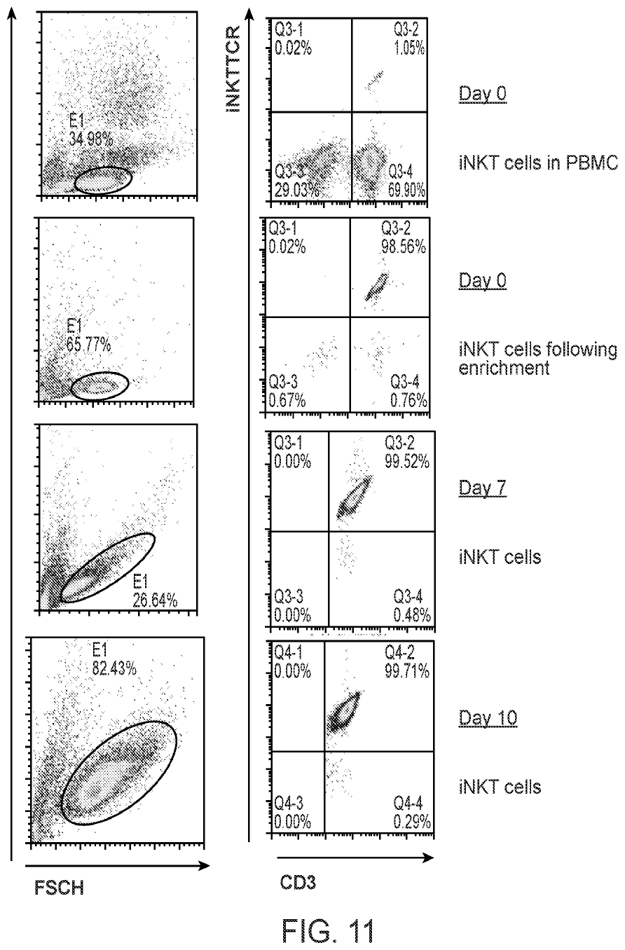

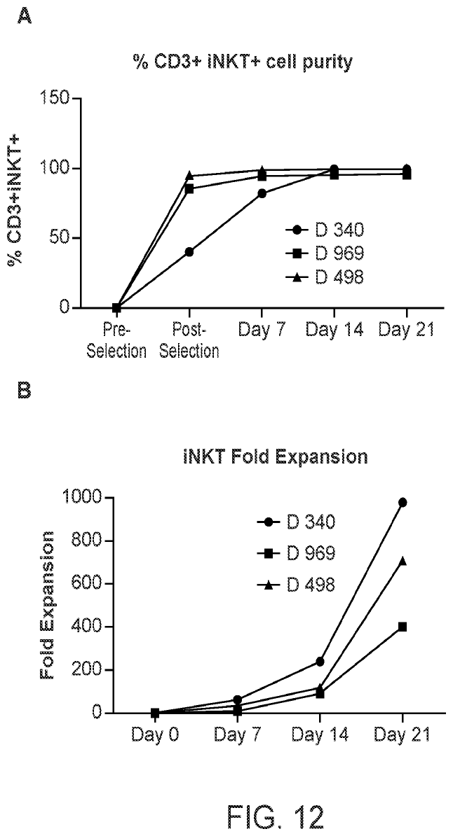

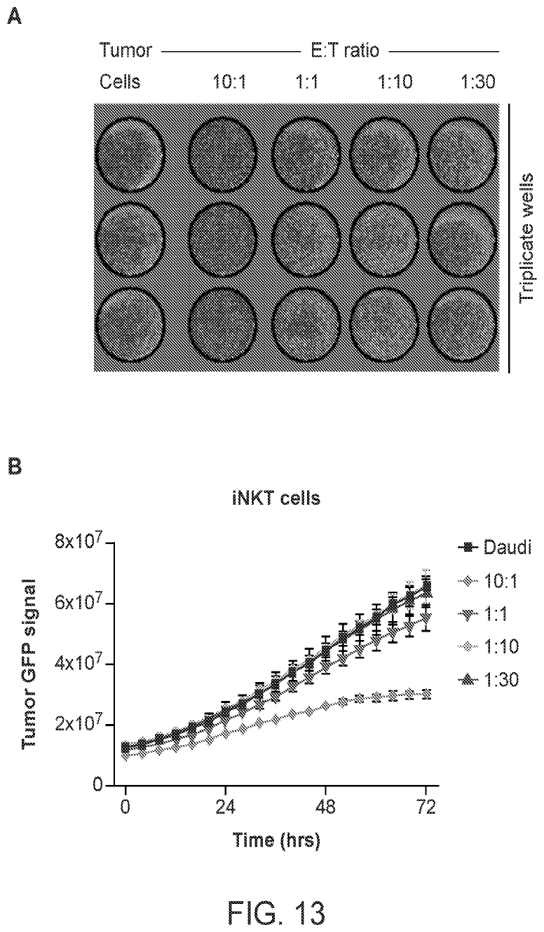

FIG. 11 shows that using the methods provided herein, a small percentage of iNKT cells in peripheral blood is significantly enriched and expanded, resulting in a pure population of iNKT cells (representative data from one of the donors: #498). FIG. 12 shows the expansion of pure iNKT cells during in vitro stimulation, culture, isolation and expansion processes. (A) Percentage of CD3⁺ iNKT+ cells over 21-day culture. (B) Expansion-fold of CD3+iNKT+ T cells. FIG. 13 demonstrates that in vitro expanded iNKT cells show cytotoxicity against Daudi cancer cells. (A) Day 3 imaging of Daudi-eGFP tumor cells cocultured at various E to T ratios of 14-day expanded iNKT cells (Donor #537, increased fluorescence depicted as brighter and lighter dots in grayscale). (B) In vitro Daudi-eGFP tumor cell growth kinetics in the presence of iNKT cells (E:T ratios depicted on the right). Data were analyzed by IncuCyte® S3 Live-Cell Analysis System.

### Example 7: Transduction of iNKT Cells with Retrovirus

### CAR Retrovirus Packaging

(a) 0.5-1 million Lenti-X 293T cells were plated in 10 ml DMEM+10% FBS+P/S for 24 h before transfection.
(b) On the 2^{nd} day, Optima® Medium 470 μl plus Genejuice 30 μl was added to the cells, mixed, and incubated at room temperature (RT) incubation for 5 minutes.
(c) A total of 10 μg DNA (3.75 μg pEQ-Pam3 (-e) package plasmid pKB0032+2.5 μg RD114 package plasmid pKB0033+3.75 μg SFG plus CAR plasmid, e.g., pKB113, pKB115 or other known CAR plasmid or provided herein (3:2:3 w/w/w ratio)) was added to the cells, mixed, and incubated at room temperature (RT) incubation for 15 minutes.
(d) The final mixture (500 μl) was added dropwise to the Lenti-X 293T cells, gently rocking the plate for even mixing. The cells were then cultured for about 4-6 hours followed by the addition of 5 ml more prewarmed DMEM complete medium to the cells.
(e) The plate was maintained in culture in incubation for 2-3 days. The supernatant was harvested and filtered (48 hours) and was ready for transduction or for freezing down at −80° C. for later use. 15 ml fresh DMEM+10% FBS+P/S was added and culture was continued for another 24 hours before harvesting/filtering the supernatant again (72 hours).
   iNKT Cell Transduction
(1) 6-well non-TC treated plates were coated with retronectin (10 μg/ml) in 2 ml PBS per well and maintained at 4° C., overnight.
(2) iNKT cells from Part D on day 10 were used for transduction:
   (a) Retrovirus supernatant was added at 5 ml/well and spun at 2000×g for 2 hours at 32° C.
   (b) The supernatant was removed, and the wells were washed with RPMI culture medium; the medium was then removed.
   (c) iNKT cells were then added at 0.5-1 million cells per well in CTS Optimizer complete media plus 200 IU/ml hIL-2, 10 ng/ml IL-21. Cells were spun at 1000×g for 10 minutes at 32° C.
   (d) The cells were expanded by adding fresh medium with hIL-2 and hIL-21 every 2 days.
   (e) The transduction efficiency was determined on day 3-7 post-transduction.
      CAR iNKT Cell Cytotoxicity Test
(1) GFP-expressing tumor cell lines (e.g., IsoMLSN-eGFP Hela cells) were plated at 10,000 cells in 100 μl of CTS Optimizer media+5% HS+1% P/S+1% Glutamax per well in a 96-well TC-treated flat-bottom plate. The cells were cultured in an incubator overnight.
(2) On the following day (2^{nd} day), CAR iNKT cells or non-transduced iNKT cells from different donors were harvested and resuspended in in CTS media+5% HS+1% P/S+1% Glutamax at different densities. 100 μl complete culture medium containing different numbers of CAR iNKT cells were added into the wells containing tumor cells, resulting in different E:T (effector to target) ratios (e.g., 5:1, 1:1, 1:10, 1:30, etc.).
(3) The plates were incubated in Incucyte S3 and live imaging for GFP (tumor cells) was initiated for a period of over 3-7 days.
(4) CAR-specific iNKT cell cytotoxicity was measured by the decrease of GFP⁺ tumor cells compared to non-transduced iNKT cells or tumor cells alone.
   Efficiency of iNKT Cell Enrichment and iNKT Cell Transduction:

On day 7 post α-GC+IL-2 stimulation, CD3⁺iNKT⁺ cells are expected to be between 80-90% in cultured PBMCs. After a 14-day expansion, cells should be >97% CD3+ iNKT+ and <0.5% CD3+ iNKT− cells. Total expansion-fold is from 300 to 1000-fold, depending on day 0 iNKT number.

The transduction efficiency of anti-MSLN CAR was found to be around 30-60%, depending on the CAR constructs and donors.

Non-transduced iNKT cells may show some anti-tumor toxicity at high E to T (effector to target) ratios (e.g., 5:1, 1:1). CAR iNKT cells (e.g., iNKT cells transduced with the pKB113 retroviral construct—see FIG. 8) show target-specific cytotoxicity compared to non-transduced γδT cells even at lower E to T ratios (e.g., 1:5, 1:10).

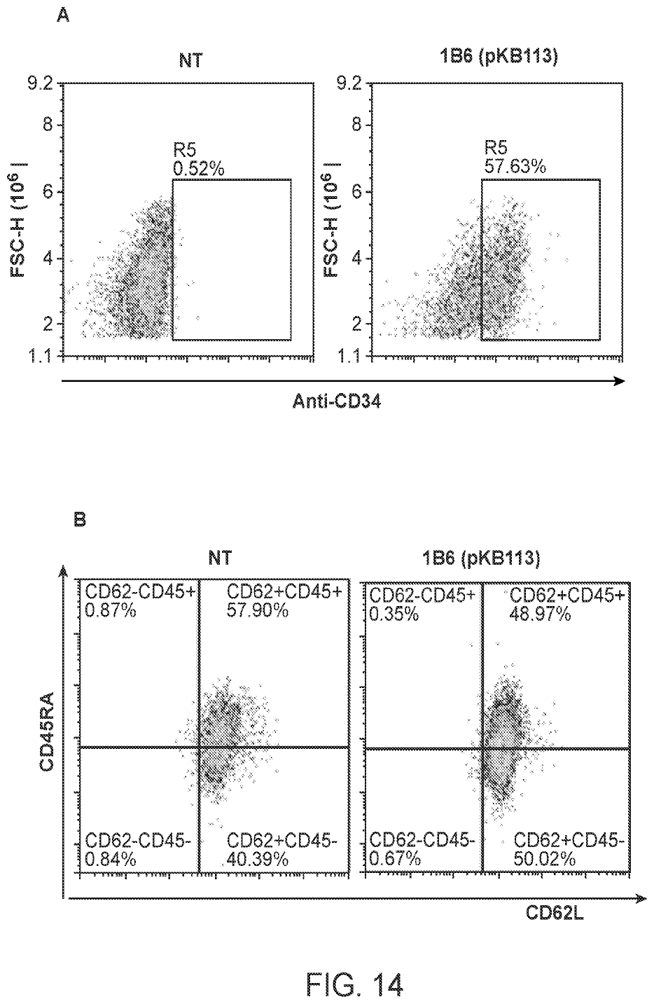

FIG. 14 depicts viral transduction of iNKT cells with the anti-isomesothelin (IsoMSLN) 1B6 CAR construct pKB113, depicted in FIG. 8. (A) Retrovirus transduction efficiency of iNKT cells with anti-IsoMSLN CAR (1B6) evaluated at 5 days post-transduction. Transduction efficiency was determined by flow cytometry staining of the CAR-embedded anti-CD34 epitope (NT: nontransduced; 1B6: transduced with CAR). (B) Initial characterization of iNKTs suggests a central memory phenotype in partial cell population (CD45RA-CD62L+) (NT: nontransduced; 1B6: transduced with CAR).

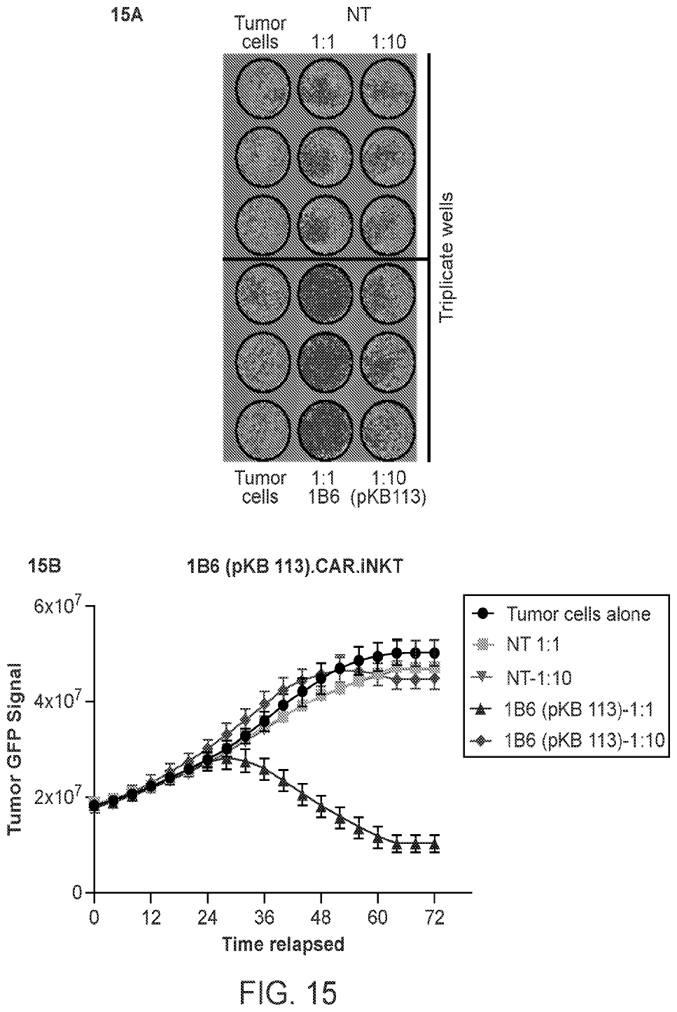

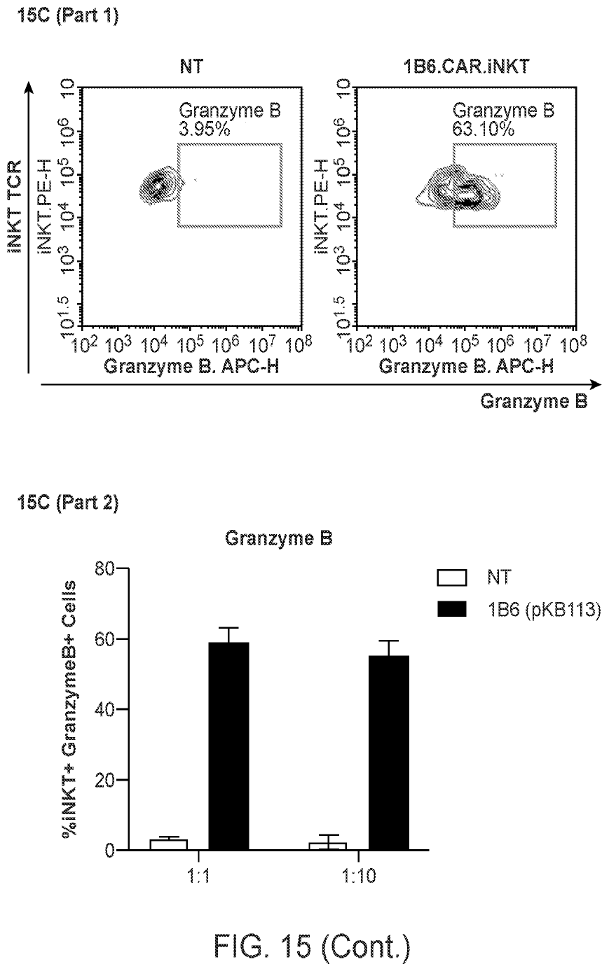

FIG. 15 demonstrates that anti-Isomesothelin CAR iNK-T cells show enhanced in vitro cytotoxicity against Iso-mesothelin-expressing tumor cells. (A) Day 3 imaging of human mesothelioma cell line (NCI-H226) cocultured with various E:T ratios of iNK T cells with or without anti-Iso MSLN CAR (pKB113) expression (increased fluorescence depicted as brighter and lighter dots in grayscale). (B) NCI-H226 tumor cell growth kinetics in the presence of CAR.iNKT cells. Data were analyzed using IncuCyte® S3 Live-Cell Analysis System. (C) Intracellular staining of Granzyme B and % iNKT+ Granzyme B+ cells in the co-culture.

The results demonstrate the following:
1. This method of iNKT cell expansion yields a highly pure population of of iNKT cells, with over 99% purity for CD3⁺ iNKT+ cells.
2. This approach has the potential to produce sufficient iNKT cells for clinical use.
3. Genetic modification of expanded iNKT cells show high cytotoxic potential against Isomesothelin (IsoMSLN), as measured by in vitro killing and Granzyme B staining.
4. The central memory phenotype of CARiNKTs suggests their better persistence in vivo

### Example 8: Design and Construction of a Chimeric PD1 Receptor and Activity Against Tumor Cells

Provided is an example of a chimeric PD1 receptor as described elsewhere herein. The chimeric PD1 receptor, namely chPD1, is a fusion protein containing the extracellular domain of the PD1 protein, which includes the binding sites for PD-ligands, a delta-CD28 extracellular domain, the CD28 transmembrane (TM) domain, the DAP10 cytoplasmic (used interchangeably herein with cytoplasmatic) region, and the CD3z cytoplasmic (used interchangeably herein with cytoplasmatic) region. The delta-CD28 extracellular region connects the PD1 extracellular region with the CD28 transmembrane domain but does not provide any signalling, because it does not contain the binding sites for CD80 and CD86. The chimeric molecule is transported to the cell surface by the PD1 native membrane signal sequence.

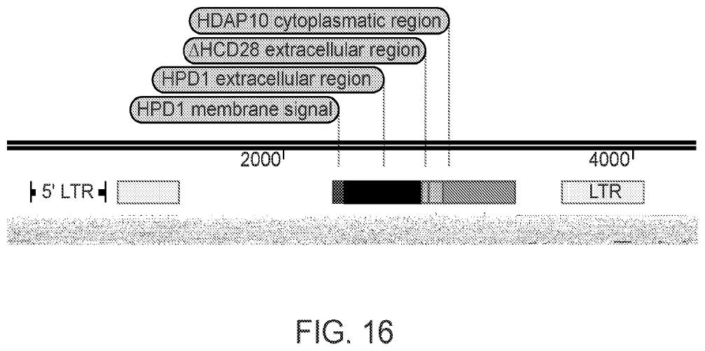

A schematic representation of the chPD1 chimeric receptor and its relationship with the gamma-retroviral vector used for stable transfection of the T cells is shown in FIG. 16 and depicts the following 6 regions, from N- to C-terminus (left to right):

chPD1 structure, from N- to C-terminus:

| | | |
|---|---|---|
| 1. | HPD1 signal | residues 1-23 of human PD1 (CD279) |
| 2. | HPD1 | human PD1 extracellular region (CD279), |
| | | residues 24-170 |
| 3. | ΔHCD28 | residues 141-151 of human CD28 |
| 4. | HCD28 TM | residues 153-179 of human CD28 |
| 5. | HDAP10 | residues 70-93 of human DAP10 |
| 6. | HCD3z | residues 52-164 of human CD3z (CD247) |

The polynucleotide and polypeptide sequences of each of the components of the above elements of the chPD1 structure, and of the structure as a whole, are presented in SEQ ID NOS: provided above as a Table of Sequences. All sequence identifiers are described in the Table of Sequences.

The DNA regulatory elements are as follows:

| | |
|---|---|
| MMLV-Psi | packaging signal of Moloney murine leukemia virus (MMLV) |
| gagpol | promoter of the gagpol gene from MMLV |
| Kozak | ribosome binding site for initiation of translation, GCCACCATGC (SEQ |
| | ID NO: 216) |
| Stop | 2x stop codons, TGA-TAA |
| LTR | long terminal repeat from Moloney murine leukemia virus. The 5′-LTR |
| | serves as the promoter of transcription |

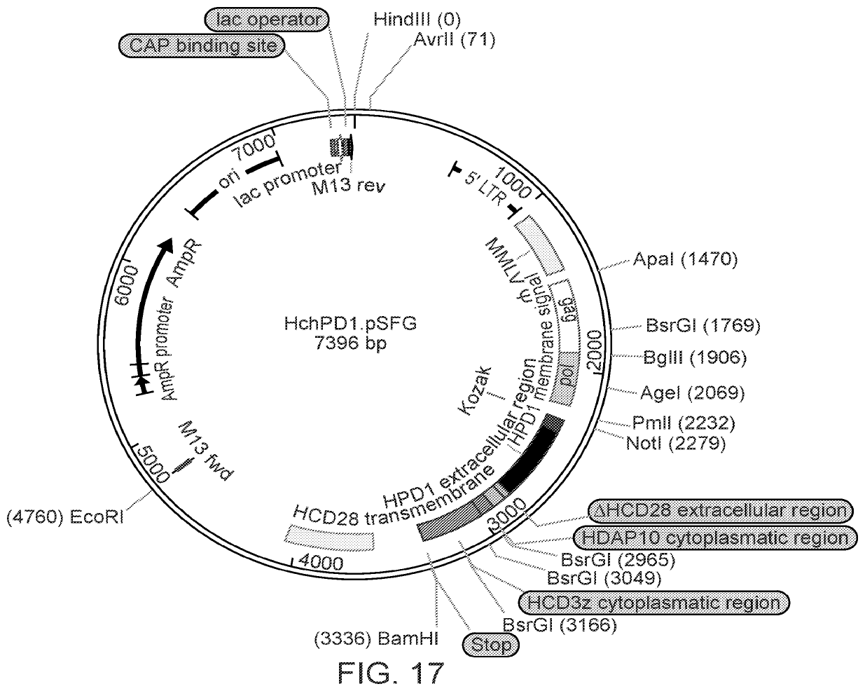

An example of a construct encoding the chPD1 chimeric receptor is depicted in FIG. 17.

DAP10-containing chPD1 CAR T cells have showed prolonged persistence, development of a central memory phenotype and enhanced anti-tumor activity in vivo compared with the same chPD1-CAR cells containing a CD28 co-stimulation domain (Lynch, A. et al. Adoptive transfer of murine T cells expressing a chimeric-PD1-Dap10 receptor as an immunotherapy for lymphoma. Immunology 152, 472-483, doi:10.1111/imm.12784 (2017)). Mechanistically, the stimulation of NKG2D/DAP10, unlike that of CD28, induces the activation of mTOR and supports the development of a central memory phenotype (McQueen, B., Trace, K., Whitman, E., Bedsworth, T. & Barber, A. Natural killer group 2D and CD28 receptors differentially activate mammalian/mechanistic target of rapamycin to alter murine effector CD8+ T-cell differentiation. Immunology 147, 305-320, doi:10.1111/imm.12563 (2016).

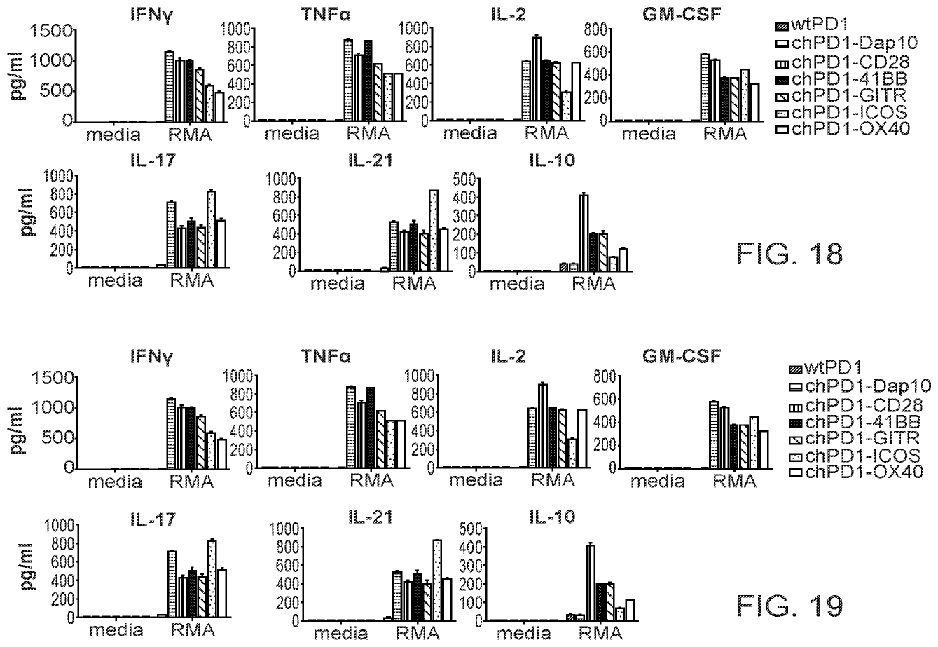

Comparing the cytokine profile of different CAR cells, secreted cytokines usually include pro-inflammatory IFN-γ, TNF, IL-2, GM-CSF, IL-17, and IL-21, as well as anti-inflammatory IL-10 (Kintz, H., Nylen, E. & Barber, A. Inclusion of Dap10 or 4-11BB costimulation domains in the chPD1 receptor enhances anti-tumor efficacy of T cells in murine models of lymphoma and melanoma. Cell Immunol 351, 104069, doi:10.1016/j.cellimm.2020.104069 (2020)). Unlike most other signaling domains, DAP10 was shown not to induce IL-10 secretion, but to strongly enhance T cell effector functions via pro-inflammatory cytokines (FIG. 18) (Kintz, H., Nylen, E. & Barber, A. Inclusion of Dap10 or 4-1 BB costimulation domains in the chPD1 receptor enhances anti-tumor efficacy of T cells in murine models of lymphoma and melanoma. Cell Immunol 351, 104069, doi:10.1016/j.cellimm.2020.104069 (2020); Maasho, K., Opoku-Anane, J., Marusina, A. I., Coligan, J. E. & Borrego, F. NKG2D is a costimulatory receptor for human naive CD8+ T cells. J Immunol 174, 4480-4484, doi:10.4049/jimmunol.174.8.4480 (2005)).

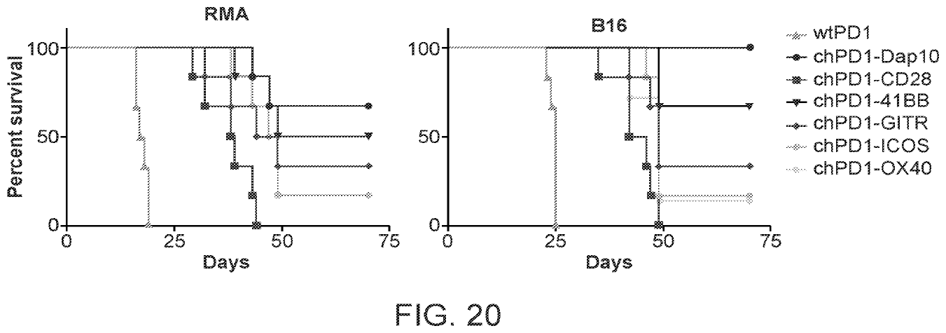

Similar to CD28-bearing receptors, DAP10 promoted differentiation towards an effectormemory phenotype in T cells (FIG. 19), which was associated with enhanced persistence in vivo (FIG. 20) (Barber, A. & Sentman, C. L. NKG2D receptor regulates human effector T-cell cytokine production. Blood 117, 6571-6581, doi:10.1182/blood-2011-01-329417 (2011); Lynch, A. et al. Adoptive transfer of murine T cells expressing a chimeric-PD1-Dap10 receptor as an immunotherapy for lymphoma. Immunology 152, 472-483, doi:10.1111/imm.12784 (2017); Whitman, E. & Barber, A. NKG2D receptor activation of NF-kappaB enhances inflammatory cytokine production in murine effector CD8(+) T cells. Mol Immunol 63, 268-278, doi:10.1016/j.molimm.2014.07.015 (2015)).

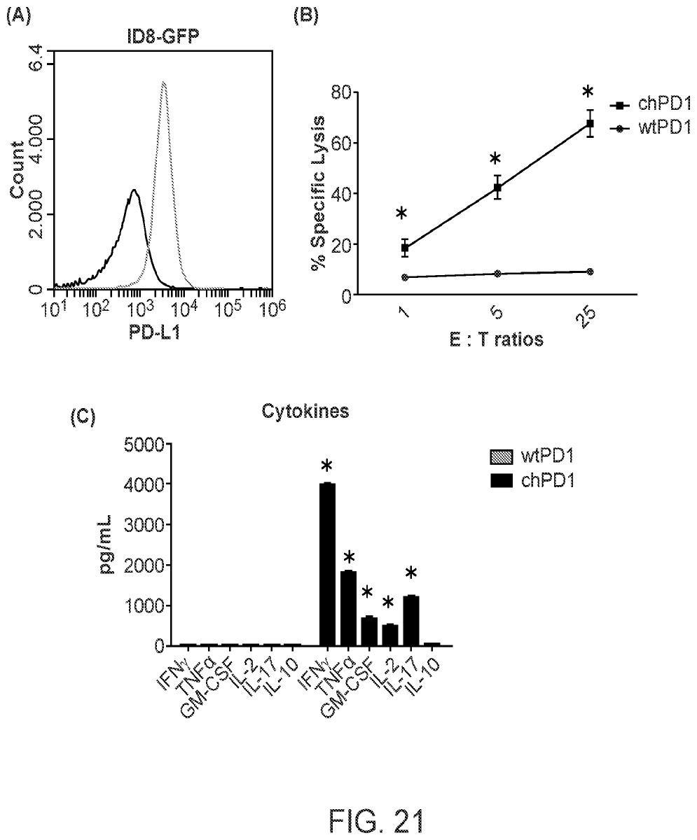

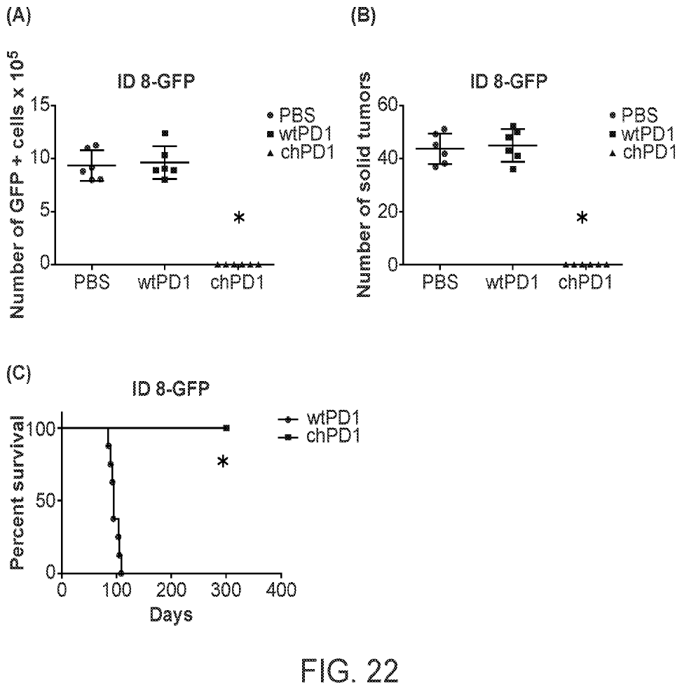

After selecting the DAP10-CD3z signaling domains as the optimal configuration for the chimeric PD1 switch receptor, validation of the strategy was investigated in a model of advanced ovarian cancer with malignant ascites. Peritoneal invasion by ovarian cancer (OC) cells is frequently associated with induction of PD-L1 expression, which promotes peritoneal dissemination by suppressing T cell cytotoxic functions (Abiko, K. et al. PD-L1 on tumor cells is induced in ascites and promotes peritoneal dissemination of ovarian cancer through CTL dysfunction. Clin Cancer Res 19, 1363-1374, doi:10.1158/1078-0432.CCR-12-2199 (2013). As shown in FIG. 21, the murine OC cell line, ID8, expressed high levels of PD-L1 on the plasma membrane, and it is susceptible to cytolysis by chPD1-transduced T cells in vitro, which produce a panel of pro-inflammatory cytokines. When tested in vivo, chPD1 but not wild type PD1 transduced T cells protected tumor bearing mice and extended the overall survival by dramatically reducing tumor cells in the peritoneal cavity (FIG. 22).

In conclusion, Gamma delta T cells from healthy donors were expanded ex vivo and stably transduced with a gamma-retrovirus-derived viral vector carrying the chimeric construct (FIG. 17). Cells were tested for killing efficiency, cytokines secretion, and memory differentiation in vitro, and the in vivo efficacy and tolerability was evaluated in murine xenografts. The results demonstrated that chPD1-gdT cells selectively killed PDL-1+ tumor cells in vitro and in vivo, with minimal on-target/off-tumor toxicities and without off-target toxicities. Cells were well tolerated in mice, without damage to normal PD-L1+ cells. Upon contact with PD-L1+ cancers, chPD1-gdT cells but not untransduced gdT cells expressed markers of memory phenotype and secreted inflammatory cytokines. Thus, chPD1-gdT cells are potent and safe in vitro and in vivo and will be assessed in a Phase I/II clinical trial.

### Example 9: Pharmacology and Toxicology Evaluation of Anti-IsoMSLN CAR Cells in Nude Mice

Provided are results of a study to determine whether: 1) Treatment with human gamma delta T cells expressing the anti-IsoMSLN CAR decreases tumor burden in the IsoMSLN+ NCI-H226 model of human mesothelioma; and 2) Treatment with human gamma delta T cells expressing the anti-IsoMSLN CAR is safe and well tolerated in the NCI-H226 model of human mesothelioma.

### 1. Materials and Equipment

All materials and equipment utilized in this study are listed in the Table below:

### Materials and Equipment

| Product | Company | Catalog # |
|---|---|---|
| Human IL-2 | Miltenyi Biotec | 130-097-748 |
| Zoledronic acid | Sigma | 1724827 |
| TCRγ/δ+ T Cell Isolation Kit, human | Miltenyi Biotec | 130-092-892 |
| MACS LS column | Miltenyi Biotec | 130-042-401 |
| PerCP anti-human TCR Vδ2 Antibody | Biolegend | 331410 |
| PE anti-human TCR Vγ9 Antibody | Biolegend | 331308 |
| FITC anti-human CD3 Antibody | Biolegend | 300440 |
| PE-anti-human CD34 antibody | Invitrogen | FAB7227P |
| Human Fc block | Miltenyi Biotec | 130-059-901 |
| CTS OpTmize T-Cell Expansion SFM | Gibco | A1048501 |
| Human AB Serum | Valley Biomedical | HP1022 |
| Fetal Bovine Serum, heat activated | Gibco | 10100147 |
| Pencillin and Streptomycin | Gibco | 15140122 |
| GlutaMAX Supplement | Gibco | 35050079 |
| Vectofusion-1 | Miltenyi Biotec | 130-111-163 |
| RetroNectin | Takara | T100B |
| Mirus transit LT1 | Mirus Bio | MIR 2305 |
| Retro-X Concentrator | Takara | 631455 |
| Opti-MEM I Medium | Gibco | A4124801 |
| Matrigel Membrane Matrix | Corning | CB-40234 |
| Human PBMC | ZenBio | NC0449441 |
| Uric Acid Reagents | ThermoScientific | TR24321 |
| AST/GOT Reagent | ThermoScientific | TR70121 |
| ALT/GPT Reagent | ThermoScientific | TR71121 |
| Mouse Creatinine Assay Kit | Crystal Chem | 50-194-7694 |
| Mouse SAA-3 ELISA | EMD Millipore | EZMSAA3-12K |
| Bilirubin Assay Kit | Abcam | Ab235627 |
| Urea Assay Kit | Abcam | Ab83362 |
| Accuri C6 Flow Cytometer | BD Biosciences | |
| Calipers | Bel-Art | 12-122 |
| Isotemp Water Jacketed CO2 Incubator | Fisherbrand | 11-676-600 |
| Thermo Scientific Sorvall ST 16R Centrifuge | Thermo Scientific | 75004381 |
| Eppendorf 5424R Microcentrifuge | Eppendorf | 5404000537 |

### 2. Methods

### 2.1. Donor

Peripheral blood mononuclear cells were purchased from ZenBio.

### 2.2. Preparation of Retroviral Vectors

24 h before transfection, 4 million Lenti-X 293T cells were plated and cultured in 37° C. plus 5% CO₂. On the day of cell transfection, 1 ml Opti-MEM Medium was mixed with a total of 10 μg DNA (3.75 ug pKB32+2.5 ug pKB33+3.75 ug SFG plus CAR plasmid (3:2:3 ratio) and mixed well. Then, 30 μl Mirus TranslT-LT1 was directly added into the Optima/DNA mixture, mixed, and incubated at room temperature for 30 min. The mixture was added dropwise to 10-cm dishes with the Lenti-X 293T cells, and the plate was rocked gently for even mixing. The cells were incubated at 37° C. for 2-3 days. The supernatant was harvested 2-days and 3-days post transfection and filtered with a 0.45 μM filter. The virus was concentrated using Retro-X Concentrator and stored at −80° C. in single-use aliquots.

### 2.3. gdT Cells Manufacturing

The donor who was selected had 1.6% CD3+Vγ9+δ2+ T cells in total CD3+ T cells, based on flow cytometry, for further processing. 20 million PBMC were suspended in culture medium with addition of human IL-2 (IL-2) and Zoledronic Acid (ZA) to a final concentration of 300 IU/ml and 5 μM, respectively. 20 ml culture medium was used for a concentration of 1×10⁶ cells/ml. Cells were cultured at 37° C. with 5% CO₂. After 3 days, 10 ml complete culture medium containing human IL-2 at 300 IU/ml and no ZA was added to the cells. On day 6, 15 ml complete culture medium containing human IL-2 300 IU/ml plus 5 μM ZA was added. On day 7 after PBMC culture and stimulation, cells were harvested by centrifuge at 400×g×5 min. γδT cells were isolated using the γδT cell isolation kit (Miltenyi Biotec #130-092-892). After γδT cell enrichment, the cells were either subjected to virus transduction to express the anti-IsoMSLN receptor, or continued to be expanded in the complete culture medium with hIL-2 300 IU/ml at 0.5×10⁶ cells/ml) for another 7 days.

Half of the volume of fresh culture medium (containing 300 IU/ml hIL-2) was added every 2 to 3 days to maintain a cell density at no more than 2×10⁶ cells/ml. For cells that were transduced to express the anti-IsoMSLN receptor, purified γδT cells were mixed with retrovirus supernatant (5 ml/well) pre-mixed with Vectofusion-1 and centrifuged at 400×g for 2 hours at 32° C. followed by static incubation at 37° C. After 8 hours, the supernatant was removed, and the wells were washed with 2 ml culture medium. The cells were cultured for 1 day and were split by adding fresh medium with 300 IU/ml hIL-2 every 2-3 days. The cells were transduced with the anti-IsoMSLN molecule as described in Example 3 or Example 5.

2.4. Flow cytometry

Prior to injection, gamma delta T cells were analyzed by flow cytometry. Purity of gamma delta T cells was analyzed by staining for CD3 and the Vγ2 receptor. Transduction with the anti-IsoMSLN receptor was assessed using staining for CD34 and was compared to staining with isotype control antibodies.

### 2.5. Animal Model

One tumor model was used: NCI-H226 human mesothelioma cell line in athymic BALB/c Nude mice. NCI-H226 (1×10⁶) was mixed with 50% Matrigel solution and administered subcutaneously.

After tumor inoculation, human gamma delta T cells were transferred to mice via intravenous injection. There were three treatment groups—no treatment, gamma delta T cells, and gamma delta T cells expressing the human anti-isoMSLN receptor.

Animals were monitored after injections to ensure that they continue to move about the cage (e.g., no limping, bleeding, labored breathing, etc.).

Based on previous results (Lam, S. K. et al. Growth suppressive effect of pegylated arginase in malignant pleural mesothelioma xenografts. Respiratory Research 18, 80, doi:10.1186/s12931-017-0564-3 (2017)), the NCI-H226 tumor model requires 30-40 days before control mice succumb to the tumor. Health conditions of mice were monitored daily, and mice were sacrificed upon first signs of distress due to tumor growth (shallow breathing, hunched back, ruffled fur, lethargy, diarrhea, difficulty walking/moving, weight loss, or large ulcerated tumor). For these models, tumor burden and survival experiments were conducted in the same set of mice. Tumor area was calculated every other day by measuring width and length, using calipers. Body weight was also measured every other day. For survival experiments, death was not used as an endpoint, but instead mice were sacrificed when the tumor volume reached 2000 mm³ or upon the first signs of distress (see symptoms above).

At the time of sacrifice, mice were euthanized by carbon dioxide (CO₂). This method of sacrifice is frequently used for small laboratory animals due to its rapid onset of action, safety, low cost, and ready availability. It also minimizes pain, fear, or other significant stress prior to animal death.

The cage was slowly filled with 100% CO₂ over five minutes, per the Office of Laboratory Animal Welfare (OLAW) standards. After five minutes, death was confirmed by observing vital signs and then by cervical dislocation. After sacrifice, mice were washed in 70% ethanol and various tissues were removed. Blood was collected from sacrificed animals.

### 2.6. Measurement of Tumor Burden

Tumor burden was measured every other day or daily, to assess the growth of the subcutaneous tumors. Tumor length and width were measured using a caliper and then tumor volume was calculated using the formula V=(L×W×W)/2, where V is tumor volume, W is tumor width, and L is tumor length. Mice were sacrificed when the tumor volume reached 2000 mm³, or upon the first signs of distress as described above.

### 2.7. Cytokine Assays

Blood was collected from the tail vein into a sterile empty tube. The tube was incubated at room temperature for 30 minutes, centrifuged at 1500×g for 10 minutes at 4° C., and serum was removed. Isolated serum was analyzed for levels of human cytokines using Eve Technologies' Human Cytokine 48-Plex Discovery Assay. Analytes included were:

sCD40L, EGF, Eotaxin, FGF-2, Flt-3 ligand, Fractalkine, G-CSF, GM-CSF, GROα, IFNα2, IFNγ, IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL-27, IP-10, MCP-1, MCP-3, M-CSF, MDC (CCL22), MIG, MIP-1α, MIP-1β, PDGF-AA, PDGF-AB/BB, RANTES, TGFα, TNFα, TNFβ and VEGF-A.

### 2.8. General Health Monitoring

Health conditions of mice were monitored daily, and mice were sacrificed upon first signs of distress due to tumor growth (shallow breathing, hunched back, ruffled fur, lethargy, diarrhea, difficulty walking/moving, weight loss, or large ulcerated tumor). Mice were weighed every other day to monitor weight loss.

### 2.9. Serological Analysis of Tissue Damage

Blood collected on the day of infusion of the gdT cells and on days 1, 3, 7, 12, and 19 after the infusion was used to prepare serum for the analysis of biomarkers of organ damage, as follows: liver and kidney function markers, namely AST/ALT/Bil (Aspartate transaminase/Alanine Transaminase/Total Bilirubin, for liver) and Uric acid, BUN/Cre (Blood Urea Nitrogen/Creatinine, for kidney), SAA (Serum amyloid A) for inflammatory response.

### 2.10. In Vivo Tumor-Rechallenge Experiments

60 days after original tumor injection, NCI-H226 (1×10⁶) was mixed with 50% Matrigel solution and administered subcutaneously in athymic BALB/c Nude mice.

There were two treatment groups—naïve or surviving mice previously treated with gamma delta T cells expressing the human anti-isoMSLN receptor. Tumor burden and health monitoring was performed as described. Circulating gd T cells were monitored in the blood by flow cytometry.

### 2.11. In Vivo Pharmacokinetics (Circulating gdT Cells)

Blood collected on days 3, 7, and 12 after the infusion was prepared for analysis of gamma delta T

cell circulation and CD34 expression on the circulating gd T cells. The number of circulating gd T

cells was analyzed by flow cytometry using staining for the gamma delta TCR and CD34 receptor.

### 2.12. Post-Mortem Pharmacokinetics (gdT Cells Tissue Distribution)

Upon sacrifice (19 days after T cell injection), the number of CD34-expressing human gamma delta T cells in the spleen, lymph nodes, and bone marrow were analyzed. The organs were removed, processed into a single-cell suspension, stained with antibodies specific for the human gd TCR and PD1, and analyzed using flow cytometry.

### 3. Results

### 3.1. Purity and Transduction of T Cells

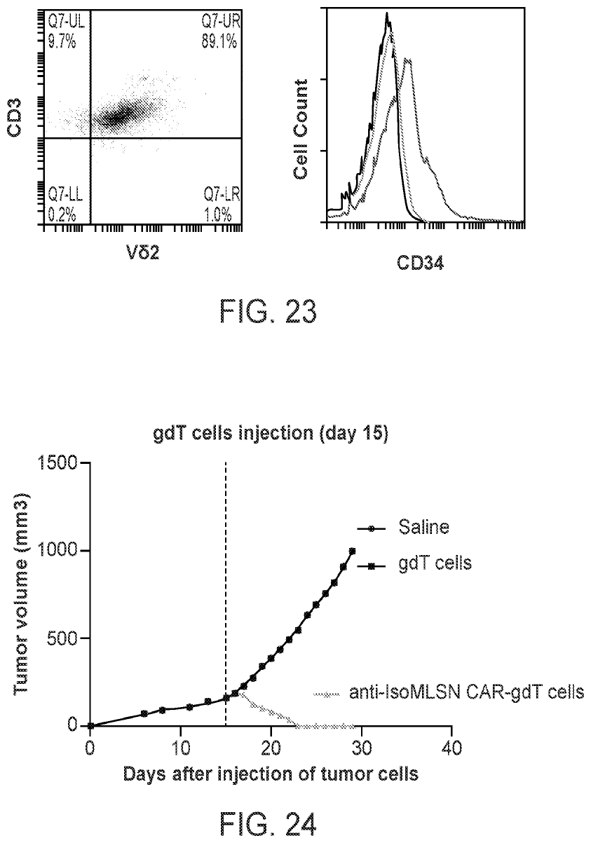

gdT cells used for this study were evaluated by flow-cytometry to determine purity and transduction efficiency at 48 hours after exposure to the retroviral vector. FIG. 23 shows that the purity of gdT cells was over 89%, and 69% of the cells were positive for the CD34 tag (CD34 minimal epitope tagging the N-terminus of the CAR molecule).

### 3.2. Tumor Growth

In vivo efficacy and tolerability of a high dose of gdT cells was tested in nude mice subcutaneously implanted with the Iso-MSLN+ cell line, NCI-H226, according to the method described by Lam et al. (Lam, S. K. et al. Growth suppressive effect of pegylated arginase in malignant pleural mesothelioma xenografts. Respiratory Research 18, 80, doi:10.1186/s12931-017-0564-3 (2017)). Fifteen days after tumor cell implantation, sub-cutaneous tumors subsequently became palpable and mice with comparable tumor volumes were then divided into 3 groups (n=10 mice/group): i) injected with gdT cells, ii) injected with CAR gdT cells, iii) injected with saline solution. Group 4 consisted of tumor-free, untreated mice (n=5). Tumor volumes and weight of the mice were measured daily for an additional 19 days, after which the mice were sacrificed. The results showed that CAR transduced gdT cells, but not non-transduced gdT cells, significantly controlled tumor growth starting 4 days after injection, and completely eliminated tumors within 10 days in 100% of the treated mice (FIG. 24).

### 3.3. Weight and General Health

No adverse reactions were observed over the course of the study in the tumor-bearing mice treated with CAR− expressing human gamma delta T cells, compared to non-tumor bearing mice as healthy animal controls. Adverse reactions that were monitored included presence of labored breathing, ruffled fur, reduced appetite, lethargy, or hunched posture. Furthermore, the mice treated with CAR− expressing human gamma delta T cells did not experience any weight loss over the course of the study.

### 3.4. Serological Tests of Liver and Kidney Damage

Serum samples prepared from the mice before the infusion of gdT cells and at the indicated time points after the infusion of gdT cells were used to measure the above-indicated markers of liver and kidney damage. The results showed no statistically significant difference between groups, with the exception of SAA (Serum Amyloid A marker), which indicated an acute inflammatory response to the infusion that peaked at day 3 post-infusion in the CAR gdT cell treatment group, and rapidly declined to baseline within 10 days.

### 3.5. In Vivo Pharmacokinetics

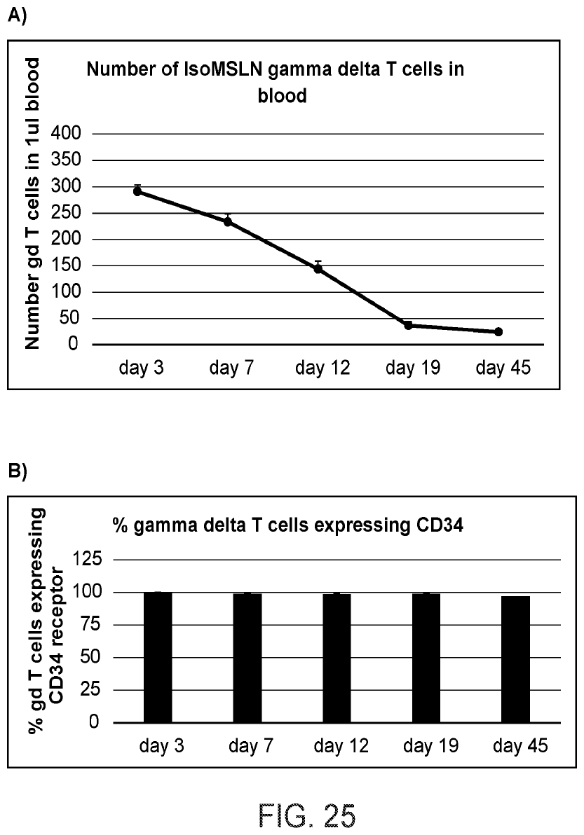

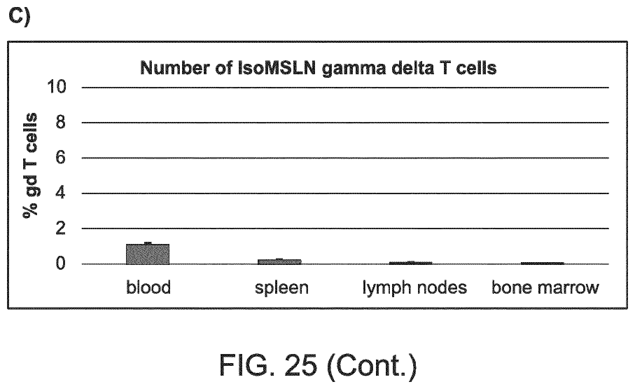

CAR-expressing human gamma delta T cell numbers were analyzed by flow cytometry. The peak number of CAR-expressing human gamma delta T cells in the blood was seen three days after T cell injection (FIG. 25). This peak T cell number correlates with the timing of when the tumor burden began to shrink. The human gamma delta T cells in the blood retained cell surface expression of CD34, which is a marker for CAR construct expression. Nineteen days after T cell injection, there were very low numbers of CAR-expressing human gamma delta T cells in the blood, spleen, lymph nodes, and bone marrow of the mice. CD34+ gamma delta T cells were still detectable in the blood 45 days after T cell injection.

### 3.6. Persistence of gdT Cells In Vivo Measured by Tumor Re-Challenging

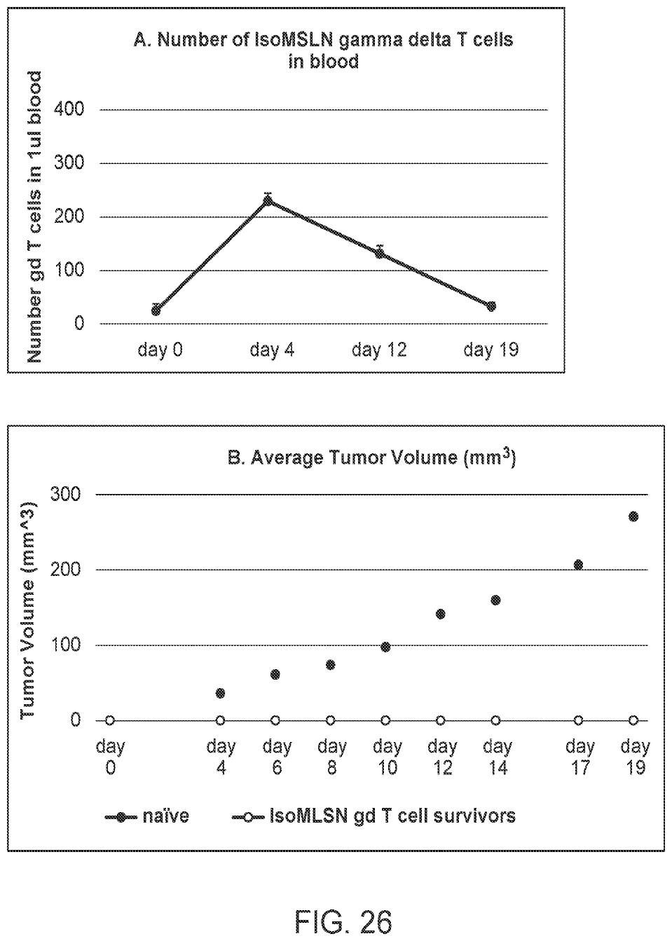

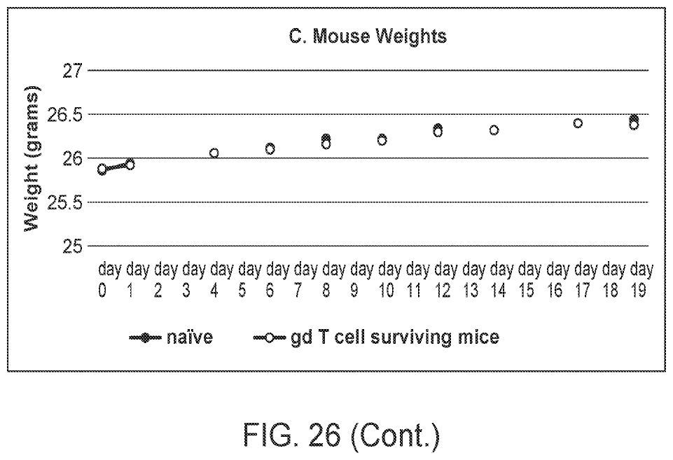

60 days after the injection of the tumor cells, 5 mice from the CAR gdT cells treatment group, and 5 mice from the tumor-free group (naïve mice) were injected with NCI-H226 cells, following the same protocol as the first tumor injection. Blood was drawn for the measurement of circulating CAR gdT cells, while tumor volume and mice weight was measured as indicated above. The results (FIG. 26) showed that tumor injection causes a temporary peak in circulating CAR gdT cells in 4 days, similar to what was shown following the CAR gdT cell infusion. The tumors grew only in naïve mice, while animals previously treated with CAR gdT cells were able to completely reject tumor cells.

### 3.7. Survival

No differences in survival were noted: all mice (CAR gdT cells treated, untransduced gdT cell treated and non-treated) survived until the end of the experiments.

### 3.8. Serum Cytokines

Blood was drawn 72 hours after the injection of the effector cells, which corresponds to the maximum blood concentration of the effectors. With the exception of GM-CSF, cytokine levels were not significantly different in CAR gdT cell treated mice compared with untreated mice or untransduced gdT cell treated mice. No alterations were detected of the relevant cytokines that could indicate the risk and severity of CRS in patients, i.e., IFNg (interferon gamma), IL-10, and IL-6. The results demonstrate that anti-IsoMSLN gdT cells can be well tolerated, with a minimal risk of CRS.

### Conclusion

These experiments demonstrate that a dose of 5 million CAR gdT cells in a single i.v. injection is safe and effective in controlling tumor growth, with a 100% response rate. Pharmacokinetic and histopathology analyses show that the engineered cells initially persist for about 20 days after the administration. Tumor re-challenge experiments indicate a prolonged persistency of the effector cells in vivo.

### Example 10: Killing of PDL-1 Positive Cancer Cells Using chPD1-Transduced gdT Cells

This experiment demonstrates that chimeric PD1 transduced gdT cells can efficiently and selectively kill PDL-1 positive cells in vitro.

Transduced Human Gamma Delta T Cells Express chPD1 Receptor and Expand In Vitro.

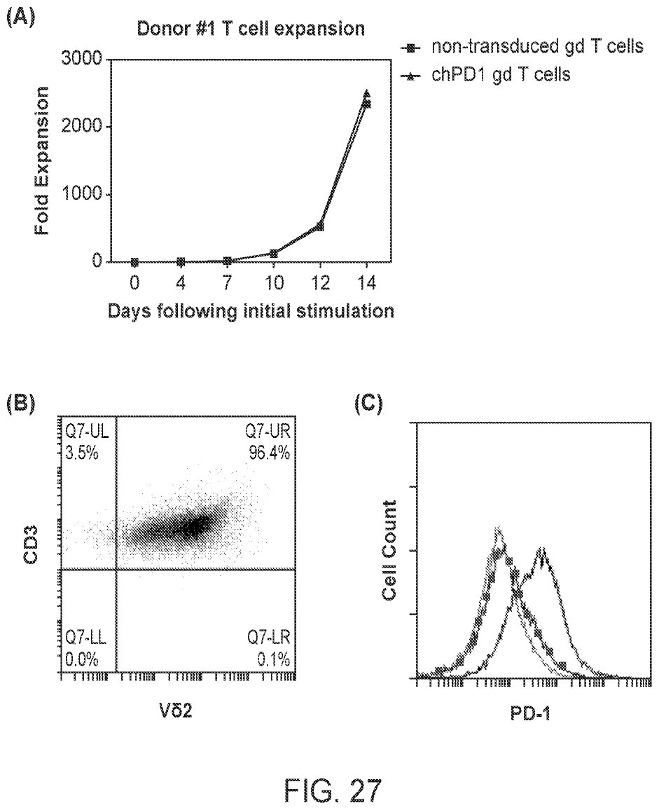

The anti-tumor activity of a human chPD1-DAP10 receptor expressed in γδT cells was assessed. Human γδT cells were isolated from cryopreserved PBMCs and were retrovirally transduced to express the chPD1-DAP10 receptor. Similar to non-transduced T cells, transduced chPD1-DAP10 γδT cells expanded more than 2,000-fold over a 14-day period in vitro (FIG. 27A). After in vitro expansion, the chPD1-DAP10 T cell population consisted of 99.9% CD3+ cells and >95% Vδ2+ and Vγ9+ cells (FIG. 27B). Seven days after transduction, chPD1-DAP10 γδT cells had increased cell surface expression of PD1 compared to non-transduced γδT cells, indicating that the chPD1-DAP10 receptor was expressed on the γδT cells (FIG. 27C). These data demonstrate that the human chPD1-DAP10 receptor can be expressed on PBMC-derived γδT cells and that expression of this receptor does not alter γδT cell expansion in vitro.

Human Gamma Delta chPD1-Expressing T Cells Lyse PD-L1-Expressing Tumor Cells.

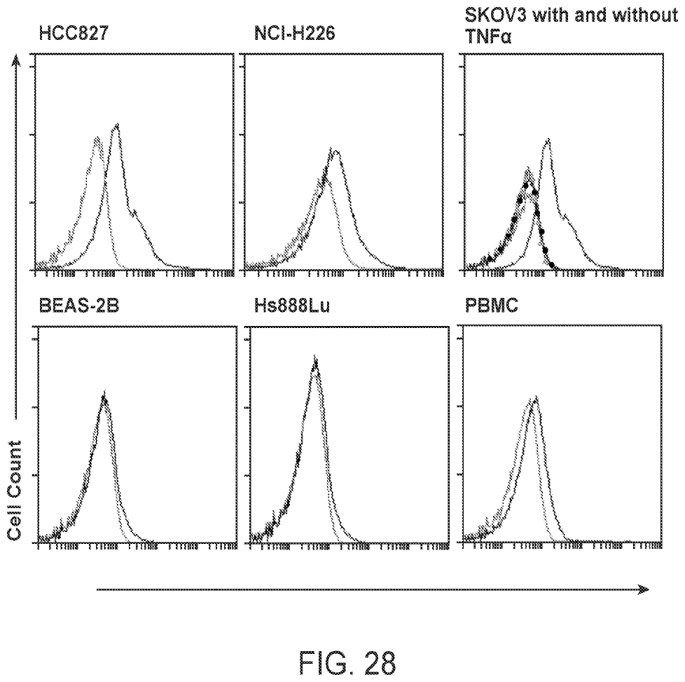

ChPD1-DAP10 γδT cell responses against human tumor cell lines was determined. HCC827 (human lung adenocarcinoma) and NCI-H226 (human mesothelioma) cell lines expressed cell surface PD-L1, as determined by flow cytometry (FIG. 28). The SKOV3 (human ovarian cancer) cell line did not express high levels of PD-L1; however, incubation of SKOV3 cells with 50 pg/mL TNFα increased cell surface expression of PD-L1 after 48 hours. Compared to human cancer cell lines, human lung epithelial cells (BEAS-2B), human lung fibroblasts (Hs888Lu), and human PBMCs did not have high expression of cell surface PD-L1. These data suggest that the PD-L1-positive human cancer cell lines, but not normal human tissues, may be potential targets for chPD1-DAP10 γδT cells.

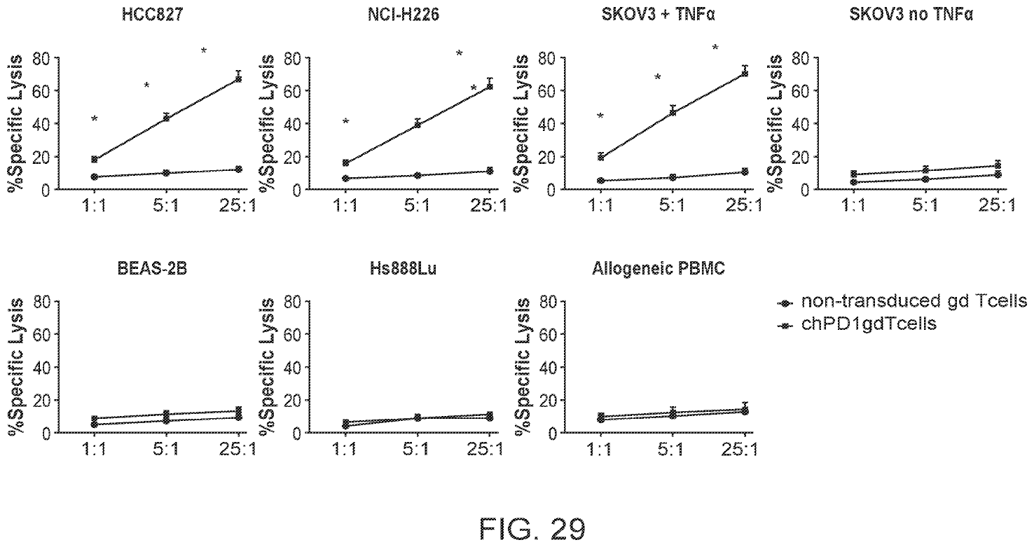

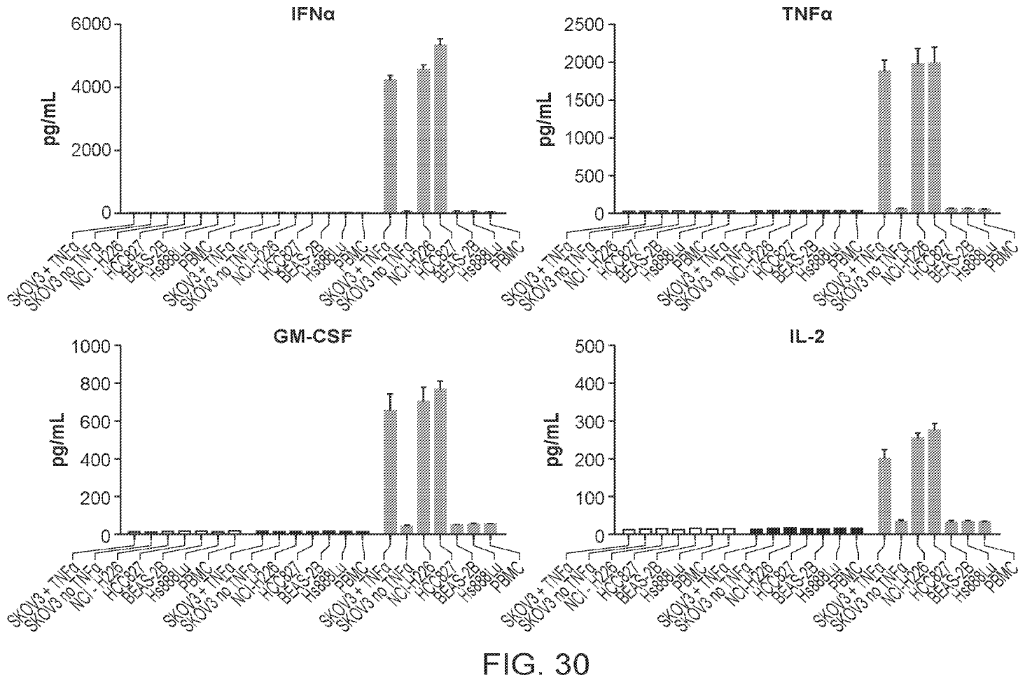

Next, the ability of human chPD1-DAP10 γδT cells to lyse PD-L1 positive tumor cells was assessed. Compared to non-transduced γδT cells, chPD1-DAP10 γδT cells demonstrated significant lysis of PD-L1-positive tumor cells HCC827 and NCI-H226 (FIG. 29). ChPD1-DAP10 γδT cells also lysed SKOV3 cells that were preincubated with TNFα but did not lyse SKOV3 cells without TNFα pre-treatment. Furthermore, chPD1-DAP10 γδT cells did not show significant lysis of human lung epithelial cells, lung fibroblasts, allogeneic PBMCs, or autologous PBMCs (FIG. 29 and data not shown). This suggests that chPD1-DAP10 γδT cells lyse PD-L1 positive cells but do not lyse cells that have low or no cell surface expression of PD-L1.

Human Gamma Delta chPD1-Expressing T Cells Secrete Proinflammatory Cytokines in Response to Tumor Cells.

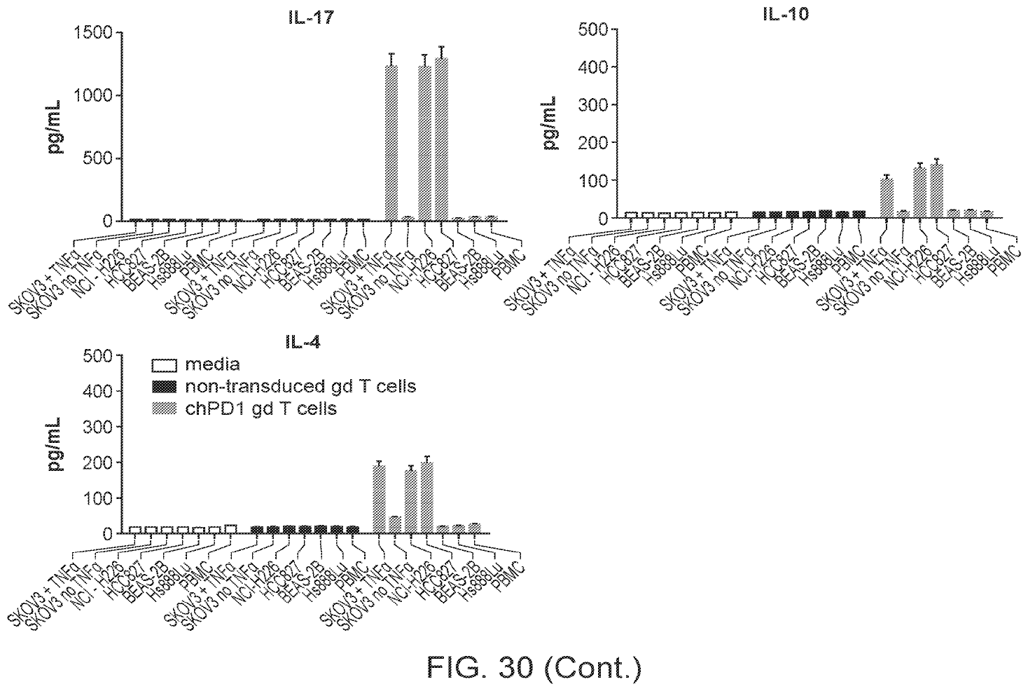

Cytokine secretion from chPD1-DAP10 γδT cells was also analyzed. ChPD1-DAP10 γδT cells secreted proinflammatory cytokines IFNγ, TNFα, GM-CSF, IL-2, and IL-17 in response to PD-L1 positive HCC827 cells, NCI-H226 cells, and SKOV3 cells pre-treated with TNFα. (FIG. 30). Unlike chPD1-DAP10 αβT cells, chPD1-DAP10 γδT cells also secreted low levels of IL-10 and IL-4 when cultured with the tumor cell lines (Parriott, G. et al. T-cells expressing a chimeric-PD1-Dap10-CD3zeta receptor reduce tumour burden in multiple murine syngeneic models of solid cancer. Immunology 160, 280-294, doi:10.1111/imm.13187 (2020)). In comparison, culture with normal human lung epithelial cells, lung fibroblasts, allogeneic PBMCs, or autologous PBMCs did not induce cytokine secretion from chPD1-DAP10 γδT cells (FIG. 30 and data not shown).

Non-transduced γδT cells did not secrete significant levels of cytokines in response to any of the cell lines tested. Overall, these data demonstrate that expression of the chPD1-DAP10 receptor in human γδT cells induced tumor lysis and cytokine secretion in response to PD-L1 positive tumor cells but not to healthy PD-L1 low or PD-L1 negative cells.

Human Gamma Delta chPD1 T Cells Express Central Memory Differentiation Markers.

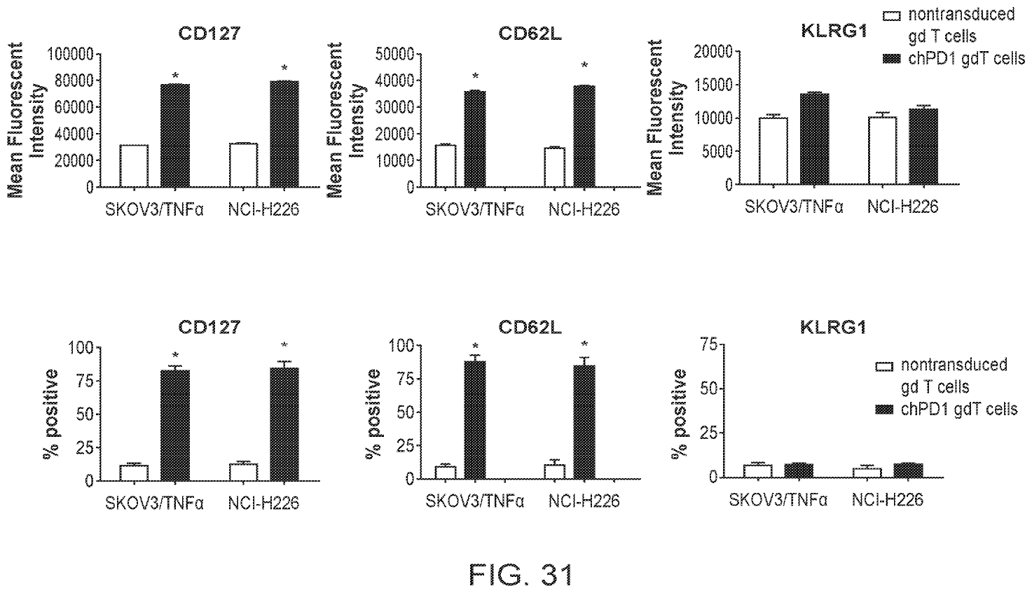

T cell differentiation is an important factor for CAR T cell efficacy. It has been previously shown that expression of the chPD1-DAP10 CAR in murine αβT cells induced a central memory phenotype and enhanced persistence in vivo^{14,19}. (Parriott, G. et al. T-cells expressing a chimeric-PD1-Dap10-CD3zeta receptor reduce tumour burden in multiple murine syngeneic models of solid cancer. Immunology 160, 280-294, doi:10.1111/imm.13187 (2020); Kintz, H., Nylen, E. & Barber, A. Inclusion of Dap10 or 4-1BB costimulation domains in the chPD1 receptor enhances anti-tumor efficacy of T cells in murine models of lymphoma and melanoma. Cell Immunol 351, 104069, doi:10.1016/j.cellimm.2020.104069 (2020)). However, the phenotype of chPD1-DAP10 γδT cells is not known. When cultured with NCI-H226 cells or SKOV3 cells preincubated with TNFα, chPD1-DAP10 γδT cells expressed cell surface markers associated with a central memory phenotype (CD127^{hi}, CD62L^{hi}, KLRG1^{lo}) (FIG. 31). These data indicate that expression of the chPD1-DAP10 receptor in human γδT cells may induce memory T cell phenotype, which could alter T cell persistence and in vivo anti-tumor efficacy.

The results demonstrate that chimeric PD1 transduced gdT cells can efficiently and selectively kill PDL-1 positive cancer cells. Because γδT cells exhibit their activity independent of HLA-peptide complexes, they can be suitable for allogenic administration, thereby allowing for “off-the-shelf” therapies.

### Example 11: Pharmacology and Toxicology Evaluation of chPD1 gdT Cells in Nude Mice

Provided are results of a study to determine whether: 1) Treatment with human gamma delta T cells expressing the chPD1 receptor decreases tumor burden in the NCI-H226 model of human mesothelioma; and 2) treatment with human gamma delta T cells expressing the chPD1 is safe and well tolerated in the NCI-H226 model of human mesothelioma.

### 1. Materials and Equipment

All materials and equipment utilized in this study are listed in the Table below:

### Materials and Equipment

| Product | Specifications | Company | Catalog # |
|---|---|---|---|
| Human IL-2 | | Miltenyi Biotec | 130-097-748 |
| Zoledronic acid | | Sigma | 1724827 |
| TCRγ/δ+ T Cell Isolation Kit, human | | Miltenyi Biotec | 130-092-892 |
| MACS LS column | | Miltenyi Biotec | 130-042-401 |
| PerCP anti-human TCR Vδ2 Antibody | | Biolegend | 331410 |
| PE anti-human TCR Vγ9 Antibody | | Biolegend | 331308 |
| FITC anti-human CD3 Antibody | | Biolegend | 300440 |
| APC-anti-human PD1 antibody | | Biolegend | 621610 |
| APC-anti-human PDL1 antibody | | Biolegend | 329708 |
| Human Fc block | | Miltenyi Biotec | 130-059-901 |
| CTS OpTmize T-Cell Expansion SFM | Grade, sterility, etc. | Gibco | A1048501 |
| Human AB Serum | | Valley Biomedical | HP1022 |
| Fetal Bovine Serum, heat inactivated | | Gibco | 10100147 |
| Penicillin and Streptomycin | | Gibco | 15140122 |
| GlutaMAX Supplement | | Gibco | 35050079 |
| Retronectin | | Takara | T100B |
| Mirus transit LT1 | | Mirus Bio | MIR 2305 |
| Retro-X Concentrator | | Takara | 631455 |
| Opti-MEM I Medium | | Gibco | A4124801 |
| Matrigel Membrane Matrix | | Corning | CB-40234 |
| Human PBMC | | ZenBio | NC0449441 |
| Uric Acid Reagents | | ThermoScientific | TR24321 |
| AST/GOT Reagent | | ThermoScientific | TR70121 |
| ALT/GPT Reagent | | ThermoScientific | TR71121 |
| Mouse Creatinine Assay Kit | | Crystal Chem | 50-194-7694 |
| Mouse SAA-3 ELISA | | EMD Millipore | EZMSAA3-12K |
| Bilirubin Assay Kit | | Abcam | Ab235627 |
| Urea Assay Kit | | Abcam | Ab83362 |
| Accuri C6 Flow Cytometer | | BD Biosciences | |
| Calipers | | Bel-Art | 12-122 |
| Isotemp Water Jacketed CO2 Incubator | | Fisherbrand | 11-676-600 |
| Thermo Scientific Sorvall ST 16R Centrifuge | | Thermo Scientific | 75004381 |
| Eppendorf 5424R Microcentrifuge | | Eppendorf | 5404000537 |

### 2. Methods

### 2.1 Donor

Peripheral blood mononuclear cells were isolated from buffy coats obtained through ZenBio.

### 2.2. Preparation of Retroviral Vectors

24 h before transfection, 4 million Lenti-X 293T cells were plated and cultured in 37° C. plus 5% CO₂. On the day of cell transfection, 1 ml Opti-MEM Medium was mixed with a total of 10 μg DNA (3.75 ug pKB32+2.5 ug pKB33+3.75 ug SFG plus CAR plasmid (3:2:3 ratio) and mixed well. Then, 30 μl Mirus TransIT-LT1 was directly added into the Optima/DNA mixture, mixed, and incubated at room temperature for 30 min. The mixture was added dropwise to 10-cm dishes with the Lenti-X 293T cells, and the plate was rocked gently for even mixing. The cells were incubated at 37° C. for 2-3 days. The supernatant was harvested 2-days and 3-days post transfection and filtered with a 0.45 μM filter. The virus was concentrated using Retro-X Concentrator and stored at −80° C. in single-use aliquots.

### 2.3 gdT Cells Manufacturing

A donor was selected who had 1.3% CD3+Vγ9+δ2+ T cells in the total CD3+ T cell population, based on flow cytometry, for further processing. 20 million PBMC were suspended in culture medium with the addition of human IL-2 (IL-2) and Zoledronic Acid (ZA), to a final concentration of 300 IU/ml and 5 μM, respectively. 20 ml culture medium was used for a final concentration of 1×10⁶ cells/ml. Cells were cultured at 37° C. with 5% CO₂. After 3 days, 10 ml complete culture medium containing human IL-2 at 300 IU/ml with no ZA was added to the cells. On day 6, 15 ml complete culture medium containing human IL-2 300 IU/ml plus 5 μM ZA was added. On day 7 after PBMC culture and stimulation, cells were harvested by centrifuging at 400×g×5 min. γδT cells were isolated using the γδT cell isolation kit (Miltenyi Biotec #130-092-892). After γδT cell enrichment, the cells were either subjected to virus transduction to express the chPD1 receptor, or continued to be expanded in the complete culture medium with hIL-2 300 IU/ml at 0.5×10⁶ cells/ml) for another 7 days.

Half of the volume of fresh culture medium (containing 300 IU/ml hIL-2) was added every 2 to 3 days to maintain a cell density at no more than 2×10⁶ cells/ml. For cells that were transduced to express the chPD1 receptor, the day before transduction, a 6-well non-tissue-culture treated plate was coated with retronectin (10 μg/ml) in 2 ml PBS per well at 4° C. overnight. Purified γδT cells were mixed with retrovirus supernatant (5 ml/well) and centrifuged at 2000×g for 2 h at 32° C. The supernatant was removed, and the wells were washed with 2 ml culture medium.

The cells were cultured for 1 day and were split by adding fresh medium with 300 IU/ml hIL-2 every 2-3 days.

### 2.4 Flow Cytometry

Prior to injection, gamma delta T cells were analyzed by flow cytometry. The purity of gamma delta T cells was analyzed by staining for CD3 and the Vγ2 receptor. Expression of the chPD1 receptor was assessed using staining for PD1 and was compared to staining with isotype control antibodies. Expression of PD-L1 on NCI-H226 cells was also assessed using flow cytometry.

### 2.5 Animal Model

One tumor model was used: NCI-H226 human mesothelioma cell line in athymic BALB/c Nude mice. NCI-H226 (1×10⁶) was mixed with 50% Matrigel solution and administered subcutaneously. After tumor inoculation, human gamma delta T cells were transferred to mice via intravenous injection. There were three treatment groups—no treatment, gamma delta T cells, and gamma delta T cells expressing the human chPD1 receptor.

Animals were monitored after injections to ensure that they continued to move about the cage (e.g., no limping, bleeding, labored breathing etc.).

Based on previous results (Lam, S. K. et al. Growth suppressive effect of pegylated arginase in malignant pleural mesothelioma xenografts. Respiratory Research 18, 80, doi:10.1186/s12931-017-0564-3 (2017)), the NCI-H226 tumor model requires 30-40 days before control mice succumb to the tumor. Health conditions of mice were monitored daily, and mice were sacrificed upon the first signs of distress due to tumor growth (shallow breathing, hunched back, ruffled fur, lethargy, diarrhea, difficulty walking/moving, weight loss, or large ulcerated tumor). For these models, tumor burden and survival experiments were conducted in the same set of mice. Tumor area was calculated every other day by measuring width and length using calipers. Body weight was also measured every other day. For survival experiments, death was not used as an endpoint, but instead mice were sacrificed when the tumor volume reached 2000 mm³ or upon the first signs of distress (see symptoms above).

At the time of sacrifice, mice were euthanized by carbon dioxide (CO₂). This method of sacrifice is frequently used for small laboratory animals due to its rapid onset of action, safety, low cost, and ready availability. It also minimizes pain, fear, or other significant stress prior to animal death. The cage was slowly filled with 100% CO₂ over five minutes per the Office of Laboratory Animal Welfare (OLAW) standards. After five minutes, death was confirmed by observing vital signs and then by cervical dislocation. After sacrifice, mice were washed in 70% ethanol and various tissues were removed. Blood was collected from sacrificed animals.

### 2.6 Measurement of Tumor Burden

Tumor burden was measured every other day daily to assess the growth of the subcutaneous tumors. Tumor length and width were measured using a caliber and then tumor volume was calculated using the formula V=(L×W×W)/2, where V is tumor volume, W is tumor width, L is tumor length. Mice were sacrificed when the tumor volume reached 2000 mm³ or upon the first signs of distress (see above).

### 2.7 Cytokine Assays

Blood was collected from the tail vein into a sterile empty tube. The tube was incubated at room temperature for 30 minutes, centrifuged at 1500×g for 10 minutes at 4° C., and serum was removed. Isolated serum was analyzed for levels of human cytokines using Eve Technologies' Human Cytokine 48-Plex Discovery Assay. Analytes included were:

sCD40L, EGF, Eotaxin, FGF-2, Flt-3 ligand, Fractalkine, G-CSF, GM-CSF, GROα, IFNα2, IFNγ, IL-1α, IL-1β, IL-1ra, IL-2, IL-3, IL-4, IL-5, IL-6, IL-7, IL-8, IL-9, IL-10, IL-12 (p40), IL-12 (p70), IL-13, IL-15, IL-17A, IL-17E/IL-25, IL-17F, IL-18, IL-22, IL-27, IP-10, MCP-1, MCP-3, M-CSF, MDC (CCL22), MIG, MIP-1α, MIP-1β, PDGF-AA, PDGF-AB/BB, RANTES, TGFα, TNFα, TNFβ and VEGF-A.

### 2.8 General Health Monitoring

Health conditions of the mice were monitored daily, and mice were sacrificed upon the first signs of distress due to tumor growth (shallow breathing, hunched back, ruffled fur, lethargy, diarrhea, difficulty walking/moving, weight loss, or large ulcerated tumor). Mice were weighed every other day to monitor weight loss.

2.9 Serological Analysis of Tissue Damage Blood collected on the day of infusion with gdT cells and on days 1, 3, 7, 12, and 19 after the infusion was used to prepare serum for the analysis of biomarkers of organ damage, as follows: liver and kidney function markers, namely: AST/ALT/Bil (Aspartate transaminase/Alanine Transaminase/Total Bilirubin, for liver); Uric acid, BUN/Cre (Blood Urea Nitrogen/Creatinine, for kidney); SAA (Serum amyloid A) for inflammatory response.

### 2.10 In Vivo Pharmacokinetics (Circulating gdT Cells)

Blood collected on days 3, 7, and 12 after the infusion was prepared for analysis of gamma delta T cell circulation and chPD1 expression on the circulating gd T cells. The number of circulating gd T cells was analyzed by flow cytometry, using staining for the gamma delta TCR and the PD1 receptor.

### 2.11 Post-Mortem Pharmacokinetics (gdT Cells Tissue Distribution)

Upon sacrifice (19 days after T cell injection), the number of chPD1-expressing human gamma delta T cells in the spleen, lymph nodes, and bone marrow were analyzed. The organs were removed, processed into a single-cell suspension, stained with antibodies specific for the human gd TCR and PD1, and analyzed using flow cytometry.

### 3. Results

### 3.1 Purity and Transduction of T Cells

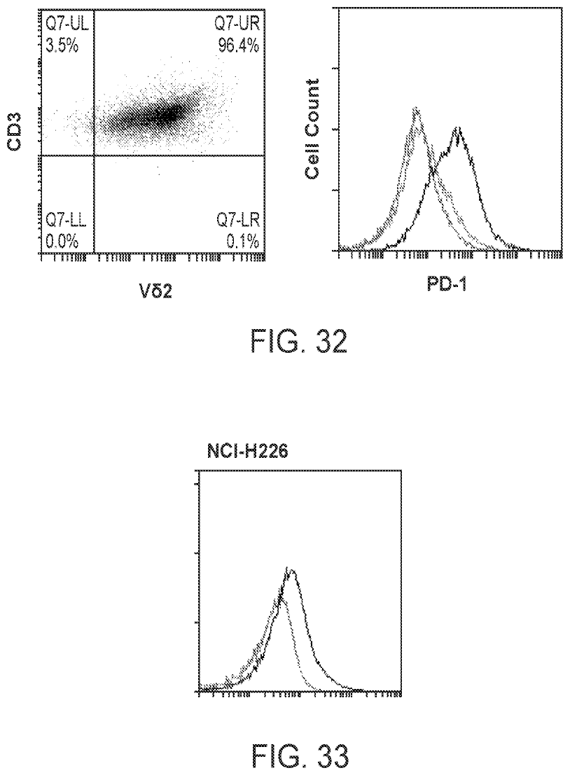

gdT cells used for this study were evaluated by flow-cytometry to determine purity and transduction efficiency at 48 hours after exposure to the retroviral vector. FIG. 32 shows that the purity of gdT cells was over 96%, and 68% of the cells were positive for PD1.

### 3.2 Evaluation of PDL1 Expression by Tumor Cells

Target tumor cell expression of PD-L1 was measured by flow cytometry. FIG. 33 shows that the tumor cell line, NCI-H226, was 58% positive for PD-L1.

### 3.3 Tumor Growth

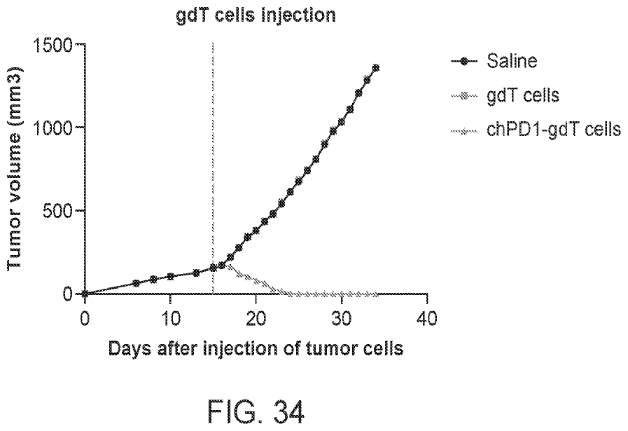

We tested the in vivo efficacy and tolerability of a high dose of gdT cell in nude mice subcutaneously implanted with the PDL1+ cell line, NCI-H226, according to the method described by Lam et al. (Lam, S. K. et al. Growth suppressive effect of pegylated arginase in malignant pleural mesothelioma xenografts. Respiratory Research 18, 80, doi:10.1186/s12931-017-0564-3 (2017)). Fifteen days after tumor cell implantation, subcutaneous tumors subsequently became palpable, and mice with comparable tumor volumes were then divided into 3 groups (n=10 mice/group): i) injected with gdT cells, ii) injected with chPD1 gdT cells, iii) injected with saline solution. Group 4 consisted of tumor-free, untreated mice (n=5). Tumor volumes and mice weight were measured daily for an additional 19 days, after which the mice were sacrificed. The results showed that chPD1 transduced gdT cells, but not non-transduced gdT cells, significantly controlled tumor growth starting 4 days after injection, and completely eliminated tumors within 10 days in 100% of the treated mice (FIG. 34).

### 3.4 Weight and General Health

No adverse reactions were observed over the course of the study in the tumor-bearing mice treated with chPD1-expressing human gamma delta T cells. Adverse reactions that were monitored included presence of labored breathing, ruffled fur, reduced appetite, lethargy, or hunched posture. Furthermore, the mice treated with chPD1-expressing human gamma delta T cells did not experience any weight loss over the course of the study. Non-tumor bearing mice were included as a healthy animal control, for comparison to the tumor-bearing mice.

### 3.5 Serological Tests of Liver and Kidney Damage

Serum samples prepared from the mice before the infusion of gdT cells, and at the indicated time points after the infusion of gdT cells, were used to measure the indicated markers of liver and kidney damage (see above). The results showed that there was no statistically significant difference between groups, with the exception of SAA (Serum Amyloid A marker), which indicated an acute inflammatory response to the infusion that peaked at day 3 post-infusion in the chPD1-expressing gdT cell treatment group, and rapidly declined to baseline within 10 days.

### 3.6 In Vivo Pharmacokinetics

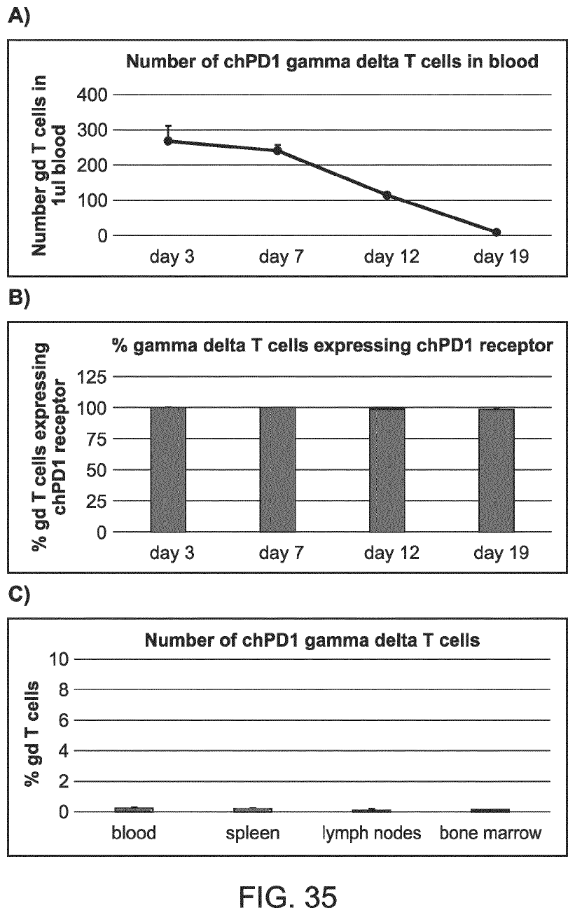

ChPD1-expressing human gamma delta T cell numbers were analyzed by flow cytometry. The peak number of chPD1-expressing human gamma delta T cells in the blood was three days after T cell injection (FIG. 35). This peak T cell number correlates with the timing of when the tumor burden began to shrink. The human gamma delta T cells in the blood retained cell surface expression of the chPD1 receptor. Nineteen days after T cell injection, there were very low numbers of chPD1-expressing human gamma delta T cells in the blood, spleen, lymph nodes, and bone marrow of the mice.

### 3.7 Survival

No differences in survival were noted: all mice (chPD1-expressing gdT cells treated, untransduced gdT cell treated and non-treated) survived until the end of the experiments.

### 3.8 Serum Cytokines

Blood was drawn 72 hours after the injection of the effector cells, which corresponds to the maximum blood concentration of the effectors. Except for IFNg (interferon gamma) and IL-6, cytokine levels were not significantly different in CAR gdT cell treated mice compared with untreated mice or untransduced gdT cell treated mice. Of the relevant cytokines that could indicate the risk and severity of CRS in patients, i.e., IFNg, IL-10, and IL-6, IL-10 was not detected, while IL-6 was close to the lower detection limit of the assay (0.13 pg/mL). IFNg levels were elevated in chPD1 gdT cells-treated mice. Although IFNg levels were above the cutoff of 75 pg/mL found by Teachey et al. (Teachey, D. T. et al. Identification of Predictive Biomarkers for Cytokine Release Syndrome after Chimeric Antigen Receptor T-cell Therapy for Acute Lymphoblastic Leukemia. Cancer Discov 6, 664-679, doi:10.1158/2159-8290.CD-16-0040 (2016) to be a predictor of severe CRS in combination with >60 pg/mL I-10, in this case it is not correlated with IL-10, as no IL-10 was detected. Therefore, the chPD1 gdT cells will likely be well-tolerated, with a minimal risk of CRS.

### 4 Conclusion

These experiments demonstrate that a dose of 5 million chPD1 gdT cells in a single i.v. injection is safe and effective in controlling tumor growth, with a 100% response rate. Pharmacokinetic and histopathology analyses show that the engineered cells initially persist for about 20 days after the administration.

### Example 12: Effect of IL-7 in the Culture Conditions for gdT Cell Expansion and Enrichment on Transduction Efficiency

Provided are results of a study to determine the effect of including IL-7 in the culture conditions for expansion and enrichment of a gdT cell population on the efficiency of transduction of the gdT cells.

### Reagents

| Name | Manufactor/Vendor | Cat # |
|---|---|---|
| Zoledronic acid | sigma aldrich | 1724827 |
| Recombinant Human IL-2 | Miltenyi Biotec | 130-097-748 |
| TCRγ/δ+ T Cell Isolation Kit, human | Miltenyi Biotec | 130-092-892 |
| TCRα/β Antibody, anti-human, Biotin | Miltenyi Biotec | 130-113-529 |
| Anti-Biotin MicroBeads Ultrapure | Miltenyi Biotec | 130-105-637 |
| LS Columns | Miltenyi Biotec | 130-042-401 |
| CTS ™ OpTmizer ™ T-Cell Expansion SFM | Gibco | A1048501 |
| MACS IL-15 | Miltenyi Biotec | 130-095-762 |
| MACS IL-7 | Miltenyi Biotec | 130-095-361 |
| Human AB Serum | Valley Biomedical | HP1022 |
| MACS buffer + 1% BSA | Miltenyi Biotec | |
| Penicillin-Streptomycin (10,000 U/mL) | Gibco | 15140122 |
| GlutaMAX ™ Supplement | Gibco | 35050079 |

### A Alpha.Beta T Cell (αβ-T Cell) Depletion

Alpha.beta T cell depletion was performed, using LS MACS columns, as follows:
1. On Day 0, PBMC were isolated from different donors and the cell numbers were counted.
2. The cell suspensions were centrifuged at 400×g for 5 minutes, and the supernatants were aspirated.
3. The cell pellets were resuspended in 80 μL of MACS buffer per 10⁷ total cells.
4. For αβ-T cell depletion, 5 μL Biotin-anti-human TCR α/β Antibody was added per 10⁷ total cells.
5. The antibody-cell combination was mixed well and refrigerated for 10 minutes (4-8° C.).
6. The cells were washed by adding 1-2 mL of MACS buffer per 10⁷ cells.
7. The cells were centrifuged at 400×g for 5 minutes, and the supernatants were aspirated.
8. 80 μL of MACS buffer was added per 10⁷ total cells.
9. 5 μL of Anti-Biotin microbeads was added per 10⁷ total cells.
10. The microbead-cell combination was mixed well and refrigerated for an additional 15 minutes (4-8° C.).
11. The cells were washed by adding 1-2 mL of MACS buffer per 10⁷ cells.
12. The cells were centrifuged at 400×g for 5 minutes, and the supernatants were aspirated.
13. Up to 10⁸ cells were resuspended in 500 μL of MACS buffer.
14. The resuspended cells were subjected to magnetic separation using MACS Columns of suitable size so that the cell numbers did not exceed the capacity of the columns (see Table regarding column sizing in Example 3).
15. The columns were prepared by rinsing with an appropriate amount of buffer (LS/LD: 3 ml), and the resuspended cells were applied to the columns.
16. The cells were allowed to pass through and effluent was collected as the fraction with unlabeled cells, representing the cells with αβ-T cell depletion fraction.
17. Washing steps were performed by adding buffer twice. New buffer was added when the column reservoir was empty (LS/LD: 2×3 m—twice for each of three donor samples)
18. The total effluent was collected; these are the desired unlabeled cell fractions.
19. If desired, the depletion procedure (steps 3-18) can be repeated.
20. The effluent was centrifuged at 400×g for 5 minutes, and the supernatant was aspired.

The cell pellets were resuspended in FACS buffer, and the cells were counted (a small aliquot containing 1 million cells was stained for flow cytometry analysis to detect residual aβ-T cells).

### B Determination of Gamma.Delta T Cell (γδ-T Cell) Purity

Cell staining and flow cytometry was conducted to determine γδ-T cell purity. Briefly, for each sample, 0.2 million cells were stained in 100 ul FACS staining buffer (DPBS+2% FBS) containing 1 ul human Fc block for 10-15 minutes at 4° C. Without washing, staining antibodies were added as shown in the Table below:

| | Clone | | | |
|---|---|---|---|---|
| Antibody | name | Isotype | Manufacturer | Dilutions |
| PerCP anti-human TCR Vδ2 Antibody | B6 | Mouse IgG1, κ | Biolegend | 2 μl per 0.2 million |
| PE anti-human TCR Vy9 Antibody | B3 | Mouse IgG1, κ | Biolegend | cells in 100 μl |
| FITC anti-human CD3 Antibody | UCHT1 | Mouse IgG1, κ | Biolegend | staining volume |
| APC/Fire ™ 750 anti-human TCR α/β antibody | IP26 | Mouse IgG1, κ | Biolegend | |

Samples were incubated at 4° C. for 30 minutes. The cells were spun down, and the cells were washed with FACS buffer twice. The cells were resuspended in 100 ul FACS buffer, and the samples were analyzed using a Novocyte 3000.

### C Gamma.Delta T Cell (γδ-T Cell) Expansion and Enrichment

Alpha.beta T cell-depleted cells, prepared according to Part A above, were obtained from three different donors: 290, 497 and 500. On Day 0, for Donor 290 and Donor 497, for each 10 cm culture dish, 10 million cells were suspended in 10 ml complete culture medium (1×10⁶ cells/ml). On Day 0, for Donor 500, for each 7 cm culture dish, 7 million cells were suspended in 7 ml complete culture medium (1×10⁶ cells/ml). The culture medium contained CTS medium+2% human serum+1% P/S+1% GlutaMAX+ZA (50 uM). Four culture dishes were set up for each donor (i.e., a total of 4×3 donors=12 culture dishes). The cells were cultured at 37° C. with 5% CO₂.

On Day 3, the culture dishes were fed with 5 ml complete culture medium, ZA (final concentration 5 μM) and four different cytokine treatments (four treatments: Group 1, Group 2, Group 3 and Group 4 for each Donor) as follows:

| Group Number | Cytokines in Culture Conditions |
|---|---|
| 1 | hIL-2 300 IU/ml |
| 2 | hIL-2 300 IU/ml |
| | hIL-7 10 ng/ml |
| 3 | hIL-2 300 IU/ml |
| | hIL-15 10 ng/ml |
| 4 | hIL-2 300 IU/ml |
| | IL-7 10 ng/ml |
| | hIL-15 10 ng/ml |

On Day 6, the cells were fed with 5 ml complete culture medium containing the cytokines for each group as described above.

D Transduction of γδ-T Cells with CAR Constructs

### (1) CAR Retrovirus Packaging

4-4.5 million Lenti-X 293T cells were plated in 10 ml DMEM+10% FBS+P/S one day before transfection. On the 2nd day, Optima® Medium (470 ul) plus Genejuice (30 ul) was added, mixed with the cells and the resulting mixture incubated at room temperature for 5 minutes.

A total of 10 ug DNA (3.75 ug pEQ-Pam3 package plasmid pKB0032+2.5 ug RD114 package plasmid pKB0033+3.75 ug SFG plus CAR plasmid construct (1B6 or 11C11, depicted above; 3:2:3 ratio) was mixed together and incubated at room temperature for 15 minutes. The final mixture (500 ul) was added dropwise to the Lenti-X 293T cells, gently rocking the plate for even mixing. The cells were cultured for about 4-6 h, then 5 ml more of prewarmed DMEM complete medium was added to the cells. The plates were kept incubated in culture for 2-3 days. The (CAR packaged retroviral) supernatant was harvested/filtered (48 h) and was ready for immediate transduction or for freezing at −80° C. for later use. 15 ml fresh DMEM+10% FBS+P/S was added to the cells and culture continued for another 24 h, following which the supernatant was harvested/filtered again (72 h).

### (2) γδ-T Cell Transduction

10 ug/ml retronectin in 2 ml PBS was coated per well of a 6-well non-TC treated plate overnight at 4° C. Day 6's PBMC gd (γδ)-T cells in culture were added with half volume of RPMI complete medium with 5 uM ZA plus cytokine treatment conditions according to each of Groups 1-4, as described above. On Day 7, gd-T cells purified according to Step 1b in Example 3 above added, followed by the addition of retrovirus supernatant: 5 ml/well, spin 2000×g for 2 h at 32° C. The supernatant was removed, the wells were washed with RPMI culture medium, the medium was removed, and purified gd-T cells (2 million per well) were added in RPMI complete medium plus cytokine treatment conditions according to each of Groups 1-4, as described above, spun at 1000×g for 10 minutes at 32° C. The cells were cultured in an incubator for 1 day and the transduction process was repeated on the second day. The cells were expanded by adding fresh medium with cytokine treatment conditions according to each of Groups 1-4, as described above, every 2-3 days. The transduction efficiency was measured on day 3-7 post transduction.

### (3) γδ-T Cell Transduction Efficiency

The transduction efficiency was analyzed by flow cytometry to determine the percentage of gdT cells carrying the CD34 tag (indicative of transduction of the CAR construct). The percent transduction efficiencies obtained for each donor, for the four groups of cytokine treatment conditions, are shown in the Table below:

| | hIL-2 | hIL-2/hIL-7 | hIL-2/hIL-15 | hIL-2/hIL-7/hIL-15 |
|---|---|---|---|---|
| Donor | Group 1 | Group 2 | Group 3 | Group 4 |
| 290 | 44.54% | 50.59% | 41.82% | 40.21% |
| 497 | 48.05% | 54.40% | 49.71% | 45.88% |
| 500 | 23.11% | 48.18% | 36.36% | 57.28% |

The results demonstrate that culture conditions that include IL-7 increases transduction efficiency compared to IL-2 alone, or IL-2+IL-15. For donors that show low transduction efficiency when exposed to IL-2 alone (e.g., due to lower numbers of retroviral receptors on their cells), the percent increase in transduction efficiency is more dramatic when IL-7 is included in the culture conditions (see, e.g., results for Donor 500).

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described, the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claims that follow.

What is claimed is:

1. A binding molecule that specifically binds to a polypeptide having the sequence set forth in SEQ ID NO:129, wherein the binding molecule comprises the CDR sequences set forth in SEQ ID NOS:3-5 and SEQ ID NOS:12-14.

2. A binding molecule that specifically binds to a polypeptide epitope that includes SEQ ID NO:131 or SEQ ID NO:132, comprising the CDR3 of SEQ ID NO:2 and the CDR3 of SEQ ID NO:11.

3. The binding molecule of claim 2, comprising the CDR1 and CDR2 of SEQ ID NO:2 and the CDR1 and CDR2 of SEQ ID NO:11.

4. The binding molecule of claim 2 or claim 3, comprising a heavy chain variable domain about 95% or more identical to the heavy chain variable domain of SEQ ID NO:2.

5. The binding molecule of any one of claims 2-4, comprising a light chain variable domain about 95% or more identical to the light chain variable domain of SEQ ID NO:11.

6. The binding molecule of any one of claims 1-5, comprising the heavy chain variable domain of SEQ ID NO:2 and the light chain variable domain of SEQ ID NO:11.

7. A binding molecule that specifically binds to a polypeptide having the sequence set forth in SEQ ID NO:129, wherein the binding molecule comprises the CDR sequences set forth in SEQ ID NOS:39-41 and SEQ ID NOS:48-50.

8. A binding molecule that specifically binds to a polypeptide epitope that includes SEQ ID NO:131 or SEQ ID NO:132, comprising the CDR3 of SEQ ID NO:38 and the CDR3 of SEQ ID NO:47.

9. The binding molecule of claim 8, comprising the CDR1 and CDR2 of SEQ ID NO:38 and the CDR1 and CDR2 of SEQ ID NO:47.

10. The binding molecule of claim 8 or claim 9, comprising a heavy chain variable domain about 95% or more identical to the heavy chain variable domain of SEQ ID NO:38.

11. The binding molecule of any one of claims 8-10, comprising a light chain variable domain about 95% or more identical to the light chain variable domain of SEQ ID NO:47.

12. The binding molecule of any one of claims 7-11, comprising the heavy chain variable domain of SEQ ID NO:38 and the light chain variable domain of SEQ ID NO:47.

13. The binding molecule of any one of claims 1-12, comprising an antibody, antibody fragment, single-chain antibody, diabody, or BiTe.

14. The binding molecule of any one of claims 1-13, which is a chimeric antigen receptor molecule.

15. The chimeric antigen receptor molecule of claim 14 that is of the formula:
5′-(CD8 signal)-(Linker 1)-(CD34 tag)-(Linker 2)-(VH Domain)-(Linker 3)-(VL Domain)-(Linker 4)-(CD8 stalk region)-(CD8 transmembrane region)-(Linker 5)-(CD28 cytoplasmic region)-(CD3-zeta cytoplasmic region)-3′.

16. A nucleic acid comprising a polynucleotide that encodes a binding molecule of any one of claims 1-15.

17. The nucleic acid of claim 16, wherein the polynucleotide comprises the sequence set forth in SEQ ID NO:74, a sequence that is 95% or more identical to the sequence set forth in SEQ ID NO:74, the sequence set forth in SEQ ID NO:102, or a sequence that is 95% or more identical to the sequence set forth in SEQ ID NO:102.

18. A cell, comprising:
one or more binding molecules of any one of claims 1-15; and/or
one or more nucleic acids each encoding one or more binding molecules of claim 16 or claim 17.

19. The cell of claim 18 that is an iNKT cell.

20. The cell of claim 18 that is a γδ-T cell.

21. A method for determining the presence, absence or amount of a mesothelin isoform-2 polypeptide comprising SEQ ID NO:129, or a polynucleotide encoding the polypeptide comprising SEQ ID NO:129, comprising contacting a biological sample or biological preparation with (i) a binding molecule that specifically binds to the mesothelin isoform-2 polypeptide, and/or (ii) a polynucleotide complementary to the polynucleotide encoding the mesothelin isoform-2 polypeptide or complement thereof.

22. The method of claim 21, wherein the binding molecule is a binding molecule of any one of claims 1-15.

23. The binding molecule of any one of claims 1-15, or the cell of claim 19 or claim 20, for use for treating a cancer.
